(19) United States
(12) Patent Application Publication
(10) Pub. No.: US 2023/0192704 A1
(43) Pub. Date: Jun. 22, 2023

(54) MONOCARBOXYLIC ACID TRANSPORTER 4 (MCT4) MODULATORS AND USES THEREOF

(71) Applicant: Teon Therapeutics, Inc., Redwood City, CA (US)

(72) Inventor: Jiwen Liu, Redwood City, CA (US)

(21) Appl. No.: 17/926,347

(22) PCT Filed: May 20, 2021
(86) PCT No.: PCT/US2021/033336

§ 371 Date: Nov. 18, 2022

Related U.S. Application Data
(60) Provisional application No. 63/028,403, filed on May 21, 2020.

(51) Int. Cl. C07D 487/04; C07D 519/00; A61P 35/00

(52) U.S. Cl. CPC C07D 487/04; C07D 519/00; A61P 35/00

(57) ABSTRACT

Disclosed herein are compounds, compositions, and methods for modulating the monocarboxylic acid transporter 4 (MCT4) with the compounds and compositions disclosed herein. Also described are methods of treating diseases or conditions that are mediated by the action of monocarboxylic acid transporter 4 (MCT4) or that we benefit from modulating the monocarboxylic acid transporter 4 (MCT4).

### CROSS REFERENCE TO RELATED APPLICATIONS

This application claims the benefit under 35 U.S.C. § 119(e) of U.S. Provisional Application No. 63/028,403 filed May 21, 2020, which is incorporated herein by reference in its entirety.

### FIELD

Described herein are compounds, methods of making such compounds, pharmaceutical compositions and medicaments comprising such compounds, and methods of using such compounds for the treatment of conditions, diseases, or disorders that would benefit from reduction or inhibition of monocarboxylic acid transporter 4 (MCT4) activity.

### BACKGROUND

Monocarboxylic acid transporters (MCTs) modulate metabolism in the tumour microenvironment, supporting the higher metabolic demands of cancer cells. MCTs are involved in protecting cancer cells from increased levels of metabolic by-products, such as small ketone bodies (e.g., lactic acid and pyruvic acid), as a result of the higher rate of glycolysis, effluxing excessive lactate out of the tumor cells to avoid the decrease in intracellular pH, which can lead to apoptosis. Therefore, MCT antagonism should prevent cancer cells from restoring functional pH levels, leading to apoptosis of cancer cells. Developing MCT antagonists constitute a novel approach to treat cancer by disrupting the tumour microenvironment.

### SUMMARY

In one aspect, described herein is a compound that has the structure of Formula (I-A), or a stereoisomer, or pharmaceutically acceptable salt or solvate thereof:

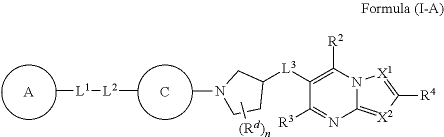

wherein:
X¹ is N or CR¹;
X² is N or CR¹; provided that both X¹ and X² are not N at the same time;
each of R¹, R², R³, and R⁴ is independently H, halogen, —CN, —OH, C₁-C₅alkyl, C₁-C₅alkoxy, C₁-C₅fluoroalkyl, or C₁-C₅fluoroalkoxy;
Ring A is a monocyclic C₃-C₈heterocycloalkyl with at least one N atom, or a bicyclic C₅-C₁₀heterocycloalkyl with at least one N atom, wherein ring A is unsubstituted or substituted with 1, 2, 3, or 4 R^{a};
   each R^{a} is independently selected from the group consisting of hydrogen, —CN, halogen, —OR¹¹, —N(R¹¹)₂, —S(═O)₂(R¹²), —N(R¹¹)—S(═O)₂(R¹²), —C(═O)N(R¹¹)₂, —NR¹¹—C(═O)R¹¹, —C(═O)R¹¹, —C(═O)OR¹¹, —NR¹¹—C(═O)OR¹¹, —OC(═O)N(R¹¹)₂, C₁-C₄alkyl, C₂-C₄alkenyl, C₂-C₄alkynyl, C₁-C₄deuteroalkyl, C₁-C₄deuteroalkoxy, C₁-C₄fluoroalkyl, C₁-C₄fluoroalkoxy, C₁-C₄heteroalkyl, or substituted or unsubstituted monocyclic C₂-C₅heterocycloalkyl;
   or two R^{a} that are attached to the same carbon atom are taken together with the carbon atom to form a substituted or unsubstituted monocyclic C₃-C₆cycloalkyl or a substituted or unsubstituted monocyclic C₃-C₈heterocycloalkyl;
L¹ is —CR⁵R⁶— or —NR⁷—;
   R⁵ and R⁶ are each independently H or C₁-C₅alkyl;
   or R⁵ and R⁶ are taken together with the carbon atom to which they are attached to form —C(═O)—;
   R⁷ is H or C₁-C₅alkyl;
L² is C₁-C₄alkyl, C₂-C₄alkenyl, or C₂-C₄alkynyl;
or L² is a ring B;
   ring B is a monocyclic C₃-C₈cycloalkyl, phenyl, bicyclic C₅-C₁₂cycloalkyl, monocyclic C₃-C₈heterocycloalkyl, bicyclic C₅-C₁₀heterocycloalkyl, or monocyclic heteroaryl, wherein B is unsubstituted or is substituted with 1, 2, 3, or 4 R^{b};
   each R^{b} is independently selected from the group consisting of hydrogen, —CN, halogen, —OR¹¹, —N(R¹¹)₂, —S(═O)₂(R¹²), —N(R¹¹)—S(═O)₂(R¹²), —C(═O)N(R¹¹)₂, —NR¹¹—C(═O)R¹¹, —C(═O)R¹¹, —C(═O)OR¹¹, —NR¹¹—C(═O)OR¹¹, —OC(═O)N(R¹¹)₂, C₁-C₄alkyl, C₂-C₄alkenyl, C₂-C₄alkynyl, C₁-C₄deuteroalkyl, C₁-C₄deuteroalkoxy, C₁-C₄fluoroalkyl, C₁-C₄fluoroalkoxy, C₁-C₄heteroalkyl, or substituted or unsubstituted monocyclic C₂-C₅heterocycloalkyl;
Ring C is selected from the group consisting of phenyl or a 6-membered heteroaryl with 1-3N atoms, wherein Ring C is unsubstituted or is substituted with 1, 2, 3, or 4 R^{c};
   each R^{c} is independently selected from the group consisting of hydrogen, —CN, halogen, —OR¹¹, —N(R¹¹)₂, —S(═O)₂(R¹²), —N(R¹¹)—S(═O)₂(R¹²), —C(═O)N(R¹¹)₂, —NR¹¹—C(═O)R¹¹, —C(═O)R¹¹, —C(═O)OR¹¹, —NR¹¹—C(═O)OR¹¹, —OC(═O)N(R¹¹)₂, C₁-C₄alkyl, C₂-C₄alkenyl, C₂-C₄alkynyl, C₁-C₄deuteroalkyl, C₁-C₄deuteroalkoxy, C₁-C₄fluoroalkyl, C₁-C₄fluoroalkoxy, C₁-C₄heteroalkyl, or substituted or unsubstituted monocyclic C₃-C₅heterocycloalkyl;
each R^{d} is independently selected from the group consisting of hydrogen, —CN, halogen, —OR¹¹, C₁-C₄alkyl, C₂-C₄alkenyl, C₂-C₄alkynyl, C₁-C₄deuteroalkyl, C₁-C₄deuteroalkoxy, C₁-C₄fluoroalkyl, C₁-C₄fluoroalkoxy, or C₁-C₄heteroalkyl;
L³ is —O—, —NR⁸—, or —CR⁹R¹⁰—;
R⁸ is H or C₁-C₅alkyl;
R⁹ and R¹⁰ are each independently H or C₁-C₅alkyl;
each R¹¹ is independently selected from the group consisting of hydrogen, C₁-C₄alkyl, C₁-C₄deuteroalkyl, C₁-C₄fluoroalkyl, C₁-C₄heteroalkyl, substituted or unsubstituted C₃-C₆cycloalkyl, substituted or unsubstituted C₃-C₅heterocycloalkyl, substituted or unsubstituted phenyl, or substituted or unsubstituted monocyclic heteroaryl;
each R¹² is independently selected from the group consisting of C₁-C₄alkyl, C₁-C₄deuteroalkyl, C₁-C₄fluoroalkyl, C₁-C₄heteroalkyl, substituted or unsubstituted C₃-C₆cycloalkyl, substituted or unsubstituted C₃-C₅heterocycloalkyl, substituted or unsubstituted phenyl, or substituted or unsubstituted monocyclic heteroaryl; and
n is 0, 1, or 2.

In another aspect, described herein is a compound that has the structure of Formula (I-B), or a stereoisomer, or pharmaceutically acceptable salt or solvate thereof:

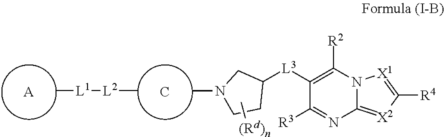

wherein:
X¹ is N or CR¹;
X² is N or CR¹;
each of R¹, R², R³, and R⁴ is independently H, halogen, —CN, —OH, C₁-C₅alkyl, C₁-C₅alkoxy, C₁-C₅fluoroalkyl, or C₁-C₅fluoroalkoxy;
Ring A is a monocyclic C₃-C₈heterocycloalkyl with at least one N atom, or a bicyclic C₅-C₁₀heterocycloalkyl with at least one N atom, wherein ring A is unsubstituted or substituted with 1, 2, 3, or 4 R^{a};
   each R^{a} is independently selected from the group consisting of hydrogen, —CN, halogen, —OR¹¹, —N(R¹¹)₂, —S(═O)₂(R¹²), —N(R¹¹)—S(═O)₂(R¹²), —C(═O)N(R¹¹)₂, —NR¹¹—C(═O)R¹¹, —C(═O)R¹¹, —C(═O)OR¹¹, —NR¹¹—C(═O)OR¹¹, —OC(═O)N(R¹¹)₂, C₁-C₄alkyl, C₂-C₄alkenyl, C₂-C₄alkynyl, C₁-C₄deuteroalkyl, C₁-C₄deuteroalkoxy, C₁-C₄fluoroalkyl, C₁-C₄fluoroalkoxy, C₁-C₄heteroalkyl, or substituted or unsubstituted monocyclic C₂-C₅heterocycloalkyl;
   or two R^{a} that are attached to the same carbon atom are taken together with the carbon atom to form a substituted or unsubstituted monocyclic C₃-C₆cycloalkyl or a substituted or unsubstituted monocyclic C₃-C₅heterocycloalkyl;
L¹ is —CR⁵R⁶— or —NR⁷—;
   R⁵ and R⁶ are each independently H or C₁-C₅alkyl;
   or R⁵ and R⁶ are taken together with the carbon atom to which they are attached to form —C(═O)—;
L² is C₁-C₄alkyl, C₂-C₄alkenyl, C₂-C₄alkynyl;
or L² is a ring B;
   ring B is a monocyclic C₃-C₈cycloalkyl, bicyclic C₅-C₁₂cycloalkyl, monocyclic C₃-C₈heterocycloalkyl, or bicyclic C₅-C₁₀heterocycloalkyl, wherein ring B is unsubstituted or is substituted with 1, 2, 3, or 4 R^{b};
   or ring B is phenyl or monocyclic heteroaryl, wherein ring B is unsubstituted or is substituted with 1, 2, 3, or 4 R^{b}; and L¹ is —CR⁵R⁶— or —NR¹⁷—, and R⁵ and R⁶ are taken together with the carbon atom to which they are attached to form —C(═O)—;
R⁷ is H or C₁-C₅alkyl;
ring B is a monocyclic C₃-C₈cycloalkyl, phenyl, bicyclic C₅-C₁₂cycloalkyl, monocyclic C₃-C₈heterocycloalkyl, bicyclic C₅-C₁₀heterocycloalkyl, or monocyclic heteroaryl, wherein ring B is unsubstituted or is substituted with 1, 2, 3, or 4 R^{b};
   each R^{b} is independently selected from the group consisting of hydrogen, —CN, halogen, —OR¹¹, —N(R¹¹)₂, —S(═O)₂(R¹²), —N(R¹¹)—S(═O)₂(R¹²), —C(═O)N(R¹¹)₂, —NR¹¹—C(═O)R¹¹, —C(═O)R¹¹, —C(═O)OR¹¹, —NR¹¹—C(═O)OR¹¹, —OC(═O)N(R¹¹)₂, C₁-C₄alkyl, C₂-C₄alkenyl, C₂-C₄alkynyl, C₁-C₄deuteroalkyl, C₁-C₄deuteroalkoxy, C₁-C₄fluoroalkyl, C₁-C₄fluoroalkoxy, C₁-C₄heteroalkyl, or substituted or unsubstituted monocyclic C₂-C₅heterocycloalkyl;
Ring C is selected from the group consisting of phenyl or a 6-membered heteroaryl with 1-3N atoms, wherein Ring C is unsubstituted or is substituted with 1, 2, 3, or 4 R^{c};
   each R^{c} is independently selected from the group consisting of hydrogen, —CN, halogen, —OR¹¹, —N(R¹¹)₂, —S(═O)₂(R¹²), —N(R¹¹)—S(═O)₂(R¹²), —C(═O)N(R¹¹)₂, —NR¹¹—C(═O)R¹¹, —C(═O)R¹¹, —C(═O)OR¹¹, —NR¹¹—C(═O)OR¹¹, —OC(═O)N(R¹¹)₂, C₁-C₄alkyl, C₂-C₄alkenyl, C₂-C₄alkynyl, C₁-C₄deuteroalkyl, C₁-C₄deuteroalkoxy, C₁-C₄fluoroalkyl, C₁-C₄fluoroalkoxy, C₁-C₄heteroalkyl, or substituted or unsubstituted monocyclic C₃-C₅heterocycloalkyl;
each R^{d} is independently selected from the group consisting of hydrogen, —CN, halogen, —OR¹¹, C₁-C₄alkyl, C₂-C₄alkenyl, C₂-C₄alkynyl, C₁-C₄deuteroalkyl, C₁-C₄deuteroalkoxy, C₁-C₄fluoroalkyl, C₁-C₄fluoroalkoxy, or C₁-C₄heteroalkyl;
L³ is —O—, —NR⁸—, or —CR⁹R¹⁰—;
R⁸ is H or C₁-C₅alkyl;
R⁹ and R¹⁰ are each independently H or C₁-C₅alkyl;
each R¹¹ is independently selected from the group consisting of hydrogen, C₁-C₄alkyl, C₁-C₄deuteroalkyl, C₁-C₄fluoroalkyl, C₁-C₄heteroalkyl, substituted or unsubstituted C₃-C₆cycloalkyl, substituted or unsubstituted C₃-C₅heterocycloalkyl, substituted or unsubstituted phenyl, or substituted or unsubstituted monocyclic heteroaryl;
each R¹² is independently selected from the group consisting of C₁-C₄alkyl, C₁-C₄deuteroalkyl, C₁-C₄fluoroalkyl, C₁-C₄heteroalkyl, substituted or unsubstituted C₃-C₆cycloalkyl, substituted or unsubstituted C₃-C₅heterocycloalkyl, substituted or unsubstituted phenyl, or substituted or unsubstituted monocyclic heteroaryl; and
n is 0, 1, or 2;
provided that when X¹ and X² are both N, R², R³, and R⁴ are each methyl, L¹ is —CR⁵R⁶—, R⁵ and R⁶ are each independently H or C₁-C₅alkyl, L² is ring B and L³ is —O—, then
   (i) ring A is a 4-membered heterocycloalkyl or a spiro bicyclic C₅-C₈heterocycloalkyl, or (ii) ring B is not phenyl or monocyclic heteroaryl.

In another aspect, described herein is a compound that has the structure of Formula (I-C), or a stereoisomer, or pharmaceutically acceptable salt or solvate thereof:

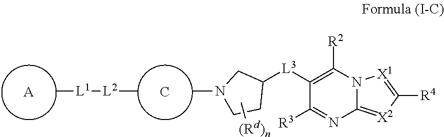

wherein:
X¹ is N or CR¹;
X² is N or CR¹;
each of R¹, R², R³, and R⁴ is independently H, halogen, —CN, —OH, C₁-C₅alkyl, C₁-C₅alkoxy, C₁-C₅fluoroalkyl, or C₁-C₅fluoroalkoxy;
Ring A is a monocyclic C₃-C₈heterocycloalkyl with at least one N atom, or a bicyclic C₅-C₁₀heterocycloalkyl with at least one N atom, wherein ring A is unsubstituted or substituted with 1, 2, 3, or 4 R^{a};
   each R^{a} is independently selected from the group consisting of hydrogen, —CN, halogen, —OR¹¹, —N(R¹¹)₂, —S(═O)₂(R¹²), —N(R¹¹)—S(═O)₂(R¹²), —C(═O)N(R¹¹)₂, —NR¹¹—C(═O)R¹¹, —C(═O)R¹¹, —C(═O)OR¹¹, —NR¹¹—C(═O)OR¹¹, —OC(═O)N(R¹¹)₂, C₁-C₄alkyl, C₂-C₄alkenyl, C₂-C₄alkynyl, C₁-C₄deuteroalkyl, C₁-C₄deuteroalkoxy, C₁-C₄fluoroalkyl, C₁-C₄fluoroalkoxy, C₁-C₄heteroalkyl, or substituted or unsubstituted monocyclic C₂-C₅heterocycloalkyl;
   or two R^{a} that are attached to the same carbon atom are taken together with the carbon atom to form a substituted or unsubstituted monocyclic C₃-C₆cycloalkyl or a substituted or unsubstituted monocyclic C₃-C₅heterocycloalkyl;
L¹ is —CR⁵R⁶— or —NR⁷—;
   R⁵ and R⁶ are taken together with the carbon atom to which they are attached to form —C(═O)—;
   R⁷ is H or C₁-C₅alkyl;
L² is C₁-C₄alkyl, C₂-C₄alkenyl, or C₂-C₄alkynyl;
or L² is a ring B;
   ring B is a monocyclic C₃-C₈cycloalkyl, phenyl, bicyclic C₅-C₁₂cycloalkyl, monocyclic C₃-C₈heterocycloalkyl, bicyclic C₅-C₁₀heterocycloalkyl, or monocyclic heteroaryl, wherein B is unsubstituted or is substituted with 1, 2, 3, or 4 R^{b};
   each R^{b} is independently selected from the group consisting of hydrogen, —CN, halogen, —OR¹¹, —N(R¹¹)₂, —S(═O)₂(R¹²), —N(R¹¹)—S(═O)₂(R¹²), —C(═O)N(R¹¹)₂, —NR¹¹—C(═O)R¹¹, —C(═O)R¹¹, —C(═O)OR¹¹, —NR¹¹—C(═O)OR¹¹, —OC(═O)N(R¹¹)₂, C₁-C₄alkyl, C₂-C₄alkenyl, C₂-C₄alkynyl, C₁-C₄deuteroalkyl, C₁-C₄deuteroalkoxy, C₁-C₄fluoroalkyl, C₁-C₄fluoroalkoxy, C₁-C₄heteroalkyl, or substituted or unsubstituted monocyclic C₂-C₅heterocycloalkyl;
Ring C is selected from the group consisting of phenyl or a 6-membered heteroaryl with 1-3N atoms, wherein Ring C is unsubstituted or is substituted with 1, 2, 3, or 4 R^{c};
   each R^{c} is independently selected from the group consisting of hydrogen, —CN, halogen, —OR¹¹, —N(R¹¹)₂, —S(═O)₂(R¹²), —N(R¹¹)—S(═O)₂(R¹²), —C(═O)N(R¹¹)₂, —NR¹¹—C(═O)R¹¹, —C(═O)R¹¹, —C(═O)OR¹¹, —NR¹¹—C(═O)OR¹¹, —OC(═O)N(R¹¹)₂, C₁-C₄alkyl, C₂-C₄alkenyl, C₂-C₄alkynyl, C₁-C₄deuteroalkyl, C₁-C₄deuteroalkoxy, C₁-C₄fluoroalkyl, C₁-C₄fluoroalkoxy, C₁-C₄heteroalkyl, or substituted or unsubstituted monocyclic C₃-C₅heterocycloalkyl;
each R^{d} is independently selected from the group consisting of hydrogen, —CN, halogen, —OR¹¹, C₁-C₄alkyl, C₂-C₄alkenyl, C₂-C₄alkynyl, C₁-C₄deuteroalkyl, C₁-C₄deuteroalkoxy, C₁-C₄fluoroalkyl, C₁-C₄fluoroalkoxy, or C₁-C₄heteroalkyl;
L³ is —O—, —NR⁸—, or —CR⁹R¹⁰—;
R⁸ is H or C₁-C₅alkyl;
R⁹ and R¹⁰ are each independently H or C₁-C₅alkyl;
each R¹¹ is independently selected from the group consisting of hydrogen, C₁-C₄alkyl, C₁-C₄deuteroalkyl, C₁-C₄fluoroalkyl, C₁-C₄heteroalkyl, substituted or unsubstituted C₃-C₆cycloalkyl, substituted or unsubstituted C₃-C₅heterocycloalkyl, substituted or unsubstituted phenyl, or substituted or unsubstituted monocyclic heteroaryl;
each R¹² is independently selected from the group consisting of C₁-C₄alkyl, C₁-C₄deuteroalkyl, C₁-C₄fluoroalkyl, C₁-C₄heteroalkyl, substituted or unsubstituted C₃-C₆cycloalkyl, substituted or unsubstituted C₃-C₅heterocycloalkyl, substituted or unsubstituted phenyl, or substituted or unsubstituted monocyclic heteroaryl; and
n is 0, 1, or 2.

In another aspect, described herein is a compound that has the structure of Formula (I-D), or a stereoisomer, or pharmaceutically acceptable salt or solvate thereof:

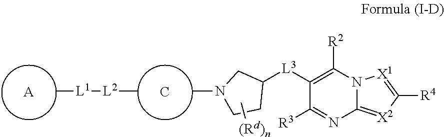

wherein:
each of X¹ and X² is independently N or CR¹;
each of R¹, R², R³, and R⁴ is independently H, halogen, —CN, —OH, C₁-C₅alkyl, C₁-C₅alkoxy, C₁-C₅fluoroalkyl, or C₁-C₅fluoroalkoxy;
Ring A is an azetidinyl or spiro bicyclic C₅-C₈heterocycloalkyl, wherein ring A is unsubstituted or substituted with 1, 2, 3, or 4 R^{a};
   each R^{a} is independently selected from the group consisting of hydrogen, —CN, halogen, —OR¹¹, —N(R¹¹)₂, —S(═O)₂(R¹²), —N(R¹¹)—S(═O)₂(R¹²), —C(═O)N(R¹¹)₂, —NR¹¹—C(═O)R¹¹, —C(═O)R¹¹, —C(═O)OR¹¹, —NR¹¹—C(═O)OR¹¹, —OC(═O)N(R¹¹)₂, C₁-C₄alkyl, C₂-C₄alkenyl, C₂-C₄alkynyl, C₁-C₄deuteroalkyl, C₁-C₄deuteroalkoxy, C₁-C₄fluoroalkyl, C₁-C₄fluoroalkoxy, C₁-C₄heteroalkyl, or substituted or unsubstituted monocyclic C₂-C₅heterocycloalkyl;
   or two R^{a} that are attached to the same carbon atom are taken together with the carbon atom to form a substituted or unsubstituted monocyclic C₃-C₆cycloalkyl or a substituted or unsubstituted monocyclic C₃-C₅heterocycloalkyl;
L¹ is —CR⁵R⁶— or —NR⁷—;
   R⁵ and R⁶ are each independently H or C₁-C₅alkyl;
   or R⁵ and R⁶ are taken together with the carbon atom to which they are attached to form —C(═O)—;
   R⁷ is H or C₁-C₅alkyl;
L² is C₁-C₄alkyl, C₂-C₄alkenyl, or C₂-C₄alkynyl;
or L² is a ring B;
   ring B is a monocyclic C₃-C₈cycloalkyl, phenyl, bicyclic C₅-C₁₂cycloalkyl, monocyclic C₃-C₈heterocycloalkyl, bicyclic C₅-C₁₀heterocycloalkyl, or monocyclic heteroaryl, wherein B is unsubstituted or is substituted with 1, 2, 3, or 4 R^{b};
   each R^{b} is independently selected from the group consisting of hydrogen, —CN, halogen, —OR¹¹, —N(R¹¹)₂, —S(═O)₂(R¹²), —N(R¹¹)—S(═O)₂(R¹²), —C(═O)N(R¹¹)₂, —NR¹¹—C(═O)R¹¹, —C(═O)R¹¹, —C(═O)OR¹¹, —NR¹¹—C(═O)OR¹¹, —OC(═O)N(R¹¹)₂, C₁-C₄alkyl, C₂-C₄alkenyl, C₂-C₄alkynyl, C₁-C₄deuteroalkyl, C₁-C₄deuteroalkoxy, C₁-C₄fluoroalkyl, C₁-C₄fluoroalkoxy, C₁-C₄heteroalkyl, or substituted or unsubstituted monocyclic C₂-C₅heterocycloalkyl;
Ring C is selected from the group consisting of phenyl or a 6-membered heteroaryl with 1-3N atoms, wherein Ring C is unsubstituted or is substituted with 1, 2, 3, or 4 R^{c};
   each R^{c} is independently selected from the group consisting of hydrogen, —CN, halogen, —OR¹¹, —N(R¹¹)₂, —S(═O)₂(R¹²), —N(R¹¹)—S(═O)₂(R¹²), —C(═O)N(R¹¹)₂, —NR¹¹—C(═O)R¹¹, —C(═O)R¹¹, —C(═O)OR¹¹, —NR¹¹—C(═O)OR¹¹, —OC(═O)N(R¹¹)₂, C₁-C₄alkyl, C₂-C₄alkenyl, C₂-C₄alkynyl, C₁-C₄deuteroalkyl, C₁-C₄deuteroalkoxy, C₁-C₄fluoroalkyl, C₁-C₄fluoroalkoxy, C₁-C₄heteroalkyl, or substituted or unsubstituted monocyclic C₃-C₅heterocycloalkyl;
each R^{d} is independently selected from the group consisting of hydrogen, —CN, halogen, —OR¹¹, C₁-C₄alkyl, C₂-C₄alkenyl, C₂-C₄alkynyl, C₁-C₄deuteroalkyl, C₁-C₄deuteroalkoxy, C₁-C₄fluoroalkyl, C₁-C₄fluoroalkoxy, or C₁-C₄heteroalkyl;
L³ is —O—, —NR⁸—, or —CR⁹R¹⁰—;
R⁸ is H or C₁-C₅alkyl;
R⁹ and R¹⁰ are each independently H or C₁-C₅alkyl;
each R¹¹ is independently selected from the group consisting of hydrogen, C₁-C₄alkyl, C₁-C₄deuteroalkyl, C₁-C₄fluoroalkyl, C₁-C₄heteroalkyl, substituted or unsubstituted C₃-C₆cycloalkyl, substituted or unsubstituted C₃-C₅heterocycloalkyl, substituted or unsubstituted phenyl, or substituted or unsubstituted monocyclic heteroaryl;
each R¹² is independently selected from the group consisting of C₁-C₄alkyl, C₁-C₄deuteroalkyl, C₁-C₄fluoroalkyl, C₁-C₄heteroalkyl, substituted or unsubstituted C₃-C₆cycloalkyl, substituted or unsubstituted C₃-C₅heterocycloalkyl, substituted or unsubstituted phenyl, or substituted or unsubstituted monocyclic heteroaryl; and
n is 0, 1, or 2.

In another aspect, described herein is a pharmaceutical composition comprising a compound described herein, or a stereoisomer, or pharmaceutically acceptable salt or solvate thereof, and at least one pharmaceutically acceptable excipient.

In some embodiments, the pharmaceutical composition is formulated for administration to a mammal by oral administration, intravenous administration, or subcutaneous administration.

In some embodiments, the pharmaceutical composition is in the form of a tablet, a pill, a capsule, a liquid, a suspension, a dispersion, a solution, or an emulsion.

In another aspect, described herein is a method of modulating the activity of the monocarboxylic acid transporter 4 (MCT4) in a mammal comprising administering to the mammal a compound described herein, or any pharmaceutically acceptable salt or solvate thereof.

In another aspect, described herein is a method of treating or preventing a disease or disorder in a mammal that is mediated by the action of the monocarboxylic acid transporter 4 (MCT4) comprising administering to the mammal a compound described herein, or any pharmaceutically acceptable salt or solvate thereof.

In another aspect, described herein is a method for treating cancer in a mammal, the method comprising administering to the mammal a monocarboxylic acid transporter 4 (MCT4) modulator. In some embodiments, the monocarboxylic acid transporter 4 (MCT4) modulator is a monocarboxylic acid transporter 4 (MCT4) antagonist. In some embodiments, the monocarboxylic acid transporter 4 (MCT4) modulator is a compound of Formula (I-A), Formula (I-B), Formula (I-C), or Formula (I-D), or any pharmaceutically acceptable salt or solvate thereof.

In another aspect, described herein is a method for treating cancer in a mammal, the method comprising administering to the mammal a compound of Formula (I-A), Formula (I-B), Formula (I-C), or Formula (I-D), or any pharmaceutically acceptable salt or solvate thereof. In some embodiments, the cancer is a solid tumor. In some embodiments, the solid tumor is a glycolytic tumor. In some embodiments, the cancer is bladder cancer, colon cancer, brain cancer, breast cancer, endometrial cancer, heart cancer, kidney cancer, lung cancer, liver cancer, uterine cancer, blood and lymphatic cancer, ovarian cancer, pancreatic cancer, prostate cancer, thyroid cancer, or skin cancer. In some embodiments, the cancer is bladder cancer, breast cancer, colon cancer, or lung cancer. In some embodiments, the cancer is a sarcoma, carcinoma, or lymphoma.

In some embodiments, the method further comprises administering at least one additional therapy to the mammal.

In some embodiments, the mammal is a human.

In any of the aforementioned aspects are further embodiments in which an effective amount of the compound described herein, or a pharmaceutically acceptable salt thereof, is: (a) systemically administered to the mammal; and/or (b) administered orally to the mammal; and/or (c) intravenously administered to the mammal; and/or (d) administered by injection to the mammal.

In any of the aforementioned aspects are further embodiments comprising single administrations of an effective amount of the compound, including further embodiments in which the compound is administered once a day to the mammal or the compound is administered to the mammal multiple times over the span of one day. In some embodiments, the compound is administered on a continuous dosing schedule. In some embodiments, the compound is administered on a continuous daily dosing schedule.

Articles of manufacture, which include packaging material, a formulation within the packaging material (e.g. a formulation suitable for topical administration), and a label that indicates that the compound or composition, or pharmaceutically acceptable salt, or solvate thereof, is used for modulating MCT4 activity, or for the treatment, prevention or amelioration of one or more symptoms of a disease or disorder that is associated with MCT4 activity or that would benefit from MCT4 modulation, are provided.

Other objects, features and advantages of the compounds, methods and compositions described herein will become apparent from the following detailed description. It should be understood, however, that the detailed description and the specific examples, while indicating specific embodiments, are given by way of illustration only, since various changes and modifications within the spirit and scope of the instant disclosure will become apparent to those skilled in the art from this detailed description.

### DETAILED DESCRIPTION

Tumors are metabolic masses that comprise cancer and host cells. The metabolic activity of tumors are affected by their access to nutrients, biological activities, and spatiotemporal localization. While cells in an individual are oxidative and fully oxidize glucose to CO₂, cells (e.g., proliferating cells) exposed to hypoxia (e.g., a local concentration of 02 below physiological cell needs) convert glucose to lactate (e.g., anaerobic glycolysis and aerobic glycolysis). In solid tumors, the glycolytic switches associated with adaptation to hypoxia and cell proliferation operate via different mechanisms. Hypoxic adaptation is a survival mechanism that involves hypoxia-inducible transcription factors (HIFs), whereas metabolic adaptation to cell proliferation involves growth factors and their effectors, such as c-Myc and Ras. While other cancer cells are oxidative, at the whole tumor level, increased conversion of glucose to lactate associated with a high glycolytic rate generates, for example, millimolar concentrations of lactic acid that is released to the extracellular compartment.

Recent data has indicated that lactate plays an important role in regulating immune cell function. Lactate has been shown to inhibit the activity of immune effector cells such as T cells and NK cells. Lactic acid suppresses the proliferation and activation of human T cells ex vivo (Fisher et al. (2007) Blood 109:3812-3819; Haas et al. (2015) PLoS Biol 13). Husain et al. have demonstrated that NK cells from Lactate dehydrogenase A (LDHA) depleted tumours showed improved cytolytic function and lactate treatment of NK cells reduced their cytotoxic activity (Husain et al. (2013) J. Immunol. 191:1486-1495). Furthermore, Brand et al. demonstrated that in immunocompetent mice, knock-down of LDHA reduced lactic acid production and an increased infiltration of IFN-γ-producing T and NK cells was observed in tumours (Brand et al. (2016) Cell Metab. 24:657-671). Lactate has also been shown to inhibit monocyte activation and dendritic cell differentiation (Gottfried et al. (2006) Blood 107:2013-2021; Dietl et al. (2010) J. Immunol. 184:1200-1209) and also induce M2 (immunosuppressive) tumour associated macrophage polarisation (Colegio et al. (2014) Nature 513:559-563). Taken together, these data support the hypothesis that lactate produced as a by-product of the glycolytic phenotype of tumours drives an immunosuppressive effect in the tumour microenvironment. Lactate accumulation in the tumour microenvironment is accompanied by acidosis (due to the co-transport with protons). A low pH in the tumour microenvironment has been associated with extracellular matrix degradation and migration of tumour cells (Gillies and Gatenby (2015) Cancer J. 21: 88-96).

Monocarboxylic acid transporters is one of the metabolic targets wherein the flux of small ketone bodies such as lactic acid and pyruvic acid occurs to support metabolic demands in cancer cells. Monocarboxylic acid transporters (MCTs) are members of the solute carrier family 16 (SCL16 family) and consist of 14 known isoforms. MCTs are present in the cell membrane and are centrally involved in glycolysis to efflux the end product lactate out of the tumor cells to avoid the decrease in intracellular pH which may lead to apoptosis.

In particular, MCT4 is a HIF target gene and is up-regulated by hypoxia and is required to export lactate from glycolytic tumours (Ullah et al. (2006) J. Biol. Chem. 281:9030-9037). The kinetic properties of MCT4 are tuned to its role in exporting lactic acid derived from glycolysis because of its very high Km for pyruvate (150 mM) ensures that pyruvate is not exported from the cell. This is essential because NADH derived from reduction of pyruvate to lactate is required to drive glycolytic flux (Halestrap and Wilson (2012) IUMBM Life 64: 109-119). MCT4 is over-expressed in a range of solid tumours compared to normal epithelium including renal tumours (Fisel et al. (2013) Clin. Cancer Res. 19: 5170-5181; Gerlinger et al. J. Pathol. 227: 146-156), pancreatic tumours (Baek et al. (2014) Cell Rep. 9:2233-2249), colorectal tumours (Pinheiro et al., Virchows Arch. (2008) 452; 139-146), HNSCC (Zhu et al. (2014) PLoS One 9:e87904), breast cancer (Doyen et al. (2014) Biochem. Biophys. Res. Commun. 451:54-61), prostate cancer (Pertega-Gomes et al. BMC Cancer (2011) 11:312) and liver cancer (Gao et al. (2015) J Cancer Res. 141: 1151-1162).

The correlation of MCT4 expression and poor cancer outcome appears to be of significant functional consequence in multiple cancer models. Stable expression of MCT4 is highly tumorigenic in a respiration-impaired, Ras-transformed fibroblast xenograft model. Conversely, MCT4 silencing slows or ablates tumor growth in xenograft models of breast cancer, colorectal cancer, and glioma. MCT4 expression is required for inflammatory cytokine IL-8-mediated angiogenesis in breast and colon cancer xenograft models. MCT4 has also been shown to play important roles in cancer cell migration, invasion, and various aspects of the Warburg effect (e.g., proliferation on glucose, extracellular acidification, and lactate secretion). Thus, there is a need for potent and selective MCT4 inhibitors for use in the treatment or prevention of cancer. Developing MCT4 antagonists constitutes a novel approach to treat cancer by inhibiting lactate efflux.

### Cancer

In some embodiments, disclosed herein are methods of treating cancer with a MCT4 modulator described herein, or a pharmaceutically acceptable salt or solvate thereof.

The term “cancer” as used herein, refers to an abnormal growth of cells that tend to proliferate in an uncontrolled way and, in some cases, to metastasize (spread). Types of cancer include, but are not limited to, solid tumors (such as those of the bladder, bowel, brain, breast, endometrium, heart, kidney, lung, liver, uterus, lymphatic tissue (lymphoma), ovary, pancreas or other endocrine organ (thyroid), prostate, skin (melanoma or basal cell cancer) or hematological tumors (such as the leukemias and lymphomas) at any stage of the disease with or without metastases.

In some embodiments, a mammal treated with a compound described herein has a disease or disorder that is or is associated with a cancer or tumor. Thus, in some embodiments, the mammal is a human that is an oncology patient. Such diseases and disorders and cancers include carcinomas, sarcomas, benign tumors, primary tumors, tumor metastases, solid tumors, non-solid tumors, blood tumors, leukemias and lymphomas, and primary and metastatic tumors.

In some embodiments, the MCT4 receptor modulators described herein are used in the treatment of solid tumours. A solid tumor is an abnormal mass of tissue that usually does not contain cysts or liquid areas. Solid tumors may be benign (not cancer), or malignant (cancer).

Different types of solid tumors are named for the type of cells that form them. Examples of solid tumors are carcinomas, sarcomas, and lymphomas.

Carcinomas include, but are not limited to, esophageal carcinoma, hepatocellular carcinoma, basal cell carcinoma, squamous cell carcinoma, bladder carcinoma, bronchogenic carcinoma, colon carcinoma, colorectal carcinoma, gastric carcinoma, lung carcinoma, including small cell carcinoma and non-small cell carcinoma of the lung, adrenocortical carcinoma, thyroid carcinoma, pancreatic carcinoma, breast carcinoma, ovarian carcinoma, prostate carcinoma, adenocarcinoma, renal cell carcinoma, Wilm's tumor, cervical carcinoma, uterine carcinoma, testicular carcinoma, osteogenic carcinoma, epithelial carcinoma, and nasopharyngeal carcinoma.

Sarcomas include, but are not limited to, fibrosarcoma, myxosarcoma, liposarcoma, chondrosarcoma, chordoma, osteogenic sarcoma, osteosarcoma, angiosarcoma, endotheliosarcoma, lymphangiosarcoma, lymphangioendotheliosarcoma, synovioma, mesothelioma, Ewing's sarcoma, leiomyosarcoma, rhabdomyosarcoma, and other soft tissue sarcomas.

Leukemias include, but are not limited to, a) chronic myeloproliferative syndromes (neoplastic disorders of multipotential hematopoietic stem cells); b) acute myelogenous leukemias; c) chronic lymphocytic leukemias (CLL), including B-cell CLL, T-cell CLL prolymphocyte leukemia, and hairy cell leukemia; and d) acute lymphoblastic leukemias (characterized by accumulation of lymphoblasts). Lymphomas include, but are not limited to, B-cell lymphomas (e.g., Burkitt's lymphoma); Hodgkin's lymphoma; and the like.

Benign tumors include, e.g., hemangiomas, hepatocellular adenoma, cavernous hemangioma, focal nodular hyperplasia, acoustic neuromas, neurofibroma, bile duct adenoma, bile duct cystanoma, fibroma, lipomas, leiomyomas, mesotheliomas, teratomas, myxomas, nodular regenerative hyperplasia, trachomas and pyogenic granulomas.

Primary and metastatic tumors include, e.g., lung cancer; breast cancer; colorectal cancer; anal cancer; pancreatic cancer; prostate cancer; ovarian carcinoma; liver and bile duct carcinoma; esophageal carcinoma; bladder carcinoma; carcinoma of the uterus; glioma, glioblastoma, medulloblastoma, and other tumors of the brain; kidney cancers; cancer of the head and neck; cancer of the stomach; multiple myeloma; testicular cancer; germ cell tumor; neuroendocrine tumor; cervical cancer; carcinoids of the gastrointestinal tract, breast, and other organs.

In one aspect, a MCT4 modulator described herein, or a stereoisomer, or pharmaceutically acceptable salt or solvate thereof, reduces, ameliorates or inhibits immunosuppression and cell proliferation associated with cancers.

### Compounds

Compounds described herein, including pharmaceutically acceptable salts, prodrugs, active metabolites and solvates thereof, are monocarboxylic acid transporter 4 (MCT4) modulators. In some embodiments, the MCT4 modulators are MCT4 antagonists.

In one aspect, described herein is a compound of Formula (I-A), or a stereoisomer, or pharmaceutically acceptable salt or solvate thereof.

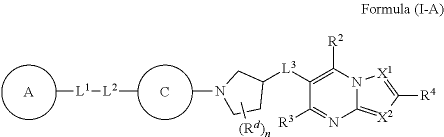

wherein:
X¹ is N or CR¹;
X² is N or CR¹; provided that both X¹ and X² are not N at the same time;
each of R¹, R², R³, and R⁴ is independently H, halogen, —CN, —OH, C₁-C₅alkyl, C₁-C₅alkoxy, C₁-C₅fluoroalkyl, or C₁-C₅fluoroalkoxy;
Ring A is a monocyclic C₃-C₈heterocycloalkyl with at least one N atom, or a bicyclic C₅-C₁₀heterocycloalkyl with at least one N atom, wherein ring A is unsubstituted or substituted with 1, 2, 3, or 4 R^{a};
   each R^{a} is independently selected from the group consisting of hydrogen, —CN, halogen, —OR¹¹, —N(R¹¹)₂, —S(═O)₂(R¹²), —N(R¹¹)—S(═O)₂(R¹²), —C(═O)N(R¹¹)₂, —NR¹¹—C(═O)R¹¹, —C(═O)R¹¹, —C(═O)OR¹¹, —NR¹¹—C(═O)OR¹¹, —OC(═O)N(R¹¹)₂, C₁-C₄alkyl, C₂-C₄alkenyl, C₂-C₄alkynyl, C₁-C₄deuteroalkyl, C₁-C₄deuteroalkoxy, C₁-C₄fluoroalkyl, C₁-C₄fluoroalkoxy, C₁-C₄heteroalkyl, or substituted or unsubstituted monocyclic C₂-C₅heterocycloalkyl;
   or two R^{a} that are attached to the same carbon atom are taken together with the carbon atom to form a substituted or unsubstituted monocyclic C₃-C₆cycloalkyl or a substituted or unsubstituted monocyclic C₃-C₅heterocycloalkyl;
L¹ is —CR⁵R⁶— or —NR⁷—;
   R⁵ and R⁶ are each independently H or C₁-C₅alkyl;
   or R⁵ and R⁶ are taken together with the carbon atom to which they are attached to form —C(═O)—;
   R⁷ is H or C₁-C₅alkyl;
L² is C₁-C₄alkyl, C₂-C₄alkenyl, or C₂-C₄alkynyl;
or L² is a ring B;
   ring B is a monocyclic C₃-C₈cycloalkyl, phenyl, bicyclic C₅-C₁₂cycloalkyl, monocyclic C₃-C₈heterocycloalkyl, bicyclic C₅-C₁₀heterocycloalkyl, or monocyclic heteroaryl, wherein B is unsubstituted or is substituted with 1, 2, 3, or 4 R^{b};
   each R^{b} is independently selected from the group consisting of hydrogen, —CN, halogen, —OR¹¹, —N(R¹¹)₂, —S(═O)₂(R¹²), —N(R¹¹)—S(═O)₂(R¹²), —C(═O)N(R¹¹)₂, —NR¹¹—C(═O)R¹¹, —C(═O)R¹¹, —C(═O)OR¹¹, —NR¹¹—C(═O)OR¹¹, —OC(═O)N(R¹¹)₂, C₁-C₄alkyl, C₂-C₄alkenyl, C₂-C₄alkynyl, C₁-C₄deuteroalkyl, C₁-C₄deuteroalkoxy, C₁-C₄fluoroalkyl, C₁-C₄fluoroalkoxy, C₁-C₄heteroalkyl, or substituted or unsubstituted monocyclic C₂-C₅heterocycloalkyl;
Ring C is selected from the group consisting of phenyl or a 6-membered heteroaryl with 1-3N atoms, wherein Ring C is unsubstituted or is substituted with 1, 2, 3, or 4 R^{c};
   each R^{c} is independently selected from the group consisting of hydrogen, —CN, halogen, —OR¹¹, —N(R¹¹)₂, —S(═O)₂(R¹²), —N(R¹¹)—S(═O)₂(R¹²), —C(═O)N(R¹¹)₂, —NR¹¹—C(═O)R¹¹, —C(═O)R¹¹, —C(═O)OR¹¹, —NR¹¹—C(═O)OR¹¹, —OC(═O)N(R¹¹)₂, C₁-C₄alkyl, C₂-C₄alkenyl, C₂-C₄alkynyl, C₁-C₄deuteroalkyl, C₁-C₄deuteroalkoxy, C₁-C₄fluoroalkyl, C₁-C₄fluoroalkoxy, C₁-C₄heteroalkyl, or substituted or unsubstituted monocyclic C₃-C₅heterocycloalkyl;
each R^{d} is independently selected from the group consisting of hydrogen, —CN, halogen, —OR¹¹, C₁-C₄alkyl, C₂-C₄alkenyl, C₂-C₄alkynyl, C₁-C₄deuteroalkyl, C₁-C₄deuteroalkoxy, C₁-C₄fluoroalkyl, C₁-C₄fluoroalkoxy, or C₁-C₄heteroalkyl;
L³ is —O—, —NR⁸—, or —CR⁹R¹⁰—;
R⁸ is H or C₁-C₅alkyl;
R⁹ and R¹⁰ are each independently H or C₁-C₅alkyl;
each R¹¹ is independently selected from the group consisting of hydrogen, C₁-C₄alkyl, C₁-C₄deuteroalkyl, C₁-C₄fluoroalkyl, C₁-C₄heteroalkyl, substituted or unsubstituted C₃-C₆cycloalkyl, substituted or unsubstituted C₃-C₅heterocycloalkyl, substituted or unsubstituted phenyl, or substituted or unsubstituted monocyclic heteroaryl;
each R¹² is independently selected from the group consisting of C₁-C₄alkyl, C₁-C₄deuteroalkyl, C₁-C₄fluoroalkyl, C₁-C₄heteroalkyl, substituted or unsubstituted C₃-C₆cycloalkyl, substituted or unsubstituted C₃-C₅heterocycloalkyl, substituted or unsubstituted phenyl, or substituted or unsubstituted monocyclic heteroaryl; and
n is 0, 1, or 2.

For any and all of the embodiments, substituents are selected from among a subset of the listed alternatives. For example, in some embodiments, X¹ is N. In some embodiments, X¹ is CR¹. In some embodiments, X² is N. In some embodiments, X² is CR¹. In some embodiments, X¹ is N and X² is CR¹. In some embodiments, X¹ is CR¹ and X² is N. In some embodiments, X¹ is CR¹ and X² is CR¹.

In some embodiments, R¹ is H, C₁-C₅alkyl, C₁-C₅alkoxy, C₁-C₅fluoroalkyl, or C₁-C₅fluoroalkoxy. In some embodiments, R¹ is H or C₁-C₅alkyl. In some embodiments, R¹ is H. In some embodiments, R¹ is C₁-C₅alkyl. In some embodiments, R¹ is methyl.

In another aspect, described herein is a compound of Formula (I-B), or a stereoisomer, or pharmaceutically acceptable salt or solvate thereof:

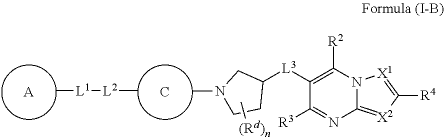

wherein:
X¹ is N or CR¹;
X² is N or CR¹;
each of R¹, R², R³, and R⁴ is independently H, halogen, —CN, —OH, C₁-C₅alkyl, C₁-C₅alkoxy, C₁-C₅fluoroalkyl, or C₁-C₅fluoroalkoxy;
Ring A is a monocyclic C₃-C₈heterocycloalkyl with at least one N atom, or a bicyclic C₅-C₁₀heterocycloalkyl with at least one N atom, wherein ring A is unsubstituted or substituted with 1, 2, 3, or 4 R^{a};
   each R^{a} is independently selected from the group consisting of hydrogen, —CN, halogen, —OR¹¹, —N(R¹¹)₂, —S(═O)₂(R¹²), —N(R¹¹)—S(═O)₂(R¹²), —C(═O)N(R¹¹)₂, —NR¹¹—C(═O)R¹¹, —C(═O)R¹¹, —C(═O)OR¹¹, —NR¹¹—C(═O)OR¹¹, —OC(═O)N(R¹¹)₂, C₁-C₄alkyl, C₂-C₄alkenyl, C₂-C₄alkynyl, C₁-C₄deuteroalkyl, C₁-C₄deuteroalkoxy, C₁-C₄fluoroalkyl, C₁-C₄fluoroalkoxy, C₁-C₄heteroalkyl, or substituted or unsubstituted monocyclic C₂-C₅heterocycloalkyl;
   or two R^{a} that are attached to the same carbon atom are taken together with the carbon atom to form a substituted or unsubstituted monocyclic C₃-C₆cycloalkyl or a substituted or unsubstituted monocyclic C₃-C₅heterocycloalkyl;
L¹ is —CR⁵R⁶— or —NR⁷—;
   R⁵ and R⁶ are each independently H or C₁-C₅alkyl;
   or R⁵ and R⁶ are taken together with the carbon atom to which they are attached to form —C(═O)—;
L² is C₁-C₄alkyl, C₂-C₄alkenyl, C₂-C₄alkynyl;
or L² is a ring B;
   ring B is a monocyclic C₃-C₈cycloalkyl, bicyclic C₅-C₁₂cycloalkyl, monocyclic C₃-C₈heterocycloalkyl, or bicyclic C₅-C₁₀heterocycloalkyl, wherein ring B is unsubstituted or is substituted with 1, 2, 3, or 4 R^{b};
   or ring B is phenyl or monocyclic heteroaryl, wherein ring B is unsubstituted or is substituted with 1, 2, 3, or 4 R^{b}; and L¹ is —CR⁵R⁶— or —NR⁷—, and R⁵ and R⁶ are taken together with the carbon atom to which they are attached to form —C(═O)—;
R⁷ is H or C₁-C₅alkyl;
ring B is a monocyclic C₃-C₈cycloalkyl, phenyl, bicyclic C₅-C₁₂cycloalkyl, monocyclic C₃-C₈heterocycloalkyl, bicyclic C₅-C₁₀heterocycloalkyl, or monocyclic heteroaryl, wherein ring B is unsubstituted or is substituted with 1, 2, 3, or 4 R^{b};
each R^{b} is independently selected from the group consisting of hydrogen, —CN, halogen, —OR¹¹, —N(R¹¹)₂, —S(═O)₂(R¹²), —N(R¹¹)—S(═O)₂(R¹²), —C(═O)N(R¹¹)₂, —NR¹¹—C(═O)R¹¹, —C(═O)R¹¹, —C(═O)OR¹¹, —NR¹¹—C(═O)OR¹¹, —OC(═O)N(R¹¹)₂, C₁-C₄alkyl, C₂-C₄alkenyl, C₂-C₄alkynyl, C₁-C₄deuteroalkyl, C₁-C₄deuteroalkoxy, C₁-C₄fluoroalkyl, C₁-C₄fluoroalkoxy, C₁-C₄heteroalkyl, or substituted or unsubstituted monocyclic C₂-C₅heterocycloalkyl;
Ring C is selected from the group consisting of phenyl or a 6-membered heteroaryl with 1-3N atoms, wherein Ring C is unsubstituted or is substituted with 1, 2, 3, or 4 R^{c};
   each R^{c} is independently selected from the group consisting of hydrogen, —CN, halogen, —OR¹¹, —N(R¹¹)₂, —S(═O)₂(R¹²), —N(R¹¹)—S(═O)₂(R¹²), —C(═O)N(R¹¹)₂, —NR¹¹—C(═O)R¹¹, —C(═O)R¹¹, —C(═O)OR¹¹, —NR¹¹—C(═O)OR¹¹, —OC(═O)N(R¹¹)₂, C₁-C₄alkyl, C₂-C₄alkenyl, C₂-C₄alkynyl, C₁-C₄deuteroalkyl, C₁-C₄deuteroalkoxy, C₁-C₄fluoroalkyl, C₁-C₄fluoroalkoxy, C₁-C₄heteroalkyl, or substituted or unsubstituted monocyclic C₃-C₅heterocycloalkyl;
each R^{d} is independently selected from the group consisting of hydrogen, —CN, halogen, —OR¹¹, C₁-C₄alkyl, C₂-C₄alkenyl, C₂-C₄alkynyl, C₁-C₄deuteroalkyl, C₁-C₄deuteroalkoxy, C₁-C₄fluoroalkyl, C₁-C₄fluoroalkoxy, or C₁-C₄heteroalkyl;
L³ is —O—, —NR⁸—, or —CR⁹R¹⁰—;
R⁸ is H or C₁-C₅alkyl;
R⁹ and R¹⁰ are each independently H or C₁-C₅alkyl;
each R¹¹ is independently selected from the group consisting of hydrogen, C₁-C₄alkyl, C₁-C₄deuteroalkyl, C₁-C₄fluoroalkyl, C₁-C₄heteroalkyl, substituted or unsubstituted C₃-C₆cycloalkyl, substituted or unsubstituted C₃-C₅heterocycloalkyl, substituted or unsubstituted phenyl, or substituted or unsubstituted monocyclic heteroaryl;
each R¹² is independently selected from the group consisting of C₁-C₄alkyl, C₁-C₄deuteroalkyl, C₁-C₄fluoroalkyl, C₁-C₄heteroalkyl, substituted or unsubstituted C₃-C₆cycloalkyl, substituted or unsubstituted C₃-C₅heterocycloalkyl, substituted or unsubstituted phenyl, or substituted or unsubstituted monocyclic heteroaryl; and
n is 0, 1, or 2.

In some embodiments of Formula (I-B), when X¹ and X² are both N, R², R³, and R⁴ are each methyl, L¹ is —CR⁵R⁶—, R⁵ and R⁶ are each independently H or C₁-C₅alkyl, L² is ring B and L³ is —O—, then (i) ring A is a 4-membered heterocycloalkyl or a spiro bicyclic C₅-C₈heterocycloalkyl, or (ii) ring B is not phenyl or monocyclic heteroaryl.

In some embodiments of Formula (I-B), at least one of ring B or ring C is phenyl, pyridinyl, pyrazinyl, pyrimidinyl, or pyridazinyl.

In some embodiments, ring B is a monocyclic C₃-C₈cycloalkyl, bicyclic C₅-C₁₂cycloalkyl, monocyclic C₃-C₈heterocycloalkyl, or bicyclic C₅-C₁₀heterocycloalkyl, wherein ring B is unsubstituted or is substituted with 1, 2, 3, or 4 R^{b}. In some embodiments, ring B is a monocyclic C₃-C₈cycloalkyl, bicyclic C₅-C₁₂cycloalkyl, monocyclic C₃-C₈heterocycloalkyl, or bicyclic C₅-C₁₀heterocycloalkyl, wherein ring B is unsubstituted or is substituted with 1, 2, 3, or 4 R^{b}, and L¹ is —CR⁵R⁶—. In some embodiments, R⁵ and R⁶ are each independently H or C₁-C₅alkyl. In some embodiments, R⁵ and R⁶ are taken together with the carbon atom to which they are attached to form —C(═O)—. In some embodiments, ring B is a monocyclic C₃-C₈cycloalkyl, bicyclic C₅-C₁₂cycloalkyl, monocyclic C₃-C₈heterocycloalkyl, or bicyclic C₅-C₁₀heterocycloalkyl, wherein ring B is unsubstituted or is substituted with 1, 2, 3, or 4 R^{b}, and L¹ is —NR⁷—.

In some embodiments, ring B is phenyl or monocyclic heteroaryl, wherein ring B is unsubstituted or is substituted with 1, 2, 3, or 4 R^{b}; and L¹ is —CR⁵R⁶— or —NR⁷—, and R⁵ and R⁶ are taken together with the carbon atom to which they are attached to form —C(═O)—. In some embodiments, ring B is phenyl or monocyclic heteroaryl, wherein ring B is unsubstituted or is substituted with 1, 2, 3, or 4 R^{b}; and L¹ is —C(═O)—. In some embodiments, ring B is phenyl or monocyclic heteroaryl, wherein ring B is unsubstituted or is substituted with 1, 2, 3, or 4 R^{b}; and L¹ is —NR⁷—.

In another aspect, described herein is a compound of Formula (I-C), or a stereoisomer, or pharmaceutically acceptable salt or solvate thereof:

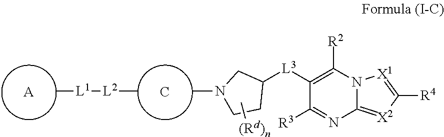

wherein:
X¹ is N or CR¹;
X² is N or CR¹;
each of R¹, R², R³, and R⁴ is independently H, halogen, —CN, —OH, C₁-C₅alkyl, C₁-C₅alkoxy, C₁-C₅fluoroalkyl, or C₁-C₅fluoroalkoxy;
Ring A is a monocyclic C₃-C₈heterocycloalkyl with at least one N atom, or a bicyclic C₅-C₁₀heterocycloalkyl with at least one N atom, wherein ring A is unsubstituted or substituted with 1, 2, 3, or 4 R^{a};
   each R^{a} is independently selected from the group consisting of hydrogen, —CN, halogen, —OR¹¹, —N(R¹¹)₂, —S(═O)₂(R¹²), —N(R¹¹)—S(═O)₂(R¹²), —C(═O)N(R¹¹)₂, —NR¹¹—C(═O)R¹¹, —C(═O)R¹¹, —C(═O)OR¹¹, —NR¹¹—C(═O)OR¹¹, —OC(═O)N(R¹¹)₂, C₁-C₄alkyl, C₂-C₄alkenyl, C₂-C₄alkynyl, C₁-C₄deuteroalkyl, C₁-C₄deuteroalkoxy, C₁-C₄fluoroalkyl, C₁-C₄fluoroalkoxy, C₁-C₄heteroalkyl, or substituted or unsubstituted monocyclic C₂-C₅heterocycloalkyl;
   or two R^{a} that are attached to the same carbon atom are taken together with the carbon atom to form a substituted or unsubstituted monocyclic C₃-C₆cycloalkyl or a substituted or unsubstituted monocyclic C₃-C₅heterocycloalkyl;
L¹ is —CR⁵R⁶— or —NR⁷—;
   R⁵ and R⁶ are taken together with the carbon atom to which they are attached to form —C(═O)—;
   R⁷ is H or C₁-C₅alkyl;
L² is C₁-C₄alkyl, C₂-C₄alkenyl, or C₂-C₄alkynyl;
or L² is a ring B;
   ring B is a monocyclic C₃-C₈cycloalkyl, phenyl, bicyclic C₅-C₁₂cycloalkyl, monocyclic C₃-C₈heterocycloalkyl, bicyclic C₅-C₁₀heterocycloalkyl, or monocyclic heteroaryl, wherein B is unsubstituted or is substituted with 1, 2, 3, or 4 R^{b};
   each R^{b} is independently selected from the group consisting of hydrogen, —CN, halogen, —OR¹¹, —N(R¹¹)₂, —S(═O)₂(R¹²), —N(R¹¹)—S(═O)₂(R¹²), —C(═O)N(R¹¹)₂, —NR¹¹—C(═O)R¹¹, —C(═O)R¹¹, —C(═O)OR¹¹, —NR¹¹—C(═O)OR¹¹, —OC(═O)N(R¹¹)₂, C₁-C₄alkyl, C₂-C₄alkenyl, C₂-C₄alkynyl, C₁-C₄deuteroalkyl, C₁-C₄deuteroalkoxy, C₁-C₄fluoroalkyl, C₁-C₄fluoroalkoxy, C₁-C₄heteroalkyl, or substituted or unsubstituted monocyclic C₂-C₅heterocycloalkyl;
Ring C is selected from the group consisting of phenyl or a 6-membered heteroaryl with 1-3N atoms, wherein Ring C is unsubstituted or is substituted with 1, 2, 3, or 4 R^{c};
   each R^{c} is independently selected from the group consisting of hydrogen, —CN, halogen, —OR¹¹, —N(R¹¹)₂, —S(═O)₂(R¹²), —N(R¹¹)—S(═O)₂(R¹²), —C(═O)N(R¹¹)₂, —NR¹¹—C(═O)R¹¹, —C(═O)R¹¹, —C(═O)OR¹¹, —NR¹¹—C(═O)OR¹¹, —OC(═O)N(R¹¹)₂, C₁-C₄alkyl, C₂-C₄alkenyl, C₂-C₄alkynyl, C₁-C₄deuteroalkyl, C₁-C₄deuteroalkoxy, C₁-C₄fluoroalkyl, C₁-C₄fluoroalkoxy, C₁-C₄heteroalkyl, or substituted or unsubstituted monocyclic C₃-C₅heterocycloalkyl;
each R^{d} is independently selected from the group consisting of hydrogen, —CN, halogen, —OR¹¹, C₁-C₄alkyl, C₂-C₄alkenyl, C₂-C₄alkynyl, C₁-C₄deuteroalkyl, C₁-C₄deuteroalkoxy, C₁-C₄fluoroalkyl, C₁-C₄fluoroalkoxy, or C₁-C₄heteroalkyl;
L³ is —O—, —NR⁸—, or —CR⁹R¹⁰—;
R⁸ is H or C₁-C₅alkyl;
R⁹ and R¹⁰ are each independently H or C₁-C₅alkyl;
each R¹¹ is independently selected from the group consisting of hydrogen, C₁-C₄alkyl, C₁-C₄deuteroalkyl, C₁-C₄fluoroalkyl, C₁-C₄heteroalkyl, substituted or unsubstituted C₃-C₆cycloalkyl, substituted or unsubstituted C₃-C₅heterocycloalkyl, substituted or unsubstituted phenyl, or substituted or unsubstituted monocyclic heteroaryl;
each R¹² is independently selected from the group consisting of C₁-C₄alkyl, C₁-C₄deuteroalkyl, C₁-C₄fluoroalkyl, C₁-C₄heteroalkyl, substituted or unsubstituted C₃-C₆cycloalkyl, substituted or unsubstituted C₃-C₅heterocycloalkyl, substituted or unsubstituted phenyl, or substituted or unsubstituted monocyclic heteroaryl; and
n is 0, 1, or 2.

In some embodiments, L¹ is —C(═O)—. In some embodiments, L¹ is —NR⁷—.

In some embodiments, ring A is azetidinyl, pyrrolidinyl, or a monocyclic 6-membered C₄-C₅heterocycloalkyl, or a bicyclic C₅-C₁₀heterocycloalkyl that is a fused bicyclic C₅-C₈heterocycloalkyl, bridged bicyclic C₅-C₈heterocycloalkyl, or spiro bicyclic C₅-C₈heterocycloalkyl, wherein ring A is unsubstituted or substituted with 1, 2, 3, or 4 R^{a}. In some embodiments, ring A is azetidinyl, pyrrolidinyl, morpholinyl, thiomorpholinyl, piperidinyl, piperazinyl, or a bicyclic C₅-C₁₀heterocycloalkyl that is a fused bicyclic C₅-C₈heterocycloalkyl, bridged bicyclic C₅-C₈heterocycloalkyl, or spiro bicyclic C₅-C₈heterocycloalkyl, wherein ring A is unsubstituted or substituted with 1, 2, 3, or 4 R^{a}. In some embodiments, ring A is azetidinyl, morpholinyl, piperidinyl, piperazinyl, or a spiro bicyclic C₅-C₈heterocycloalkyl, wherein ring A is unsubstituted or substituted with 1, 2, 3, or 4 R^{a}.

In some embodiments, ring A is morpholinyl, piperidinyl, or piperazinyl, wherein ring A is unsubstituted or substituted with 1, 2, 3, or 4 R^{a}. In some embodiments, ring A is

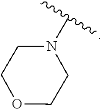 [SMILES: *N1CCOCC1]

In some embodiments, ring A is

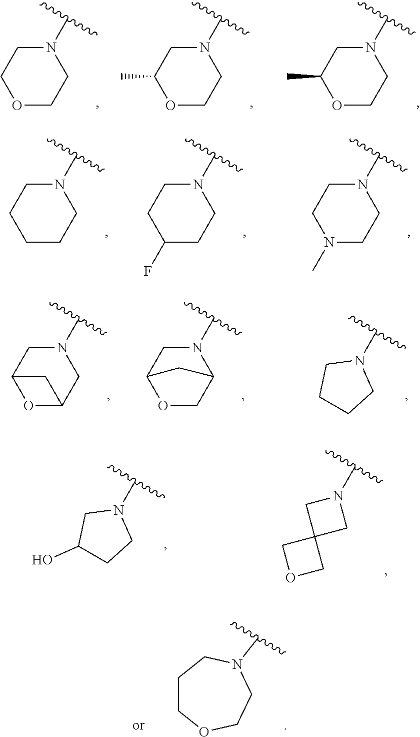

In some embodiments, ring A is unsubstituted. In some embodiments, ring A is substituted with 1, 2, 3, or 4 R^{a}. In some embodiments, ring A is substituted with 1 or 2 R^{a}. In some embodiments, ring A is substituted with 1 R^{a}. In some embodiments, ring A is substituted with 2 R^{a}. In some embodiments, each R^{a} is independently selected from the group consisting of hydrogen, halogen, —OR¹¹, —N(R¹¹)₂, —S(═O)₂(R¹²), —C(═O)N(R¹¹)₂, —NR¹¹—C(═O)R¹¹, C₁-C₄alkyl, C₁-C₄fluoroalkyl, C₁-C₄fluoroalkoxy, C₁-C₄heteroalkyl, or substituted or unsubstituted monocyclic C₂-C₅heterocycloalkyl. In some embodiments, two R^{a} that are attached to the same carbon atom are taken together with the carbon atom to form a substituted or unsubstituted monocyclic C₃-C₆cycloalkyl or a substituted or unsubstituted monocyclic C₃-C₅heterocycloalkyl.

In some embodiments, ring A is

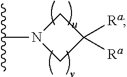

u is 1 or 2; and v is 1 or 2.

In some embodiments, when two R^{a} are present, then one R^{a} is H irrespective of the identity of the other R^{a}.

In another aspect, described herein is a compound of Formula (I-D), or a stereoisomer, or pharmaceutically acceptable salt or solvate thereof:

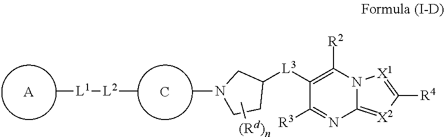

wherein:
each of X¹ and X² is independently N or CR¹;
each of R¹, R², R³, and R⁴ is independently H, halogen, —CN, —OH, C₁-C₅alkyl, C₁-C₅alkoxy, C₁-C₅fluoroalkyl, or C₁-C₅fluoroalkoxy;
Ring A is an azetidinyl or spiro bicyclic C₅-C₈heterocycloalkyl, wherein ring A is unsubstituted or substituted with 1, 2, 3, or 4 R^{a};
   each R^{a} is independently selected from the group consisting of hydrogen, —CN, halogen, —OR¹¹, —N(R¹¹)₂, —S(═O)₂(R¹²), —N(R¹¹)—S(═O)₂(R¹²), —C(═O)N(R¹¹)₂, —NR¹¹—C(═O)R¹¹, —C(═O)R¹¹, —C(═O)OR¹¹, —NR¹¹—C(═O)OR¹¹, —OC(═O)N(R¹¹)₂, C₁-C₄alkyl, C₂-C₄alkenyl, C₂-C₄alkynyl, C₁-C₄deuteroalkyl, C₁-C₄deuteroalkoxy, C₁-C₄fluoroalkyl, C₁-C₄fluoroalkoxy, C₁-C₄heteroalkyl, or substituted or unsubstituted monocyclic C₂-C₅heterocycloalkyl;
   or two R^{a} that are attached to the same carbon atom are taken together with the carbon atom to form a substituted or unsubstituted monocyclic C₃-C₆cycloalkyl or a substituted or unsubstituted monocyclic C₃-C₅heterocycloalkyl;
L¹ is —CR⁵R⁶— or —NR⁷—;
   R⁵ and R⁶ are each independently H or C₁-C₅alkyl;
   or R⁵ and R⁶ are taken together with the carbon atom to which they are attached to form —C(═O)—;
   R⁷ is H or C₁-C₅alkyl;
L² is C₁-C₄alkyl, C₂-C₄alkenyl, or C₂-C₄alkynyl;
or L² is a ring B;
   ring B is a monocyclic C₃-C₈cycloalkyl, phenyl, bicyclic C₅-C₁₂cycloalkyl, monocyclic C₃-C₈heterocycloalkyl, bicyclic C₅-C₁₀heterocycloalkyl, or monocyclic heteroaryl, wherein B is unsubstituted or is substituted with 1, 2, 3, or 4 R^{b};
   each R^{b} is independently selected from the group consisting of hydrogen, —CN, halogen, —OR¹¹, —N(R¹¹)₂, —S(═O)₂(R¹²), —N(R¹¹)—S(═O)₂(R¹²), —C(═O)N(R¹¹)₂, —NR¹¹—C(═O)R¹¹, —C(═O)R¹¹, —C(═O)OR¹¹, —NR¹¹—C(═O)OR¹¹, —OC(═O)N(R¹¹)₂, C₁-C₄alkyl, C₂-C₄alkenyl, C₂-C₄alkynyl, C₁-C₄deuteroalkyl, C₁-C₄deuteroalkoxy, C₁-C₄fluoroalkyl, C₁-C₄fluoroalkoxy, C₁-C₄heteroalkyl, or substituted or unsubstituted monocyclic C₂-C₅heterocycloalkyl;
Ring C is selected from the group consisting of phenyl or a 6-membered heteroaryl with 1-3N atoms, wherein Ring C is unsubstituted or is substituted with 1, 2, 3, or 4 R^{c};
   each R^{c} is independently selected from the group consisting of hydrogen, —CN, halogen, —OR¹¹, —N(R¹¹)₂, —S(═O)₂(R¹²), —N(R¹¹)—S(═O)₂(R¹²), —C(═O)N(R¹¹)₂, —NR¹¹—C(═O)R¹¹, —C(═O)R¹¹, —C(═O)OR¹¹, —NR¹¹—C(═O)OR¹¹, —OC(═O)N(R¹¹)₂, C₁-C₄alkyl, C₂-C₄alkenyl, C₂-C₄alkynyl, C₁-C₄deuteroalkyl, C₁-C₄deuteroalkoxy, C₁-C₄fluoroalkyl, C₁-C₄fluoroalkoxy, C₁-C₄heteroalkyl, or substituted or unsubstituted monocyclic C₃-C₅heterocycloalkyl;
each R^{d} is independently selected from the group consisting of hydrogen, —CN, halogen, —OR¹¹, C₁-C₄alkyl, C₂-C₄alkenyl, C₂-C₄alkynyl, C₁-C₄deuteroalkyl, C₁-C₄deuteroalkoxy, C₁-C₄fluoroalkyl, C₁-C₄fluoroalkoxy, or C₁-C₄heteroalkyl;
L³ is —O—, —NR⁸—, or —CR⁹R¹⁰—;
R⁸ is H or C₁-C₅alkyl;
R⁹ and R¹⁰ are each independently H or C₁-C₅alkyl;
each R¹¹ is independently selected from the group consisting of hydrogen, C₁-C₄alkyl, C₁-C₄deuteroalkyl, C₁-C₄fluoroalkyl, C₁-C₄heteroalkyl, substituted or unsubstituted C₃-C₆cycloalkyl, substituted or unsubstituted C₃-C₅heterocycloalkyl, substituted or unsubstituted phenyl, or substituted or unsubstituted monocyclic heteroaryl;
each R¹² is independently selected from the group consisting of C₁-C₄alkyl, C₁-C₄deuteroalkyl, C₁-C₄fluoroalkyl, C₁-C₄heteroalkyl, substituted or unsubstituted C₃-C₆cycloalkyl, substituted or unsubstituted C₃-C₅heterocycloalkyl, substituted or unsubstituted phenyl, or substituted or unsubstituted monocyclic heteroaryl; and
n is 0, 1, or 2.

In some embodiments, X¹ is N. In some embodiments, X¹ is CR¹. In some embodiments, X² is N. In some embodiments, X² is CR¹. In some embodiments, X¹ is N and X² is N. In some embodiments, X¹ is N and X² is CR¹. In some embodiments, X¹ is CR¹ and X² is N. In some embodiments, X¹ is CR¹ and X² is CR¹.

In some embodiments, each of R¹, R², R³, and R⁴ is independently H, halogen, C₁-C₅alkyl, C₁-C₅alkoxy, C₁-C₅fluoroalkyl, or C₁-C₅fluoroalkoxy. In some embodiments, each of R¹, R², R³, and R⁴ is independently H, halogen, C₁-C₅alkyl, or C₁-C₅fluoroalkyl. In some embodiments, each of R¹, R², R³, and R⁴ is independently H or C₁-C₅alkyl. In some embodiments, each of R¹, R², R³, and R⁴ is independently C₁-C₅alkyl. In some embodiments, each of R¹, R², R³, and R⁴ is independently C₁-C₃alkyl. In some embodiments, each of R¹, R², R³, and R⁴ is methyl.

In some embodiments, R¹ is H, C₁-C₅alkyl, C₁-C₅alkoxy, C₁-C₅fluoroalkyl, or C₁-C₅fluoroalkoxy. In some embodiments, R¹ is H or C₁-C₅alkyl. In some embodiments, R¹ is H. In some embodiments, R¹ is C₁-C₅alkyl. In some embodiments, R¹ is methyl.

In some embodiments, R¹ is H and each of R², R³, and R⁴ is methyl. In some embodiments, R¹ is methyl and each of R², R³, and R⁴ is methyl.

In some embodiments, L³ is —O—. In some embodiments, L³ is —NR⁸—. In some embodiments, L³ is —CR⁹R¹⁰—.

In some embodiments, R⁸ is H or C₁-C₅alkyl. In some embodiments, R⁹ and R¹⁰ are each independently H or C₁-C₅alkyl. In some embodiments, R⁹ and R¹⁰ are each independently H. In some embodiments, R⁹ and R¹⁰ are each independently C₁-C₅alkyl.

In some embodiments, n is 0 or 1. In some embodiments, n is 0.

In some embodiments, the compound of Formula (I-A) has the following structure of Formula (I-E), or a stereoisomer, or pharmaceutically acceptable salt or solvate thereof:

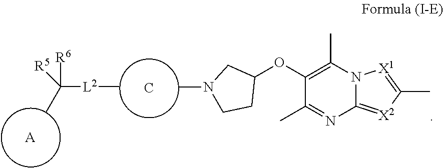

In some embodiments, R⁵ and R⁶ are each independently H or —CH₃. In some embodiments, R⁵ and R⁶ are each H. In some embodiments, R⁵ and R⁶ are taken together with the carbon atom to which they are attached to form —C(═O)—.

In some embodiments, the compound of Formula (I-A) has the following structure of Formula (I-F), or a stereoisomer, or pharmaceutically acceptable salt or solvate thereof:

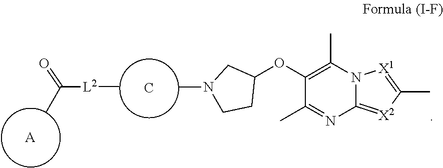

In some embodiments, ring A is:

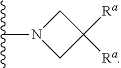

In some embodiments, one R^{a} is H, and the other R^{a} is H, halogen, —CN, —OH, —N(R¹³)₂, —OC(═O)(R¹²), —CO₂R¹³, —C(═O)N(R¹³)₂, —NR ¹³C(═O)(R¹²), —NR¹⁵C(═O)O(R¹²), —OC(═O)N(R¹³)₂, —NR¹³C(═O)N(R¹³)₂, C₁-C₄alkyl, C₂-C₄alkenyl, C₂-C₄alkynyl, C₁-C₄alkoxy, C₁-C₄deuteroalkyl, C₁-C₄deuteroalkoxy, C₁-C₄fluoroalkyl, C₁-C₄fluoroalkoxy, C₁-C₄heteroalkyl, or substituted or unsubstituted monocyclic C₂-C₅heterocycloalkyl; or both R^{a} are taken together with the carbon atom to form a C₃-C₆cycloalkyl or a C₂-C₅heterocycloalkyl.

In some embodiments, ring A is:

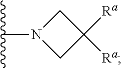

and one R^{a} is H, and the other R^{a} is H, halogen, —CN, —OH, —N(R¹³)₂, —OC(═O)(R¹²), —CO₂R¹³, —C(═O)N(R¹³)₂, —NR¹³C(═O)(R¹²), —NR¹⁵C(═O)O(R¹²), —OC(═O)N(R¹³)₂, —NR¹³C(═O)N(R¹³)₂, C₁-C₄alkyl, C₂-C₄alkenyl, C₂-C₄alkynyl, C₁-C₄alkoxy, C₁-C₄deuteroalkyl, C₁-C₄deuteroalkoxy, C₁-C₄fluoroalkyl, C₁-C₄fluoroalkoxy, C₁-C₄heteroalkyl, or substituted or unsubstituted monocyclic C₂-C₅heterocycloalkyl; or both R^{a} are taken together with the carbon atom to form a C₃-C₆cycloalkyl or a C₂-C₅heterocycloalkyl.

In some embodiments, ring A is:

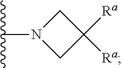

and one R^{a} is H, and the other R^{a} is —N(R¹³)₂, —OC(═O)(R¹²), —CO₂R¹³, —C(═O)N(R¹³)₂, —NR¹³C(═O)(R¹²), C₁-C₄alkyl, C₁-C₄alkoxy, C₁-C₄deuteroalkyl, C₁-C₄deuteroalkoxy, C₁-C₄fluoroalkyl, C₁-C₄fluoroalkoxy, or C₁-C₄heteroalkyl; or both R^{a} are taken together with the carbon atom to form a C₃-C₆cycloalkyl or a C₂-C₅heterocycloalkyl.

In some embodiments, ring A is:

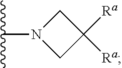

and one R^{a} is H, and the other R^{a} is H, F, Cl, Br, —CN, —OH, —OCH₃, —OCD₃, —OCFH₂, —OCHF₂, —OCF₃, —S(═O)₂CH₃, —NH₂, —NH(CH₃), —N(CH₃)₂, —C(═O)NH₂, —C(═O)N(CH₃)₂, —CH₃, —CH₂CH₃, —CH(CH₃)₂, —C(CH₃)₃, —CD₃, —CFH₂, —CHF₂, or —CF₃; or both R^{a} are taken together with the carbon atom to form a cyclobutyl, cyclopentyl, cyclohexyl, azetidinyl, oxetanyl, thietanyl, pyrrolidinyl, or piperidinyl.

In some embodiments ring A is:

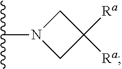

and one R^{a} is H, and the other R^{a} is hydrogen, —OCH₃, —OCD₃, —OCF₃, —S(═O)₂CH₃, —NH₂, —NH(CH₃), —N(CH₃)₂, —C(═O)NH₂, —C(═O)N(CH₃)₂, —CH₃, —CD₃, or —CF₃.

In some embodiments,

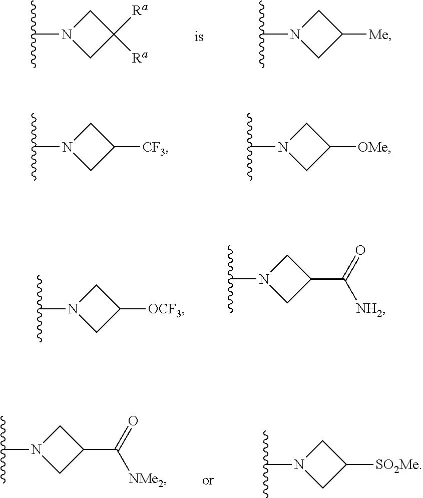

In some embodiments,

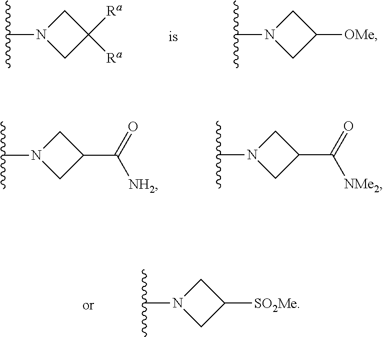

In some embodiments, ring A is:

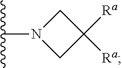

and both R^{a} are taken together with the carbon atom to form a cyclobutyl, cyclopentyl, azetidinyl, oxetanyl, thietanyl, or pyrrolidinyl.

In some embodiments

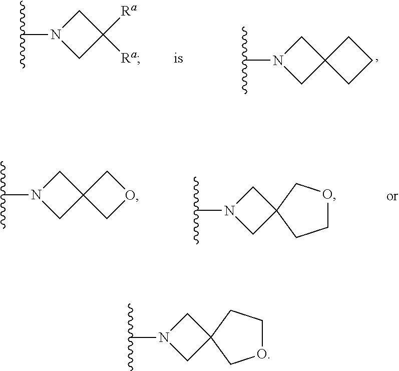

In some embodiments,

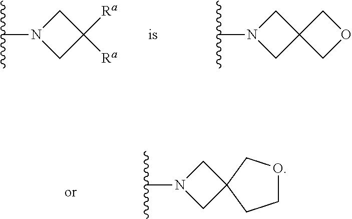

In some embodiments, L² is C₁-C₄alkyl or C₂-C₄alkynyl. In some embodiments, L² is C₂-C₄alkynyl. In some embodiments, L² is

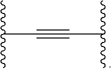 [SMILES: *C#C*]

In some embodiments, L² is a ring B. In some embodiments, ring B is a monocyclic C₃-C₈cycloalkyl, bicyclic C₅-C₁₂cycloalkyl, monocyclic C₃-C₈heterocycloalkyl, or bicyclic C₅-C₁₀heterocycloalkyl, wherein ring B is unsubstituted or is substituted with 1, 2, 3, or 4 R^{b}.

In some embodiments, ring B is:

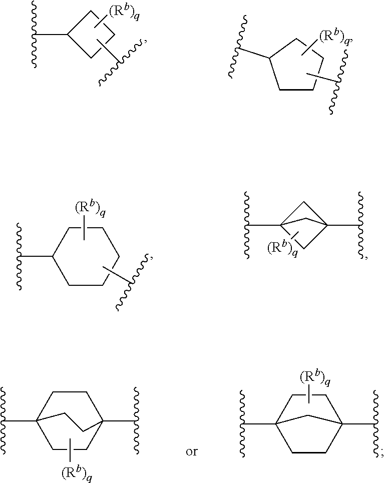

and q is 0, 1, 2, 3, or 4.

In some embodiments, ring B is

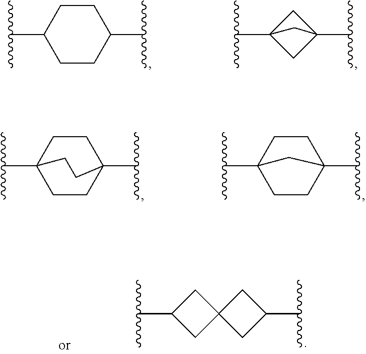 [SMILES: *C1CCC(*)CC1 ; *C12CC(*)(C1)C2 ; *C12CCC(*)(CC1)CC2 ; *C12CCC(*)(CC1)C2 ; *C1CC2(C1)CC(*)C2]

In some embodiments, ring B is:

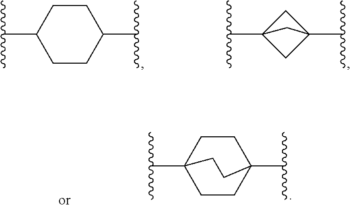 [SMILES: *C1CCC(*)CC1 ; *C12CC(*)(C1)C2 ; *C12CCC(*)(CC1)CC2]

In some embodiments, L² is a ring B and ring B is phenyl or 6-membered monocyclic heteroaryl, wherein ring B is unsubstituted or is substituted with 1, 2, 3, or 4 R^{b}.

In some embodiments, ring B is:

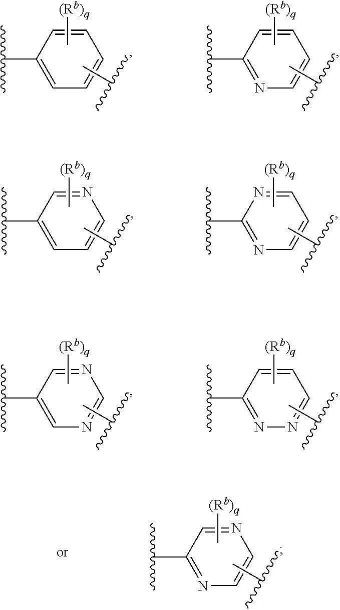

and q is 0, 1, or 2.

In some embodiments, ring B is:

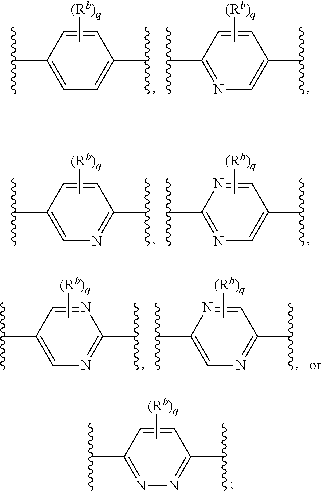

and q is 0, 1, or 2.

In some embodiments, ring B is:

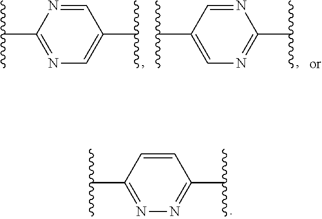

In some embodiments, q is 0 or 1. In some embodiments, q is 0.

In some embodiments, Ring C is phenyl, pyridinyl, pyrazinyl, pyrimidinyl, or pyridazinyl, wherein Ring C is unsubstituted or is substituted with 1, 2, 3, or 4 R^{c}. In some embodiments, Ring C is phenyl or pyrimidinyl, wherein Ring C is unsubstituted or is substituted with 1, 2, 3, or 4 R^{c}. In some embodiments, Ring C is phenyl, wherein Ring C is unsubstituted or is substituted with 1, 2, 3, or 4 R^{c}. In some embodiments, Ring C is pyrimidinyl, wherein Ring C is unsubstituted or is substituted with 1, 2, 3, or 4 R^{c}. In some embodiments, Ring C is unsubstituted.

In some embodiments, Ring C is:

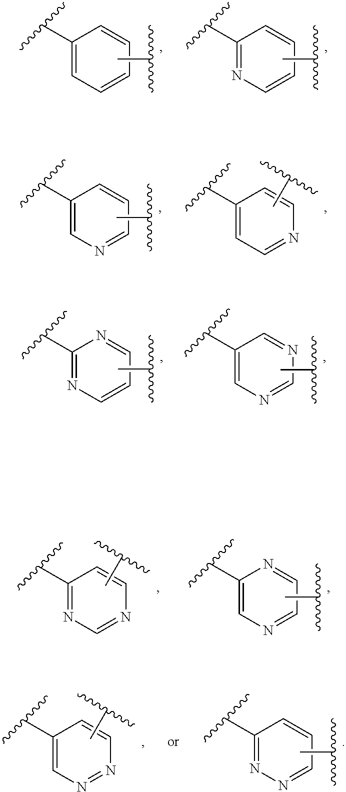

In some embodiments, Ring C is:

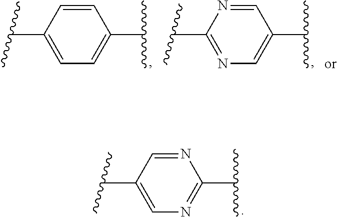

In some embodiments, L² is

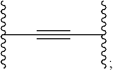 [SMILES: *C#C*]

### and Ring C is

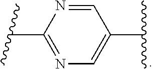 [SMILES: *c1cnc(*)nc1]

In some embodiments, L¹ is CH₂ or C(═O); L² is

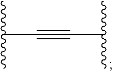 [SMILES: *C#C*]

### and Ring C is

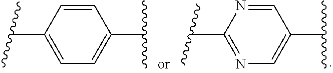

In some embodiments, L² is

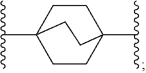 [SMILES: *C12CCC(*)(CC1)CC2]

### and Ring C is

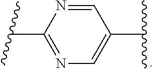 [SMILES: *c1cnc(*)nc1]

In some embodiments, L¹ is CH₂ or C(═O); L² is

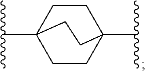 [SMILES: *C12CCC(*)(CC1)CC2]

### and Ring C is

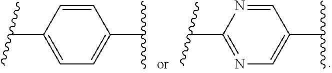

In some embodiments, L² is

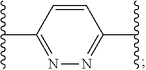 [SMILES: *c1ccc(*)nn1]

### and Ring C is

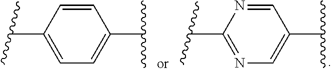

In some embodiments, L¹ is CH₂ or C(═O); L² is

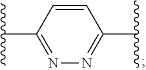 [SMILES: *c1ccc(*)nn1]

### and Ring C is

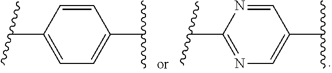

In some embodiments, L² is

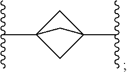 [SMILES: *C12CC(*)(C1)C2]

### and Ring C is

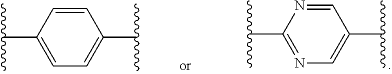 [SMILES: *c1ccc(*)cc1 ; *c1cnc(*)nc1]

In some embodiments, L¹ is CH₂ or C(═O); L² is

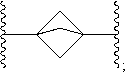 [SMILES: *C12CC(*)(C1)C2]

### and Ring C is

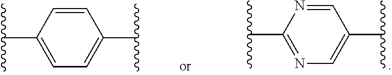 [SMILES: *c1ccc(*)cc1 ; *c1cnc(*)nc1]

In some embodiments, L² is

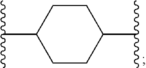 [SMILES: *C1CCC(*)CC1]

### an Ring C is

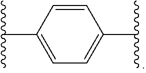 [SMILES: *c1ccc(*)cc1]

In some embodiments, L¹ is C(═O); L² is

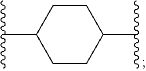 [SMILES: *C1CCC(*)CC1]

### and Ring C is

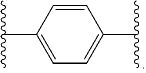 [SMILES: *c1ccc(*)cc1]

Representative compounds of any one of Formula (I-A), Formula (I-B), Formula (I-C), Formula (I-D), Formula (I-E), and/or Formula (I-F), include, but are not limited to compounds in Table 2, or a stereoisomer, or pharmaceutically acceptable salt or solvate thereof. In some embodiments, provided herein are compounds 1-35 of Table 1, or a stereoisomer, or pharmaceutically acceptable salt or solvate thereof. In some embodiments, provided herein are compounds 2-35 of Table 1, or a stereoisomer, or pharmaceutically acceptable salt or solvate thereof. In some embodiments, provided herein are compounds 2-61 of Table 1, or a stereoisomer, or pharmaceutically acceptable salt or solvate thereof.

TABLE 1

| No | Structure | Name |
|---|---|---|
| 1. | 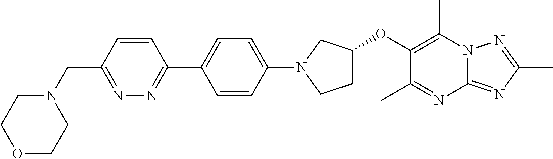 [SMILES: Cc1nc2nc(C)c(O[C@@H]3CCN(c4ccc(-c5ccc(CN6CCOCC6)nn5)cc4)C3)c(C)n2n1] | (R)-4-((6-(4-(3-((2,5,7- trimethyl- [1,2,4]triazolo[1,5- a]pyrimidin-6- yl)oxy)pyrrolidin-1- yl)phenyl)pyridazin-3- yl)methyl)morpholine |
| 2. | 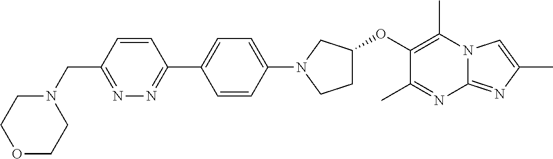 [SMILES: Cc1cn2c(C)c(O[C@@H]3CCN(c4ccc(-c5ccc(CN6CCOCC6)nn5)cc4)C3)c(C)nc2n1] | (R)-4-((6-(4-(3-((2,5,7- trimethylimidazo[1,2- a]pyrimidin-6- yl)oxy)pyrrolidin-1- yl)phenyl)pyridazin-3- yl)methyl)morpholine |
| 3. | 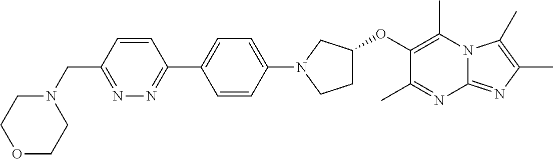 [SMILES: Cc1nc2nc(C)c(C)n2c(C)c1O[C@@H]1CCN(c2ccc(-c3ccc(CN4CCOCC4)nn3)cc2)C1] | (R)-4-((6-(4-(3-((2,3,5,7- tetramethylimidazo[1,2- a]pyrimidin-6- yl)oxy)pyrrolidin-1- yl)phenyl)pyridazin-3- yl)methyl)morpholine |
| 4. | 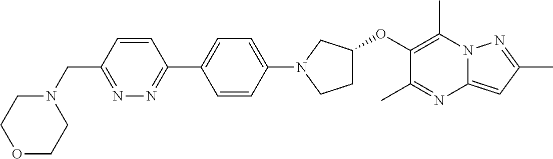 [SMILES: Cc1cc2nc(C)c(O[C@@H]3CCN(c4ccc(-c5ccc(CN6CCOCC6)nn5)cc4)C3)c(C)n2n1] | (R)-4-((6-(4-(3-((2,5,7- trimethylpyrazolo[1,5- a]pyrimidin-6- yl)oxy)pyrrolidin-1- yl)phenyl)pyridazin-3- yl)methyl)morpholine |
| 5. | 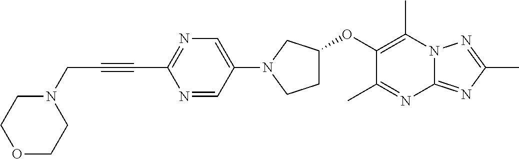 [SMILES: Cc1nc2nc(C)c(O[C@@H]3CCN(c4cnc(C#CCN5CCOCC5)nc4)C3)c(C)n2n1] | (R)-4-(3-(5-(3-((2,5,7- trimethyl- [1,2,4]triazolo[1,5- a]pyrimidin-6- yl)oxy)pyrrolidin-1- yl)pyrimidin-2-yl)prop-2- yn-1-yl)morpholine |
| 6. | 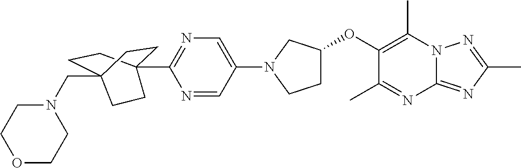 [SMILES: Cc1nc2nc(C)c(O[C@@H]3CCN(c4cnc(C56CCC(CN7CCOCC7)(CC5)CC6)nc4)C3)c(C)n2n1] | (R)-4-((4-(5-(3-((2,5,7- trimethyl- [1,2,4]triazolo[1,5- a]pyrimidin-6- yl)oxy)pyrrolidin-1- yl)pyrimidin-2- yl)bicyclo[2.2.2]octan- 1-yl)methyl)morpholine |
| 7. | 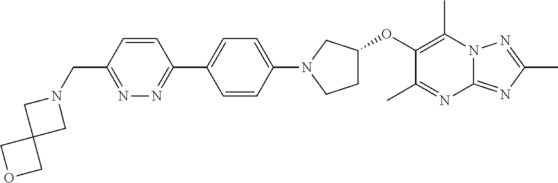 [SMILES: Cc1nc2nc(C)c(O[C@@H]3CCN(c4ccc(-c5ccc(CN6CC7(COC7)C6)nn5)cc4)C3)c(C)n2n1] | (R)-6-((6-(4-(3-((2,5,7- trimethyl- [1,2,4]triazolo[1,5- a]pyrimidin-6- yl)oxy)pyrrolidin-1- yl)phenyl)pyridazin-3- yl)methyl)-2-oxa-6- azaspiro[3.3]heptane |
| 8. | 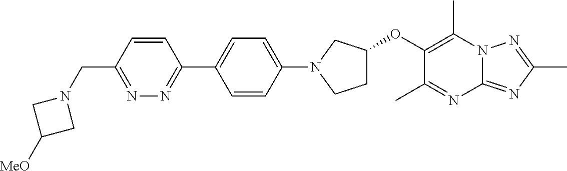 [SMILES: COC1CN(Cc2ccc(-c3ccc(N4CC[C@@H](Oc5c(C)nc6nc(C)nn6c5C)C4)cc3)nn2)C1] | (R)-6-((1-(4-(6-((3- methoxyazetidin-1- yl)methyl)pyridazin-3- yl)phenyl)pyrrolidin-3- yl)oxy)-2,5,7-trimethyl- [1,2,4]triazolo[1,5- a]pyrimidine |
| 9. | 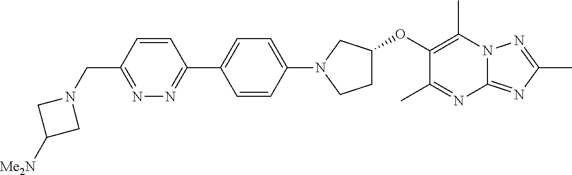 [SMILES: Cc1nc2nc(C)c(O[C@@H]3CCN(c4ccc(-c5ccc(CN6CC(N(C)C)C6)nn5)cc4)C3)c(C)n2n1] | (R)-N,N-dimethyl-1-((6- (4-(3-((2,5,7-trimethyl- [1,2,4]triazolo[1,5- a]pyrimidin-6- yl)oxy)pyrrolidin-1- yl)phenyl)pyridazin-3- yl)methyl)azetidin-3- amine |
| 10. | 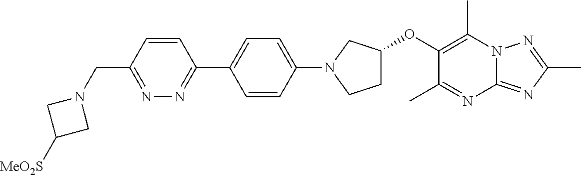 [SMILES: Cc1nc2nc(C)c(O[C@@H]3CCN(c4ccc(-c5ccc(CN6CC(S(C)(=O)=O)C6)nn5)cc4)C3)c(C)n2n1] | (R)-2,5,7-trimethyl-6-((1- (4-(6-((3- (methylsulfonyl)azetidin- 1-yl)methyl)pyridazin-3- yl)phenyl)pyrrolidin-3- yl)oxy)-[1,2,4]triazolo [1,5-a]pyrimidine |
| 11. | 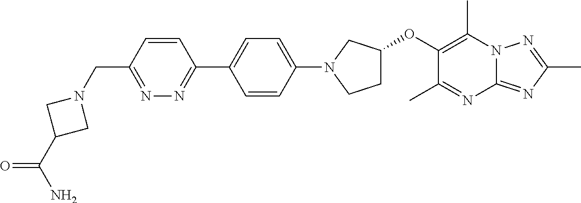 [SMILES: Cc1nc2nc(C)c(O[C@@H]3CCN(c4ccc(-c5ccc(CN6CC(C(N)=O)C6)nn5)cc4)C3)c(C)n2n1] | (R)-1-((6-(4-(3-((2,5,7- trimethyl- [1,2,4]triazolo[1,5- a]pyrimidin-6- yl)oxy)pyrrolidin-1- yl)phenyl)pyridazin-3- yl)methyl)azetidine-3- carboxamide |
| 12. | 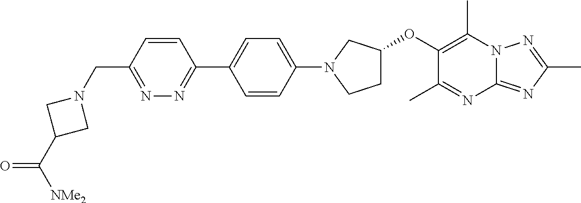 [SMILES: Cc1nc2nc(C)c(O[C@@H]3CCN(c4ccc(-c5ccc(CN6CC(C(=O)N(C)C)C6)nn5)cc4)C3)c(C)n2n1] | (R)-N,N-dimethyl-1-((6- (4-(3-((2,5,7-trimethyl- [1,2,4]triazolo[1,5- a]pyrimidin-6- yl)oxy)pyrrolidin-1- yl)phenyl)pyridazin-3- yl)methyl)azetidine-3- carboxamide |
| 13. | 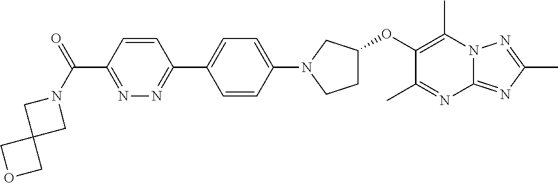 [SMILES: Cc1nc2nc(C)c(O[C@@H]3CCN(c4ccc(-c5ccc(C(=O)N6CC7(COC7)C6)nn5)cc4)C3)c(C)n2n1] | (R)-(2-oxa-6- azaspiro[3.3]heptan-6- yl)(6-(4-(3-((2,5,7- trimethyl- [1,2,4]triazolo[1,5- a]pyrimidin-6- yl)oxy)pyrrolidin-1- yl)phenyl)pyridazin-3- yl)methanone |
| 14. | 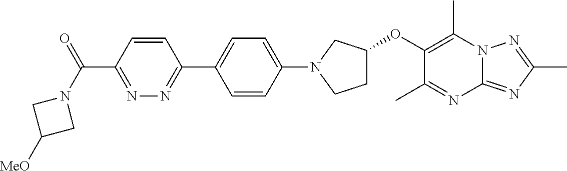 [SMILES: COC1CN(C(=O)c2ccc(-c3ccc(N4CC[C@@H](Oc5c(C)nc6nc(C)nn6c5C)C4)cc3)nn2)C1] | (R)-(3-methoxyazetidin- 1-yl)(6-(4-(3-((2,5,7- trimethyl- [1,2,4]triazolo[1,5- a]pyrimidin-6- yl)oxy)pyrrolidin-1- yl)phenyl)pyridazin-3- yl)methanone |
| 15. | 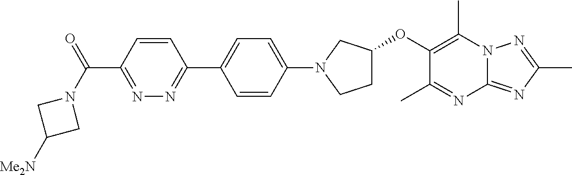 [SMILES: Cc1nc2nc(C)c(O[C@@H]3CCN(c4ccc(-c5ccc(C(=O)N6CC(N(C)C)C6)nn5)cc4)C3)c(C)n2n1] | (R)-(3- (dimethylamino)azetidin- 1-yl)(6-(4-(3-((2,5,7- trimethyl- [1,2,4]triazolo[1,5- a]pyrimidin-6- yl)oxy)pyrrolidin-1- yl)phenyl)pyridazin-3- yl)methanone |
| 16. | 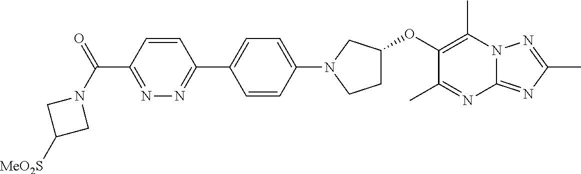 [SMILES: Cc1nc2nc(C)c(O[C@@H]3CCN(c4ccc(-c5ccc(C(=O)N6CC(S(C)(=O)=O)C6)nn5)cc4)C3)c(C)n2n1] | (R)-(3- (methylsulfonyl)azetidin- 1-yl)(6-(4-(3-((2,5,7- trimethyl- [1,2,4]triazolo[1,5- a]pyrimidin-6- yl)oxy)pyrrolidin-1- yl)phenyl)pyridazin-3- yl)methanone |
| 17. | 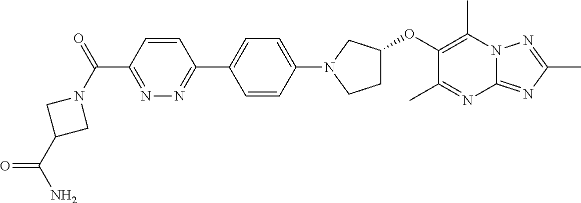 [SMILES: Cc1nc2nc(C)c(O[C@@H]3CCN(c4ccc(-c5ccc(C(=O)N6CC(C(N)=O)C6)nn5)cc4)C3)c(C)n2n1] | (R)-1-(6-(4-(3-((2,5,7- trimethyl- [1,2,4]triazolo[1,5- a]pyrimidin-6- yl)oxy)pyrrolidin-1- yl)phenyl)pyridazine-3- carbonyl)azetidine-3- carboxamide |
| 18. | 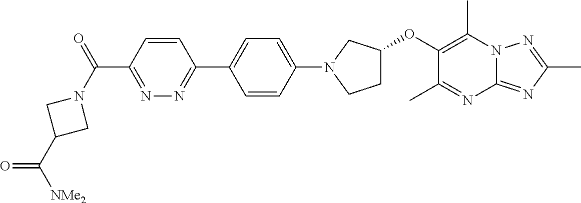 [SMILES: Cc1nc2nc(C)c(O[C@@H]3CCN(c4ccc(-c5ccc(C(=O)N6CC(C(=O)N(C)C)C6)nn5)cc4)C3)c(C)n2n1] | (R)-N,N-dimethyl-1-(6- (4-(3-((2,5,7-trimethyl- [1,2,4]triazolo[1,5- a]pyrimidin-6- yl)oxy)pyrrolidin-1- yl)phenyl)pyridazine-3- carbonyl)azetidine-3- carboxamide |
| 19. | 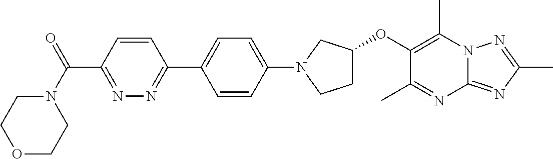 [SMILES: Cc1nc2nc(C)c(O[C@@H]3CCN(c4ccc(-c5ccc(C(=O)N6CCOCC6)nn5)cc4)C3)c(C)n2n1] | (R)-morpholino(6-(4- (3-((2,5,7-trimethyl- [1,2,4]triazolo[1,5- a]pyrimidin-6- yl)oxy)pyrrolidin-1- yl)phenyl)pyridazin-3- yl)methanone |
| 20. | 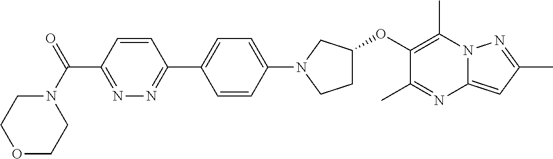 [SMILES: Cc1cc2nc(C)c(O[C@@H]3CCN(c4ccc(-c5ccc(C(=O)N6CCOCC6)nn5)cc4)C3)c(C)n2n1] | (R)-morpholino(6-(4-(3- ((2,5,7- trimethylpyrazolo[1,5- a]pyrimidin-6- yl)oxy)pyrrolidin-1- yl)phenyl)pyridazin-3- yl)methanone |
| 21. | 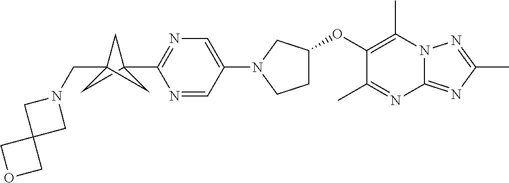 [SMILES: Cc1nc2nc(C)c(O[C@@H]3CCN(c4cnc(C56CC(CN7CC8(COC8)C7)(C5)C6)nc4)C3)c(C)n2n1] | (R)-6-((3-(5-(3-((2,5,7- trimethyl- [1,2,4]triazolo[1,5- a]pyrimidin-6- yl)oxy)pyrrolidin-1- yl)pyrimidin-2- yl)bicyclo[1.1.1]pentan- 1-yl)methyl)-2-oxa-6- azaspiro[3.3]heptane |
| 22. | 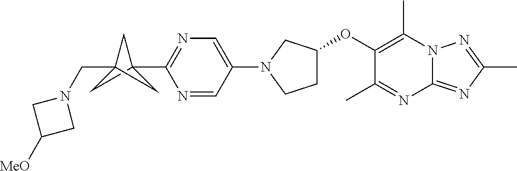 [SMILES: COC1CN(CC23CC(c4ncc(N5CC[C@@H](Oc6c(C)nc7nc(C)nn7c6C)C5)cn4)(C2)C3)C1] | (R)-6-((1-(2-(3-((3- methoxyazetidin-1- yl)methyl)bicyclo[1.1.1] pentan-1-yl)pyrimidin-5- yl)pyrrolidin-3-yl)oxy)- 2,5,7-trimethyl- [1,2,4]triazolo[1,5- a]pyrimidine |
| 23. | 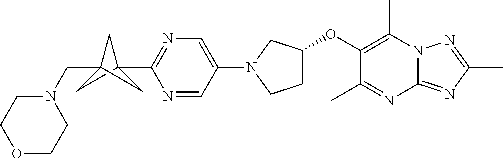 [SMILES: Cc1nc2nc(C)c(O[C@@H]3CCN(c4cnc(C56CC(CN7CCOCC7)(C5)C6)nc4)C3)c(C)n2n1] | (R)-4-((3-(5-(3-((2,5,7- trimethyl- [1,2,4]triazolo[1,5- a]pyrimidin-6- yl)oxy)pyrrolidin-1- yl)pyrimidin-2- yl)bicyclo[1.1.1]pentan- 1-yl)methyl)morpholine |
| 24. | 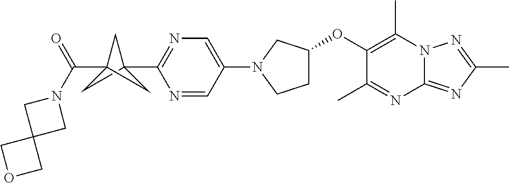 [SMILES: Cc1nc2nc(C)c(O[C@@H]3CCN(c4cnc(C56CC(C(=O)N7CC8(COC8)C7)(C5)C6)nc4)C3)c(C)n2n1] | (R)-(2-oxa-6- azaspiro[3.3]heptan-6- yl)(3-(5-(3-((2,5,7- trimethyl- [1,2,4]triazolo[1,5- a]pyrimidin-6- yl)oxy)pyrrolidin-1- yl)pyrimidin-2- yl)bicyclo[1.1.1]pentan- 1-yl)methanone |
| 25. | 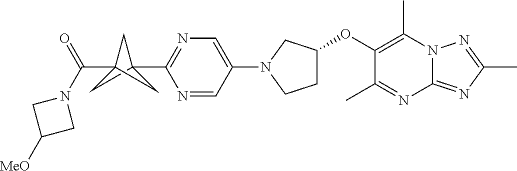 [SMILES: COC1CN(C(=O)C23CC(c4ncc(N5CC[C@@H](Oc6c(C)nc7nc(C)nn7c6C)C5)cn4)(C2)C3)C1] | (R)-(3-methoxyazetidin- 1-yl)(3-(5-(3-((2,5,7- trimethyl- [1,2,4]triazolo[1,5- a]pyrimidin-6- yl)oxy)pyrrolidin-1- yl)pyrimidin-2- yl)bicyclo[1.1.1]pentan- 1-yl)methanone |
| 26. | 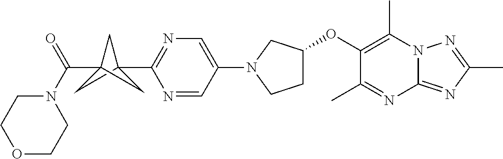 [SMILES: Cc1nc2nc(C)c(O[C@@H]3CCN(c4cnc(C56CC(C(=O)N7CCOCC7)(C5)C6)nc4)C3)c(C)n2n1] | (R)-morpholino(3-(5-(3- ((2,5,7-trimethyl- [1,2,4]triazolo[1,5- a]pyrimidin-6- yl)oxy)pyrrolidin-1- yl)pyrimidin-2- yl)bicyclo[1.1.1]pentan- 1-yl)methanone |
| 27. | 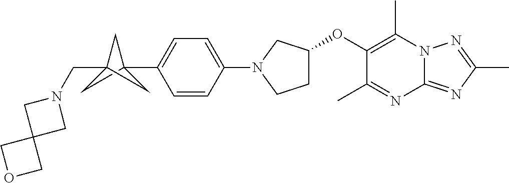 [SMILES: Cc1nc2nc(C)c(O[C@@H]3CCN(c4ccc(C56CC(CN7CC8(COC8)C7)(C5)C6)cc4)C3)c(C)n2n1] | (R)-6-((3-(4-(3-((2,5,7- trimethyl- [1,2,4]triazolo[1,5- a]pyrimidin-6- yl)oxy)pyrrolidin-1- yl)phenyl)bicyclo[1.1.1] pentan-1-yl)methyl)-2- oxa-6-azaspiro[3.3] heptane |
| 28. | 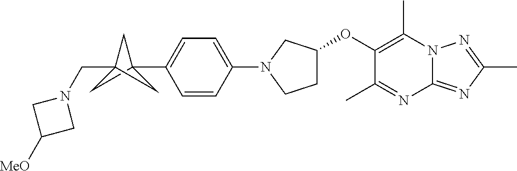 [SMILES: COC1CN(CC23CC(c4ccc(N5CC[C@@H](Oc6c(C)nc7nc(C)nn7c6C)C5)cc4)(C2)C3)C1] | (R)-6-((1-(4-(3-((3- methoxyazetidin-1- yl)methyl)bicyclo[1.1.1] pentan-1- yl)phenyl)pyrrolidin-3- yl)oxy)-2,5,7-trimethyl- [1,2,4]triazolo[1,5- a]pyrimidine |
| 29. | 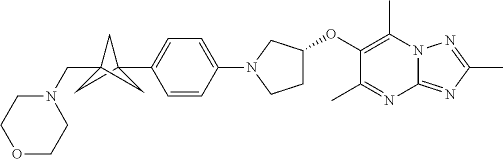 [SMILES: Cc1nc2nc(C)c(O[C@@H]3CCN(c4ccc(C56CC(CN7CCOCC7)(C5)C6)cc4)C3)c(C)n2n1] | (R)-4-((3-(4-(3-((2,5,7- trimethyl- [1,2,4]triazolo[1,5- a]pyrimidin-6- yl)oxy)pyrrolidin-1- yl)phenyl)bicyclo[1.1.1] pentan-1- yl)methyl)morpholine |
| 30. | 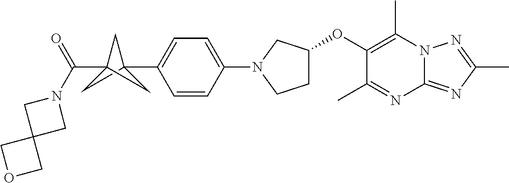 [SMILES: Cc1nc2nc(C)c(O[C@@H]3CCN(c4ccc(C56CC(C(=O)N7CC8(COC8)C7)(C5)C6)cc4)C3)c(C)n2n1] | (R)-(2-oxa-6- azaspiro[3.3]heptan-6- yl)(3-(4-(3-((2,5,7- trimethyl- [1,2,4]triazolo[1,5- a]pyrimidin-6- yl)oxy)pyrrolidin-1- yl)phenyl)bicyclo[1.1.1] pentan-1-yl) methanone |
| 31. | 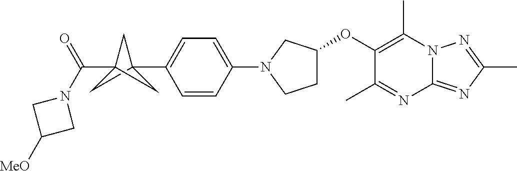 [SMILES: COC1CN(C(=O)C23CC(c4ccc(N5CC[C@@H](Oc6c(C)nc7nc(C)nn7c6C)C5)cc4)(C2)C3)C1] | (R)-(3-methoxyazetidin- 1-yl)(3-(4-(3-((2,5,7- trimethyl- [1,2,4]triazolo[1,5- a]pyrimidin-6- yl)oxy)pyrrolidin-1- yl)phenyl)bicyclo[1.1.1] pentan-1-yl)methanone |
| 32. | 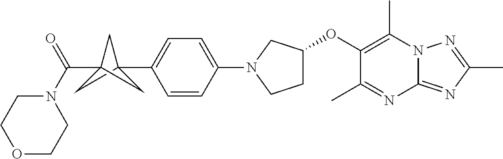 [SMILES: Cc1nc2nc(C)c(O[C@@H]3CCN(c4ccc(C56CC(C(=O)N7CCOCC7)(C5)C6)cc4)C3)c(C)n2n1] | (R)-morpholino(3-(4-(3- ((2,5,7-trimethyl- [1,2,4]triazolo[1,5- a]pyrimidin-6- yl)oxy)pyrrolidin-1- yl)phenyl)bicyclo[1.1.1] pentan-1-yl)methanone |
| 33. | 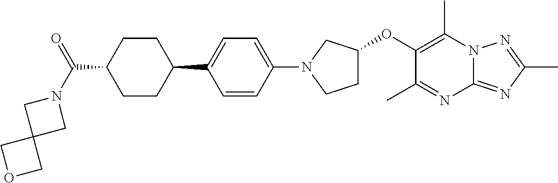 [SMILES: Cc1nc2nc(C)c(O[C@@H]3CCN(c4ccc([C@H]5CC[C@H](C(=O)N6CC7(COC7)C6)CC5)cc4)C3)c(C)n2n1] | (2-oxa-6- azaspiro[3.3]heptan-6- yl)((1R,4r)-4-(4-((R)-3- ((2,5,7-trimethyl- [1,2,4]triazolo[1,5- a]pyrimidin-6- yl)oxy)pyrrolidin-1- yl)phenyl)cyclohexyl) methanone |
| 34. | 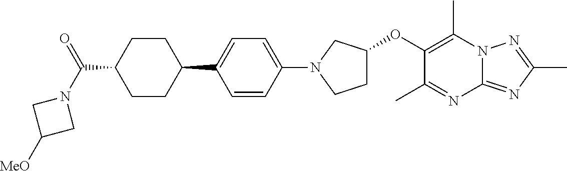 [SMILES: COC1CN(C(=O)[C@H]2CC[C@H](c3ccc(N4CC[C@@H](Oc5c(C)nc6nc(C)nn6c5C)C4)cc3)CC2)C1] | (3-methoxyazetidin-1- yl)((1R,4r)-4-(4-((R)-3- ((2,5,7-trimethyl- [1,2,4]triazolo[1,5- a]pyrimidin-6- yl)oxy)pyrrolidin-1- yl)phenyl)cyclohexyl) methanone |
| 35. | 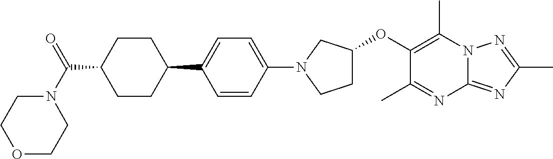 [SMILES: Cc1nc2nc(C)c(O[C@@H]3CCN(c4ccc([C@H]5CC[C@H](C(=O)N6CCOCC6)CC5)cc4)C3)c(C)n2n1] | morpholino((1R,4r)-4-(4- ((R)-3-((2,5,7-trimethyl- [1,2,4]triazolo[1,5- a]pyrimidin-6- yl)oxy)pyrrolidin-1- yl)phenyl)cyclohexyl) methanone |
| 36. | 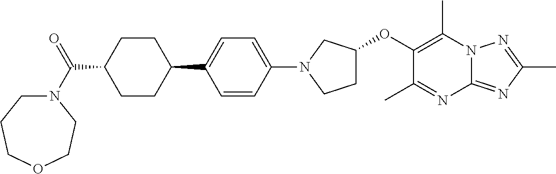 [SMILES: Cc1nc2nc(C)c(O[C@@H]3CCN(c4ccc([C@H]5CC[C@H](C(=O)N6CCCOCC6)CC5)cc4)C3)c(C)n2n1] | (1,4-oxazepan-4- yl)((1R,4r)-4-(4-((R)-3- ((2,5,7-trimethyl- [1,2,4]triazolo[1,5- a]pyrimidin-6- yl)oxy)pyrrolidin-1- yl)phenyl)cyclohexyl) methanone |
| 37. | 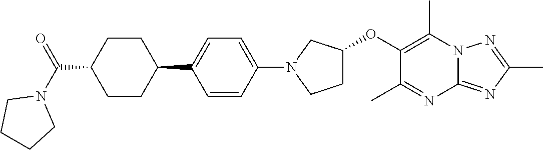 [SMILES: Cc1nc2nc(C)c(O[C@@H]3CCN(c4ccc([C@H]5CC[C@H](C(=O)N6CCCC6)CC5)cc4)C3)c(C)n2n1] | pyrrolidin-1-yl((1R,4r)- 4-(4-((R)-3-((2,5,7- trimethyl- [1,2,4]triazolo[1,5- a]pyrimidin-6- yl)oxy)pyrrolidin-1- yl)phenyl)cyclohexyl) methanone |
| 38. | 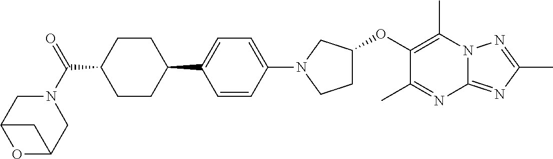 [SMILES: Cc1nc2nc(C)c(O[C@@H]3CCN(c4ccc([C@H]5CC[C@H](C(=O)N6CC7CC(C6)O7)CC5)cc4)C3)c(C)n2n1] | (6-oxa-3- azabicyclo[3.1.1]heptan- 3-yl)((1R,4r)-4-(4-((R)- 3-((2,5,7-trimethyl- [1,2,4]triazolo[1,5- a]pyrimidin-6- yl)oxy)pyrrolidin-1- yl)phenyl)cyclohexyl) methanone |
| 39. | 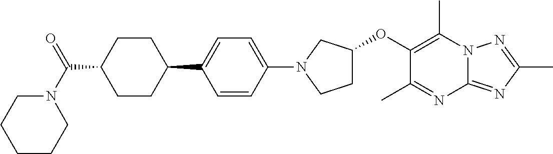 [SMILES: Cc1nc2nc(C)c(O[C@@H]3CCN(c4ccc([C@H]5CC[C@H](C(=O)N6CCCCC6)CC5)cc4)C3)c(C)n2n1] | piperidin-1-yl((1R,4r)-4- (4-((R)-3-((2,5,7- trimethyl- [1,2,4]triazolo[1,5- a]pyrimidin-6- yl)oxy)pyrrolidin-1- yl)phenyl)cyclohexyl) methanone |
| 40. | 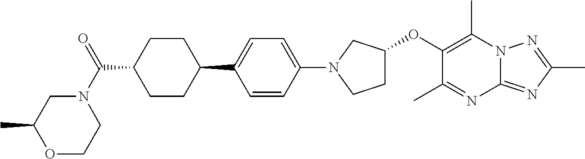 | ((S)-2- methylmorpholino) ((1R,4S)-4-(4-((R)-3- ((2,5,7-trimethyl- [1,2,4]triazolo[1,5- a]pyrimidin-6- yl)oxy)pyrrolidin-1- yl)phenyl)cyclohexyl) methanone |
| 41. | 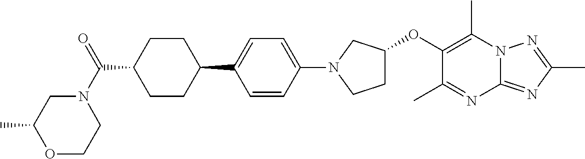 | ((R)-2- methylmorpholino) ((1R,4R)-4-(4-((R)-3- ((2,5,7-trimethyl- [1,2,4]triazolo[1,5- a]pyrimidin-6- yl)oxy)pyrrolidin-1- yl)phenyl)cyclohexyl) methanone |
| 42. | 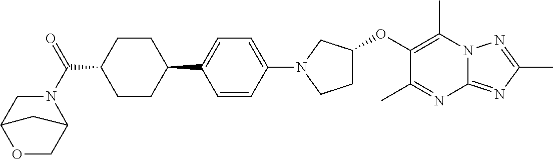 [SMILES: Cc1nc2nc(C)c(O[C@@H]3CCN(c4ccc([C@H]5CC[C@H](C(=O)N6CC7CC6CO7)CC5)cc4)C3)c(C)n2n1] | (2-oxa-5- azabicyclo[2.2.1] heptan-5-yl) ((1R,4r)-4-(4-((R)- 3-((2,5,7-trimethyl- [1,2,4]triazolo[1,5- a]pyrimidin-6- yl)oxy)pyrrolidin-1- yl)phenyl)cyclohexyl) methanone |
| 43. | 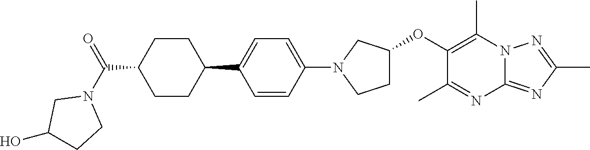 [SMILES: Cc1nc2nc(C)c(O[C@@H]3CCN(c4ccc([C@H]5CC[C@H](C(=O)N6CCC(O)C6)CC5)cc4)C3)c(C)n2n1] | (3-hydroxypyrrolidin- 1-yl)((1R,4r)-4-(4- ((R)-3-((2,5,7-trimethyl- [1,2,4]triazolo[1,5- a]pyrimidin-6- yl)oxy)pyrrolidin-1- yl)phenyl)cyclohexyl) methanone |
| 44. | 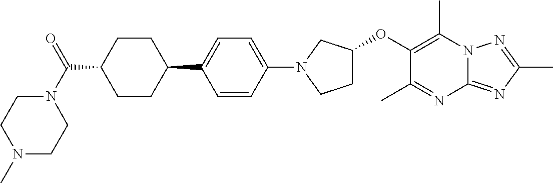 [SMILES: Cc1nc2nc(C)c(O[C@@H]3CCN(c4ccc([C@H]5CC[C@H](C(=O)N6CCN(C)CC6)CC5)cc4)C3)c(C)n2n1] | (4-methylpiperazin-1- yl)((1R,4r)-4-(4-((R)- 3-((2,5,7-trimethyl- [1,2,4]triazolo[1,5- a]pyrimidin-6- yl)oxy)pyrrolidin-1- yl)phenyl)cyclohexyl) methanone |
| 45. | 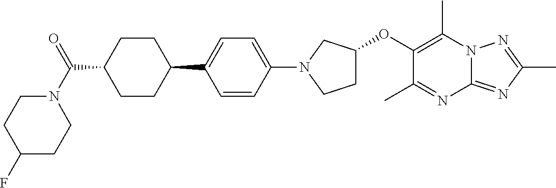 [SMILES: Cc1nc2nc(C)c(O[C@@H]3CCN(c4ccc([C@H]5CC[C@H](C(=O)N6CCC(F)CC6)CC5)cc4)C3)c(C)n2n1] | (4-fluoropiperidin-1- yl)((1R,4r)-4-(4-((R)- 3-((2,5,7-trimethyl- [1,2,4]triazolo[1,5- a]pyrimidin-6- yl)oxy)pyrrolidin-1- yl)phenyl)cyclohexyl) methanone |
| 46. | 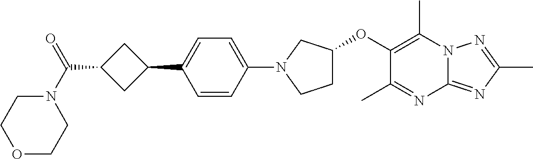 [SMILES: Cc1nc2nc(C)c(O[C@@H]3CCN(c4ccc([C@H]5C[C@H](C(=O)N6CCOCC6)C5)cc4)C3)c(C)n2n1] | morpholino((1R,3r)-3-(4- ((R)-3-((2,5,7-trimethyl- [1,2,4]triazolo[1,5- a]pyrimidin-6- yl)oxy)pyrrolidin-1- yl)phenyl)cyclobutyl) methanone |
| 47. | 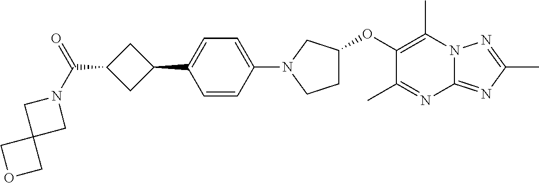 [SMILES: Cc1nc2nc(C)c(O[C@@H]3CCN(c4ccc([C@H]5C[C@H](C(=O)N6CC7(COC7)C6)C5)cc4)C3)c(C)n2n1] | (2-oxa-6- azaspiro[3.3]heptan-6- yl)((1R,3r)-3-(4-((R)-3- ((2,5,7-trimethyl- [1,2,4]triazolo[1,5- a]pyrimidin-6- yl)oxy)pyrrolidin-1- yl)phenyl)cyclobutyl) methanone |
| 48. | 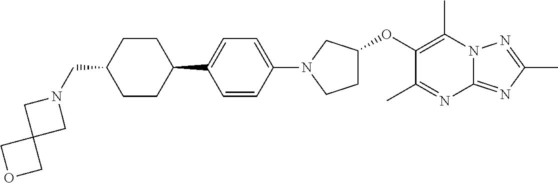 [SMILES: Cc1nc2nc(C)c(O[C@@H]3CCN(c4ccc([C@H]5CC[C@H](CN6CC7(COC7)C6)CC5)cc4)C3)c(C)n2n1] | 6-(((1R,4r)-4-(4-((R)-3- ((2,5,7-trimethyl- [1,2,4]triazolo[1,5- a]pyrimidin-6- yl)oxy)pyrrolidin-1- yl)phenyl)cyclohexyl) methyl)-2-oxa-6- azaspiro[3.3]heptane |
| 49. | 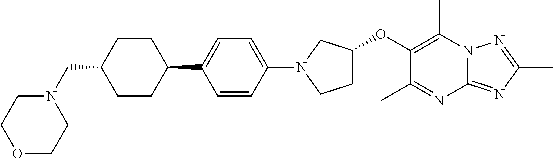 [SMILES: Cc1nc2nc(C)c(O[C@@H]3CCN(c4ccc([C@H]5CC[C@H](CN6CCOCC6)CC5)cc4)C3)c(C)n2n1] | 4-(((1R,4r)-4-(4-((R)-3- ((2,5,7-trimethyl- [1,2,4]triazolo[1,5- a]pyrimidin-6- yl)oxy)pyrrolidin-1- yl)phenyl)cyclohexyl) methyl)morpholine |
| 50. | 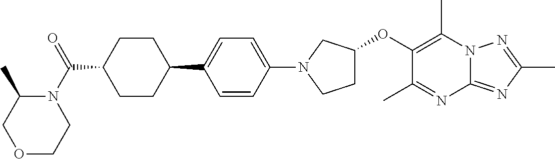 [SMILES: Cc1nc2nc(C)c(O[C@@H]3CCN(c4ccc([C@H]5CC[C@H](C(=O)N6CCOC[C@H]6C)CC5)cc4)C3)c(C)n2n1] | ((R)-3- methylmorpholino) ((1R,4R)- 4-(4-((R)-3-((2,5,7- trimethyl- [1,2,4]triazolo[1,5- a]pyrimidin-6- yl)oxy)pyrrolidin-1- yl)phenyl)cyclohexyl) methanone |
| 51. | 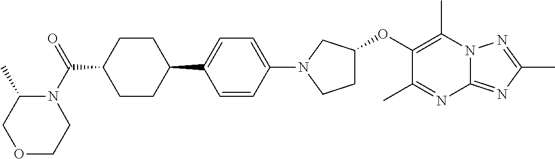 [SMILES: Cc1nc2nc(C)c(O[C@@H]3CCN(c4ccc([C@H]5CC[C@H](C(=O)N6CCOC[C@@H]6C)CC5)cc4)C3)c(C)n2n1] | ((S)-3- methylmorpholino) ((1R,4S)-4-(4-((R)-3- ((2,5,7-trimethyl- [1,2,4]triazolo[1,5- a]pyrimidin-6- yl)oxy)pyrrolidin-1- yl)phenyl)cyclohexyl) methanone |
| 52. | 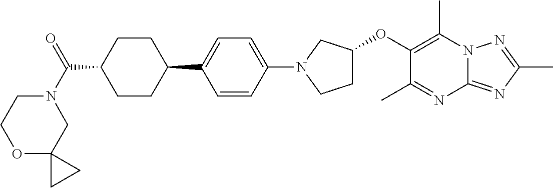 [SMILES: Cc1nc2nc(C)c(O[C@@H]3CCN(c4ccc([C@H]5CC[C@H](C(=O)N6CCOC7(CC7)C6)CC5)cc4)C3)c(C)n2n1] | (4-oxa-7- azaspiro[2.5]octan-7- yl)((1R,4r)-4-(4-((R)-3- ((2,5,7-trimethyl- [1,2,4]triazolo[1,5- a]pyrimidin-6- yl)oxy)pyrrolidin-1- yl)phenyl)cyclohexyl) methanone |
| 53. | 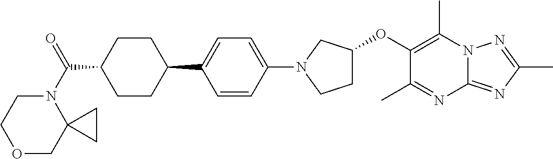 [SMILES: Cc1nc2nc(C)c(O[C@@H]3CCN(c4ccc([C@H]5CC[C@H](C(=O)N6CCOCC67CC7)CC5)cc4)C3)c(C)n2n1] | (7-oxa-4- azaspiro[2.5]octan-4- yl)((1R,4r)-4-(4-((R)-3- ((2,5,7-trimethyl- [1,2,4]triazolo[1,5- a]pyrimidin-6- yl)oxy)pyrrolidin-1- yl)phenyl)cyclohexyl) methanone |
| 54. | 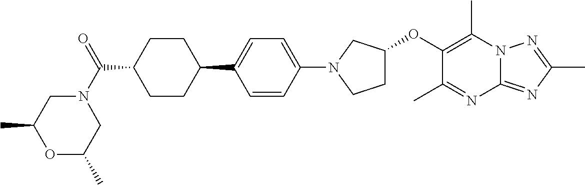 | ((2S,6S)-2,6- dimethylmorpholino) ((1R,4S)-4-(4-((R)-3- ((2,5,7-trimethyl- [1,2,4]triazolo[1,5- a]pyrimidin-6- yl)oxy)pyrrolidin-1- yl)phenyl)cyclohexyl) methanone |
| 55. | 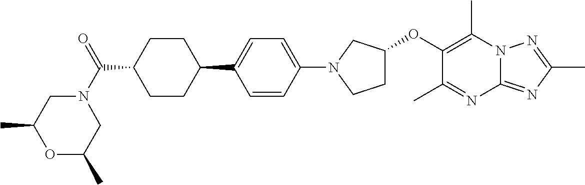 | ((2S,6R)-2,6- dimethylmorpholino) ((1R,4R)-4-(4-((R)-3- ((2,5,7-trimethyl- [1,2,4]triazolo[1,5- a]pyrimidin-6- yl)oxy)pyrrolidin-1- yl)phenyl)cyclohexyl) methanone |
| 56. | 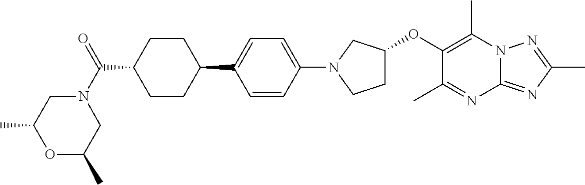 | ((2R,6R)-2,6- dimethylmorpholino) ((1R,4R)-4-(4-((R)-3- ((2,5,7-trimethyl- [1,2,4]triazolo[1,5- a]pyrimidin-6- yl)oxy)pyrrolidin-1- yl)phenyl)cyclohexyl) methanone |
| 57. | 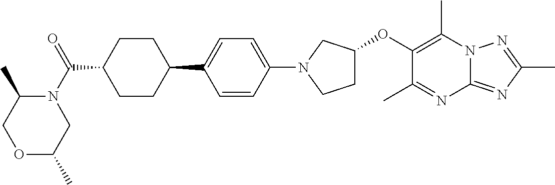 [SMILES: Cc1nc2nc(C)c(O[C@@H]3CCN(c4ccc([C@H]5CC[C@H](C(=O)N6C[C@H](C)OC[C@H]6C)CC5)cc4)C3)c(C)n2n1] | ((2S,5R)-2,5- dimethylmorpholino) ((1R,4R)-4-(4-((R)-3- ((2,5,7-trimethyl- [1,2,4]triazolo[1,5- a]pyrimidin-6- yl)oxy)pyrrolidin-1- yl)phenyl)cyclohexyl) methanone |
| 58. | 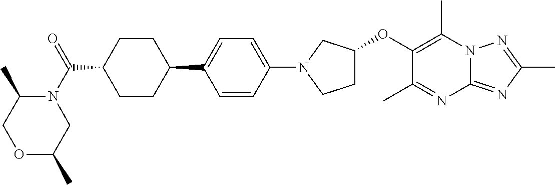 [SMILES: Cc1nc2nc(C)c(O[C@@H]3CCN(c4ccc([C@H]5CC[C@H](C(=O)N6C[C@@H](C)OC[C@H]6C)CC5)cc4)C3)c(C)n2n1] | ((2R,5R)-2,5- dimethylmorpholino) ((1R,4R)-4-(4-((R)-3- ((2,5,7-trimethyl- [1,2,4]triazolo[1,5- a]pyrimidin-6- yl)oxy)pyrrolidin-1- yl)phenyl)cyclohexyl) methanone |
| 59. | 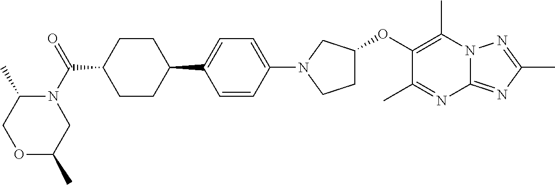 [SMILES: Cc1nc2nc(C)c(O[C@@H]3CCN(c4ccc([C@H]5CC[C@H](C(=O)N6C[C@@H](C)OC[C@@H]6C)CC5)cc4)C3)c(C)n2n1] | ((2R,5S)-2,5- dimethylmorpholino) ((1R,4S)-4-(4-((R)-3- ((2,5,7-trimethyl- [1,2,4]triazolo[1,5- a]pyrimidin-6- yl)oxy)pyrrolidin-1- yl)phenyl)cyclohexyl) methanone |
| 60. | 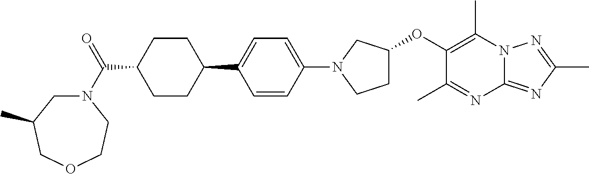 | ((S)-6-methyl-1,4- oxazepan-4-yl)((1R,4S)- 4-(4-((R)-3-((2,5,7- trimethyl- [1,2,4]triazolo[1,5- a]pyrimidin-6- yl)oxy)pyrrolidin-1- yl)phenyl)cyclohexyl) methanone |
| 51. | 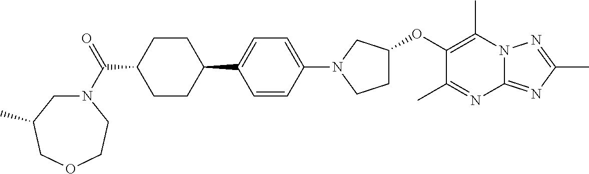 | ((R)-6-methyl-1,4- oxazepan-4-yl)((1R,4R)- 4-(4-((R)-3-((2,5,7- trimethyl- [1,2,4]triazolo[1,5- a]pyrimidin-6- yl)oxy)pyrrolidin-1- yl)phenyl)cyclohexyl) methanone |

In some embodiments, provided herein are compounds of Formula (II):

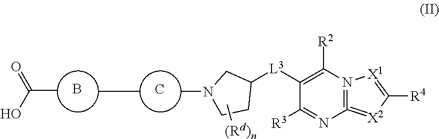

wherein X¹, X², R², R³, R⁴, L³, R^{d}, n, ring B and ring C are as defined herein in some or any embodiments. In some embodiments, examples of compounds of Formula (II) include and are not limited to

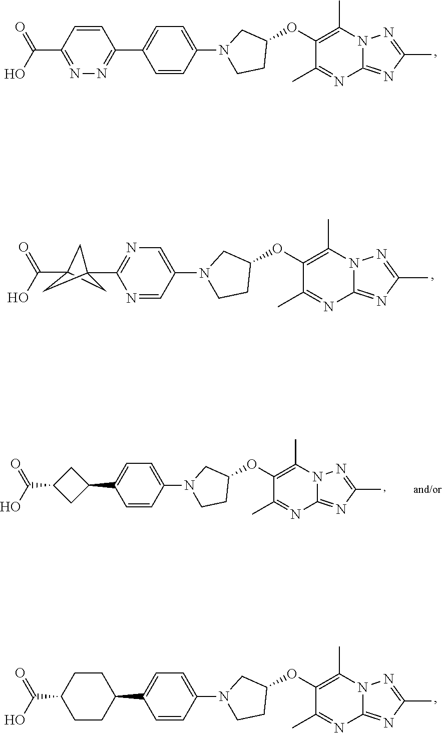 [SMILES: Cc1nc2nc(C)c(O[C@@H]3CCN(c4ccc(-c5ccc(C(=O)O)nn5)cc4)C3)c(C)n2n1 ; Cc1nc2nc(C)c(O[C@@H]3CCN(c4cnc(C56CC(C(=O)O)(C5)C6)nc4)C3)c(C)n2n1 ; Cc1nc2nc(C)c(O[C@@H]3CCN(c4ccc([C@H]5C[C@H](C(=O)O)C5)cc4)C3)c(C)n2n1 ; Cc1nc2nc(C)c(O[C@@H]3CCN(c4ccc([C@H]5CC[C@H](C(=O)O)CC5)cc4)C3)c(C)n2n1]

In some embodiments, compounds of Formula (II) are used to prepare compounds of any one of Formula (I-A), Formula (I-B), Formula (I-C), Formula (I-D), Formula (I-E), and/or Formula (I-F) as described herein in Schemes 1-5 and in the Examples section.

### Further Forms of Compounds

In one aspect, compounds described herein are in the form of pharmaceutically acceptable salts. As well, active metabolites of these compounds having the same type of activity are included in the scope of the present disclosure. In addition, the compounds described herein can exist in unsolvated as well as solvated forms with pharmaceutically acceptable solvents such as water, ethanol, and the like. The solvated forms of the compounds presented herein are also considered to be disclosed herein.

“Pharmaceutically acceptable,” as used herein, refers a material, such as a carrier or diluent, which does not abrogate the biological activity or properties of the compound, and is relatively nontoxic, i.e., the material is administered to an individual without causing undesirable biological effects or interacting in a deleterious manner with any of the components of the composition in which it is contained.

The term “pharmaceutically acceptable salt” refers to a form of a therapeutically active agent that consists of a cationic form of the therapeutically active agent in combination with a suitable anion, or in alternative embodiments, an anionic form of the therapeutically active agent in combination with a suitable cation. Handbook of Pharmaceutical Salts: Properties, Selection and Use. International Union of Pure and Applied Chemistry, Wiley-VCH 2002. S. M. Berge, L. D. Bighley, D. C. Monkhouse, J. Pharm. Sci. 1977, 66, 1-19. P. H. Stahl and C. G. Wermuth, editors, Handbook of Pharmaceutical Salts: Properties, Selection and Use, Weinheim/Zurich:Wiley-VCH/VHCA, 2002. Pharmaceutical salts typically are more soluble and more rapidly soluble in stomach and intestinal juices than non-ionic species and so are useful in solid dosage forms. Furthermore, because their solubility often is a function of pH, selective dissolution in one or another part of the digestive tract is possible and this capability can be manipulated as one aspect of delayed and sustained release behaviors. Also, because the salt-forming molecule can be in equilibrium with a neutral form, passage through biological membranes can be adjusted.

In some embodiments, pharmaceutically acceptable salts are obtained by reacting a compound of Formula (I-A), Formula (I-B), Formula (I-C), Formula (I-D), Formula (I-E), or Formula (I-F) with an acid. In some embodiments, the compound of Formula (I-A), Formula (I-B), Formula (I-C), Formula (I-D), Formula (I-E), or Formula (I-F) (i.e. free base form) is basic and is reacted with an organic acid or an inorganic acid. Inorganic acids include, but are not limited to, hydrochloric acid, hydrobromic acid, sulfuric acid, phosphoric acid, nitric acid, and metaphosphoric acid. Organic acids include, but are not limited to, 1-hydroxy-2-naphthoic acid; 2,2-dichloroacetic acid; 2-hydroxyethanesulfonic acid; 2-oxoglutaric acid; 4-acetamidobenzoic acid; 4-aminosalicylic acid; acetic acid; adipic acid; ascorbic acid (L); aspartic acid (L); benzenesulfonic acid; benzoic acid; camphoric acid (+); camphor-10-sulfonic acid (+); capric acid (decanoic acid); caproic acid (hexanoic acid); caprylic acid (octanoic acid); carbonic acid; cinnamic acid; citric acid; cyclamic acid; dodecylsulfuric acid; ethane-1,2-disulfonic acid; ethanesulfonic acid; formic acid; fumaric acid; galactaric acid; gentisic acid; glucoheptonic acid (D); gluconic acid (D); glucuronic acid (D); glutamic acid; glutaric acid; glycerophosphoric acid; glycolic acid; hippuric acid; isobutyric acid; lactic acid (DL); lactobionic acid; lauric acid; maleic acid; malic acid (−L); malonic acid; mandelic acid (DL); methanesulfonic acid; naphthalene-1,5-disulfonic acid; naphthalene-2-sulfonic acid; nicotinic acid; oleic acid; oxalic acid; palmitic acid; pamoic acid; phosphoric acid; proprionic acid; pyroglutamic acid (−L); salicylic acid; sebacic acid; stearic acid; succinic acid; sulfuric acid; tartaric acid (+L); thiocyanic acid; toluenesulfonic acid (p); and undecylenic acid.

In some embodiments, pharmaceutically acceptable salts are obtained by reacting a compound of Formula (I-A), Formula (I-B), Formula (I-C), Formula (I-D), Formula (I-E), or Formula (I-F) with a base. In some embodiments, the compound of Formula (IA), Formula (I-B), Formula (I-C), Formula (I-D), Formula (I-E), or Formula (I-F) is acidic and is reacted with a base. In such situations, an acidic proton of the compound of Formula (I-A), Formula (I-B), Formula (I-C), Formula (I-D), Formula (I-E), or Formula (I-F) is replaced by a metal ion, e.g., lithium, sodium, potassium, magnesium, calcium, or an aluminum ion. In some cases, compounds described herein coordinate with an organic base, such as, but not limited to, ethanolamine, diethanolamine, triethanolamine, tromethamine, meglumine, N-methylglucamine, dicyclohexylamine, tris(hydroxymethyl)methylamine. In other cases, compounds described herein form salts with amino acids such as, but not limited to, arginine, lysine, and the like. Acceptable inorganic bases used to form salts with compounds that include an acidic proton, include, but are not limited to, aluminum hydroxide, calcium hydroxide, potassium hydroxide, sodium carbonate, potassium carbonate, sodium hydroxide, lithium hydroxide, and the like. In some embodiments, the compounds provided herein are prepared as a sodium salt, calcium salt, potassium salt, magnesium salt, meglumine salt, N-methylglucamine salt or ammonium salt.

It should be understood that a reference to a pharmaceutically acceptable salt includes the solvent addition forms. In some embodiments, solvates contain either stoichiometric or non-stoichiometric amounts of a solvent, and are formed during the process of crystallization with pharmaceutically acceptable solvents such as water, ethanol, and the like. Hydrates are formed when the solvent is water, or alcoholates are formed when the solvent is alcohol. Solvates of compounds described herein are conveniently prepared or formed during the processes described herein. In addition, the compounds provided herein optionally exist in unsolvated as well as solvated forms.

The methods and formulations described herein include the use of N-oxides (if appropriate), or pharmaceutically acceptable salts of compounds having the structure of Formula (I-A), Formula (I-B), Formula (I-C), Formula (I-D), Formula (I-E), or Formula (I-F), as well as active metabolites of these compounds having the same type of activity.

In some embodiments, sites on the organic radicals (e.g. alkyl groups, aromatic rings) of compounds of Formula (I-A), Formula (I-B), Formula (I-C), Formula (I-D), Formula (I-E), or Formula (I-F) are susceptible to various metabolic reactions. Incorporation of appropriate substituents on the organic radicals will reduce, minimize or eliminate this metabolic pathway. In specific embodiments, the appropriate substituent to decrease or eliminate the susceptibility of the aromatic ring to metabolic reactions is, by way of example only, a halogen, deuterium, an alkyl group, a haloalkyl group, or a deuteroalkyl group.

In another embodiment, the compounds described herein are labeled isotopically (e.g. with a radioisotope) or by another other means, including, but not limited to, the use of chromophores or fluorescent moieties, bioluminescent labels, or chemiluminescent labels.

Compounds described herein include isotopically-labeled compounds, which are identical to those recited in the various formulae and structures presented herein, but for the fact that one or more atoms are replaced by an atom having an atomic mass or mass number different from the atomic mass or mass number usually found in nature. Examples of isotopes that can be incorporated into the present compounds include isotopes of hydrogen, carbon, nitrogen, oxygen, sulfur, fluorine chlorine, iodine, phosphorus, such as, for example, ²H, ³H, ¹³C, ¹⁴C, ¹⁵N, ¹⁸O, ¹⁷O, ³⁵S, ¹⁸F, ³⁶Cl, ¹²³I, ¹²⁴I, ¹²⁵I, ¹³¹I, ³²P and ³³P. In one aspect, isotopically-labeled compounds described herein, for example those into which radioactive isotopes such as ³H and ¹⁴C are incorporated, are useful in drug and/or substrate tissue distribution assays. In one aspect, substitution with isotopes such as deuterium affords certain therapeutic advantages resulting from greater metabolic stability, such as, for example, increased in vivo half-life or reduced dosage requirements. In some embodiments, one or more hydrogens of the compounds of Formula (I-A), Formula (I-B), Formula (I-C), Formula (I-D), Formula (I-E), or Formula (I-F) are replaced with deuterium.

In some embodiments, the compounds of Formula (I-A), Formula (I-B), Formula (I-C), Formula (I-D), Formula (I-E), or Formula (I-F) possess one or more stereocenters and each stereocenter exists independently in either the R or S configuration. In some embodiments, the compound of Formula (I-A), Formula (I-B), Formula (I-C), Formula (I-D), Formula (I-E), or Formula (I-F) exists in the R configuration. In some embodiments, the compound of Formula (I-A), Formula (I-B), Formula (I-C), Formula (I-D), Formula (I-E), or Formula (I-F) exists in the S configuration. The compounds presented herein include all diastereomeric, individual enantiomers, atropisomers, and epimeric forms as well as the appropriate mixtures thereof. The compounds and methods provided herein include all cis, trans, syn, anti, entgegen (E), and zusammen (Z) isomers as well as the appropriate mixtures thereof.

Individual stereoisomers are obtained, if desired, by methods such as, stereoselective synthesis and/or the separation of stereoisomers by chiral chromatographic columns or the separation of diastereomers by either non-chiral or chiral chromatographic columns or crystallization and recrystallization in a proper solvent or a mixture of solvents. In certain embodiments, compounds of Formula (I-A), Formula (I-B), Formula (I-C), Formula (I-D), Formula (I-E), or Formula (I-F) are prepared as their individual stereoisomers by reacting a racemic mixture of the compound with an optically active resolving agent to form a pair of diastereoisomeric compounds/salts, separating the diastereomers and recovering the optically pure individual enantiomers. In some embodiments, resolution of individual enantiomers is carried out using covalent diastereomeric derivatives of the compounds described herein. In another embodiment, diastereomers are separated by separation/resolution techniques based upon differences in solubility. In other embodiments, separation of stereoisomers is performed by chromatography or by the forming diastereomeric salts and separation by recrystallization, or chromatography, or any combination thereof. Jean Jacques, Andre Collet, Samuel H. Wilen, “Enantiomers, Racemates and Resolutions”, John Wiley and Sons, Inc., 1981. In some embodiments, stereoisomers are obtained by stereoselective synthesis.

In some embodiments, compounds described herein are prepared as prodrugs. A “prodrug” refers to an agent that is converted into the parent drug in vivo. Prodrugs are often useful because, in some situations, they are easier to administer than the parent drug. They are, for instance, bioavailable by oral administration whereas the parent is not. Further or alternatively, the prodrug also has improved solubility in pharmaceutical compositions over the parent drug. In some embodiments, the design of a prodrug increases the effective water solubility. An example, without limitation, of a prodrug is a compound described herein, which is administered as an ester (the “prodrug”) but then is metabolically hydrolyzed to provide the active entity. A further example of a prodrug is a short peptide (polyaminoacid) bonded to an acid group where the peptide is metabolized to reveal the active moiety. In certain embodiments, upon in vivo administration, a prodrug is chemically converted to the biologically, pharmaceutically or therapeutically active form of the compound. In certain embodiments, a prodrug is enzymatically metabolized by one or more steps or processes to the biologically, pharmaceutically or therapeutically active form of the compound.

Prodrugs of the compounds described herein include, but are not limited to, esters, ethers, carbonates, thiocarbonates, N-acyl derivatives, N-acyloxyalkyl derivatives, N-alkyloxyacyl derivatives, quaternary derivatives of tertiary amines, N-Mannich bases, Schiff bases, amino acid conjugates, phosphate esters, and sulfonate esters. See for example Design of Prodrugs, Bundgaard, A. Ed., Elseview, 1985 and Method in Enzymology, Widder, K. et al., Ed.; Academic, 1985, vol. 42, p. 309-396; Bundgaard, H. “Design and Application of Prodrugs” in A Textbook of Drug Design and Development, Krosgaard-Larsen and H. Bundgaard, Ed., 1991, Chapter 5, p. 113-191; and Bundgaard, H., Advanced Drug Delivery Review, 1992, 8, 1-38, each of which is incorporated herein by reference. In some embodiments, a hydroxyl group in the compounds disclosed herein is used to form a prodrug, wherein the hydroxyl group is incorporated into an acyloxyalkyl ester, alkoxycarbonyloxyalkyl ester, alkyl ester, aryl ester, phosphate ester, sugar ester, ether, and the like. In some embodiments, a hydroxyl group in the compounds disclosed herein is a prodrug wherein the hydroxyl is then metabolized in vivo to provide a carboxylic acid group. In some embodiments, a carboxyl group is used to provide an ester or amide (i.e. the prodrug), which is then metabolized in vivo to provide a carboxylic acid group. In some embodiments, compounds described herein are prepared as alkyl ester prodrugs.

Prodrug forms of the herein described compounds, wherein the prodrug is metabolized in vivo to produce a compound of Formula (I-A), Formula (I-B), Formula (I-C), Formula (I-D), Formula (I-E), or Formula (I-F) as set forth herein are included within the scope of the claims. In some cases, some of the herein-described compounds is a prodrug for another derivative or active compound.

In some embodiments, any one of the hydroxyl group(s), amino group(s) and/or carboxylic acid group(s) are functionalized in a suitable manner to provide a prodrug moiety. In some embodiments, the prodrug moiety is as described above.

In additional or further embodiments, the compounds described herein are metabolized upon administration to an organism in need to produce a metabolite that is then used to produce a desired effect, including a desired therapeutic effect.

A “metabolite” of a compound disclosed herein is a derivative of that compound that is formed when the compound is metabolized. The term “active metabolite” refers to a biologically active derivative of a compound that is formed when the compound is metabolized. The term “metabolized,” as used herein, refers to the sum of the processes (including, but not limited to, hydrolysis reactions and reactions catalyzed by enzymes) by which a particular substance is changed by an organism. Thus, enzymes may produce specific structural alterations to a compound. For example, cytochrome P450 catalyzes a variety of oxidative and reductive reactions while uridine diphosphate glucuronyltransferases catalyze the transfer of an activated glucuronic-acid molecule to aromatic alcohols, aliphatic alcohols, carboxylic acids, amines and free sulphydryl groups. Metabolites of the compounds disclosed herein are optionally identified either by administration of compounds to a host and analysis of tissue samples from the host, or by incubation of compounds with hepatic cells in vitro and analysis of the resulting compounds.

### Synthesis of Compounds

Compounds of Formula (I-A), Formula (I-B), Formula (I-C), Formula (I-D), Formula (I-E), or Formula (I-F) described herein are synthesized using standard synthetic techniques or using methods known in the art in combination with methods described herein.

Unless otherwise indicated, conventional methods of mass spectroscopy, NMR, HPLC, protein chemistry, biochemistry, recombinant DNA techniques and pharmacology are employed.

Compounds are prepared using standard organic chemistry techniques such as those described in, for example, March's Advanced Organic Chemistry, 6^{th} Edition, John Wiley and Sons, Inc. Alternative reaction conditions for the synthetic transformations described herein may be employed such as variation of solvent, reaction temperature, reaction time, as well as different chemical reagents and other reaction conditions.

In some embodiments, compounds described herein are synthesized as outlined in the Examples.

### Certain Terminology

Unless otherwise stated, the following terms used in this application have the definitions given below. The use of the term “including” as well as other forms, such as “include”, “includes,” and “included,” is not limiting. The section headings used herein are for organizational purposes only and are not to be construed as limiting the subject matter described.

As used herein, C₁-Cₓ includes C₁-C₂, C₁-C₃ . . . C₁-Cₓ. By way of example only, a group designated as “C₁-C₄” indicates that there are one to four carbon atoms in the moiety, i.e. groups containing 1 carbon atom, 2 carbon atoms, 3 carbon atoms or 4 carbon atoms. Thus, by way of example only, “C₁-C₄ alkyl” indicates that there are one to four carbon atoms in the alkyl group, i.e., the alkyl group is selected from among methyl, ethyl, propyl, iso-propyl, n-butyl, iso-butyl, sec-butyl, and t-butyl.

An “alkyl” group refers to an aliphatic hydrocarbon group. The alkyl group is branched or straight chain. In some embodiments, the “alkyl” group has 1 to 10 carbon atoms, i.e. a C₁-C₁₀alkyl. Whenever it appears herein, a numerical range such as “1 to 10” refers to each integer in the given range; e.g., “1 to 10 carbon atoms” means that the alkyl group consist of 1 carbon atom, 2 carbon atoms, 3 carbon atoms, etc., up to and including 10 carbon atoms, although the present definition also covers the occurrence of the term “alkyl” where no numerical range is designated. In some embodiments, an alkyl is a C₁-C₆alkyl. In one aspect the alkyl is methyl, ethyl, propyl, iso-propyl, n-butyl, iso-butyl, sec-butyl, or t-butyl. Typical alkyl groups include, but are in no way limited to, methyl, ethyl, propyl, isopropyl, butyl, isobutyl, sec-butyl, tertiary butyl, pentyl, neopentyl, or hexyl.

An “alkylene” group refers to a divalent alkyl radical. Any of the above mentioned monovalent alkyl groups may be an alkylene by abstraction of a second hydrogen atom from the alkyl. In some embodiments, an alkylene is a C₁-C₆alkylene. In other embodiments, an alkylene is a C₁-C₄alkylene. Typical alkylene groups include, but are not limited to, —CH₂—, —CH(CH₃)—, —C(CH₃)₂—, —CH₂CH₂—, —CH₂CH(CH₃)—, —CH₂C(CH₃)₂—, —CH₂CH₂CH₂—, —CH₂CH₂CH₂CH₂—, and the like.

The term “alkenyl” refers to a type of alkyl group in which at least one carbon-carbon double bond is present. In one embodiment, an alkenyl group has the formula —C(R)═CR₂, wherein R refers to the remaining portions of the alkenyl group, which may be the same or different. In some embodiments, R is H or an alkyl. Non-limiting examples of an alkenyl group include —CH═CH₂, —C(CH₃)═CH₂, —CH═CHCH₃, —C(CH₃)═CHCH₃, and —CH₂CH═CH₂.

The term “alkynyl” refers to a type of alkyl group in which at least one carbon-carbon triple bond is present. In one embodiment, an alkenyl group has the formula —C≡C—R, wherein R refers to the remaining portions of the alkynyl group. In some embodiments, R is H or an alkyl. Non-limiting examples of an alkynyl group include —C≡CH, —C≡CCH₃—C≡CCH₂CH₃, —CH₂C≡CH.

An “alkoxy” group refers to a (alkyl)O— group, where alkyl is as defined herein.

The term “alkylamine” refers to —NH(alkyl), or —N(alkyl)₂.

The term “aromatic” refers to a planar ring having a delocalized π-electron system containing 4n+2π electrons, where n is an integer. The term “aromatic” includes both carbocyclic aryl (“aryl”, e.g., phenyl) and heterocyclic aryl (or “heteroaryl” or “heteroaromatic”) groups (e.g., pyridine). The term includes monocyclic or fused-ring polycyclic (i.e., rings which share adjacent pairs of carbon atoms) groups.

The term “carbocyclic” or “carbocycle” refers to a ring or ring system where the atoms forming the backbone of the ring are all carbon atoms. The term thus distinguishes carbocyclic from “heterocyclic” rings or “heterocycles” in which the ring backbone contains at least one atom which is different from carbon. In some embodiments, at least one of the two rings of a bicyclic carbocycle is aromatic. In some embodiments, both rings of a bicyclic carbocycle are aromatic.

As used herein, the term “aryl” refers to an aromatic ring wherein each of the atoms forming the ring is a carbon atom. In one aspect, aryl is phenyl or a naphthyl. In some embodiments, an aryl is a phenyl. In some embodiments, an aryl is a C₆-C₁₀aryl. Depending on the structure, an aryl group is a monoradical or a diradical (i.e., an arylene group).

The term “cycloalkyl” refers to a monocyclic or polycyclic aliphatic, non-aromatic radical, wherein each of the atoms forming the ring (i.e. skeletal atoms) is a carbon atom. In some embodiments, cycloalkyls are spirocyclic or bridged compounds. In some embodiments, cycloalkyls are optionally fused with an aromatic ring, and the point of attachment is at a carbon that is not an aromatic ring carbon atom. Cycloalkyl groups include groups having from 3 to 10 ring atoms. In some embodiments, cycloalkyl groups are selected from among cyclopropyl, cyclobutyl, cyclopentyl, cyclopentenyl, cyclohexyl, cyclohexenyl, cycloheptyl, cyclooctyl, spiro[2.2]pentyl, norbornyl and bicycle[1.1.1]pentyl, bicyclo[3.3.0]octane, bicyclo[4.3.0]nonane, cis-decalin, trans-decalin, bicyclo[2.1.1]hexane, bicyclo[2.2.1]heptane, bicyclo[2.2.2]octane, bicyclo[3.2.2]nonane, and bicyclo[3.3.2]decane, adamantyl, norbornyl, and decalinyl. In some embodiments, a cycloalkyl is a C₃-C₆cycloalkyl.

“Deuteroalkyl” refers to an alkyl group as defined herein, in which at least one H is replaced by an isotope of hydrogen, i.e., by deuterium (²H) or tritium (³H).

“Deuteroalkoxy” refers to an alkoxy group as defined herein, in which at least one H is replaced by an isotope of hydrogen, i.e., by deuterium (²H) or tritium (³H).

The term “halo” or, alternatively, “halogen” or “halide” means fluoro, chloro, bromo or iodo. In some embodiments, halo is fluoro, chloro, or bromo.

The term “fluoroalkyl” refers to an alkyl in which one or more hydrogen atoms are replaced by a fluorine atom. In one aspect, a fluoroalkyl is a C₁-C₆fluoroalkyl.

“Fluoroalkoxy” refers to an alkoxy group as defined herein, in which at least one H is replaced by a fluorine atom.

The term “heteroalkyl” refers to an alkyl group in which one or more skeletal atoms of the alkyl are selected from an atom other than carbon, e.g., oxygen, nitrogen (e.g. —NH—, —N(alkyl)-, sulfur, or combinations thereof. A heteroalkyl is attached to the rest of the molecule at a carbon atom of the heteroalkyl. In one aspect, a heteroalkyl is a C₁-C₆heteroalkyl.

Examples of such heteroalkyl are, for example, —CH₂OCH₃, —CH₂CH₂OCH₃, —CH₂CH₂OCH₂CH₂OCH₃, —CH(CH₃)OCH₃, —CH₂NHCH₃, —CH₂N(CH₃)₂, and —CH₂SCH₃.

The term “heterocycle” or “heterocyclic” refers to heteroaromatic rings (also known as heteroaryls) and heterocycloalkyl rings (also known as heteroalicyclic groups) containing one to four heteroatoms in the ring(s), where each heteroatom in the ring(s) is selected from O, S and N, wherein each heterocyclic group has from 3 to 10 atoms in its ring system, and with the proviso that any ring does not contain two adjacent O or S atoms. Non-aromatic heterocyclic groups (also known as heterocycloalkyls) include rings having 3 to 10 atoms in its ring system and aromatic heterocyclic groups include rings having 5 to 10 atoms in its ring system. The heterocyclic groups include benzo-fused ring systems. Examples of non-aromatic heterocyclic groups are pyrrolidinyl, tetrahydrofuranyl, dihydrofuranyl, tetrahydrothienyl, oxazolidinonyl, tetrahydropyranyl, dihydropyranyl, tetrahydrothiopyranyl, piperidinyl, morpholinyl, thiomorpholinyl, thioxanyl, piperazinyl, aziridinyl, azetidinyl, oxetanyl, thietanyl, homopiperidinyl, oxepanyl, thiepanyl, oxazepinyl, diazepinyl, thiazepinyl, 1,2,3,6-tetrahydropyridinyl, pyrrolin-2-yl, pyrrolin-3-yl, indolinyl, 2H-pyranyl, 4H-pyranyl, dioxanyl, 1,3-dioxolanyl, pyrazolinyl, dithianyl, dithiolanyl, dihydropyranyl, dihydrothienyl, dihydrofuranyl, pyrazolidinyl, imidazolinyl, imidazolidinyl, 3-azabicyclo[3.1.0]hexanyl, 3-azabicyclo[4.1.0]heptanyl, 3H-indolyl, indolin-2-onyl, isoindolin-1-onyl, isoindoline-1,3-dionyl, 3,4-dihydroisoquinolin-1(2H)-onyl, 3,4-dihydroquinolin-2(1H)-onyl, isoindoline-1,3-dithionyl, benzo[d]oxazol-2(3H)-onyl, 1H-benzo[d]imidazol-2(3H)-onyl, benzo[d]thiazol-2(3H)-onyl, and quinolizinyl. Examples of aromatic heterocyclic groups are pyridinyl, imidazolyl, pyrimidinyl, pyrazolyl, triazolyl, pyrazinyl, tetrazolyl, furyl, thienyl, isoxazolyl, thiazolyl, oxazolyl, isothiazolyl, pyrrolyl, quinolinyl, isoquinolinyl, indolyl, benzimidazolyl, benzofuranyl, cinnolinyl, indazolyl, indolizinyl, phthalazinyl, pyridazinyl, triazinyl, isoindolyl, pteridinyl, purinyl, oxadiazolyl, thiadiazolyl, furazanyl, benzofurazanyl, benzothiophenyl, benzothiazolyl, benzoxazolyl, quinazolinyl, quinoxalinyl, naphthyridinyl, and furopyridinyl. The foregoing groups are either C-attached (or C-linked) or N-attached where such is possible. For instance, a group derived from pyrrole includes both pyrrol-1-yl (N-attached) or pyrrol-3-yl (C-attached). Further, a group derived from imidazole includes imidazol-1-yl or imidazol-3-yl (both N-attached) or imidazol-2-yl, imidazol-4-yl or imidazol-5-yl (all C-attached). The heterocyclic groups include benzo-fused ring systems. Non-aromatic heterocycles are optionally substituted with one or two oxo (═O) moieties, such as pyrrolidin-2-one. In some embodiments, at least one of the two rings of a bicyclic heterocycle is aromatic. In some embodiments, both rings of a bicyclic heterocycle are aromatic.

The terms “heteroaryl” or, alternatively, “heteroaromatic” refers to an aryl group that includes one or more ring heteroatoms selected from nitrogen, oxygen and sulfur. Illustrative examples of heteroaryl groups include monocyclic heteroaryls and bicyclic heteroaryls. Monocyclic heteroaryls include pyridinyl, imidazolyl, pyrimidinyl, pyrazolyl, triazolyl, pyrazinyl, tetrazolyl, furyl, thienyl, isoxazolyl, thiazolyl, oxazolyl, isothiazolyl, pyrrolyl, pyridazinyl, triazinyl, oxadiazolyl, thiadiazolyl, and furazanyl. Bicyclic heteroaryls include indolizine, indole, benzofuran, benzothiophene, indazole, benzimidazole, purine, quinolizine, quinoline, isoquinoline, cinnoline, phthalazine, quinazoline, quinoxaline, 1,8-naphthyridine, and pteridine. In some embodiments, a heteroaryl contains 0-4N atoms in the ring. In some embodiments, a heteroaryl contains 1-4N atoms in the ring. In some embodiments, a heteroaryl contains 0-4N atoms, 0-1 O atoms, and 0-1 S atoms in the ring. In some embodiments, a heteroaryl contains 1-4N atoms, 0-1 O atoms, and 0-1 S atoms in the ring. In some embodiments, heteroaryl is a C₁-C₉heteroaryl. In some embodiments, monocyclic heteroaryl is a C₁-C₅heteroaryl. In some embodiments, monocyclic heteroaryl is a 5-membered or 6-membered heteroaryl. In some embodiments, bicyclic heteroaryl is a C₆-C₉heteroaryl.

A “heterocycloalkyl” or “heteroalicyclic” group refers to a cycloalkyl group that includes at least one heteroatom selected from nitrogen, oxygen and sulfur. In some embodiments, a heterocycloalkyl is fused with an aryl or heteroaryl. In some embodiments, the heterocycloalkyl is oxazolidinonyl, pyrrolidinyl, tetrahydrofuranyl, tetrahydrothienyl, tetrahydropyranyl, tetrahydrothiopyranyl, piperidinyl, morpholinyl, thiomorpholinyl, piperazinyl, piperidin-2-onyl, pyrrolidine-2,5-dithionyl, pyrrolidine-2,5-dionyl, pyrrolidinonyl, imidazolidinyl, imidazolidin-2-onyl, or thiazolidin-2-onyl. The term heteroalicyclic also includes all ring forms of the carbohydrates, including but not limited to the monosaccharides, the disaccharides and the oligosaccharides. In one aspect, a heterocycloalkyl is a C₂-C₁₀heterocycloalkyl. In another aspect, a heterocycloalkyl is a C₄-C₁₀heterocycloalkyl. In some embodiments, a heterocycloalkyl contains 0-2N atoms in the ring. In some embodiments, a heterocycloalkyl contains 0-2N atoms, 0-2O atoms and 0-1S atoms in the ring.

The term “bond” or “single bond” refers to a chemical bond between two atoms, or two moieties when the atoms joined by the bond are considered to be part of larger substructure. In one aspect, when a group described herein is a bond, the referenced group is absent thereby allowing a bond to be formed between the remaining identified groups.

The term “moiety” refers to a specific segment or functional group of a molecule. Chemical moieties are often recognized chemical entities embedded in or appended to a molecule.

The term “optionally substituted” or “substituted” means that the referenced group is optionally substituted with one or more additional group(s) individually and independently selected from halogen, —CN, —NH₂, —NH(alkyl), —N(alkyl)₂, —OH, —CO₂H, —CO₂alkyl, —C(═O)NH₂, —C(═O)NH(alkyl), —C(═O)N(alkyl)₂, —S(═O)₂NH₂, —S(═O)₂NH(alkyl), —S(═O)₂N(alkyl)₂, alkyl, cycloalkyl, fluoroalkyl, heteroalkyl, alkoxy, fluoroalkoxy, heterocycloalkyl, aryl, heteroaryl, aryloxy, alkylthio, arylthio, alkylsulfoxide, arylsulfoxide, alkylsulfone, and arylsulfone. In some other embodiments, optional substituents are independently selected from halogen, —CN, —NH₂, —NH(CH₃), —N(CH₃)₂, —OH, —CO₂H, —CO₂(C₁-C₄alkyl), —C(═O)NH₂, —C(═O)NH(C₁-C₄alkyl), —C(═O)N(C₁-C₄alkyl)₂, —S(═O)₂NH₂, —S(═O)₂NH(C₁-C₄alkyl), —S(═O)₂N(C₁-C₄alkyl)₂, C₁-C₄alkyl, C₃-C₆cycloalkyl, C₁-C₄fluoroalkyl, C₁-C₄heteroalkyl, C₁-C₄alkoxy, C₁-C₄fluoroalkoxy, —SC₁-C₄alkyl, —S(═O)C₁-C₄alkyl, and —S(═O)₂C₁-C₄alkyl. In some embodiments, optional substituents are independently selected from halogen, —CN, —NH₂, —OH, —NH(CH₃), —N(CH₃)₂, —CH₃, —CH₂CH₃, —CF₃, —OCH₃, and —OCF₃. In some embodiments, substituted groups are substituted with one or two of the preceding groups. In some embodiments, an optional substituent on an aliphatic carbon atom (acyclic or cyclic) includes oxo (═O).

The term “acceptable” with respect to a formulation, composition or ingredient, as used herein, means having no persistent detrimental effect on the general health of the subject being treated.

The term “modulate” as used herein, means to interact with a target either directly or indirectly so as to alter the activity of the target, including, by way of example only, to enhance the activity of the target, to inhibit the activity of the target, to limit the activity of the target, or to extend the activity of the target. In some embodiments, “modulate” means to interact with a target either directly or indirectly so as to decrease or inhibit receptor activity,

The term “modulator” as used herein, refers to a molecule that interacts with a target either directly or indirectly. The interactions include, but are not limited to, the interactions of an agonist, partial agonist, an inverse agonist, antagonist, or combinations thereof. In some embodiments, a modulator is an antagonist. Receptor antagonists are inhibitors of receptor activity. Antagonists mimic ligands that bind to a receptor and prevent receptor activation by a natural ligand. Preventing activation may have many effects. If a natural agonist binding to a receptor leads to an increase in cellular function, an antagonist that binds and blocks this receptor decreases the function.

The terms “administer,” “administering”, “administration,” and the like, as used herein, refer to the methods that may be used to enable delivery of compounds or compositions to the desired site of biological action. These methods include, but are not limited to oral routes, intraduodenal routes, parenteral injection (including intravenous, subcutaneous, intraperitoneal, intramuscular, intravascular or infusion), topical and rectal administration. Those of skill in the art are familiar with administration techniques that can be employed with the compounds and methods described herein. In some embodiments, the compounds and compositions described herein are administered orally.

The terms “co-administration” or the like, as used herein, are meant to encompass administration of the selected therapeutic agents to a single patient, and are intended to include treatment regimens in which the agents are administered by the same or different route of administration or at the same or different time.

The terms “effective amount” or “therapeutically effective amount,” as used herein, refer to a sufficient amount of an agent or a compound being administered, which will relieve to some extent one or more of the symptoms of the disease or condition being treated. The result includes reduction and/or alleviation of the signs, symptoms, or causes of a disease, or any other desired alteration of a biological system. For example, an “effective amount” for therapeutic uses is the amount of the composition comprising a compound as disclosed herein required to provide a clinically significant decrease in disease symptoms. An appropriate “effective” amount in any individual case is optionally determined using techniques, such as a dose escalation study.

The terms “enhance” or “enhancing,” as used herein, means to increase or prolong either in potency or duration a desired effect. Thus, in regard to enhancing the effect of therapeutic agents, the term “enhancing” refers to the ability to increase or prolong, either in potency or duration, the effect of other therapeutic agents on a system. An “enhancing-effective amount,” as used herein, refers to an amount adequate to enhance the effect of another therapeutic agent in a desired system.

The terms “kit” and “article of manufacture” are used as synonyms.

The term “subject” or “patient” encompasses mammals. Examples of mammals include, but are not limited to, any member of the Mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. In one aspect, the mammal is a human.

The terms “treat,” “treating” or “treatment,” as used herein, include alleviating, abating or ameliorating at least one symptom of a disease or condition, preventing additional symptoms, inhibiting the disease or condition, e.g., arresting the development of the disease or condition, relieving the disease or condition, causing regression of the disease or condition, relieving a condition caused by the disease or condition, or stopping the symptoms of the disease or condition either prophylactically and/or therapeutically.

### ABBREVIATIONS

### Abbreviation Meaning

δ chemical shift (ppm)
2-MeTHF 2-methyltetrahydrofuran
Ac acetate
aq aqueous
Boc tert-butoxycarbonyl
CDCl₃ deuterated chloroform
Cpd compound
DCE 1,2-dichloroethane
DCM dichloromethane
DIAD diisopropyl azodicarboxylate
DIEA N,N-diisopropylethylamine
DMAP 4-dimethylaminopyridine
DMF N,N-dimethylformamide
DMP Dess-Martin periodinane
DMSO-d₆ deuterated dimethyl sulfoxide
ee enantiomeric excess
eq equivalent(s)
ESI electrospray ionization
EtOAc or EA ethyl acetate
¹H NMR proton nuclear magnetic resonance spectroscopy
h. or hr. hour(s)
HATU 1-[bis(dimethylamino)methylene]-1H-1,2,3-triazolo[4,5-b]pyridinium 3-oxide hexafluorophosphate
HOAc acetic acid
HPLC high performance liquid chromatography
LCMS liquid chromatography—mass spectrometry
M molar
m/z mass to charge ratio
Me methyl
min. minute(s)
MTBE methyl tert-butyl ether
N normal
Pd₂dba₃ tris(dibenzylideneacetone)dipalladium(O)
prep preparatory
RuPhos Pd G3 (2-dicyclohexylphosphino-2′,6′-diisopropoxy-1,1′-biphenyl)[2-(2′-amino-1,1′-biphenyl)]palladium(II) methanesulfonate
SFC supercritical fluid chromatography
T3P propanephosphonic acid anhydride
TBAF tetra-n-butylammonium fluoride
TBP tributyl phosphine
tBuMePhos 2-di-tert-butylphosphino-2′-methylbiphenyl
tBuONa sodium tert-butoxide
TEA triethylamine
TEMPO (2,2,6,6-tetramethylpiperidin-1-yl)oxyl
TFA trifluoroacetic acid
THE tetrahydrofuran
TLC thin layer chromatography
TosCl 4-toluenesulfonyl chloride

### Pharmaceutical Compositions

In some embodiments, the compounds described herein are formulated into pharmaceutical compositions. Pharmaceutical compositions are formulated in a conventional manner using one or more pharmaceutically acceptable inactive ingredients that facilitate processing of the active compounds into preparations that are used pharmaceutically. Proper formulation is dependent upon the route of administration chosen. A summary of pharmaceutical compositions described herein is found, for example, in Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pa. 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999), herein incorporated by reference for such disclosure.

In some embodiments, the compounds described herein are administered either alone or in combination with pharmaceutically acceptable carriers, excipients or diluents, in a pharmaceutical composition. Administration of the compounds and compositions described herein can be effected by any method that enables delivery of the compounds to the site of action. These methods include, though are not limited to delivery via enteral routes (including oral, gastric or duodenal feeding tube, rectal suppository and rectal enema), parenteral routes (injection or infusion, including intraarterial, intracardiac, intradermal, intraduodenal, intramedullary, intramuscular, intraosseous, intraperitoneal, intrathecal, intravascular, intravenous, intravitreal, epidural and subcutaneous), inhalational, transdermal, transmucosal, sublingual, buccal and topical (including epicutaneous, dermal, enema, eye drops, ear drops, intranasal, vaginal) administration, although the most suitable route may depend upon for example the condition and disorder of the recipient. By way of example only, compounds described herein can be administered locally to the area in need of treatment, by for example, local infusion during surgery, topical application such as creams or ointments, injection, catheter, or implant. The administration can also be by direct injection at the site of a diseased tissue or organ.

In some embodiments, pharmaceutical compositions suitable for oral administration are presented as discrete units such as capsules, cachets or tablets each containing a predetermined amount of the active ingredient; as a powder or granules; as a solution or a suspension in an aqueous liquid or a non-aqueous liquid; or as an oil-in-water liquid emulsion or a water-in-oil liquid emulsion. In some embodiments, the active ingredient is presented as a bolus, electuary or paste.

Pharmaceutical compositions which can be used orally include tablets, push-fit capsules made of gelatin, as well as soft, sealed capsules made of gelatin and a plasticizer, such as glycerol or sorbitol. Tablets may be made by compression or molding, optionally with one or more accessory ingredients. Compressed tablets may be prepared by compressing in a suitable machine the active ingredient in a free-flowing form such as a powder or granules, optionally mixed with binders, inert diluents, or lubricating, surface active or dispersing agents. Molded tablets may be made by molding in a suitable machine a mixture of the powdered compound moistened with an inert liquid diluent. In some embodiments, the tablets are coated or scored and are formulated so as to provide slow or controlled release of the active ingredient therein. All formulations for oral administration should be in dosages suitable for such administration. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In some embodiments, stabilizers are added. Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or Dragee coatings for identification or to characterize different combinations of active compound doses.

In some embodiments, pharmaceutical compositions are formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents. The compositions may be presented in unit-dose or multi-dose containers, for example sealed ampoules and vials, and may be stored in powder form or in a freeze-dried (lyophilized) condition requiring only the addition of the sterile liquid carrier, for example, saline or sterile pyrogen-free water, immediately prior to use. Extemporaneous injection solutions and suspensions may be prepared from sterile powders, granules and tablets of the kind previously described.

Pharmaceutical compositions for parenteral administration include aqueous and non-aqueous (oily) sterile injection solutions of the active compounds which may contain antioxidants, buffers, bacteriostats and solutes which render the formulation isotonic with the blood of the intended recipient; and aqueous and non-aqueous sterile suspensions which may include suspending agents and thickening agents. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions.

It should be understood that in addition to the ingredients particularly mentioned above, the compounds and compositions described herein may include other agents conventional in the art having regard to the type of formulation in question, for example those suitable for oral administration may include flavoring agents.

### Methods of Treatment, Dosing and Treatment Regimens

The compounds disclosed herein, or pharmaceutically acceptable salts, solvates, or stereoisomers thereof, are useful for the modulation of monocarboxylic acid transporters. In some embodiments, the monocarboxylic acid transporter modulated by the compounds and methods is the monocarboxylic acid transporter 4 (MCT4).

Provided herein are MCT4 modulators that are useful for treating one or more diseases or disorders associated with or would benefit from modulation of MCT4 activity. In some embodiments, described herein are methods for treating a disease or disorder, wherein the disease or disorder is cancer, a hyperproliferative disorder, an autoimmune disorder, or inflammatory disorder.

In one embodiment, the compounds described herein, or a pharmaceutically acceptable salt thereof, are used in the preparation of medicaments for the treatment of diseases or conditions in a mammal that would benefit from inhibition or reduction of MCT4 activity. Methods for treating any of the diseases or conditions described herein in a mammal in need of such treatment, involves administration of pharmaceutical compositions that include at least one compound described herein or a pharmaceutically acceptable salt, active metabolite, prodrug, or pharmaceutically acceptable solvate thereof, in therapeutically effective amounts to said mammal.

In certain embodiments, the compositions containing the compound(s) described herein are administered for prophylactic and/or therapeutic treatments. In certain therapeutic applications, the compositions are administered to a mammal already suffering from a disease or condition, in an amount sufficient to cure or at least partially arrest at least one of the symptoms of the disease or condition. Amounts effective for this use depend on the severity and course of the disease or condition, previous therapy, the mammal's health status, weight, and response to the drugs, and the judgment of a healthcare practitioner. Therapeutically effective amounts are optionally determined by methods including, but not limited to, a dose escalation and/or dose ranging clinical trial.

In prophylactic applications, compositions containing the compounds described herein are administered to a mammal susceptible to or otherwise at risk of a particular disease, disorder or condition. Such an amount is defined to be a “prophylactically effective amount or dose.” In this use, the precise amounts also depend on the mammal's state of health, weight, and the like. When used in mammals, effective amounts for this use will depend on the severity and course of the disease, disorder or condition, previous therapy, the mammal's health status and response to the drugs, and the judgment of a healthcare professional. In one aspect, prophylactic treatments include administering to a mammal, who previously experienced at least one symptom of the disease being treated and is currently in remission, a pharmaceutical composition comprising a compound described herein, or a pharmaceutically acceptable salt thereof, in order to prevent a return of the symptoms of the disease or condition.

In certain embodiments wherein the mammal's condition does not improve, upon the discretion of a healthcare professional the administration of the compounds are administered chronically, that is, for an extended period of time, including throughout the duration of the mammal's life in order to ameliorate or otherwise control or limit the symptoms of the mammal's disease or condition.

In certain embodiments wherein a mammal's status does improve, the dose of drug being administered is temporarily reduced or temporarily suspended for a certain length of time (i.e., a “drug holiday”). In specific embodiments, the length of the drug holiday is between 2 days and 1 year, including by way of example only, 2 days, 3 days, 4 days, 5 days, 6 days, 7 days, 10 days, 12 days, 15 days, 20 days, 28 days, or more than 28 days. The dose reduction during a drug holiday is, by way of example only, by 10%-100%, including by way of example only 10%, 15%, 20%, 25%, 30%, 35%, 40%, 45%, 50%, 55%, 60%, 65%, 70%, 75%, 80%, 85%, 90%, 95%, and 100%.

Once improvement of the patient's conditions has occurred, a maintenance dose is administered if necessary. Subsequently, in specific embodiments, the dosage or the frequency of administration, or both, is reduced, as a function of the symptoms, to a level at which the improved disease, disorder or condition is retained. In certain embodiments, however, the mammal requires intermittent treatment on a long-term basis upon any recurrence of symptoms.

The amount of a given agent that corresponds to such an amount varies depending upon factors such as the particular compound, disease condition and its severity, the identity (e.g., weight, sex) of the subject or host in need of treatment, but nevertheless is determined according to the particular circumstances surrounding the case, including, e.g., the specific agent being administered, the route of administration, the condition being treated, and the subject or host being treated.

In general, however, doses employed for adult human treatment are typically in the range of 0.01 mg-5000 mg per day. In one aspect, doses employed for adult human treatment are from about 1 mg to about 1000 mg per day. In one embodiment, the desired dose is conveniently presented in a single dose or in divided doses administered simultaneously or at appropriate intervals, for example as two, three, four or more sub-doses per day.

In one embodiment, the daily dosages appropriate for the compound described herein, or a pharmaceutically acceptable salt thereof, are from about 0.01 to about 50 mg/kg per body weight. In some embodiments, the daily dosage or the amount of active in the dosage form are lower or higher than the ranges indicated herein, based on a number of variables in regard to an individual treatment regime. In various embodiments, the daily and unit dosages are altered depending on a number of variables including, but not limited to, the activity of the compound used, the disease or condition to be treated, the mode of administration, the requirements of the individual subject, the severity of the disease or condition being treated, and the judgment of the practitioner.

Toxicity and therapeutic efficacy of such therapeutic regimens are determined by standard pharmaceutical procedures in cell cultures or experimental animals, including, but not limited to, the determination of the LD₅₀ and the ED₅₀. The dose ratio between the toxic and therapeutic effects is the therapeutic index and it is expressed as the ratio between LD₅₀ and ED₅₀. In certain embodiments, the data obtained from cell culture assays and animal studies are used in formulating the therapeutically effective daily dosage range and/or the therapeutically effective unit dosage amount for use in mammals, including humans. In some embodiments, the daily dosage amount of the compounds described herein lies within a range of circulating concentrations that include the ED₅₀ with minimal toxicity. In certain embodiments, the daily dosage range and/or the unit dosage amount varies within this range depending upon the dosage form employed and the route of administration utilized.

In any of the aforementioned aspects are further embodiments in which the effective amount of the compound described herein, or a pharmaceutically acceptable salt thereof, is: (a) systemically administered to the mammal; and/or (b) administered orally to the mammal; and/or (c) intravenously administered to the mammal; and/or (d) administered by injection to the mammal; and/or (e) administered topically to the mammal; and/or (f) administered non-systemically or locally to the mammal.

In any of the aforementioned aspects are further embodiments comprising single administrations of the effective amount of the compound, including further embodiments in which (i) the compound is administered once a day; or (ii) the compound is administered to the mammal multiple times over the span of one day.

In any of the aforementioned aspects are further embodiments comprising multiple administrations of the effective amount of the compound, including further embodiments in which (i) the compound is administered continuously or intermittently: as in a single dose; (ii) the time between multiple administrations is every 6 hours; (iii) the compound is administered to the mammal every 8 hours; (iv) the compound is administered to the mammal every 12 hours; (v) the compound is administered to the mammal every 24 hours. In further or alternative embodiments, the method comprises a drug holiday, wherein the administration of the compound is temporarily suspended or the dose of the compound being administered is temporarily reduced; at the end of the drug holiday, dosing of the compound is resumed. In one embodiment, the length of the drug holiday varies from 2 days to 1 year.

In certain instances, it is appropriate to administer at least one compound described herein, or a pharmaceutically acceptable salt thereof, in combination with one or more other therapeutic agents. In certain embodiments, the pharmaceutical composition further comprises one or more anti-cancer agents.

In one embodiment, the therapeutic effectiveness of one of the compounds described herein is enhanced by administration of an adjuvant (i.e., by itself the adjuvant has minimal therapeutic benefit, but in combination with another therapeutic agent, the overall therapeutic benefit to the patient is enhanced). Or, in some embodiments, the benefit experienced by a patient is increased by administering one of the compounds described herein with another agent (which also includes a therapeutic regimen) that also has therapeutic benefit.

In one specific embodiment, a compound described herein, or a pharmaceutically acceptable salt thereof, is co-administered with a second therapeutic agent, wherein the compound described herein, or a pharmaceutically acceptable salt thereof, and the second therapeutic agent modulate different aspects of the disease, disorder or condition being treated, thereby providing a greater overall benefit than administration of either therapeutic agent alone.

In any case, regardless of the disease, disorder or condition being treated, the overall benefit experienced by the patient is simply be additive of the two therapeutic agents or the patient experiences a synergistic benefit.

In certain embodiments, different therapeutically-effective dosages of the compounds disclosed herein will be utilized in formulating pharmaceutical composition and/or in treatment regimens when the compounds disclosed herein are administered in combination with one or more additional agent, such as an additional therapeutically effective drug, an adjuvant or the like. Therapeutically-effective dosages of drugs and other agents for use in combination treatment regimens is optionally determined by means similar to those set forth hereinabove for the actives themselves. Furthermore, the methods of prevention/treatment described herein encompasses the use of metronomic dosing, i.e., providing more frequent, lower doses in order to minimize toxic side effects. In some embodiments, a combination treatment regimen encompasses treatment regimens in which administration of a compound described herein, or a pharmaceutically acceptable salt thereof, is initiated prior to, during, or after treatment with a second agent described herein, and continues until any time during treatment with the second agent or after termination of treatment with the second agent. It also includes treatments in which a compound described herein, or a pharmaceutically acceptable salt thereof, and the second agent being used in combination are administered simultaneously or at different times and/or at decreasing or increasing intervals during the treatment period. Combination treatment further includes periodic treatments that start and stop at various times to assist with the clinical management of the patient.

It is understood that the dosage regimen to treat, prevent, or ameliorate the disease(s) for which relief is sought, is modified in accordance with a variety of factors (e.g. the disease or disorder from which the subject suffers; the age, weight, sex, diet, and medical condition of the subject). Thus, in some instances, the dosage regimen actually employed varies and, in some embodiments, deviates from the dosage regimens set forth herein.

For combination therapies described herein, dosages of the co-administered compounds vary depending on the type of co-drug employed, on the specific drug employed, on the disease or condition being treated and so forth. In additional embodiments, when co-administered with one or more other therapeutic agents, the compound provided herein is administered either simultaneously with the one or more other therapeutic agents, or sequentially.

In combination therapies, the multiple therapeutic agents (one of which is one of the compounds described herein) are administered in any order or even simultaneously. If administration is simultaneous, the multiple therapeutic agents are, by way of example only, provided in a single, unified form, or in multiple forms (e.g., as a single pill or as two separate pills).

The compounds described herein, or a pharmaceutically acceptable salt thereof, as well as combination therapies, are administered before, during or after the occurrence of a disease or condition, and the timing of administering the composition containing a compound varies. Thus, in one embodiment, the compounds described herein are used as a prophylactic and are administered continuously to subjects with a propensity to develop conditions or diseases in order to prevent the occurrence of the disease or condition. In another embodiment, the compounds and compositions are administered to a subject during or as soon as possible after the onset of the symptoms. In specific embodiments, a compound described herein is administered as soon as is practicable after the onset of a disease or condition is detected or suspected, and for a length of time necessary for the treatment of the disease. In some embodiments, the length required for treatment varies, and the treatment length is adjusted to suit the specific needs of each subject. For example, in specific embodiments, a compound described herein or a formulation containing the compound is administered for at least 2 weeks, about 1 month to about 5 years.

In some embodiments, a compound described herein, or a pharmaceutically acceptable salt thereof, is administered in combination with chemotherapy, radiation therapy, monoclonal antibodies, or combinations thereof.

Chemotherapy includes the use of anti-cancer agents.

### Methods of Synthesis

The compounds may be prepared using methods disclosed herein and routine modifications thereof, which will be apparent given the disclosure herein and methods well known in the art. Conventional and well-known synthetic methods may be used in addition to the teachings herein. The synthesis of typical compounds described herein may be accomplished as described in the following examples. If available, reagents may be purchased commercially, e.g., from Sigma Aldrich or other chemical suppliers.

Typical embodiments of compounds described herein may be synthesized using the general reaction schemes described below. It will be apparent given the description herein that the general schemes may be altered by substitution of the starting materials with other materials having similar structures, to result in products that are correspondingly different. Descriptions of syntheses follow to provide numerous examples of how the starting materials may vary to provide corresponding products. Given a desired product for which the substituent groups are defined, the necessary starting materials generally may be determined by inspection. Starting materials are typically obtained from commercial sources or synthesized using published methods. For synthesizing compounds which are embodiments described in the present disclosure, inspection of the structure of the compound to be synthesized will provide the identity of each substituent group. The identity of the final product will generally render apparent the identity of the necessary starting materials by a simple process of inspection, given the examples herein. In general, compounds described herein are typically stable and isolatable at room temperature and pressure.

Preparation of compounds as disclosed herein can involve the protection and deprotection of various chemical groups. The need for protection and deprotection, and the selection of appropriate protecting groups, can be readily determined by one skilled in the art. The chemistry of protecting groups is described, e.g., in Kocienski, Protecting Groups, (Thieme, 2007); Robertson, Protecting Group Chemistry, (Oxford University Press, 2000); Smith et al., March's Advanced Organic Chemistry: Reactions, Mechanisms, and Structure, 6^{th} Ed. (Wiley, 2007); Peturssion et al., “Protecting Groups in Carbohydrate Chemistry,” J. Chem. Educ., 1997, 74(11), 1297; and Wuts et al., Protective Groups in Organic Synthesis, 4^{th} Ed., (Wiley, 2006).

The Schemes below provide general guidance in connection with preparing the compounds described herein. One skilled in the art would understand that the preparations shown in the Schemes can be modified or optimized using general knowledge of organic chemistry to prepare various compounds.

In some embodiments, compounds of any one of Formula (I-A), Formula (I-B), Formula (I-C), Formula (I-D), Formula (I-E), and/or Formula (I-F), disclosed herein and certain intermediates can be prepared, for example, using a process as illustrated in Schemes 1-5. The variables employed in the Schemes below are as defined throughout the specification.

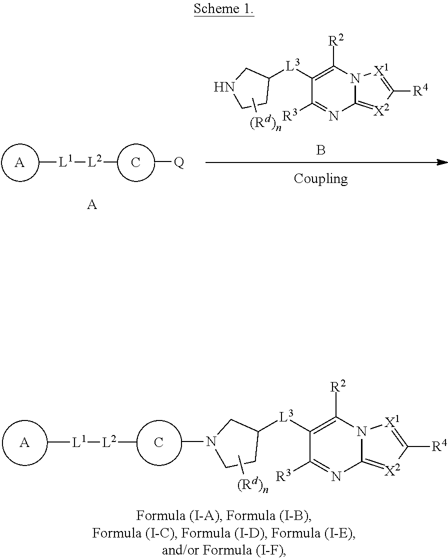

In some embodiments, as shown in Scheme 1, a compound of any one of Formula (I-A), Formula (I-B), Formula (I-C), Formula (I-D), Formula (I-E), and/or Formula (I-F), can be synthesized from linker-connected cyclic moieties of formula A and pyrrolidine B by standard methods of forming carbon-nitrogen bonds, using coupling agents appropriate for this transformation that are well known in the art, such as tris(dibenzylideneacetone)dipalladium(0) or any other suitable coupling agent, in the presence of a base, such as cesium carbonate or any other suitable base (e.g., K₂CO₃, Na₂CO₃), in organic solvents, e.g., THE or 2-MeTHF or other aprotic solvents, at suitable temperatures (e.g., by heating to a temperature above room temperature), wherein Q is a leaving group (including, but not limited to, Br, Cl, I, triflate, and the like).

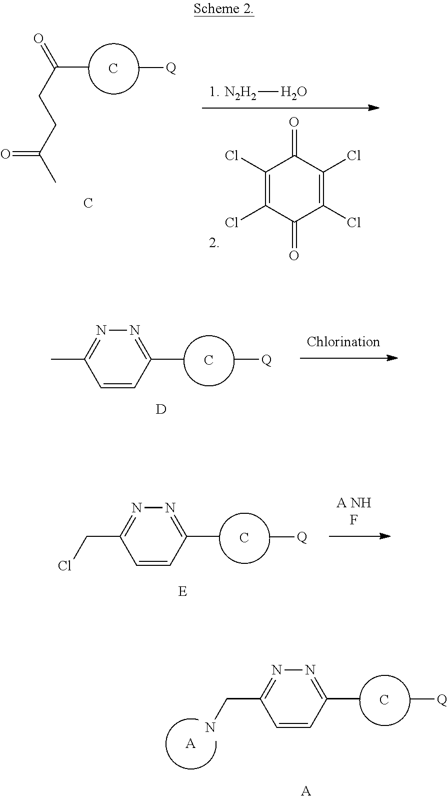

In some embodiments, as shown in Scheme 2, a compound of formula A (L¹=methylene, L²=pyridazine) can be made from a multi-step process, starting by first treating dione C, wherein Q is a leaving group (including, but not limited to, Br, Cl, I, triflate, and the like), with hydrazine in solvents, such as ethanol, at temperatures below or about room temperature. Subsequent aromatization of the resulting product with 2,3,5,6-tetrachloro-1,4-benzoquinone in organic solvents, such acetonitrile, at temperatures below or about room temperature provides a compound of formula D. Chlorination of the methyl group of compounds of formula D employing 1,3,5-trichloro-1,3,5-triazinane-2,4,6-trione in organic solvents, such as dichloromethane, at temperatures below or about room temperature gives a compound of formula E. Compounds of formula E can then undergo nucleophilic substitution with amine-containing ring systems of formula F in organic solvents, such as acetonitrile, at suitable temperatures (e.g., reflux temperature of a solvent) to provide compounds of formula A.

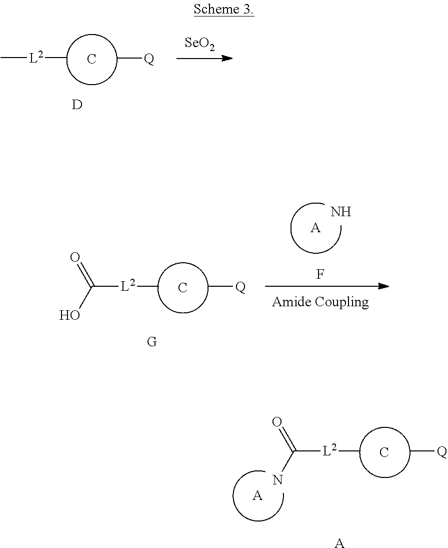

In some embodiments, as shown in Scheme 3, a compound of formula A (L¹=carbonyl) can be made from a two-step process, starting by first oxidizing the methyl on L² in compound D, wherein Q is a leaving group (including, but not limited to, Br, Cl, I, triflate, and the like), with selenium dioxide in basic organic solvents, such as pyridine, at suitable temperatures to provide a compound of formula G. In a second step, compounds of formula G can be converted to compounds of formula A by undergoing amide coupling with amine-containing ring systems of formula F by standard methods that are well known in the art, such as with HATU, in the presence of a base, such as DIEA, in organic solvents, such as DMF, at temperatures below or about room temperature.

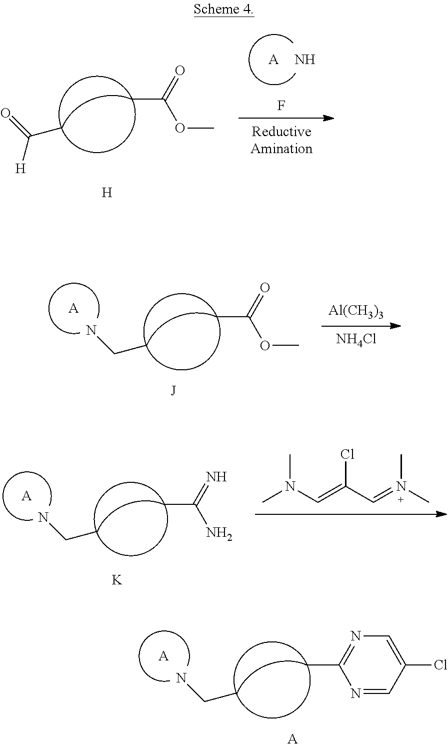

In some embodiments, as shown in Scheme 4, a compound of formula A (L¹=methylene, L²=bridged bicyclic, C=pyrimidine, Q=Cl) can be made from a multi-step process, beginning by first undergoing a reductive amination of bridged bicyclic aldehyde compound H with an amine-containing ring systems of formula F, in the presence of acetic acid and sodium triacetoxyborohydride, in organic solvents, such a DCE, at room temperature to form a compound of formula J. Amination of compounds of formula J with ammonium chloride, in the presence of trimethylaluminum, in organic solvents, such as toluene, at suitable temperatures (e.g., heating above room temperature) provides a compound of formula K. Cyclization on the imidamide moiety of compounds of formula K with [(Z)-2-chloro-3-(dimethylamino)prop-2-enylidene]-dimethyl-ammonium hexafluorophosphate, in the presence of a base, such as sodium methoxide, in organic solvents, such as dioxane, at decreased or room temperatures yields a compound of formula A. It will be understood that suitable stereoisomers can be prepared using any suitable starting material and/or chiral resolution of the final product.

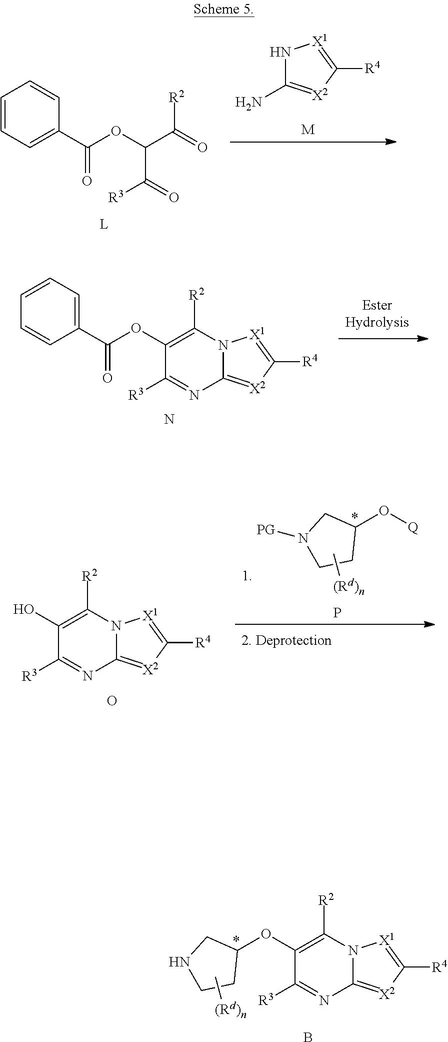

In some embodiments, as shown in Scheme 5, a compound of formula B can be made from a multi-step process, starting from dione L and heterocycle M, which cyclize in the presence of acetic acid at elevated temperatures to form a compound of formula N. Ester hydrolysis of compounds of formula N, in the presence of a base, such as sodium hydroxide, in solvents such as a mixture of methanol and water, at temperatures below or about room temperature, provide heterocyclic compounds of formula O. Compounds of formula O can be converted to protected derivates of compounds of formula B by the nucleophilic substitution of protected pyrrolidine compounds of formula P, wherein Q is a leaving group (including, but not limited to, tosylate, triflate, and the like) and PG is a protecting group (including, but not limited to, Boc and the like), in the presence of a base, such as potassium carbonate, in organic solvents, such as DMF, at elevated temperatures. Subsequent deprotection of the protected pyrrolidine moiety in the presence of an acid, such as hydrochloric acid, in organic solvents, such as ethyl acetate, at decreased or room temperatures provides a compound of formula B.

Any combination of steps described above may be used in the preparation of compounds described herein, including any procedures described in the Examples section.

The compounds of this disclosure can be prepared from readily available starting materials using, for example, the following general methods and procedures. It will be appreciated that where typical or preferred process conditions (i.e., reaction temperatures, times, mole ratios of reactants, solvents, pressures, etc.) are given, other process conditions can also be used unless otherwise stated. Optimum reaction conditions may vary with the particular reactants or solvent used, but such conditions can be determined by one skilled in the art by routine optimization procedures.

Additionally, as will be apparent to those skilled in the art, conventional protecting groups may be necessary to prevent certain functional groups from undergoing undesired reactions. Suitable protecting groups for various functional groups as well as suitable conditions for protecting and deprotecting particular functional groups are well known in the art. For example, numerous protecting groups are described in T. W. Greene and G. M. Wuts (1999) Protecting Groups in Organic Synthesis, 3rd Edition, Wiley, New York, and references cited therein.

Furthermore, the compounds of this disclosure may contain one or more chiral centers. Accordingly, if desired, such compounds can be prepared or isolated as pure stereoisomers, i.e., as individual enantiomers or diastereomers or as stereoisomer-enriched mixtures. All such stereoisomers (and enriched mixtures) are included within the scope of this disclosure, unless otherwise indicated. Pure stereoisomers (or enriched mixtures) may be prepared using, for example, optically active starting materials or stereoselective reagents well-known in the art. Alternatively, racemic mixtures of such compounds can be separated using, for example, chiral column chromatography, chiral resolving agents, and the like.

The starting materials for the following reactions are generally known compounds or can be prepared by known procedures or obvious modifications thereof. For example, many of the starting materials are available from commercial suppliers such as Aldrich Chemical Co. (Milwaukee, Wis., USA), Bachem (Torrance, Calif., USA), Emka-Chemie or Sigma (St. Louis, Mo., USA). Others may be prepared by procedures or obvious modifications thereof, described in standard reference texts such as Fieser and Fieser's Reagents for Organic Synthesis, Volumes 1-15 (John Wiley, and Sons, 1991), Rodd's Chemistry of Carbon Compounds, Volumes 1-5, and Supplementals (Elsevier Science Publishers, 1989) organic Reactions, Volumes 1-40 (John Wiley, and Sons, 1991), March's Advanced Organic Chemistry, (John Wiley, and Sons, 5^{th} Edition, 2001), and Larock's Comprehensive Organic Transformations (VCH Publishers Inc., 1989).

### EXAMPLES

The following examples are provided for illustrative purposes only and not to limit the scope of the claims provided herein. Unless stated otherwise, starting materials are commercially available.

### Example 1—Synthesis of (R)-4-((6-(4-(3-((2,5,7-trimethyl-[1,2,4]triazolo[1,5-a]pyrimidin-6-yl)oxy)pyrrolidin-1-yl)phenyl)pyridazin-3-yl)methyl)morpholine (Compound 1)

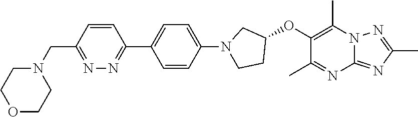 [SMILES: Cc1nc2nc(C)c(O[C@@H]3CCN(c4ccc(-c5ccc(CN6CCOCC6)nn5)cc4)C3)c(C)n2n1]

### Step 1: Preparation of 1-(4-bromophenyl)pentane-1,4-dione

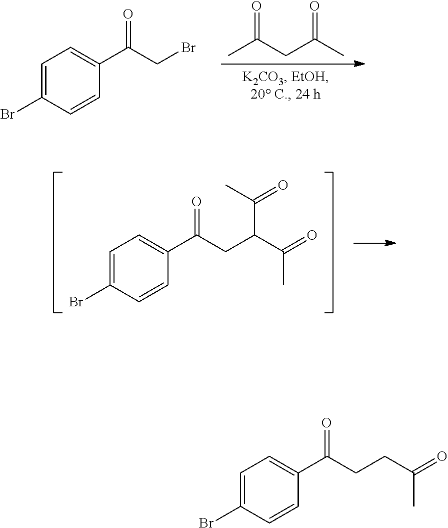

To a solution of 2-bromo-1-(4-bromophenyl)ethanone (122 g, 438.94 mmol, 1 eq) and K₂CO₃ (122.00 g, 882.74 mmol, 2.01 eq) in ethanol (720 mL) was added pentane-2,4-dione (92.00 g, 918.94 mmol, 94.36 mL, 2.09 eq) at 25° C. and stirred at 25° C. for 24 hr. TLC showed all bromoketone was consumed and one new spot generated. The mixture was poured into a mixture of Ethyl acetate/water (2 L, 1:1) under stirring. After separation, the organic phase was concentrated under reduced pressure. Then n-heptane (1 L) was added into the above residue and stirred at 25° C. for 16 h. The mixture solution was filtered and the filter cake was dried to give 1-(4-bromophenyl)pentane-1,4-dione (80 g, 313.59 mmol, 71.44% yield) as pale yellow solid.

¹H NMR (400 MHz, CDCl₃) δ ppm: 7.85 (d, J=8.8 Hz, 2H), 7.66 (d, J=8.8 Hz, 2H), 3.24 (t, J=6.8 Hz, 2H), 2.90 (t, J=6.8 Hz, 2H), 3.85 (s, 2H), 2.25 (s, 3H).

### Step 2: Preparation of 3-(4-bromophenyl)-6-methyl-4,5-dihydropyridazine

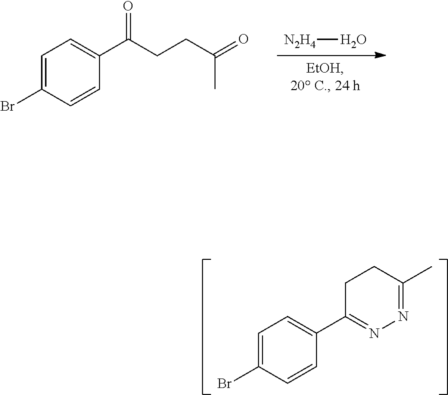

A solution of 1-(4-bromophenyl) pentane-1,4-dione (85 g, 333.19 mmol, 1 eq) and hydrazine hydrate (70.19 g, 1.40 mol, 68.15 mL, 4.21 eq) in ethanol (500 mL) was stirred at 25° C. for 48 hr. LCMS showed all of the diketone was consumed and one major peak was detected with desired Ms. The mixture was diluted with water (4 L), and the precipitate was collected by filtration. The solid was washed with water (500 mL) and petroleum ether (500 mL), and then dried under reduced pressure to give 3-(4-bromophenyl)-6-methyl-4,5-dihydropyridazine (65 g, 258.84 mmol, 77.68% yield) as yellow solid, which was used into the next step directly without further purification. LCMS for product (ESI): m/z 250.9 (M+H)⁺, 252.9 (M+H+2)⁺.

### Step 3: Preparation of 3-(4-Bromophenyl)-6-Methyl-Pyridazine

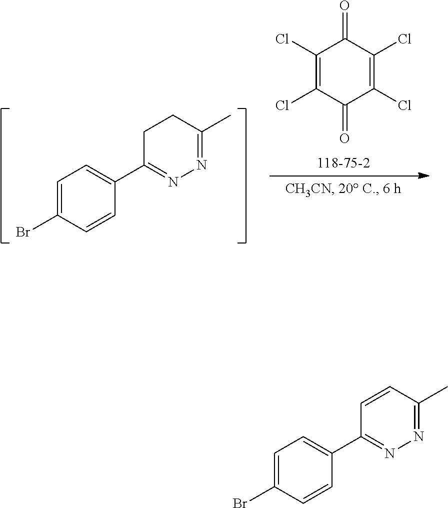

To a solution of 3-(4-bromophenyl)-6-methyl-4,5-dihydropyridazine (45 g, 179.20 mmol, 1 eq) in CH₃CN (1.5 L) was added 2,3,5,6-tetrachloro-1,4-benzoquinone (56.25 g, 228.77 mmol, 1.28 eq) at 25° C. and stirred at 20° C. for 6 hr. LCMS showed dihydropyridazine was consumed completely and desired product was detected. The reaction was quenched with 12.5% w/w sodium sulfite solution (300 mL) and the CH₃CN was removed by concentration. Then water (500 mL) and 2 M sodium hydroxide solution (500 mL) were added into the above solution and filtered. The solid was dissolved in dichloromethane (500 mL) and washed with water (3×200 mL). The organic phase was separated and concentrated under reduced pressure to give a crude product. The crude product was triturated by methyl tert-butyl ether (100 mL) to give 3-(4-bromophenyl)-6-methyl-pyridazine (33 g, 132.47 mmol, 73.93% yield) as pale yellow solid. LCMS for product (ESI): m/z 249.0 (M+H)+, 251.0 (M+H+2)+.

¹H NMR (400 MHz, DMSO-d₆) δ=8.14 (d, J=8.8 Hz, 1H), 8.09-8.07 (m 2H), 7.75-7.73 (m 2H), 7.66 (d, J=8.8 Hz, 1H), 2.66 (s, 3H).

### Step 4: Preparation of 3-(4-bromophenyl)-6-(chloromethyl)pyridazine

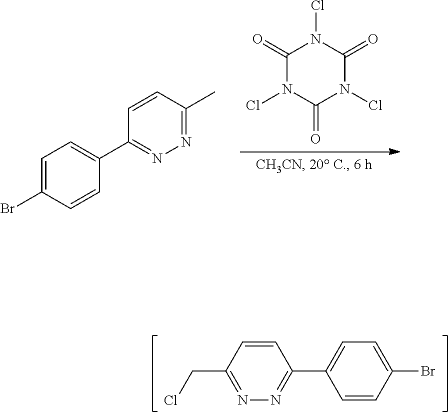

To a solution of 3-(4-bromophenyl)-6-methyl-pyridazine (32 g, 128.46 mmol, 1 eq) in DCM (500 mL) was added 1,3,5-trichloro-1,3,5-triazinane-2,4,6-trione (12 g, 51.63 mmol, 4.02e-1 eq) at 25° C. and stirred at 25° C. for 3 hr. TLC showed all methyl-pyridazine was consumed and three spots generated. The reaction was filtered and the filtrate was washed with water (50 mL). The organic phase was separated, concentrated under reduced pressure to give a crude product. The crude product was purified by column chromatography eluted with petroleum ether:ethyl acetate (100:1 to 5:1) to give 3-(4-bromophenyl)-6-(chloromethyl)pyridazine (17.5 g, 62 mmol, 48% yield) as a pale yellow solid.

¹H NMR (400 MHz, DMSO-d₆) δ=8.33 (d, J=8.8 Hz, 1H), 8.13 (d, J=8.4 Hz, 2H), 7.96 (d, J=8.8 Hz, 1H), 7.78 (d, J=8.4 Hz, 2H), 5.06 (s, 2H).

### Step 5: Preparation of 4-[[6-(4-bromophenyl)pyridazin-3-yl]methyl]morpholine

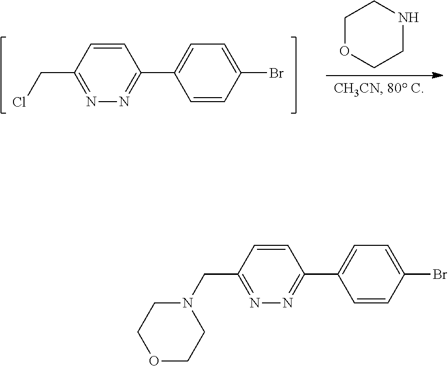

To a solution of 3-(4-bromophenyl)-6-(chloromethyl)pyridazine (12 g, 42.32 mmol, 1 eq) in CH₃CN (200 mL) was added morpholine (7.37 g, 84.64 mmol, 7.45 mL, 2 eq) at 25° C. and stirred at 60° C. for 3 hr. LCMS showed all of the chloride was consumed and one major peak was detected with desired Ms. The reaction was concentrated under reduced pressure to give a crude product. The crude product was purified by chromatography column on silica gel eluted with Petroleum ether:ethyl acetate (10:1 to 1:10) to give 4-[[6-(4-bromophenyl)pyridazin-3-yl]methyl]morpholine (12 g, 35.91 mmol, 84.84% yield) as pale yellow solid. LCMS for product (ESI): m/z 334.0 (M+H)⁺, 336.0 (M+H+2)⁺.

¹H NMR (400 MHz, CDCl₃) δ ppm: 7.91 (d, J=8.8 Hz, 2H), 7.76 (d, J=8.4 Hz, 1H), 7.67 (d, J=8.4 Hz, 1H), 7.59 (d, J=8.8 Hz, 2H), 3.85 (s, 2H), 3.67 (t, J=4.8 Hz, 4H), 2.50 (t, J=4.8 Hz, 4H).

### Step 6: Preparation of (1-acetyl-2-oxo-propyl) benzoate

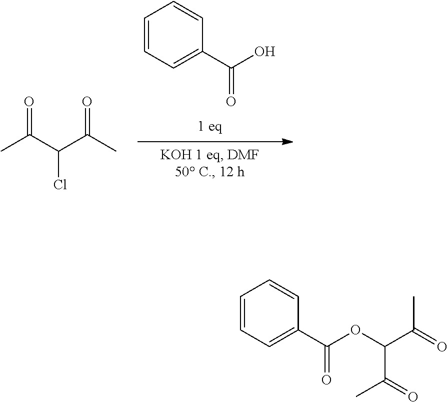

A solution of benzoic acid (75 g, 614.14 mmol, 93.75 mL, 1 eq) and KOH (34.46 g, 614.14 mmol, 1 eq) in DMF (700 mL) was stirred at 50° C. for 1 h. Then 3-chloropentane-2,4-dione (82.64 g, 614.14 mmol, 73.13 mL, 1 eq) was added into the above solution at 50° C. and stirred at 50° C. for 13 hr. TLC indicated the chloride was consumed and one major new spot with lower polarity was detected. The reaction mixture was partitioned between MTBE 500 mL and water 500 mL. The organic phase was separated, washed with brine 500 mL, dried over Na₂SO₄, filtered. The filtrate was concentrated under reduced pressure to give (1-acetyl-2-oxo-propyl) benzoate (135 g, 613.02 mmol, 99.82% yield) as yellow oil.

¹H NMR (400 MHz, DMSO-d₆) δ=8.09 (d, J=7.6 Hz, 2H), 7.78-7.71 (m, 1H), 7.63-7.57 (m, 2H), 5.93 (s, 1H), 2.34 (s, 6H).

### Step 7: Preparation of (2,5,7-trimethyl-[1,2,4]triazolo[1,5-a]pyrimidin-6-yl) benzoate

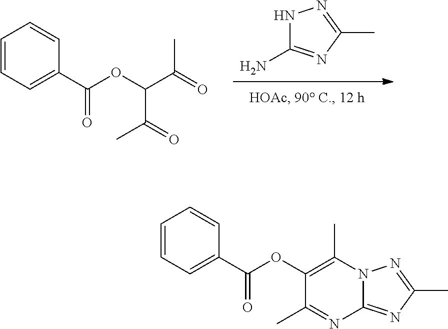

A solution of 3-methyl-1H-1,2,4-triazol-5-amine (60.14 g, 613.02 mmol, 1 eq) and (1-acetyl-2-oxo-propyl) benzoate (135 g, 613.02 mmol, 1 eq) in HOAc (1000 mL) was stirred at 90° C. for 20 h. LCMS showed all the diketone was consumed and one major peak was detected with desired Ms. The reaction mixture was concentrated to remove the solvent to get the crude product (2,5,7-trimethyl-[1,2,4]triazolo[1,5-a]pyrimidin-6-yl) benzoate (170 g, 493.81 mmol, 80.55% yield, 82% purity) as a brown oil, which was used into next step without further purification. LCMS for product (ESI): m/z 283.1 [M+H]⁺.

### Step 8: Preparation of 2,5,7-trimethyl-[1,2,4]triazolo[1,5-a]pyrimidin-6-ol

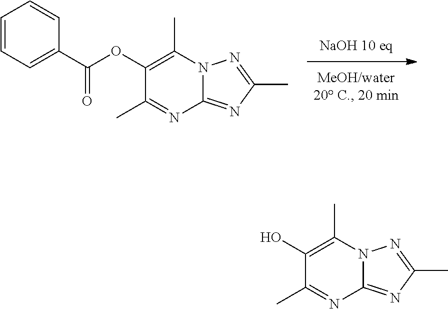 [SMILES: Cc1nc2nc(C)c(OC(=O)c3ccccc3)c(C)n2n1 ; Cc1nc2nc(C)c(O)c(C)n2n1]

A solution of (2,5,7-trimethyl-[1,2,4]triazolo[1,5-a]pyrimidin-6-yl) benzoate (170 g, 602.20 mmol, 1 eq) in MeOH (600 mL) was added NaOH (5 M, 1.2 L, 9.96 eq) at 25° C. and stirred at 20° C. for 20 h. LCMS showed all the benzoate was consumed and one major peak was detected with desired Ms. The reaction was concentrated to remove the MeOH and the aqueous layer was adjusted to pH=4 by HCl (2N). The mixture was filtered and the filter cake was dried to give 2,5,7-trimethyl-[1,2,4]triazolo[1,5-a]pyrimidin-6-ol (40 g, 193.05 mmol, 32.06% yield, 86% purity) as a yellow solid. LCMS for product (ESI): m/z 179.1 [M+H]⁺.

¹H NMR (400 MHz, DMSO-d₆) δ=9.35 (s, 1H), 2.61 (s, 3H), 2.50 (s, 3H), 2.41 (s, 3H).

### Step 9: Preparation of tert-butyl (3R)-3-[(2,5,7-trimethyl-[1,2,4]triazolo[1,5-a]pyrimidin-6-yl)oxy]pyrrolidine-1-carboxylate

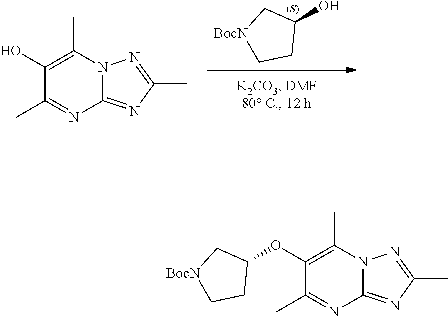

To a solution of tert-butyl (3S)-3-hydroxypyrrolidine-1-carboxylate (47.91 g, 255.90 mmol, 1.2 eq), 2,5,7-trimethyl-[1,2,4]triazolo[1,5-a]pyrimidin-6-ol (38 g, 213.25 mmol, 1 eq) and PPh₃ (61.53 g, 234.58 mmol, 1.1 eq) in THE (400 mL) was adding the solution of DIAD (60.37 g, 298.55 mmol, 58.05 mL, 1.4 eq) in THE (200 mL) in dropwise at 0° C. and then stirred at 20° C. for 20 h. LCMS showed all the starting material was consumed completely and one major peak was detected with desired Ms. The reaction mixture was partitioned between water 500 mL and EA 500 mL. The organic phase was separated, washed with brine 500 mL, dried over Na₂SO₄, filtered. The filtrate was concentrated under reduced pressure to give tert-butyl (3R)-3-[(2,5,7-trimethyl-[1,2,4]triazolo[1,5-a]pyrimidin-6-yl)oxy]pyrrolidine-1-carboxylate (74 g, 213.00 mmol, 99.88% yield, crude purity) as a brown oil, which was used into the next step directly without further purification. LCMS for product (ESI): m/z 348.2 [M+H]⁺.

### Step 10: Preparation of 2,5,7-trimethyl-6-[(3R)-pyrrolidin-3-yl]oxy-[1,2,4]triazolo[1,5-a]pyrimidine

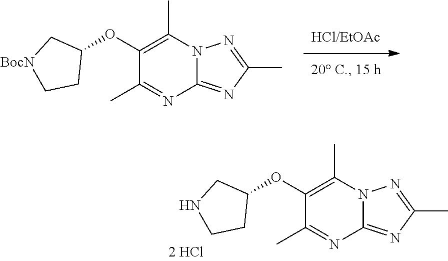 [SMILES: Cc1nc2nc(C)c(O[C@@H]3CCN(C(=O)OC(C)(C)C)C3)c(C)n2n1 ; Cc1nc2nc(C)c(O[C@@H]3CCNC3)c(C)n2n1]

A solution of tert-butyl (3R)-3-[(2,5,7-trimethyl-[1,2,4]triazolo[1,5-a]pyrimidin-6-yl)oxy]pyrrolidine-1-carboxylate (74 g, 213.00 mmol, 1 eq) in HCl/EtOAc (100 mL, 4N) was stirred at 20° C. for 20 hr. LCMS showed all the starting material was consumed completely and one major peak was detected with desired Ms. The reaction was filtered and the filter cake was dried to give 2,5,7-trimethyl-6-[(3R)-pyrrolidin-3-yl]oxy-[1,2,4]triazolo[1,5-a]pyrimidine (40 g, 124.54 mmol, 58.47% yield, 99.7% purity, 2HCl) as an orange solid. LCMS for product (ESI): m/z 501.3 [M+H]⁺.

¹H NMR (400 MHz, DMSO-d₆) δ9.97 (br s, 2H), 4.91-4.88 (m, 1H), 3.56-3.42 (m, 1H), 3.40-3.30 (m, 3H), 2.74 (s, 3H), 2.62 (s, 3H), 2.52 (s, 3H), 2.26-2.22 (m, 1H), 2.10-2.06 (m, 1H).

### Step 11: Preparation of 4-[[6-[4-[(3R)-3-[(2,5,7-trimethyl-[1,2,4]triazolo[1,5-a]pyrimidin-6-yl)oxy]pyrrolidin-1-yl]phenyl]pyridazin-3-yl]methyl] morpholine (Compound 1)

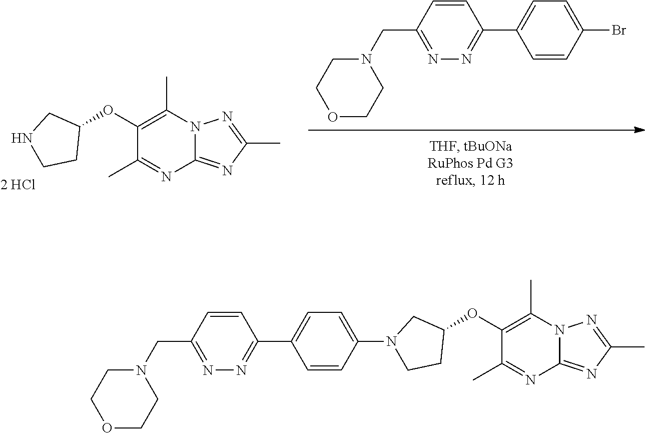 [SMILES: Cc1nc2nc(C)c(O[C@@H]3CCNC3)c(C)n2n1 ; Brc1ccc(-c2ccc(CN3CCOCC3)nn2)cc1 ; Cc1nc2nc(C)c(O[C@@H]3CCN(c4ccc(-c5ccc(CN6CCOCC6)nn5)cc4)C3)c(C)n2n1]

A solution of 4-[[6-(4-bromophenyl)pyridazin-3-yl]methyl]morpholine (12 g, 35.91 mmol, 1 eq), 2,5,7-trimethyl-6-[(3R)-pyrrolidin-3-yl]oxy-[1,2,4]triazolo[1,5-a]pyrimidine (14.95 g, 46.68 mmol, 1.3 eq, 2HCl), Cs₂CO₃ (58.49 g, 179.53 mmol, 5 eq), ditert-butyl-[2-(o-tolyl)phenyl]phosphane (1.12 g, 3.59 mmol, 0.1 eq) and Pd₂(dba)₃ (1.64 g, 1.80 mmol, 0.05 eq) in 2-MeTHF (240 mL) and H₂O (120 mL) was de-gassed and then heated at 80° C. for 24 h under N₂. LC-MS showed all the bromide was consumed completely and one major peak was detected with desired Ms. Two additional reactions were set up and combined together to work-up. The combined reaction mixture was partitioned between DCM 600 mL and water 500 mL. The organic phase was separated, washed with brine 500 mL, dried over Na₂SO₄, filtered. The filtrated was concentrated under reduced pressure to give a residue. The crude product was triturated with CAN 200 mL to give 4-[[6-[4-[(3R)-3-[(2,5,7-trimethyl-[1,2,4]triazolo[1,5-a]pyrimidin-6-yl)oxy]pyrrolidin-1-yl]phenyl]pyridazin-3-yl]methyl] morpholine (28 g, 54.81 mmol, 63.61% yield, 98% purity) as a yellow solid. LCMS for product (ESI): m/z 501.3 [M+H]⁺.

¹H NMR (400 MHz, CHLOROFORM-d) δ 8.05 (d, J=8.8 Hz, 2H), 7.77 (d, J=8.8 Hz, 1H), 7.62 (d, J=8.8 Hz, 1H), 6.68 (d, J=8.8 Hz, 2H), 4.85-4.83 (m, 1H), 3.88 (s, 2H), 3.82-3.76 (m, 1H), 3.76-3.70 (m, 4H), 3.66-3.47 (m, 3H), 2.65 (s, 3H), 2.61 (s, 3H), 2.60 (s, 3H), 2.59-2.54 (m, 4H), 2.54-2.47 (m, 1H), 2.34-2.32 (m, 1H).

### Example 2—Synthesis of (R)-4-((6-(4-(3-((2,5,7-trimethylimidazo[1,2-a]pyrimidin-6-yl)oxy)pyrrolidin-1-yl)phenyl)pyridazin-3-yl)methyl)morpholine (Compound 2)

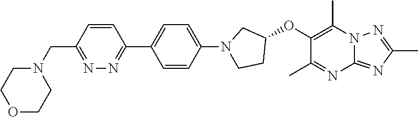 [SMILES: Cc1nc2nc(C)c(O[C@@H]3CCN(c4ccc(-c5ccc(CN6CCOCC6)nn5)cc4)C3)c(C)n2n1]

### Step 1: Preparation of tert-butyl (3S)-3-(p-tolylsulfonyloxy)pyrrolidine-1-carboxylate

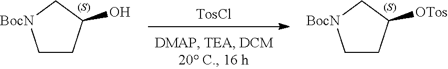

To a solution of tert-butyl(3S)-3-hydroxypyrrolidine-1-carboxylate (10 g, 53.41 mmol, 1 eq) in DCM (120 mL) was added DMAP (652.49 mg, 5.34 mmol, 0.1 eq) and TEA (16.21 g, 160.23 mmol, 22.30 mL, 3 eq) at 0° C. After addition, TosCl (20.36 g, 106.82 mmol, 2 eq) was added at 0° C. The resulting mixture was stirred at 25° C. for 16 hr. TLC showed all the alcohol was consumed completely. The residue was quenched with aq Na₂CO₃ 50 mL and the solution was separated. The aqueous phase was extracted with DCM 50 mL. The organic layers were combined and washed with brine 50 mL, dried over Na₂SO₄ and filtered. The filtrate was concentrated under reduced pressure and purified by column chromatography (SiO₂, Petroleum ether/Ethyl acetate=20/1 to 5/1). Compound tert-butyl (3S)-3-(p-tolylsulfonyloxy)pyrrolidine-1-carboxylate (15 g, 43.93 mmol, 82.26% yield) was obtained as a yellow oil.

¹H NMR (400 MHz, CHLOROFORM-d) δ=7.79 (d, J=8.0 Hz, 2H), 7.36 (d, J=7.2 Hz, 2H), 5.04 (br s, 1H), 3.50-3.36 (m, 4H), 2.46 (s, 3H), 2.10-1.91 (m, 2H), 1.43 (s, 9H).

### Step 2: Preparation of (2,5,7-trimethylimidazo[1,2-a]pyrimidin-6-yl) benzoate

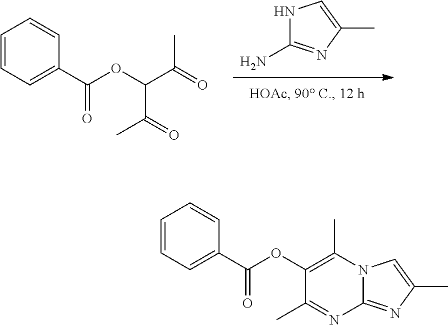

A solution of (1-acetyl-2-oxo-propyl) benzoate (2 g, 9.08 mmol, 1 eq) and 4-methyl-1H-imidazol-2-amine (882.01 mg, 9.08 mmol, 1 eq) in AcOH (20 mL) was stirred at 90° C. for 12 hr.

LC-MS showed the diketone was consumed completely and one major peak was detected with desired Ms. The reaction mixture was concentrated under reduced pressure to remove AcOH. The crude product was purified by re-crystallization from ethyl acetate (50 ml) at 25° C. to give (2,5,7-trimethylimidazo[1,2-a]pyrimidin-6-yl) benzoate (1.8 g, 6.40 mmol, 70.46% yield) as light yellow solid. LCMS for product (ESI): m/z 282.1 [M+H]⁺.

### Step 3: Preparation of 2,5,7-trimethylimidazo[1,2-a]pyrimidin-6-ol

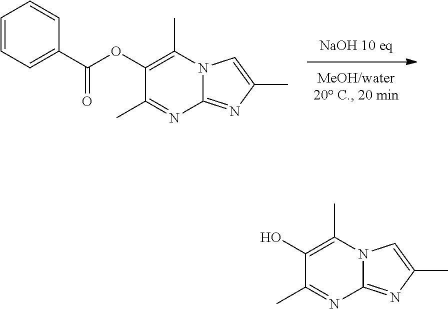 [SMILES: Cc1cn2c(C)c(OC(=O)c3ccccc3)c(C)nc2n1 ; Cc1cn2c(C)c(O)c(C)nc2n1]

A solution of (2,5,7-trimethylimidazo[1,2-a]pyrimidin-6-yl) benzoate (1.5 g, 5.33 mmol, 1 eq) and NaOH (1.07 g, 26.66 mmol, 5 eq) in H₂O (10 mL) and MeOH (10 mL) was stirred at 20° C. for 20 min. LC-MS showed all the benzoate was consumed completely and one major peak was detected with desired Ms. The reaction mixture was concentrated under reduced pressure to remove MeOH. The residue was adjusted to pH=4 by 1N HCl and filtered. The filter cake was dried to give 2,5,7-trimethylimidazo[1,2-a]pyrimidin-6-ol (850 mg, 4.80 mmol, 89.96% yield) as a yellow solid. LCMS for product (ESI): m/z 178.1 [M+H]⁺.

¹H NMR (400 MHz, DMSO-d₆) δ=6.75 (s, 1H), 2.19 (s, 3H), 2.18 (s, 3H), 2.16 (s, 3H).

### Step 4: Preparation of tert-butyl (3R)-3-(2,5,7-trimethylimidazo[1,2-a]pyrimidin-6-yl)oxypyrrolidine-1-carboxylate

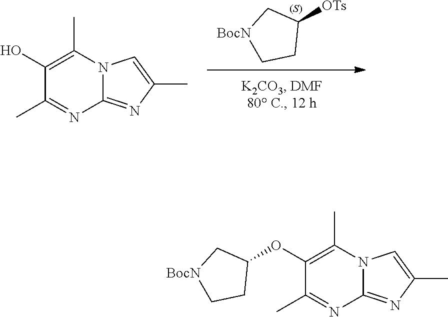

To a solution of 2,5,7-trimethylimidazo[1,2-a]pyrimidin-6-ol (300 mg, 1.69 mmol, 1 eq) and tert-butyl 3-(p-tolylsulfonyloxy)pyrrolidine-1-carboxylate (693.62 mg, 2.03 mmol, 1.2 eq) in DMF (3 mL) was added K₂CO₃ (701.96 mg, 5.08 mmol, 3 eq) at 80° C. and stirred at 80° C. for 12 hr. LC-MS showed all the starting material was consumed. The residue was diluted with H₂O 5 mL and extracted with ethyl acetate 9 mL (3 mL×3). The combined organic layers were washed with brine 5 mL, dried over Na₂SO₄ and filtered. The filtrate was concentrated under reduced pressure to give tert-butyl (3R)-3-(2,5,7-trimethylimidazo[1,2-a]pyrimidin-6-yl)oxypyrrolidine-1-carboxylate (220 mg, 635.06 μmol, 37.51% yield) as yellow oil, which was used into the next step directly without further purification. LCMS for product (ESI): m/z 347.3 [M+H]⁺.

### Step 5: Preparation of 2,5,7-trimethyl-6-[(3R)-pyrrolidin-3-yl]oxy-imidazo[1,2-a]pyrimidine

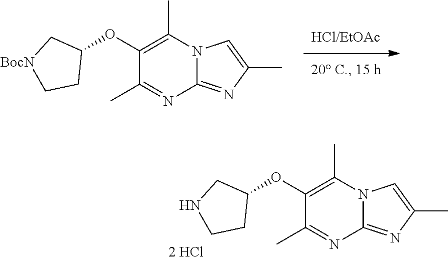 [SMILES: Cc1cn2c(C)c(O[C@@H]3CCN(C(=O)OC(C)(C)C)C3)c(C)nc2n1 ; Cc1cn2c(C)c(O[C@@H]3CCNC3)c(C)nc2n1]

A solution of tert-butyl (3R)-3-(2,5,7-trimethylimidazo[1,2-a]pyrimidin-6-yl)oxypyrrolidine-1-carboxylate (200 mg, 577.33 μmol, 1 eq) in HCl/EtOAc (4N) (5 mL) was stirred at 25° C. for 1 hr. LC-MS showed the Boc protected amine was consumed completely. The reaction mixture was concentrated under reduced pressure to remove HCl/EtOAc to give 2,5,7-trimethyl-6-[(3R)-pyrrolidin-3-yl]oxy-imidazo[1,2-a]pyrimidine (160 mg, crude) as white solid, which used directly without further purification. LCMS for product (ESI): m/z 247.3 [M+H]⁺.

### Step 6: 4-[[6-[4-[(3R)-3-(2,5,7-trimethylimidazo [1,2-a]pyrimidin-6-yl) oxypyrrolidin-1-yl]phenyl]pyridazin-3-yl]methyl] morpholine (Compound 2)

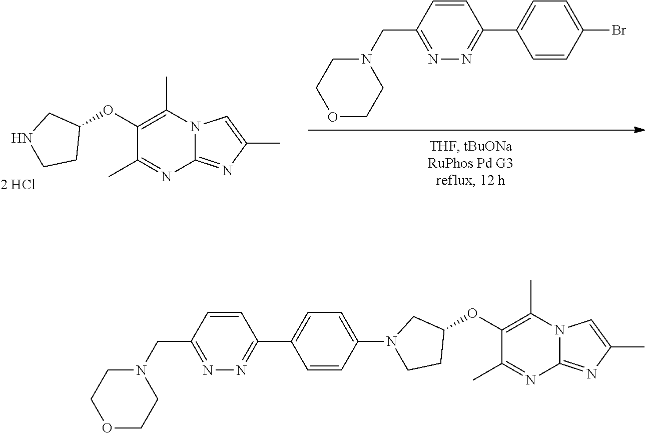 [SMILES: Cc1cn2c(C)c(O[C@@H]3CCNC3)c(C)nc2n1 ; Brc1ccc(-c2ccc(CN3CCOCC3)nn2)cc1 ; Cc1cn2c(C)c(O[C@@H]3CCN(c4ccc(-c5ccc(CN6CCOCC6)nn5)cc4)C3)c(C)nc2n1]

A mixture of 2,5,7-trimethyl-6-[(3R)-pyrrolidin-3-yl]oxy-imidazo[1,2-a]pyrimidine (70 mg, 284.20 μmol, 1 eq), 4-[[6-(4-bromophenyl)pyridazin-3-yl]methyl]morpholine (94.98 mg, 284.20 μmol, 1 eq), [2-(2-aminophenyl)phenyl]-methylsulfonyloxy-palladium; dicyclohexyl-[2-(2,6-diisopropoxyphenyl)phenyl]phosphane (23.77 mg, 28.42 μmol, 0.1 eq), tBuONa (1 M, 568.40 uL, 2 eq) in THE (1 mL) was degassed and purged with N₂ for 3 times, then the mixture was stirred at 80° C. for 12 hr under N₂ atmosphere. LC-MS showed all of Cpd 7A was consumed and desired product was detected. The residue was diluted with H₂O 3 mL and extracted with ethyl acetate 9 mL (3 mL×3). The combined organic layers were washed with brine 3 mL, dried over Na₂SO₄, filtered. The filtrate was concentrated under reduced pressure to give a residue. The residue was purified by prep-HPLC (HCl condition) to give 4-[[6-[4-[(3R)-3-(2,5,7-trimethylimidazo [1,2-a]pyrimidin-6-yl) oxypyrrolidin-1-yl]phenyl]pyridazin-3-yl]methyl] morpholine (19 mg, 36.17 μmol, 12.73% yield, 95.1% purity) as red solid. LCMS for product (ESI): m/z 500.2 [M+H]⁺.

¹H NMR (400 MHz, DMSO-d₆) δ=14.96 (br s, 1H), 11.87 (br s, 1H), 8.30 (d, J=8.8 Hz, 1H), 8.17-8.02 (m, 4H), 6.75 (d, J=8.0 Hz, 2H), 5.04 (s, 1H), 4.67 (s, 2H), 3.91 (s, 4H), 3.45 (d, J=12.8 Hz, 4H), 3.33 (s, 4H), 2.61 (s, 3H), 2.56 (s, 6H) 2.33 (br s, 2H).

### Example 3—Synthesis of (R)-4-((6-(4-(3-((2,3,5,7-tetramethylimidazo[1,2-a]pyrimidin-6-yl)oxy)pyrrolidin-1-yl)phenyl)pyridazin-3-yl)methyl)morpholine (Compound 3)

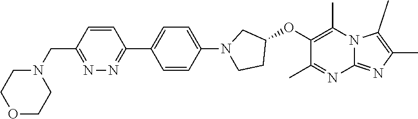 [SMILES: Cc1nc2nc(C)c(C)n2c(C)c1O[C@@H]1CCN(c2ccc(-c3ccc(CN4CCOCC4)nn3)cc2)C1]

### Step 1: Preparation of (2,3,5,7-tetramethylimidazo[1,2-a]pyrimidin-6-yl) benzoate

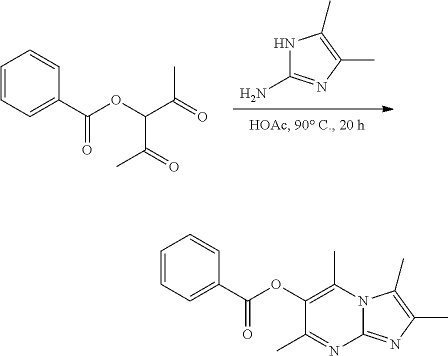

A solution of (1-acetyl-2-oxo-propyl) benzoate (2 g, 9.08 mmol, 1 eq) and 4,5-dimethyl-1H-imidazol-2-amine (1.01 g, 9.08 mmol, 1 eq) in AcOH (1 mL) at 90° C. for 24 hr. LC-MS showed desired product was detected. The reaction mixture was concentrated under reduced pressure to remove AcOH. The crude product was purified by re-crystallization from ethyl acetate (50 ml) at 25° C. to give (2,3,5,7-tetramethylimidazo[1,2-a]pyrimidin-6-yl) benzoate (1.4 g, 4.74 mmol, 52.20% yield) as yellow oil. LCMS for product (ESI): m/z 296.1 [M+H]⁺.

### Step 2: Preparation of 2,3,5,7-tetramethylimidazo[1,2-a]pyrimidin-6-ol

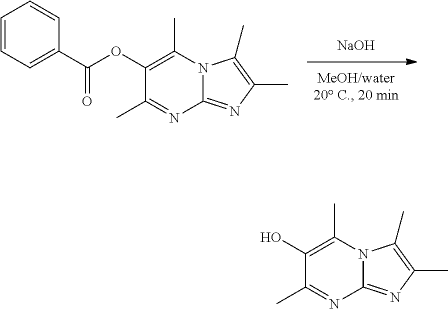 [SMILES: Cc1nc2nc(C)c(C)n2c(C)c1OC(=O)c1ccccc1 ; "NaOH" ; Cc1nc2nc(C)c(C)n2c(C)c1O]

A solution of (2,3,5,7-tetramethylimidazo[1,2-a]pyrimidin-6-yl) benzoate (1.4 g, 4.74 mmol, 1 eq) and NaOH (948.00 mg, 23.7 mmol, 5 eq) in H₂O (10 mL) and MeOH (10 mL) was stirred at 25° C. for 20 min. LC-MS showed all the benzoate was consumed and desired compound was detected. The reaction mixture was concentrated under reduced pressure to remove MeOH. The solution was adjusted to pH=4 by adding 1N HCl and filtered. The filter cake was dried to give 2,3,5,7-tetramethylimidazo[1,2-a]pyrimidin-6-ol (1.5 g, 7.84 mmol, 82.74% yield) as yellow solid.

¹H NMR (400 MHz, DMSO-d₆) δ=2.54 (s, 3H), 2.15 (s, 3H), 2.09 (s, 3H), 1.88 (s, 3H).

### Step 3: Preparation of (3R)-3-(2,3,5,7-tetramethylimidazo[1,2-a]pyrimidin-6-yl)oxypyrrolidine-1-carboxylate

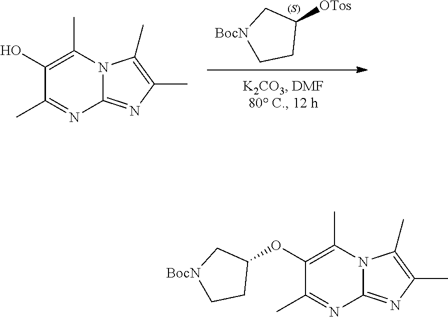

To a solution of tert-butyl 3-(p-tolylsulfonyloxy)pyrrolidine-1-carboxylate (642.75 mg, 1.88 mmol, 1.2 eq) and 2,3,5,7-tetramethylimidazo[1,2-a]pyrimidin-6-ol (300 mg, 1.57 mmol, 1 eq) in DMF (3 mL) was added K₂CO₃ (650.47 mg, 4.71 mmol, 3 eq) at 80° C. for 12 hr. LC-MS showed all the starting material was consumed and desired compound was detected. The residue was diluted with H₂O 5 mL and extracted with ethyl acetate 15 mL (5 mL×3). The combined organic layers were washed with brine 5 mL, dried over Na₂SO₄, filtered. The filtrate was concentrated under reduced pressure to give tert-butyl (3R)-3-(2,3,5,7-tetramethylimidazo[1,2-a]pyrimidin-6-yl)oxypyrrolidine-1-carboxylate (350 mg, 971.01 μmol, 61.90% yield) as yellow oil, which was used into next step directly without further purification. LCMS for product (ESI): m/z 361.3 [M+H]⁺.

### Step 4: Preparation of 2,3,5,7-tetramethyl-6-[(3R)-pyrrolidin-3-yl]oxy-imidazo[1,2-a]pyrimidine

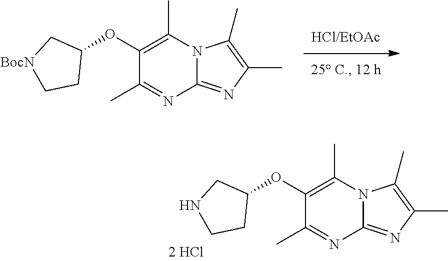 [SMILES: Cc1nc2nc(C)c(C)n2c(C)c1O[C@@H]1CCN(C(=O)OC(C)(C)C)C1 ; Cc1nc2nc(C)c(C)n2c(C)c1O[C@@H]1CCNC1]

A solution of tert-butyl (3R)-3-(2,3,5,7-tetramethylimidazo[1,2-a]pyrimidin-6-yl)oxypyrrolidine-1-carboxylate (300 mg, 832.29 μmol, 1 eq) in HCl/EtOAc (5 mL) was stirred at 25° C. for 12 hr. LC-MS showed all the Boc protected amine was consumed completely and one major peak was detected with desired Ms. The reaction mixture was concentrated under reduced pressure to remove HCl/EtOAc to give 2,3,5,7-tetramethyl-6-[(3R)-pyrrolidin-3-yl]oxy-imidazo[1,2-a]pyrimidine (250 mg, crude) as white solid, which used directly without further purification. LCMS for product (ESI): m/z 261.3 [M+H]⁺.

### Step 5: Preparation of 4-[[6-[4-[(3R)-3-(2,3,5,7-tetramethylimidazo[1,2-a] pyrimidin-6-yl)oxypyrrolidin-1-yl]phenyl] pyridazin-3-yl]methyl]morpholine (Compound 3)

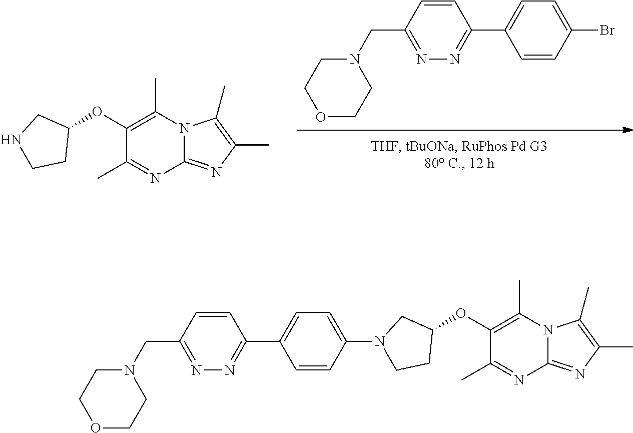 [SMILES: Cc1nc2nc(C)c(C)n2c(C)c1O[C@@H]1CCNC1 ; Brc1ccc(-c2ccc(CN3CCOCC3)nn2)cc1 ; Cc1nc2nc(C)c(C)n2c(C)c1O[C@@H]1CCN(c2ccc(-c3ccc(CN4CCOCC4)nn3)cc2)C1]

A mixture of 2,3,5,7-tetramethyl-6-[(3R)-pyrrolidin-3-yl]oxy-imidazo[1,2-a]pyrimidine (200 mg, 768.24 μmol, 1 eq), 4-[[6-(4-bromophenyl)pyridazin-3-yl]methyl]morpholine (256.76 mg, 768.24 μmol, 1 eq), tBuONa (1 M, 1.54 mL, 2 eq), [2-(2-aminophenyl)phenyl]-methylsulfonyloxy-palladium; dicyclohexyl-[2-(2,6-diisopropoxyphenyl)phenyl]phosphane (64.25 mg, 76.82 μmol, 0.1 eq) in THF (1 mL) was degassed and purged with N₂ for 3 times, and then the mixture was stirred at 80° C. for 12 hr under N₂ atmosphere. LC-MS showed all the amine was consumed and 43% of desired compound was detected. The residue was diluted with H₂O 3 mL and extracted with ethyl acetate 3 mL (1 mL×3). The combined organic layers were washed with brine 3 mL, dried over Na₂SO₄, filtered. The filtrate was concentrated under reduced pressure to give a residue. The residue was purified by prep-HPLC (HCl condition) to give 4-[[6-[4-[(3R)-3-(2,3,5,7-tetramethylimidazo[1,2-a] pyrimidin-6-yl)oxypyrrolidin-1-yl]phenyl] pyridazin-3-yl]methyl]morpholine (25 mg, 47.02 μmol, 6.12% yield, 96.6% purity) as red solid. LCMS for product (ESI): m/z 514.2 [M+H]⁺.

¹H NMR (400 MHz, DMSO-d₆) δ=14.74 (br s, 1H), 11.83 (br s, 1H), 8.31 (d, J=8.0 Hz, 1H), 8.18-8.05 (m, 3H), 6.77 (d, J=7.6 Hz, 2H), 4.97 (s, 1H), 4.67 (s, 2H), 3.91 (s, 4H), 3.47 (d, J=6.0 Hz, 4H), 3.33 (s, 4H), 2.85 (s, 3H), 2.66 (s, 3H), 2.56 (s, 3H), 2.41 (s, 3H), 2.35 (d, J=15.2 Hz, 2H).

### Example 4—Synthesis of (R)-4-((6-(4-(3-((2,5,7-trimethylpyrazolo[1,5-a]pyrimidin-6-yl)oxy)pyrrolidin-1-yl)phenyl)pyridazin-3-yl)methyl)morpholine (Compound 4)

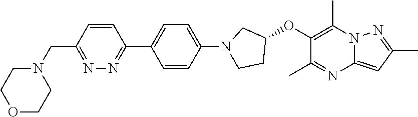 [SMILES: Cc1cc2nc(C)c(O[C@@H]3CCN(c4ccc(-c5ccc(CN6CCOCC6)nn5)cc4)C3)c(C)n2n1]

### Step 1: Preparation of (2,5,7-trimethylpyrazolo[1,5-a]pyrimidin-6-yl) benzoate

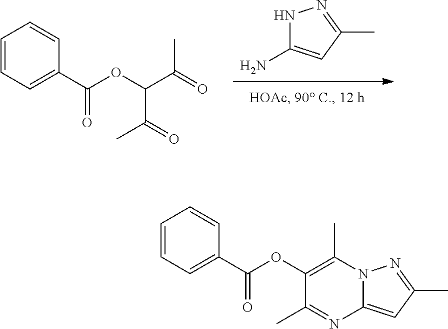

A solution of (1-acetyl-2-oxo-propyl) benzoate (2 g, 9.08 mmol, 1 eq) and 3-methyl-1H-pyrazol-5-amine (1.06 g, 10.90 mmol, 1.2 eq) in AcOH (1 mL) at 90° C. for 1 hr. LC-MS showed the diketone was consumed completely and one major peak was detected with desired Ms. The reaction mixture was concentrated under reduced pressure to remove AcOH to give (2,5,7-trimethylpyrazolo[1,5-a]pyrimidin-6-yl) benzoate (3 g, crude) as light yellow oil, which used directly without further purification. LCMS for product (ESI): m/z 282.2 [M+H]⁺.

### Step 2: Preparation of 2,5,7-trimethylpyrazolo[1,5-a]pyrimidin-6-ol

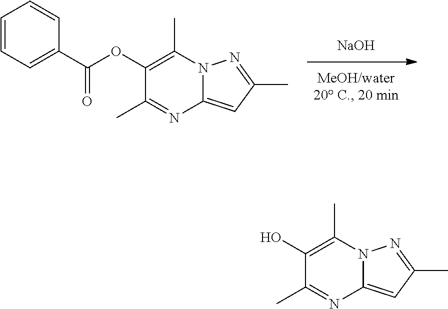 [SMILES: Cc1cc2nc(C)c(OC(=O)c3ccccc3)c(C)n2n1 ; "NaOH" ; Cc1cc2nc(C)c(O)c(C)n2n1]

To a solution of (2,5,7-trimethylpyrazolo[1,5-a]pyrimidin-6-yl) benzoate (3 g, 10.66 mmol, 1 eq) and NaOH (2.13 g, 53.32 mmol, 5 eq) in H₂O (20 mL) and MeOH (20 mL) at 25° C. for 20 min. LC-MS showed all the benzoate was consumed one major peak was detected with desired Ms. The reaction mixture was concentrated under reduced pressure to remove MeOH. The mixture was adjusted to Ph 4 with 1N HCl. The resulting product was dissolved and filtered to remove the insoluble. The filter cake was concentrated under reduced pressure to give 2,5,7-trimethylpyrazolo[1,5-a]pyrimidin-6-ol (1.35 g, 7.62 mmol, 71.44% yield) as white solid.

¹H NMR (400 MHz, DMSO-d₆) δ=5.77 (s, 1H), 2.30 (s, 3H), 2.23 (s, 3H), 2.20 (s, 3H)

### Step 3: Preparation of tert-butyl (3R)-3-(2,5,7-trimethylpyrazolo[1,5-a]pyrimidin-6-yl)oxypyrrolidine-1-carboxylate

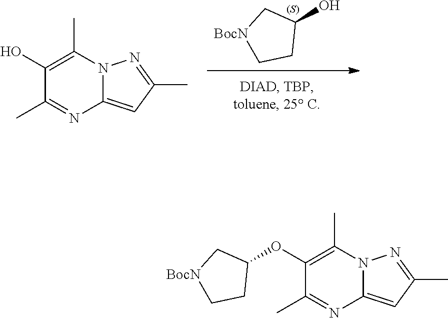

To a solution of 2,5,7-trimethylpyrazolo[1,5-a]pyrimidin-6-ol (1.2 g, 6.77 mmol, 1 eq), tert-butyl (3S)-3-hydroxypyrrolidine-1-carboxylate (1.89 g, 10.15 mmol, 1.5 eq) in toluene (6 mL) was added DIAD (2.05 g, 10.15 mmol, 1.96 mL, 1.5 eq) in toluene (6 mL) and TBP (2.05 g, 10.16 mmol, 2.49 mL, 1.5 eq) at 25° C. for 1 hr. LC-MS showed all the starting material was consumed and one major peak was detected with desired Ms. The residue was diluted with H₂O 20 mL and extracted with ethyl acetate 15 mL (5 mL×3). The combined organic layers were washed with brine 10 mL, dried over Na₂SO₄, filtered. The filtrate was concentrated under reduced pressure to give a residue. The residue was purified by column chromatography (SiO₂, Petroleum ether/Ethyl acetate=1/0 to 0/1) to give tert-butyl (3R)-3-(2,5,7-trimethylpyrazolo[1,5-a]pyrimidin-6-yl)oxypyrrolidine-1-carboxylate (2 g, 5.77 mmol, 78.70% yield) as yellow oil. LCMS for product (ESI): m/z 347.2 [M+H]⁺.

¹H NMR (400 MHz, CHLOROFORM-d) δ=6.35 (s, 1H), 4.57 (s, 1H), 4.45 (s, 1H), 3.66-3.38 (m, 6H), 2.69 (s, 3H), 2.57-2.48 (m, 6H), 1.46 (s, 9H).

### Step 4: Preparation of 2,5,7-trimethyl-6-[(3R)-pyrrolidin-3-yl]oxy-pyrazolo[1,5-a]pyrimidine

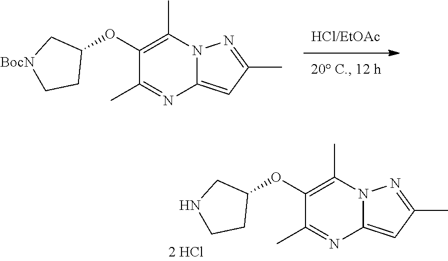 [SMILES: Cc1cc2nc(C)c(O[C@@H]3CCN(C(=O)OC(C)(C)C)C3)c(C)n2n1 ; Cc1cc2nc(C)c(O[C@@H]3CCNC3)c(C)n2n1]

A solution of tert-butyl (3R)-3-(2,5,7-trimethylpyrazolo[1,5-a]pyrimidin-6-yl)oxypyrrolidine-1-carboxylate (500 mg, 1.44 mmol, 1 eq) in HCl/EtOAc (20 mL) at 25° C. for 12 hr. LC-MS showed the Boc protected amine was consumed completely and one major peak was detected with desired Ms. The reaction mixture was concentrated under reduced pressure to remove HCl/EtOAc to give 2,5,7-trimethyl-6-[(3R)-pyrrolidin-3-yl]oxy-pyrazolo[1,5-a]pyrimidine (280 mg, 1.14 mmol, 78.76% yield) as white solid, which used directly without further purification. LCMS for product (ESI): m/z 247.2 [M+H]⁺.

### Step 5: Preparation of 4-[[6-[4-[(3R)-3-(2,5,7-trimethylpyrazolo[1,5-a]pyrimidin-6-yl) oxypyrrolidin-1-yl]phenyl] pyridazin-3-yl]methyl]morpholine (Compound 4)

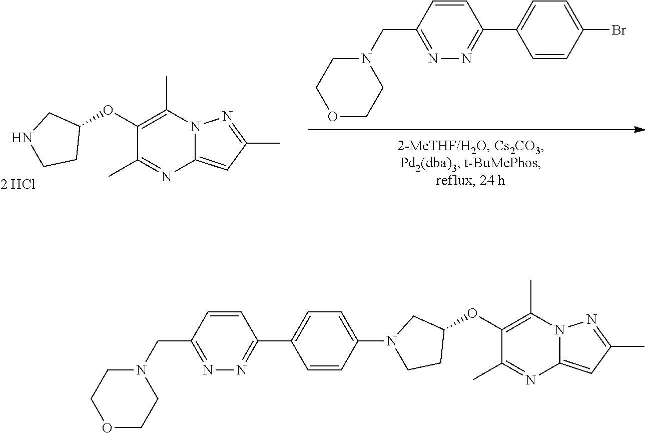 [SMILES: Cc1cc2nc(C)c(O[C@@H]3CCNC3)c(C)n2n1 ; Brc1ccc(-c2ccc(CN3CCOCC3)nn2)cc1 ; Cc1cc2nc(C)c(O[C@@H]3CCN(c4ccc(-c5ccc(CN6CCOCC6)nn5)cc4)C3)c(C)n2n1]

A mixture of 2,5,7-trimethyl-6-[(3R)-pyrrolidin-3-yl]oxy-pyrazolo[1,5-a]pyrimidine (100 mg, 406.00 μmol, 1 eq), 4-[[6-(4-bromophenyl)pyridazin-3-yl]methyl]morpholine (135.69 mg, 406.00 μmol, 1 eq), Cs₂CO₃ (529.13 mg, 1.62 mmol, 4 eq), Pd₂(dba)₃ (7.44 mg, 8.12 μmol, 0.02 eq) and ditert-butyl-[2-(o-tolyl)phenyl]phosphane (5.07 mg, 16.24 μmol, 0.04 eq) in 2-MeTHF (2 mL) and H₂O (1 mL) was degassed and purged with N₂ for 3 times, and then the mixture was stirred at 80° C. for 2 hr under N₂ atmosphere. LC-MS showed all the amine was consumed and 30% of desired compound was detected. The residue was diluted with H₂O 3 mL and extracted with ethyl acetate 3 mL (1 mL×3). The combined organic layers were washed with brine 3 mL, dried over Na₂SO₄, filtered. The filtrate was concentrated under reduced pressure to give a residue. The residue was purified by prep-HPLC (HCl condition) to give 4-[[6-[4-[(3R)-3-(2,5,7-trimethylpyrazolo[1,5-a]pyrimidin-6-yl) oxypyrrolidin-1-yl]phenyl]pyridazin-3-yl]methyl]morpholine (24 mg, 48.2 μmol, 14.68% yield, 98.2% purity) as red solid. LCMS for product (ESI): m/z 500.2 [M+H]⁺.

¹H NMR (400 MHz, DMSO-d₆) δ=11.70 (br s, 1H), 8.32 (d, J=8.8 Hz, 1H), 8.14-8.04 (m, 3H), 6.78 (d, J=8.8 Hz, 2H), 6.36 (s, 1H), 4.92 (s, 1H), 4.68 (s, 2H), 3.90 (s, 4H), 3.72-3.62 (m, 1H), 3.58 (d, J=11.6 Hz, 2H), 3.52-3.43 (m, 1H), 3.33 (s, 4H), 2.53 (s, 3H), 2.45 (s, 3H), 2.39 (s, 3H), 2.37-2.25 (m, 2H).

### Example 5—Synthesis of (R)-4-(3-(5-(3-((2,5,7-trimethyl-[1,2,4]triazolo[1,5-a]pyrimidin-6-yl)oxy)pyrrolidin-1-yl)pyrimidin-2-yl)prop-2-yn-1-yl)morpholine

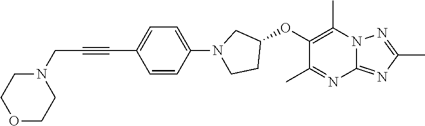 [SMILES: Cc1nc2nc(C)c(O[C@@H]3CCN(c4ccc(C#CCN5CCOCC5)cc4)C3)c(C)n2n1]

Step 1: Preparation of 4-prop-2-ynylmorpholine

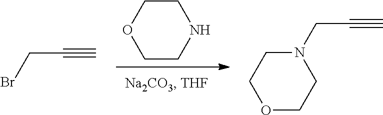

To a solution of 3-bromoprop-1-yne (15.00 g, 100.87 mmol, 10.87 mL, 80% purity, 3 eq) in THE (50 mL) was added Na₂CO₃ (7.13 g, 67.25 mmol, 2 eq) and morpholine (2.93 g, 33.62 mmol, 2.96 mL, 1 eq). The mixture was stirred at 25° C. for 12 hr. TLC showed morpholine was consumed and one major new spot with lower polarity was detected. The mixture was filtered and the filtrate was concentrated under reduced pressure to give a residue. The residue was diluted with H₂O 30 mL and extracted with EtOAc (10 mL×3). The combined organic layers were washed with brine 10 mL, dried over Na₂SO₄, filtered and concentrated under reduced pressure to give a residue. Compound 4-prop-2-ynylmorpholine (2.6 g, 20.77 mmol, 61.78% yield) was obtained as yellow oil without further purification.

¹H NMR (400 MHz, CHLOROFORM-d) δ=3.78-3.72 (m, 4H), 3.29 (d, J=2.6 Hz, 2H), 2.61-2.53 (m, 4H), 2.27 (t, J=2.4 Hz, 1H)

### Step 2: Preparation of 4-[3-(5-bromopyrimidin-2-yl)prop-2-ynyl]morpholine

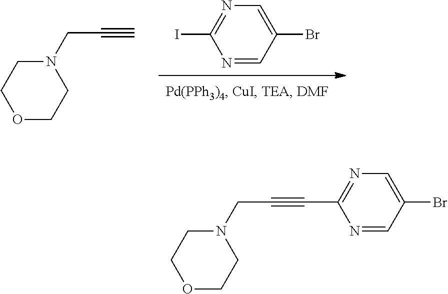

To a mixture of 4-prop-2-ynylmorpholine (966.62 mg, 7.72 mmol, 1.1 eq), 5-bromo-2-iodo-pyrimidine (2 g, 7.02 mmol, 1 eq), Et₃N (2.56 g, 25.27 mmol, 3.52 mL, 3.6 eq) in DMF (20 mL) was added CuI (66.85 mg, 351.02 μmol, 0.05 eq) and Pd(PPh₃)₂Cl₂ (246.38 mg, 351.02 mol, 0.05 eq). The mixture was degassed and purged with N₂ for 3 times, and then stirred at 25° C. for 12 hr under N₂ atmosphere. LC-MS showed all the starting material was consumed and desired compound was detected. The mixture was quenched by H₂O (20 mL) and then filtered. The filter cake was purified by flash silica gel chromatography. Compound 4-[3-(5-bromopyrimidin-2-yl)prop-2-ynyl]morpholine (800 mg, 2.84 mmol, 40.39% yield) was obtained as a yellow solid.

¹H NMR (400 MHz, CHLOROFORM-d) δ=8.79-8.74 (m, 2H), 3.80-3.73 (m, 4H), 3.59 (s, 2H), 2.72-2.65 (m, 4H)

### Step 3: Preparation of 4-[3-[5-[(3R)-3-[(2,5,7-trimethyl-[1,2,4]triazolo[1,5-a]pyrimidin-6-yl)oxy]pyrrolidin-1-yl]pyrimidin-2-yl]prop-2-ynyl]morpholine (Compound 5)

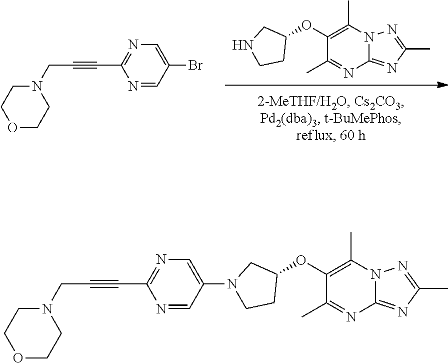 [SMILES: Brc1cnc(C#CCN2CCOCC2)nc1 ; Cc1nc2nc(C)c(O[C@@H]3CCNC3)c(C)n2n1 ; Cc1nc2nc(C)c(O[C@@H]3CCN(c4cnc(C#CCN5CCOCC5)nc4)C3)c(C)n2n1]

To a mixture of 4-[3-(5-bromopyrimidin-2-yl)prop-2-ynyl]morpholine (100 mg, 354.44 μmol, 1 eq), 2,5,7-trimethyl-6-[(3R)-pyrrolidin-3-yl]oxy-[1,2,4]triazolo[1,5-a]pyrimidine (136.20 mg, 425.33 μmol, 1.2 eq, 2HCl), Cs₂CO₃ (577.41 mg, 1.77 mmol, 5 eq) in H₂O (1 mL) and 2-MeTHF (2 mL) was added 2-ditert-butylphosphino-2′-methylbiphenyl (22.15 mg, 70.89 mol, 0.2 eq) and Pd₂(dba)₃ (32.46 mg, 35.44 μmol, 0.1 eq) in one portion. The mixture was degassed and purged with N₂ for 3 times, and then stirred at 80° C. for 60 hr under N₂ atmosphere. LC-MS showed all the bromide was consumed and desired compound was detected. The mixture was filtered and the filtrate was partitioned between H₂O 20 mL and EtOAc 5 mL. The aqueous phase was separated and concentrated under reduced pressure to give a residue. The residue was purified by prep-HPLC (neutral condition). Compound 4-[3-[5-[(3R)-3-[(2,5,7-trimethyl-[1,2,4]triazolo[1,5-a]pyrimidin-6-yl)oxy]pyrrolidin-1-yl]pyrimidin-2-yl]prop-2-ynyl]morpholine (18 mg, 38.42 μmol, 2.71% yield, 95.743% purity) was obtained as a light yellow solid. LCMS for product (ESI): m/z 449.2 [M+H]⁺.

¹H NMR (400 MHz, DMSO-d₆) δ=8.15 (s, 2H), 4.97 (br s, 1H), 3.67-3.54 (m, 7H), 3.51 (s, 2H), 3.45-3.41 (m, 1H), 2.57 (s, 3H), 2.54-2.51 (m, 7H), 2.47 (s, 3H), 2.43-2.35 (m, 1H), 2.32-2.21 (m, 1H).

### Example 6—Synthesis of (R)-4-((4-(5-(3-((2,5,7-trimethyl-[1,2,4]triazolo[1,5-a]pyrimidin-6-yl)oxy)pyrrolidin-1-yl)pyrimidin-2-yl)bicyclo[2.2.2]octan-1-yl)methyl)morpholine (Compound 6)

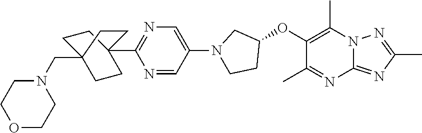 [SMILES: Cc1nc2nc(C)c(O[C@@H]3CCN(c4cnc(C56CCC(CN7CCOCC7)(CC5)CC6)nc4)C3)c(C)n2n1]

### Step 1: Preparation of methyl 1-(hydroxymethyl)bicycle [2.2.2] octane-4-carboxylate

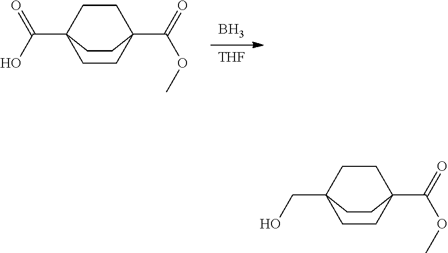

To a solution of 4-methoxycarbonylbicyclo[2.2.2]octane-1-carboxylic acid (20 g, 94.23 mmol, 1 eq) in THE (200 mL) was added BH₃-Me₂S (12 M, 23.56 mL, 3 eq) dropwise at 0° C. The mixture was stirred at 25° C. for 12 hr. TLC indicated ˜0% of the carboxylic acid was remained, and one major new spot with lower polarity was detected. The reaction mixture was quenched by addition MeOH 100 mL at 10° C., and then stirred at 25° C. for 2 h. The solution was concentrated under reduced pressure to give a residue. Compound methyl 1-(hydroxymethyl)bicycle [2.2.2] octane-4-carboxylate (18 g, 90.79 mmol, 96.35% yield) was obtained as light yellow oil without further purification.

¹H NMR (400 MHz, CHLOROFORM-d) δ=3.65 (s, 3H), 3.41 (s, 1H), 3.29 (d, J=5.2 Hz, 1H), 1.84-1.73 (m, 6H), 1.48-1.38 (m, 6H)

### Step 2: Preparation of methyl 1-formylbicyclo[2.2.2]octane-4-carboxylate

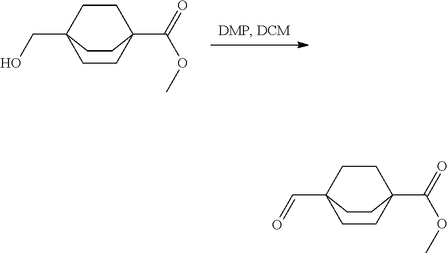

To a solution of methyl 1-(hydroxymethyl)bicyclo[2.2.2]octane-4-carboxylate (18 g, 90.79 mmol, 1 eq) in DCM (300 mL) was added DMP (50.06 g, 118.03 mmol, 36.54 mL, 1.3 eq) at 20° C. The mixture was stirred at 20° C. for 12 hr. TLC indicated one major new spot with lower polarity was detected. The mixture was filtered and the filtrate was quenched by addition sat. NaHCO₃ aq 100 mL at 0° C., and then extracted with DCM 240 mL (80 mL×3). The combined organic layers were washed with brine (100 mL), dried over Na₂SO₄, filtered and concentrated under reduced pressure to give a residue. The residue was purified by column chromatography (SiO₂, Petroleum ether/Ethyl acetate=15/1 to 10/1). Compound methyl 1-formylbicyclo[2.2.2]octane-4-carboxylate (9 g, 45.86 mmol, 50.51% yield) was obtained as colorless oil.

¹H NMR (400 MHz, CHLOROFORM-d) δ=9.41 (s, 1H), 3.64 (s, 3H), 1.85-1.80 (m, 6H), 1.70-1.63 (m, 6H)

### Step 3: Preparation of methyl 1-(morpholinomethyl)bicyclo[2.2.2]octane-4-carboxylate

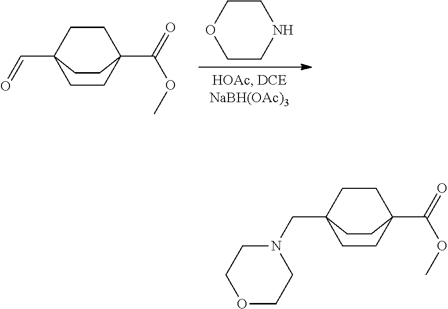

To a solution of methyl 1-formylbicyclo[2.2.2]octane-4-carboxylate (4.5 g, 22.93 mmol, 1 eq) and morpholine (4.00 g, 45.86 mmol, 4.04 mL, 2 eq) in DCE (100 mL) was added HOAc (1.38 g, 22.93 mmol, 1.31 mL, 1 eq) in one portion, and the mixture was stirred at 25° C. for 2 h, then NaBH(OAc)₃ (14.58 g, 68.79 mmol, 3 eq) was added in one portion and stirred at 25° C. for 12 h. TLC indicated ˜5% of the aldehyde was remained, and one major new spot with larger polarity was detected. The reaction mixture was quenched by addition sat. Na₂CO₃ 50 mL at 25° C., and then diluted with DCM 100 mL and extracted with DCM 150 mL (50 mL×3). The combined organic layers were washed with brine 50 mL, dried over Na₂SO₄, filtered and concentrated under reduced pressure to give a residue. The residue was purified by column chromatography (SiO₂, Petroleum ether/Ethyl acetate=12/1 to 10/1). Compound methyl 1-(morpholinomethyl)bicyclo[2.2.2]octane-4-carboxylate (7.2 g, 26.93 mmol, 58.72% yield) was obtained as a white solid.

¹H NMR (400 MHz, CHLOROFORM-d) δ=3.71-3.60 (m, 7H), 2.49-2.41 (m, 4H), 2.05 (s, 2H), 1.82-1.73 (m, 6H), 1.48-1.40 (m, 6H)

### Step 4: Preparation of 1-(morpholinomethyl)bicyclo[2.2.2]octane-4-carboxamidine

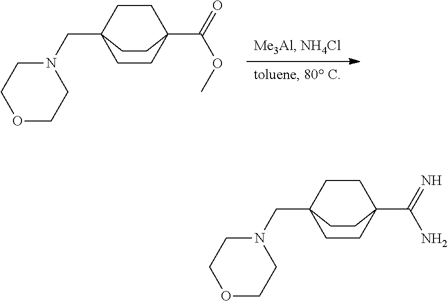

To a solution of NH₄Cl (1.00 g, 18.70 mmol, 5 eq) in toluene (10 mL) was added dropwise Al(CH₃)₃ (2 M, 9.35 mL, 5 eq) at 0° C. After addition, the mixture was stirred at 25° C. for 2 h, and then methyl 1-(morpholinomethyl)bicyclo[2.2.2]octane-4-carboxylate (1 g, 3.74 mmol, 1 eq) in toluene (10 mL) was added dropwise at 25° C. The resulting mixture was stirred at 80° C. for 12 hr. TLC indicated all the methyl ester was consumed and one major new spot with larger polarity was detected. The reaction mixture was quenched by addition MeOH 20 mL at 0° C., the white precipitate was filtered off, washed with MeOH (20 mL). The filtrate was concentrated, treated again with MeOH (20 mL), and then MeOH:DCM=1:1 (20 mL). The final filtrate was concentrated under reduced pressure to give 1-(morpholinomethyl)bicyclo[2.2.2]octane-4-carboxamidine (1 g, crude, HCl) was obtained as a light yellow solid.

### Step 5: Preparation of 4-[[4-(5-chloropyrimidin-2-yl)-1-bicyclo[2.2.2]octanyl]methyl]morpholine

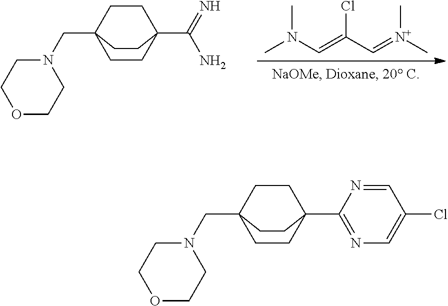

To a solution of 1-(morpholinomethyl)bicyclo[2.2.2]octane-4-carboxamidine (1 g, 3.47 mmol, 1 eq, HCl) in MeOH (30 mL) was added NaOMe (2.82 g, 15.63 mmol, 30% purity, 4.5 eq) in one portion. The mixture was stirred at 25° C. for 2 h, then [(Z)-2-chloro-3-(dimethylamino)prop-2-enylidene]-dimethyl-ammonium; hexafluorophosphate (905.48 mg, 2.95 mmol, 0.85 eq) was added in one portion. The mixture was stirred at 25° C. for 12 h. LC-MS showed all the amidine was consumed and desired compound was detected. The mixture was concentrated under reduced pressure to give a residue. The residue was purified by column chromatography (SiO₂, DCM:MeOH=40:1 to 30:1). Compound 4-[[4-(5-chloropyrimidin-2-yl)-1-bicyclo[2.2.2]octanyl]methyl]morpholine (50 mg, 155.35 μmol, 4.47% yield) was obtained as a light yellow solid. LCMS for product (ESI): m/z 322.2 [M+H]⁺.

¹H NMR (400 MHz, DMSO-d₆) δ=8.83 (s, 2H), 3.54 (t, J=4.4 Hz, 4H), 2.40 (t, J=4.4 Hz, 4H), 2.03 (s, 2H), 1.89-1.84 (m, 6H), 1.50-1.46 (m, 6H).

### Step 6: Preparation of 4-[[4-[5-[(3R)-3-[(2,5,7-trimethyl-[1,2,4]triazolo[1,5-a]pyrimidin-6-yl)oxy]pyrrolidin-1-yl]pyrimidin-2-yl]-1-bicyclo[2.2.2]octanyl]methyl]morpholine (Compound 6)

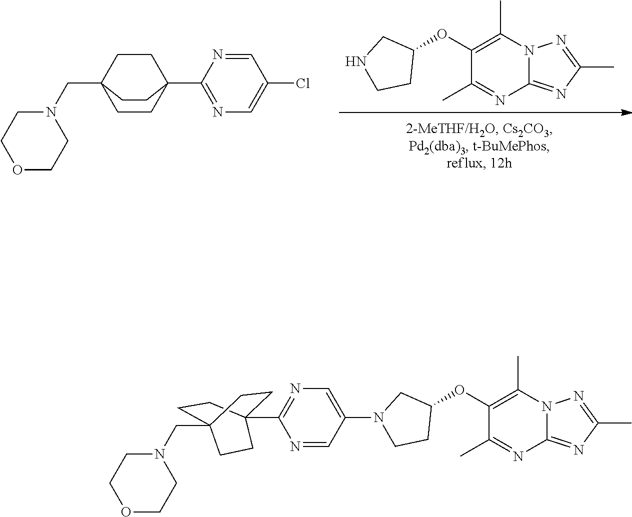 [SMILES: Clc1cnc(C23CCC(CN4CCOCC4)(CC2)CC3)nc1 ; Cc1nc2nc(C)c(O[C@@H]3CCNC3)c(C)n2n1 ; Cc1nc2nc(C)c(O[C@@H]3CCN(c4cnc(C56CCC(CN7CCOCC7)(CC5)CC6)nc4)C3)c(C)n2n1]

A mixture of 4-[[4-(5-chloropyrimidin-2-yl)-1-bicyclo[2.2.2]octanyl]methyl]morpholine (41.65 mg, 129.40 μmol, 0.8 eq), 2,5,7-trimethyl-6-[(3R)-pyrrolidin-3-yl]oxy-[1,2,4]triazolo[1,5-a]pyrimidine (40 mg, 161.75 μmol, 1 eq), Cs₂CO₃ (210.81 mg, 647.00 μmol, 4 eq), Pd₂(dba)₃ (14.81 mg, 16.17 μmol, 0.1 eq) and ditert-butyl-[2-(o-tolyl)phenyl]phosphane (10.11 mg, 32.35 μmol, 0.2 eq) in 2-MeTHF (1 mL) and H₂O (0.5 mL) was degassed and purged with N₂ for 3 times, and then the mixture was stirred at 80° C. for 2 hr under N₂ atmosphere. LC-MS showed all the pyrimidine chloride was consumed and desired compound was detected. The reaction mixture was concentrated under reduced pressure to remove 2-MeTHF. The residue was diluted with H₂O 3 mL and extracted with ethyl acetate 3 mL (3 mL×3). The combined organic layers were washed with brine 3 mL, dried over Na₂SO₄, filtered and concentrated under reduced pressure to give a residue. The residue was purified by prep-HPLC (HCl condition). Compound 4-[[4-[5-[(3R)-3-[(2,5,7-trimethyl-[1,2,4]triazolo[1,5-a]pyrimidin-6-yl)oxy]pyrrolidin-1-yl]pyrimidin-2-yl]-1-bicyclo[2.2.2]octanyl]methyl]morpholine (8 mg, 13.97 μmol, 8.64% yield, 93% purity) was obtained as a yellow solid. LCMS for product (ESI): m/z 533.3 [M+H]⁺.

¹H NMR (400 MHz, DMSO-d₆) δ=10.51 (br s, 1H), 8.35 (s, 2H), 5.03 (br s, 1H), 4.14-4.06 (m, 2H), 3.83 (d, J=12.6 Hz, 2H), 3.74-3.65 (m, 2H), 3.63-3.55 (m, 1H), 3.49-3.38 (m, 3H), 3.17-3.09 (m, 2H), 3.01 (d, J=3.6 Hz, 2H), 2.62-2.59 (m, 3H), 2.57 (s, 3H), 2.53 (s, 3H), 2.40 (br dd, J=5.6, 13.6 Hz, 1H), 2.30-2.20 (m, 1H), 1.97-1.86 (m, 6H), 1.78-1.72 (m, 6H).

### Example 7—Synthesis of (R)-6-((6-(4-(3-((2,5,7-trimethyl-[1,2,4]triazolo[1,5-a]pyrimidin-6-yl)oxy)pyrrolidin-1-yl)phenyl)pyridazin-3-yl)methyl)-2-oxa-6-azaspiro[3.3]heptane (Compound 7)

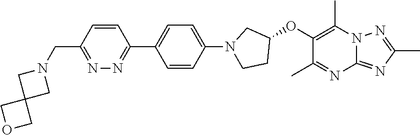 [SMILES: Cc1nc2nc(C)c(O[C@@H]3CCN(c4ccc(-c5ccc(CN6CC7(COC7)C6)nn5)cc4)C3)c(C)n2n1]

### Step 1: Preparation of 6-[[6-(4-bromophenyl)pyridazin-3-yl]methyl]-2-oxa-6-azaspiro[3.3]heptane

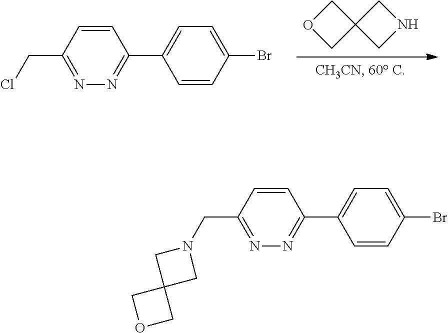

A mixture of 3-(4-bromophenyl)-6-(chloromethyl)pyridazine (200 mg, 705.34 μmol, 1 eq), 2-oxa-6-azaspiro[3.3]heptane (114.77 mg, 846.41 μmol, 1.2 eq, HCl), K₂CO₃ (194.96 mg, 1.41 mmol, 2 eq) in MeCN (5 mL) was stirred at 60° C. for 12 hr. LC-MS showed all the chloride was consumed and desired compound was detected. The reaction mixture was quenched by addition water 4 mL at 25° C., and then extracted with EtOAc (5 mL×2). The combined organic layers were washed with brine 5 mL, dried over Na₂SO₄, filtered. The filtrate was concentrated under reduced pressure to give a residue. Compound 6-[[6-(4-bromophenyl)pyridazin-3-yl]methyl]-2-oxa-6-azaspiro[3.3]heptane (244 mg, crude) as a brown solid, which was used into next step directly without further purification. LCMS for product (ESI): m/z 346.1 [M+H]⁺, 348.1 [M+H+2]⁺.

### Step 2: Preparation of 6-[[6-[4-[(3R)-3-[(2,5,7-trimethyl-[1,2,4]triazolo[1,5-a]pyrimidin-6-yl)oxy]pyrrolidin-1-yl]phenyl]pyridazin-3-yl]methyl]-2-oxa-6-azaspiro[3.3]heptane (Compound 7)

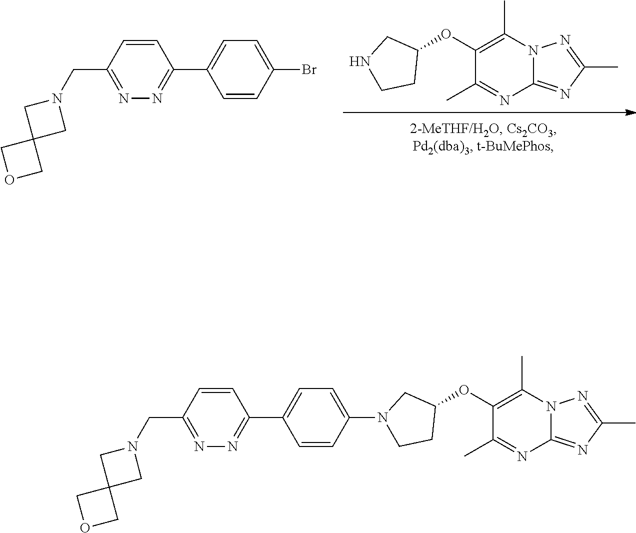 [SMILES: Brc1ccc(-c2ccc(CN3CC4(COC4)C3)nn2)cc1 ; Cc1nc2nc(C)c(O[C@@H]3CCNC3)c(C)n2n1 ; Cc1nc2nc(C)c(O[C@@H]3CCN(c4ccc(-c5ccc(CN6CC7(COC7)C6)nn5)cc4)C3)c(C)n2n1]

A mixture of 6-[[6-(4-bromophenyl)pyridazin-3-yl]methyl]-2-oxa-6-azaspiro[3.3]heptane (244 mg, 704.75 μmol, 1 eq), 2,5,7-trimethyl-6-[(3R)-pyrrolidin-3-yl]oxy-[1,2,4]triazolo[1,5-a]pyrimidine (270.81 mg, 845.70 μmol, 1.2 eq, 2HCl), Cs₂CO₃ (1.15 g, 3.52 mmol, 5 eq), Pd₂(dba)₃ (64.54 mg, 70.48 μmol, 0.1 eq) and ditert-butyl-[2-(o-tolyl)phenyl]phosphane (44.04 mg, 140.95 μmol, 0.2 eq) in 2-MeTHF (4 mL) and H₂O (2 mL) was degassed and purged with N₂ for 3 times, and then the mixture was stirred at 80° C. for 12 hr under N₂ atmosphere. LC-MS showed all the bromide was consumed and desired compound was detected. The reaction mixture was diluted with water 4 mL and extracted with EtOAc (3 mL×3). The combined organic layers were washed with brine 4 mL, dried over Na₂SO₄, filtered and concentrated under reduced pressure to give a residue. The residue was purified by prep-HPLC (neutral condition). Compound 6-[[6-[4-[(3R)-3-[(2,5,7-trimethyl-[1,2,4]triazolo[1,5-a]pyrimidin-6-yl)oxy]pyrrolidin-1-yl]phenyl]pyridazin-3-yl]methyl]-2-oxa-6-azaspiro[3.3]heptane (29 mg, 55.97 μmol, 7.94% yield, 98.939% purity) was obtained as a light yellow solid. LCMS for product (ESI): m/z 513.2 [M+H]⁺.

¹H NMR (400 MHz, DMSO-d₆) δ=8.04-8.00 (m, 3H), 7.54 (d, J=8.8 Hz, 1H), 6.73 (d, J=8.4 Hz, 2H), 4.97 (br s, 1H), 4.62 (s, 4H), 3.79 (s, 2H), 3.70-3.61 (m, 1H), 3.60-3.50 (m, 2H), 3.44 (dd, J=4.0, 12.0 Hz, 1H), 3.39 (s, 4H), 2.58 (s, 3H), 2.52 (s, 3H), 2.46 (s, 3H), 2.40 (s, 1H), 2.34-2.27 (m, 1H)

### Example 8—Synthesis of (R)-6-((1-(4-(6-((3-methoxyazetidin-1-yl)methyl)pyridazin-3-yl)phenyl)pyrrolidin-3-yl)oxy)-2,5,7-trimethyl-[1,2,4]triazolo[1,5-a]pyrimidine (Compound 8)

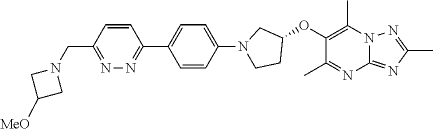 [SMILES: COC1CN(Cc2ccc(-c3ccc(N4CC[C@@H](Oc5c(C)nc6nc(C)nn6c5C)C4)cc3)nn2)C1]

### Step 1: Preparation of 3-(4-bromophenyl)-6-[(3-methoxyazetidin-1-yl)methyl]pyridazine

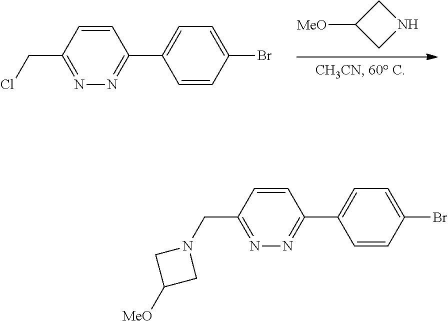

A mixture of 3-(4-bromophenyl)-6-(chloromethyl)pyridazine (100 mg, 352.67 μmol, 1 eq), 3-methoxyazetidine; hydrochloride (47.94 mg, 387.94 μmol, 1.1 eq), K₂CO₃ (146.22 mg, 1.06 mmol, 3 eq) in MeCN (2 mL) was stirred at 60° C. for 2 hr. LC-MS showed all the chloride was consumed and desired compound was detected. The reaction mixture was quenched by addition water 2 mL at 25° C., and then extracted with EtOAc (5 mL×2). The combined organic layers were washed with brine 5 mL, dried over Na₂SO₄, filtered. The filtrate was concentrated under reduced pressure to give 3-(4-bromophenyl)-6-[(3-methoxyazetidin-1-yl)methyl]pyridazine (117 mg, crude) as a brown solid, which was used into next step directly without further purification. LCMS for product (ESI): m/z 334.1 [M+H]⁺, 336.1 [M+H+2]⁺.

### Step 2: Preparation of 6-[(3R)-1-[4-[6-[(3-methoxyazetidin-1-yl)methyl]pyridazin-3-yl]phenyl]pyrrolidin-3-yl]oxy-2,5,7-trimethyl-[1,2,4]triazolo[1,5-a]pyrimidine (Compound 8)

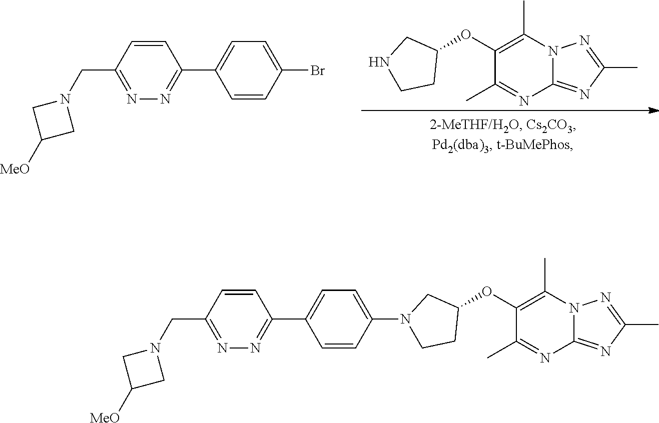 [SMILES: COC1CN(Cc2ccc(-c3ccc(Br)cc3)nn2)C1 ; Cc1nc2nc(C)c(O[C@@H]3CCNC3)c(C)n2n1 ; COC1CN(Cc2ccc(-c3ccc(N4CC[C@@H](Oc5c(C)nc6nc(C)nn6c5C)C4)cc3)nn2)C1]

A mixture of 3-(4-bromophenyl)-6-[(3-methoxyazetidin-1-yl)methyl]pyridazine (117 mg, 350.08 μmol, 1 eq), 2,5,7-trimethyl-6-[(3R)-pyrrolidin-3-yl]oxy-[1,2,4]triazolo[1,5-a]pyrimidine (134.52 mg, 420.09 μmol, 1.2 eq, 2HCl), Cs₂CO₃ (570.31 mg, 1.75 mmol, 5 eq), Pd₂(dba)₃ (32.06 mg, 35.01 μmol, 0.1 eq) and ditert-butyl-[2-(o-tolyl)phenyl]phosphane (21.87 mg, 70.02 μmol, 0.2 eq) in 2-MeTHF (2 mL) and H₂O (1 mL) was degassed and purged with N₂ for 3 times, and then the mixture was stirred at 80° C. for 24 hr under N₂ atmosphere. LC-MS showed all the bromide was consumed and desired compound was detected. The reaction mixture was diluted with water 3 mL and extracted with EtOAc (5 mL×3). The combined organic layers were washed with brine 3 mL, dried over Na₂SO₄, filtered and concentrated under reduced pressure to give a residue. The residue was purified by prep-HPLC (neutral condition). Compound 6-[(3R)-1-[4-[6-[(3-methoxyazetidin-1-yl)methyl]pyridazin-3-yl]phenyl]pyrrolidin-3-yl]oxy-2,5,7-trimethyl-[1,2,4]triazolo[1,5-a]pyrimidine (16 mg, 30.64 mol, 8.75% yield, 95.865% purity) was obtained as a light yellow solid. LCMS for product (ESI): m/z 501.2 [M+H]⁺.

1H NMR (400 MHz, DMSO-d₆) δ=8.08-8.00 (m, 3H), 7.56 (d, J=8.8 Hz, 1H), 6.73 (d, J=9.2 Hz, 2H), 4.97 (s, 1H), 4.00 (q, J=5.6 Hz, 1H), 3.89-3.83 (m, 2H), 3.70-3.60 (m, 1H), 3.60-3.56 (m, 1H), 3.55-3.51 (m, 2H), 3.48-3.41 (m, 1H), 3.29 (s, 1H), 3.15 (s, 3H), 2.98 (dd, J=5.6, 8.0 Hz, 2H), 2.58 (s, 3H), 2.52 (s, 3H), 2.46 (s, 3H), 2.43-2.33 (m, 2H).

### Example 9—Synthesis of (R)—N,N-dimethyl-1-((6-(4-(3-((2,5,7-trimethyl-[1,2,4]triazolo[1,5-a]pyrimidin-6-yl)oxy)pyrrolidin-1-yl)phenyl)pyridazin-3-yl)methyl)azetidin-3-amine (Compound 9)

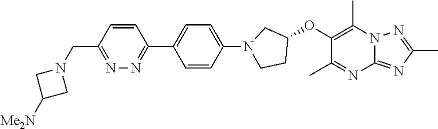 [SMILES: Cc1nc2nc(C)c(O[C@@H]3CCN(c4ccc(-c5ccc(CN6CC(N(C)C)C6)nn5)cc4)C3)c(C)n2n1]

### Step 1: Preparation of 1-[[6-(4-bromophenyl)pyridazin-3-yl]methyl]-N,N-dimethyl-azetidin-3-amine

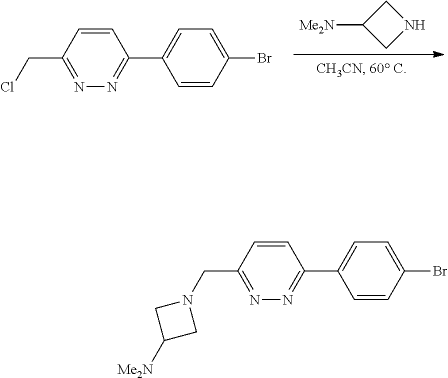 [SMILES: ClCc1ccc(-c2ccc(Br)cc2)nn1 ; CN(C)C1CNC1 ; CN(C)C1CN(Cc2ccc(-c3ccc(Br)cc3)nn2)C1]

A mixture of 3-(4-bromophenyl)-6-(chloromethyl)pyridazine (200 mg, 705.34 μmol, 1 eq), N,N-dimethylazetidin-3-amine; hydrochloride (115.64 mg, 846.41 μmol, 1.2 eq), K₂CO₃ (194.97 mg, 1.41 mmol, 2 eq) in MeCN (5 mL) was stirred at 60° C. for 12 hr. LC-MS showed all the chloride was consumed and desired compound was detected. The reaction mixture was quenched by adding water (4 mL) at 25° C., and then extracted with EtOAc (5 mL×2). The combined organic layers were washed with brine 3 mL, dried over Na₂SO₄, filtered. The filtrate was concentrated under reduced pressure to give 1-[[6-(4-bromophenyl)pyridazin-3-yl]methyl]-N,N-dimethyl-azetidin-3-amine (244 mg, crude) as a brown solid, which was used into next step directly without further purification. LCMS for product (ESI): m/z 347.1 [M+H]⁺, 349.1 [M+H+2]⁺.

### Step 2: Preparation of N,N-dimethyl-1-[[6-[4-[(3R)-3-[(2,5,7-trimethyl-[1,2,4]triazolo[1,5-a]pyrimidin-6-yl)oxy]pyrrolidin-1-yl]phenyl]pyridazin-3-yl]methyl]azetidin-3-amine (Compound 9)

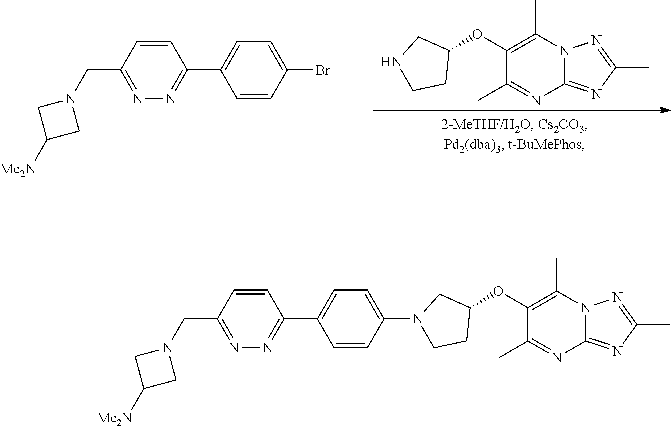 [SMILES: CN(C)C1CN(Cc2ccc(-c3ccc(Br)cc3)nn2)C1 ; Cc1nc2nc(C)c(O[C@@H]3CCNC3)c(C)n2n1 ; Cc1nc2nc(C)c(O[C@@H]3CCN(c4ccc(-c5ccc(CN6CC(N(C)C)C6)nn5)cc4)C3)c(C)n2n1]

A mixture of 1-[[6-(4-bromophenyl)pyridazin-3-yl]methyl]-N,N-dimethyl-azetidin-3-amine (244 mg, 702.66 μmol, 1 eq), 2,5,7-trimethyl-6-[(3R)-pyrrolidin-3-yl]oxy-[1,2,4]triazolo[1,5-a]pyrimidine (270.00 mg, 843.19 μmol, 1.2 eq, 2HCl), Cs₂CO₃ (1.14 g, 3.51 mmol, 5 eq), Pd₂(dba)₃ (128.69 mg, 140.53 μmol, 0.2 eq) and ditert-butyl-[2-(o-tolyl)phenyl]phosphane (87.81 mg, 281.06 μmol, 0.4 eq) in 2-MeTHF (4 mL) and H₂O (2 mL) was degassed and purged with N₂ for 3 times, and then the mixture was stirred at 80° C. for 12 hr under N₂ atmosphere. LC-MS showed all the bromide was consumed and desired compound was detected. The reaction mixture was diluted with water 6 mL and extracted with EtOAc (5 mL×3). The combined organic layers were washed with brine 5 mL, dried over Na₂SO₄, filtered and concentrated under reduced pressure to give a residue. The residue was purified by prep-HPLC (neutral condition). Compound N,N-dimethyl-1-[[6-[4-[(3R)-3-[(2,5,7-trimethyl-[1,2,4]triazolo[1,5-a]pyrimidin-6-yl)oxy]pyrrolidin-1-yl]phenyl]pyridazin-3-yl]methyl]azetidin-3-amine (20 mg, 38.53 μmol, 5.48% yield, 98.959% purity) was obtained as a light yellow solid. LCMS for product (ESI): m/z 514.3 [M+H]⁺.

¹H NMR (400 MHz, DMSO-d₆) δ=8.06-7.99 (m, 3H), 7.56 (d, J=8.8 Hz, 1H), 6.73 (d, J=8.8 Hz, 2H), 4.97 (s, 1H), 3.84 (s, 2H), 3.70-3.61 (m, 1H), 3.60-3.50 (m, 2H), 3.47-3.43 (m, 1H), 3.41-3.38 (m, 2H), 2.92 (t, J=6.8 Hz, 2H), 2.81 (q, J=6.4 Hz, 1H), 2.58 (s, 3H), 2.52 (s, 3H), 2.46 (s, 3H), 2.42-2.35 (m, 2H), 2.01 (s, 6H).

### Example 10—Synthesis of (R)-2,5,7-trimethyl-6-((1-(4-(6-((3-(methylsulfonyl)azetidin-1-yl)methyl)pyridazin-3-yl)phenyl)pyrrolidin-3-yl)oxy)-[1,2,4]triazolo[1,5-a]pyrimidine (Compound 10)

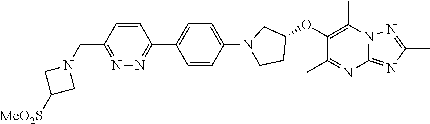 [SMILES: Cc1nc2nc(C)c(O[C@@H]3CCN(c4ccc(-c5ccc(CN6CC(S(C)(=O)=O)C6)nn5)cc4)C3)c(C)n2n1]

### Step 1: Preparation of 3-(4-bromophenyl)-6-[(3-methylsulfonylazetidin-1-yl)methyl]pyridazine

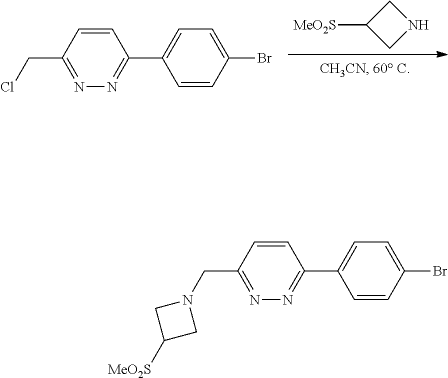 [SMILES: ClCc1ccc(-c2ccc(Br)cc2)nn1 ; CS(=O)(=O)C1CNC1 ; CS(=O)(=O)C1CN(Cc2ccc(-c3ccc(Br)cc3)nn2)C1]

A mixture of 3-(4-bromophenyl)-6-(chloromethyl)pyridazine (100 mg, 352.67 μmol, 1 eq), 3-methylsulfonylazetidine (66.59 mg, 387.94 μmol, 1.1 eq, HCl), K₂CO₃ (97.49 mg, 705.34 mol, 2 eq) in MeCN (1 mL) was stirred at 60° C. for 12 hr. LC-MS showed all the chloride was consumed and desired compound was detected. The reaction mixture was quenched by addition water 5 mL at 25° C., and then extracted with EtOAc (5 mL×2). The combined organic layers were washed with brine 5 mL, dried over Na₂SO₄, filtered. The filtrate was concentrated under reduced pressure to give 3-(4-bromophenyl)-6-[(3-methylsulfonylazetidin-1-yl)methyl]pyridazine (134 mg, crude) as a brown solid, which was used into next step directly without further purification. LCMS for product (ESI): m/z 382.1 [M+H]⁺, 384.1 [M+H+2]⁺.

### Step 2: Preparation of 2,5,7-trimethyl-6-[(3R)-1-[4-[6-[(3-methylsulfonylazetidin-1-yl)methyl]pyridazin-3-yl]phenyl]pyrrolidin-3-yl]oxy-[1,2,4]triazolo[1,5-a]pyrimidine (Compound 10)

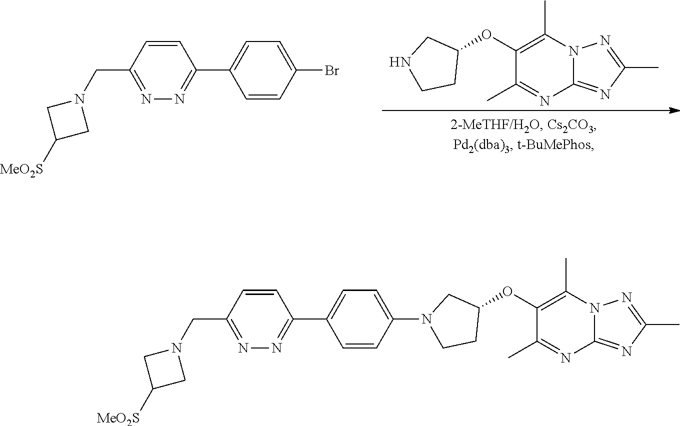 [SMILES: CS(=O)(=O)C1CN(Cc2ccc(-c3ccc(Br)cc3)nn2)C1 ; Cc1nc2nc(C)c(O[C@@H]3CCNC3)c(C)n2n1 ; Cc1nc2nc(C)c(O[C@@H]3CCN(c4ccc(-c5ccc(CN6CC(S(C)(=O)=O)C6)nn5)cc4)C3)c(C)n2n1]

A mixture of 3-(4-bromophenyl)-6-[(3-methylsulfonylazetidin-1-yl)methyl]pyridazine (134 mg, 350.53 μmol, 1 eq), 2,5,7-trimethyl-6-[(3R)-pyrrolidin-3-yl]oxy-[1,2,4]triazolo[1,5-a]pyrimidine (134.70 mg, 420.64 μmol, 1.2 eq, 2HCl), Cs₂CO₃ (571.05 mg, 1.75 mmol, 5 eq), Pd₂(dba)₃ (32.10 mg, 35.05 μmol, 0.1 eq) and ditert-butyl-[2-(o-tolyl)phenyl]phosphane (21.90 mg, 70.11 μmol, 0.2 eq) in 2-MeTHF (0.8 mL) and H₂O (0.4 mL) was degassed and purged with N₂ for 3 times, and then the mixture was stirred at 80° C. for 12 hr under N₂ atmosphere. LC-MS showed all the bromide was consumed and desired compound was detected. The reaction mixture was diluted with water 3 mL and extracted with EtOAc (3 mL×3). The combined organic layers were washed with brine 3 mL, dried over Na₂SO₄, filtered and concentrated under reduced pressure to give a residue. The residue was purified by prep-HPLC (neutral condition). Compound 2,5,7-trimethyl-6-[(3R)-1-[4-[6-[(3-methylsulfonylazetidin-1-yl)methyl]pyridazin-3-yl]phenyl]pyrrolidin-3-yl]oxy-[1,2,4]triazolo[1,5-a]pyrimidine (11 mg, 19.35 μmol, 5.52% yield, 96.513% purity) was obtained as a light yellow solid. LCMS for product (ESI): m/z 549.2 [M+H]⁺.

¹H NMR (400 MHz, DMSO-d₆) δ=8.10-8.00 (m, 3H), 7.57 (d, J=8.8 Hz, 1H), 6.74 (d, J=8.8 Hz, 2H), 4.97 (s, 1H), 4.24-4.14 (m, 1H), 3.90 (s, 2H), 3.69-3.58 (m, 3H), 3.57-3.42 (m, 5H), 2.97 (s, 3H), 2.58 (s, 3H), 2.52-2.52 (m, 3H), 2.46 (s, 3H), 2.41-2.35 (m, 2H).

### Example 11—Synthesis of (R)-1-((6-(4-(3-((2,5,7-trimethyl-[1,2,4]triazolo[1,5-a]pyrimidin-6-yl)oxy)pyrrolidin-1-yl)phenyl)pyridazin-3-yl)methyl)azetidine-3-carboxamide (Compound 11)

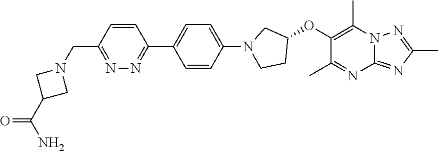 [SMILES: Cc1nc2nc(C)c(O[C@@H]3CCN(c4ccc(-c5ccc(CN6CC(C(N)=O)C6)nn5)cc4)C3)c(C)n2n1]

### Step 1: Preparation of 1-[[6-(4-bromophenyl)pyridazin-3-yl]methyl]azetidine-3-carboxamide

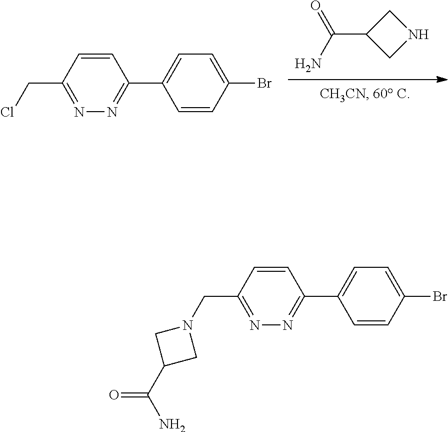 [SMILES: ClCc1ccc(-c2ccc(Br)cc2)nn1 ; NC(=O)C1CNC1 ; NC(=O)C1CN(Cc2ccc(-c3ccc(Br)cc3)nn2)C1]

A mixture of 3-(4-bromophenyl)-6-(chloromethyl)pyridazine (200 mg, 705.34 μmol, 1 eq), azetidine-3-carboxamide; hydrochloride (105.97 mg, 775.87 μmol, 1.1 eq), K₂CO₃ (292.45 mg, 2.12 mmol, 3 eq) in MeCN (4 mL) was stirred at 60° C. for 12 hr. LC-MS showed all the chloride was consumed and desired compound was detected. The reaction mixture was quenched by addition water 4 mL at 25° C., and then extracted with EtOAc (5 mL×2). The combined organic layers were washed with brine 5 mL, dried over Na₂SO₄, filtered and concentrated under reduced pressure to give a residue. Compound 1-[[6-(4-bromophenyl)pyridazin-3-yl]methyl]azetidine-3-carboxamide (244 mg, crude) was obtained as a brown solid, which was used into next step directly without further purification. LCMS for product (ESI): m/z 347.1 [M+H]⁺, 349.1 [M+H+2]⁺.

### Step 2: Preparation of 1-[[6-[4-[(3R)-3-[(2,5,7-trimethyl-[1,2,4]triazolo[1,5-a]pyrimidin-6-yl)oxy]pyrrolidin-1-yl]phenyl]pyridazin-3-yl]methyl]azetidine-3-carboxamide (Compound 11)

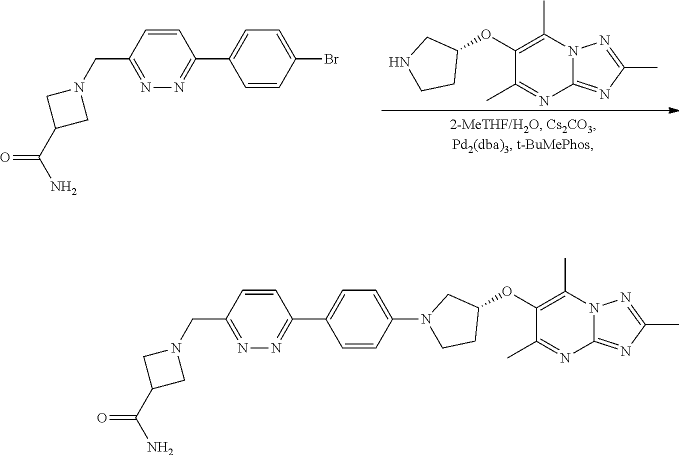 [SMILES: NC(=O)C1CN(Cc2ccc(-c3ccc(Br)cc3)nn2)C1 ; Cc1nc2nc(C)c(O[C@@H]3CCNC3)c(C)n2n1 ; Cc1nc2nc(C)c(O[C@@H]3CCN(c4ccc(-c5ccc(CN6CC(C(N)=O)C6)nn5)cc4)C3)c(C)n2n1]

A mixture of 1-[[6-(4-bromophenyl)pyridazin-3-yl]methyl]azetidine-3-carboxamide (240 mg, 691.23 μmol, 1 eq), 2,5,7-trimethyl-6-[(3R)-pyrrolidin-3-yl]oxy-[1,2,4]triazolo[1,5-a]pyrimidine (243.48 mg, 760.35 μmol, 1.1 eq, 2HCl), ditert-butyl-[2-(o-tolyl)phenyl]phosphane (43.19 mg, 138.25 μmol, 0.2 eq), Cs₂CO₃ (1.13 g, 3.46 mmol, 5 eq) and Pd₂(dba)₃ (63.30 mg, 69.12 μmol, 0.1 eq) in 2-MeTHF (4 mL) and H₂O (2 mL) was degassed and purged with N₂ for 3 times, and then the mixture was stirred at 80° C. for 12 hr under N₂ atmosphere. LC-MS showed all the bromide was consumed and desired compound was detected. The reaction mixture was diluted with water 4 mL and extracted with EtOAc (5 mL×3). The combined organic layers were washed with brine 4 mL, dried over Na₂SO₄, filtered and concentrated under reduced pressure to give a residue. The residue was purified by prep-HPLC (neutral condition). Compound 1-[[6-[4-[(3R)-3-[(2,5,7-trimethyl-[1,2,4]triazolo[1,5-a]pyrimidin-6-yl)oxy]pyrrolidin-1-yl]phenyl]pyridazin-3-yl]methyl]azetidine-3-carboxamide (3 mg, 5.57 mol, 8.06e-1% yield, 95.352% purity) was obtained as a yellow solid. LCMS for product (ESI): m/z 514.2 [M+H]⁺.

¹H NMR (400 MHz, DMSO-d₆) δ=8.08-7.98 (m, 3H), 7.56 (d, J=8.8 Hz, 1H), 7.30 (s, 1H), 6.88 (s, 1H), 6.73 (d, J=8.8 Hz, 2H), 4.96 (s, 1H), 3.82 (s, 2H), 3.69-3.61 (m, 1H), 3.60-3.50 (m, 2H), 3.47-3.38 (m, 3H), 3.26 (t, J=6.8 Hz, 2H), 3.18-3.09 (m, 1H), 2.58 (s, 3H), 2.52 (s, 3H), 2.46 (s, 3H), 2.45-2.42 (d, J=5.6 Hz, 1H), 2.36-2.32 (m, 1H).

### Example 12—Synthesis of (R)—N,N-dimethyl-1-((6-(4-(3-((2,5,7-trimethyl-[1,2,4]triazolo[1,5-a]pyrimidin-6-yl)oxy)pyrrolidin-1-yl)phenyl)pyridazin-3-yl)methyl)azetidine-3-carboxamide (Compound 12)

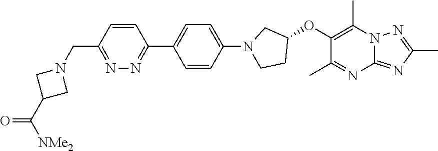 [SMILES: Cc1nc2nc(C)c(O[C@@H]3CCN(c4ccc(-c5ccc(CN6CC(C(=O)N(C)C)C6)nn5)cc4)C3)c(C)n2n1]

### Step 1: Preparation of 1-[[6-(4-bromophenyl)pyridazin-3-yl]methyl]-N,N-dimethyl-azetidine-3-carboxamide

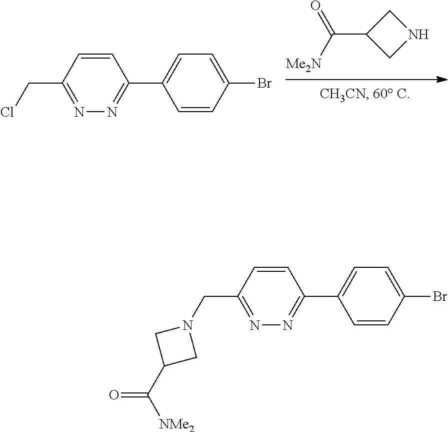 [SMILES: ClCc1ccc(-c2ccc(Br)cc2)nn1 ; CN(C)C(=O)C1CNC1 ; CN(C)C(=O)C1CN(Cc2ccc(-c3ccc(Br)cc3)nn2)C1]

A mixture of 3-(4-bromophenyl)-6-(chloromethyl)pyridazine (100 mg, 352.67 μmol, 1 eq), N,N-dimethylazetidine-3-carboxamide; hydrochloride (63.87 mg, 387.94 μmol, 1.1 eq), K₂CO₃ (97.49 mg, 705.34 μmol, 2 eq) in MeCN (3 mL) was stirred at 60° C. for 2 hr. LC-MS showed all the chloride was consumed and desired compound was detected. The reaction mixture was quenched by addition water 3 mL at 25° C., and then extracted with EtOAc (5 mL×2). The combined organic layers were washed with brine 1 mL, dried over Na₂SO₄, filtered. The filtrate was concentrated under reduced pressure to give 1-[[6-(4-bromophenyl)pyridazin-3-yl]methyl]-N,N-dimethyl-azetidine-3-carboxamide (132 mg, crude) as a brown solid, which was used into next step directly without further purification. LCMS for product (ESI): m/z 375.2 [M+H]⁺, 377.2 [M+H+2]⁺.

### Step 1: Preparation of N,N-dimethyl-1-[[6-[4-[(3R)-3-[(2,5,7-trimethyl-[1,2,4]triazolo[1,5-a]pyrimidin-6-yl)oxy]pyrrolidin-1-yl]phenyl]pyridazin-3-yl]methyl]azetidine-3-carboxamide (Compound 12)

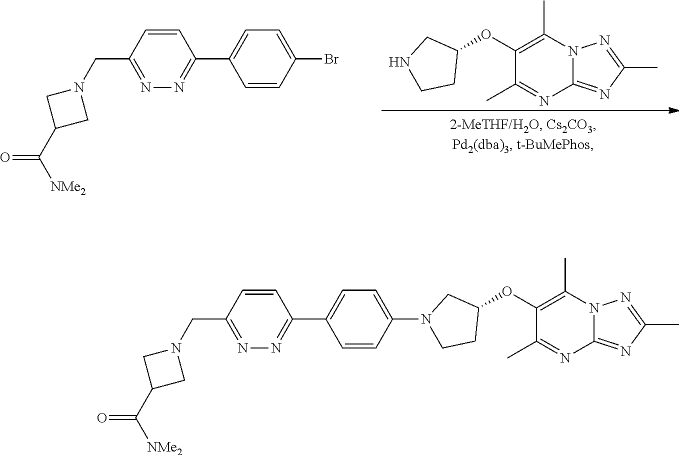 [SMILES: CN(C)C(=O)C1CN(Cc2ccc(-c3ccc(Br)cc3)nn2)C1 ; Cc1nc2nc(C)c(O[C@@H]3CCNC3)c(C)n2n1 ; Cc1nc2nc(C)c(O[C@@H]3CCN(c4ccc(-c5ccc(CN6CC(C(=O)N(C)C)C6)nn5)cc4)C3)c(C)n2n1]

A mixture of 1-[[6-(4-bromophenyl)pyridazin-3-yl]methyl]-N,N-dimethyl-azetidine-3-carboxamide (132 mg, 351.75 μmol, 1 eq), 2,5,7-trimethyl-6-[(3R)-pyrrolidin-3-yl]oxy-[1,2,4]triazolo[1,5-a]pyrimidine (135.17 mg, 422.10 μmol, 1.2 eq, 2HCl), Cs₂CO₃ (573.04 mg, 1.76 mmol, 5 eq), Pd₂(dba)₃ (32.21 mg, 35.18 μmol, 0.1 eq) and ditert-butyl-[2-(o-tolyl)phenyl]phosphane (21.98 mg, 70.35 μmol, 0.2 eq) in 2-MeTHF (2 mL) and H₂O (1 mL) was degassed and purged with N₂ for 3 times, and then the mixture was stirred at 80° C. for 12 hr under N₂ atmosphere. LC-MS showed all the bromide was consumed and desired compound was detected. The reaction mixture was diluted with water 3 mL and extracted with EtOAc (3 mL×3). The combined organic layers were washed with brine 3 mL, dried over Na₂SO₄, filtered and concentrated under reduced pressure to give a residue. The residue was purified by prep-HPLC (neutral condition) Compound N,N-dimethyl-1-[[6-[4-[(3R)-3-[(2,5,7-trimethyl-[1,2,4]triazolo[1,5-a]pyrimidin-6-yl)oxy]pyrrolidin-1-yl]phenyl]pyridazin-3-yl]methyl]azetidine-3-carboxamide (3 mg, 5.42 μmol, 1.54% yield, 97.854% purity) was obtained as a light yellow solid. LCMS for product (ESI): m/z 542.2 [M+H]⁺.

¹H NMR (400 MHz, DMSO-d₆) δ=8.08-7.98 (m, 3H), 7.56 (d, J=8.8 Hz, 1H), 6.74 (d, J=8.8 Hz, 2H), 4.97 (s, 1H), 3.81 (s, 2H), 3.70-3.61 (m, 1H), 3.60-3.54 (m, 1H), 3.54-3.47 (m, 4H), 3.46 (d, J=4.4 Hz, 1H), 3.27 (s, 2H), 2.82 (d, J=4.0 Hz, 6H), 2.58 (s, 3H), 2.52 (s, 3H), 2.46 (s, 3H), 2.43-2.34 (m, 2H).

### Example 13—Synthesis of (R)-(2-oxa-6-azaspiro[3.3]heptan-6-yl)(6-(4-(3-((2,5,7-trimethyl-[1,2,4]triazolo[1,5-a]pyrimidin-6-yl)oxy)pyrrolidin-1-yl)phenyl)pyridazin-3-yl)methanone (Compound 13)

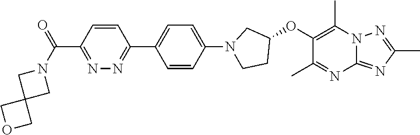 [SMILES: Cc1nc2nc(C)c(O[C@@H]3CCN(c4ccc(-c5ccc(C(=O)N6CC7(COC7)C6)nn5)cc4)C3)c(C)n2n1]

### Step 1: Preparation of 6-(4-bromophenyl)pyridazine-3-carboxylic acid

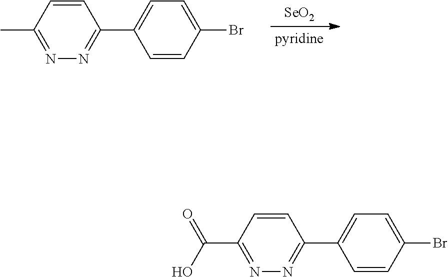

A mixture of 3-(4-bromophenyl)-6-methyl-pyridazine (1.3 g, 5.22 mmol, 1 eq), SeO₂ (1.74 g, 15.66 mmol, 1.70 mL, 3 eq) in pyridine (13 mL) was degassed and purged with N₂ for 3 times, and then the mixture was stirred at 80° C. for 12 hr. TLC indicated all the starting material was consumed, and one major new spot with larger polarity was detected. The reaction mixture was concentrated under reduced pressure to remove pyridine. The residue was diluted with water 50 mL and then filtered. The filter cake was washed with MTBE (50 mL) and concentrated under reduced pressure to give a residue. Compound 6-(4-bromophenyl)pyridazine-3-carboxylic acid (1.3 g, 4.63 mmol, 44.37% yield, 99.412% purity) was obtained as a brown solid.

¹H NMR (400 MHz, DMSO-d₆) δ=8.43 (d, J=8.8 Hz, 1H), 8.26 (d, J=8.8 Hz, 1H), 8.20 (d, J=8.8 Hz, 2H), 7.81 (d, J=8.8 Hz, 2H)

### Step 2: Preparation of [6-(4-bromophenyl)pyridazin-3-yl]-(2-oxa-6-azaspiro[3.3]heptan-6-yl)methanone

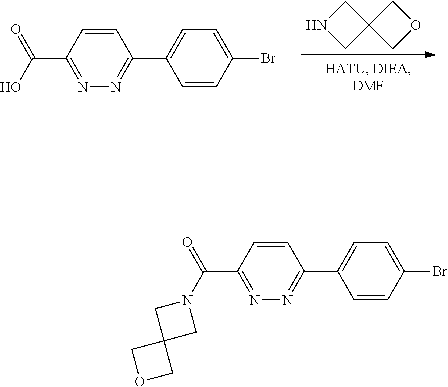 [SMILES: O=C(O)c1ccc(-c2ccc(Br)cc2)nn1 ; C1NCC12COC2 ; O=C(c1ccc(-c2ccc(Br)cc2)nn1)N1CC2(COC2)C1]

A mixture of 6-(4-bromophenyl)pyridazine-3-carboxylic acid (100 mg, 358.31 μmol, 1 eq), 2-oxa-6-azaspiro[3.3]heptane (53.44 mg, 394.14 μmol, 1.1 eq, HCl), DIEA (185.24 mg, 1.43 mmol, 249.64 uL, 4 eq), HATU (204.36 mg, 537.46 μmol, 1.5 eq) in DMF (2 mL) was stirred at 20° C. for 4 hr. LC-MS showed all the carboxylic acid was consumed and desired compound was detected. The reaction mixture was quenched by addition water 10 mL at 20° C., and then filtered. The filter cake was washed with MTBE (10 mL) and dried to give [6-(4-bromophenyl)pyridazin-3-yl]-(2-oxa-6-azaspiro[3.3]heptan-6-yl)methanone (84 mg, crude) as a brown solid, which was used into the next step without further purification. LCMS for product (ESI): m/z 360.0 [M+H]⁺, 361.9 [M+H+2]⁺.

### Step 3: Preparation of 2-oxa-6-azaspiro[3.3]heptan-6-yl-[6-[4-[(3R)-3-[(2,5,7-trimethyl-[1,2,4]triazolo[1,5-a]pyrimidin-6-yl)oxy]pyrrolidin-1-yl]phenyl]pyridazin-3-yl]methanone (Compound 13)

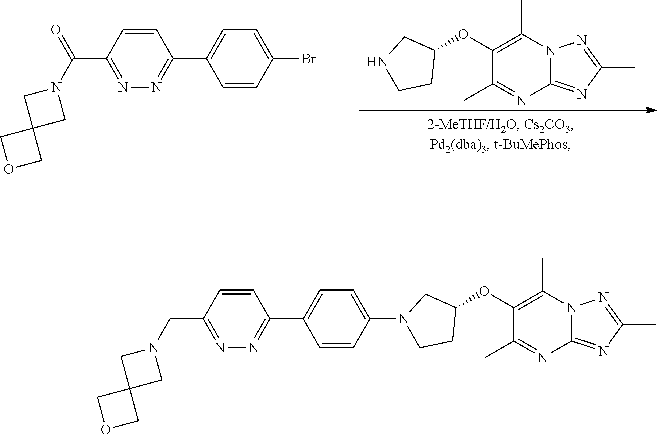 [SMILES: O=C(c1ccc(-c2ccc(Br)cc2)nn1)N1CC2(COC2)C1 ; Cc1nc2nc(C)c(O[C@@H]3CCNC3)c(C)n2n1 ; Cc1nc2nc(C)c(O[C@@H]3CCN(c4ccc(-c5ccc(CN6CC7(COC7)C6)nn5)cc4)C3)c(C)n2n1]

A mixture of [6-(4-bromophenyl)pyridazin-3-yl]-(2-oxa-6-azaspiro[3.3]heptan-6-yl)methanone (84 mg, 233.20 μmol, 1 eq), 2,5,7-trimethyl-6-[(3R)-pyrrolidin-3-yl]oxy-[1,2,4]triazolo[1,5-a]pyrimidine (89.61 mg, 279.84 μmol, 1.2 eq, 2HCl), Cs₂CO₃ (379.91 mg, 1.17 mmol, 5 eq), Pd₂(dba)₃ (42.71 mg, 46.64 μmol, 0.2 eq) and ditert-butyl-[2-(o-tolyl)phenyl]phosphane (29.14 mg, 93.28 μmol, 0.4 eq) in 2-MeTHF (1 mL) and H₂O (0.5 mL) was degassed and purged with N₂ for 3 times, and then the mixture was stirred at 80° C. for 2 hr under N₂ atmosphere. LC-MS showed all the bromide was consumed and desired compound was detected. The reaction mixture was filtered. The filter cake was purified by prep-HPLC (neutral condition). Compound 2-oxa-6-azaspiro[3.3]heptan-6-yl-[6-[4-[(3R)-3-[(2,5,7-trimethyl-[1,2,4]triazolo[1,5-a]pyrimidin-6-yl)oxy]pyrrolidin-1-yl]phenyl]pyridazin-3-yl]methanone (9 mg, 17.09 μmol, 7.33% yield, 100% purity) was obtained as a yellow solid. LCMS for product (ESI): m/z 527.2 [M+H]⁺.

¹H NMR (400 MHz, DMSO-d₆) δ=8.23 (d, J=9.2 Hz, 1H), 8.13 (d, J=8.8 Hz, 2H), 8.01 (d, J=9.2 Hz, 1H), 6.77 (d, J=8.8 Hz, 2H), 4.97 (s, 1H), 4.83 (s, 2H), 4.72 (s, 4H), 4.31 (s, 2H), 3.73-3.53 (m, 3H), 3.50-3.43 (m, 1H), 2.58 (s, 3H), 2.52 (s, 3H), 2.47 (s, 3H), 2.43-2.35 (m, 2H).

### Example 14—Synthesis of (R)-(3-methoxyazetidin-1-yl)(6-(4-(3-((2,5,7-trimethyl-[1,2,4]triazolo[1,5-a]pyrimidin-6-yl)oxy)pyrrolidin-1-yl)phenyl)pyridazin-3-yl)methanone (Compound 14)

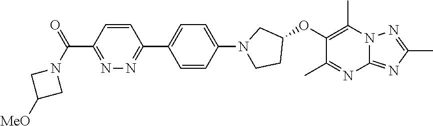 [SMILES: COC1CN(C(=O)c2ccc(-c3ccc(N4CC[C@@H](Oc5c(C)nc6nc(C)nn6c5C)C4)cc3)nn2)C1]

### Step 1: Preparation of [6-(4-bromophenyl)pyridazin-3-yl]-(3-methoxyazetidin-1-yl)methanone

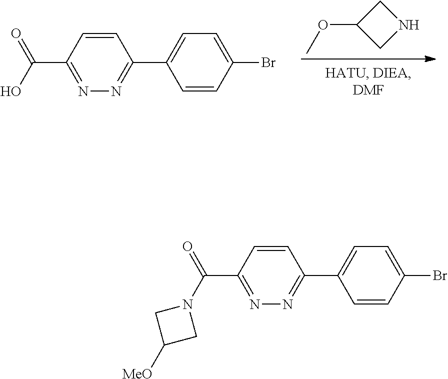

A mixture of 6-(4-bromophenyl)pyridazine-3-carboxylic acid (100 mg, 358.31 μmol, 1 eq), 3-methoxyazetidine (48.71 mg, 394.14 μmol, 1.1 eq, HCl), DIEA (185.23 mg, 1.43 mmol, 249.64 uL, 4 eq), HATU (204.36 mg, 537.46 μmol, 1.5 eq) in DMF (2 mL) was stirred at 20° C. for 1 hr. LC-MS showed all the carboxylic acid was consumed and desired compound was detected. The reaction mixture was quenched by addition 1N HCl aq. 5 mL at 20° C., and then filtered. The filter cake was washed with MTBE (10 mL) and dried to give [6-(4-bromophenyl)pyridazin-3-yl]-(3-methoxyazetidin-1-yl)methanone (80 mg, crude) as a brown solid, which was used into the next step without further purification. LCMS for product (ESI): m/z 348.0 [M+H]⁺, 350.0 [M+H+2]⁺.

Step 2: Preparation of (3-methoxyazetidin-1-yl)-[6-[4-[(3R)-3-[(2,5,7-trimethyl-[1,2,4]triazolo[1,5-a]pyrimidin-6-yl)oxy]pyrrolidin-1-yl]phenyl]pyridazin-3-yl]methanone (Compound 14)

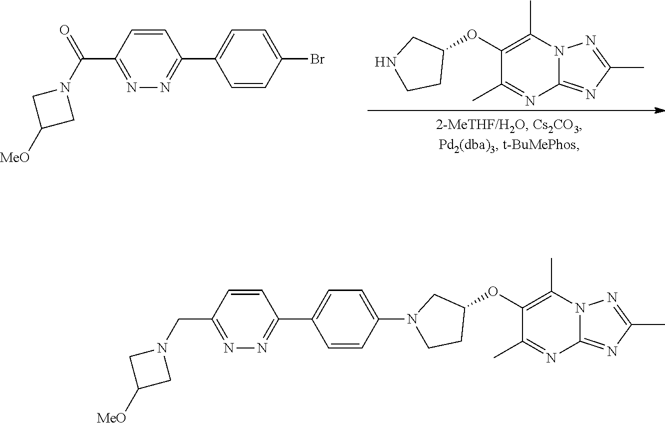 [SMILES: COC1CN(C(=O)c2ccc(-c3ccc(Br)cc3)nn2)C1 ; Cc1nc2nc(C)c(O[C@@H]3CCNC3)c(C)n2n1 ; COC1CN(Cc2ccc(-c3ccc(N4CC[C@@H](Oc5c(C)nc6nc(C)nn6c5C)C4)cc3)nn2)C1]

A mixture of [6-(4-bromophenyl)pyridazin-3-yl]-(3-methoxyazetidin-1-yl)methanone (80 mg, 229.76 μmol, 1 eq), 2,5,7-trimethyl-6-[(3R)-pyrrolidin-3-yl]oxy-[1,2,4]triazolo[1,5-a]pyrimidine (88.29 mg, 275.71 μmol, 1.2 eq, 2HCl), ditert-butyl-[2-(o-tolyl)phenyl]phosphane (28.71 mg, 91.90 μmol, 0.4 eq), Pd₂(dba)₃ (42.08 mg, 45.95 μmol, 0.2 eq) and Cs₂CO₃ (374.30 mg, 1.15 mmol, 5 eq) in 2-MeTHF (2 mL) and H₂O (1 mL) was degassed and purged with N₂ for 3 times, and then the mixture was stirred at 80° C. for 1 hr under N₂ atmosphere. LC-MS showed all the bromide was consumed and desired compound was detected. The reaction mixture was filtered. The filter cake was purified by prep-HPLC (neutral condition). Compound (3-methoxyazetidin-1-yl)-[6-[4-[(3R)-3-[(2,5,7-trimethyl-[1,2,4]triazolo[1,5-a]pyrimidin-6-yl)oxy]pyrrolidin-1-yl]phenyl]pyridazin-3-yl]methanone (8 mg, 15.47 μmol, 6.73% yield, 99.496% purity) was obtained as a yellow solid. LCMS for product (ESI): m/z 515.3 [M+H]⁺.

¹H NMR (400 MHz, DMSO-d₆) δ=8.25 (d, J=8.8 Hz, 1H), 8.14 (d, J=8.8 Hz, 2H), 8.03 (d, J=8.8 Hz, 1H), 6.76 (d, J=8.8 Hz, 2H), 4.97 (br s, 1H), 4.88-4.79 (m, 1H), 4.45 (dd, J=2.0, 10.8 Hz, 1H), 4.37-4.26 (m, 2H), 3.93 (d, J=8.4 Hz, 1H), 3.73-3.53 (m, 3H), 3.47 (dd, J=3.6, 12.0 Hz, 1H), 3.26 (s, 3H), 2.58 (s, 3H), 2.52 (s, 3H), 2.46 (s, 3H), 2.43-2.34 (m, 2H).

### Example 15—Synthesis of (R)-(3-(dimethylamino)azetidin-1-yl)(6-(4-(3-((2,5,7-trimethyl-[1,2,4]triazolo[1,5-a]pyrimidin-6-yl)oxy)pyrrolidin-1-yl)phenyl)pyridazin-3-yl)methanone (Compound 15)

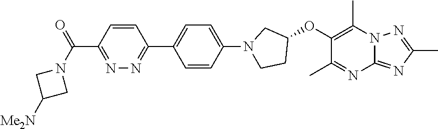 [SMILES: Cc1nc2nc(C)c(O[C@@H]3CCN(c4ccc(-c5ccc(C(=O)N6CC(N(C)C)C6)nn5)cc4)C3)c(C)n2n1]

### Step 1: Preparation of [6-(4-bromophenyl)pyridazin-3-yl]-[3-(dimethylamino)azetidin-1-yl]methanone

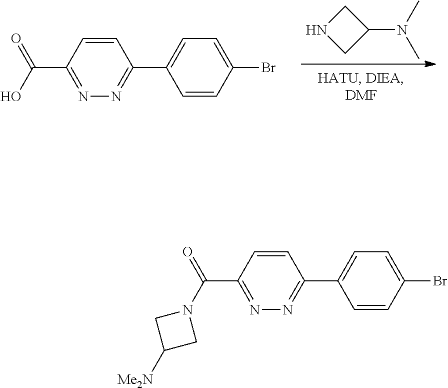

A mixture of 6-(4-bromophenyl)pyridazine-3-carboxylic acid (100 mg, 358.31 μmol, 1 eq), N,N-dimethylazetidin-3-amine; hydrochloride (53.85 mg, 394.14 μmol, 1.1 eq), DIEA (185.24 mg, 1.43 mmol, 249.64 uL, 4 eq), HATU (204.36 mg, 537.46 μmol, 1.5 eq) and in DMF (2 mL) was stirred at 20° C. for 4 hr. LC-MS showed all the carboxylic acid was consumed and desired compound was detected. The reaction mixture was quenched by addition water 10 mL at 20° C., and then filtered. The filter cake was washed with MTBE (10 mL) and dried to give [6-(4-bromophenyl)pyridazin-3-yl]-[3-(dimethylamino)azetidin-1-yl]methanone (94 mg, crude) as a brown solid, which was used into the next step without further purification. LCMS for product (ESI): m/z 361.0 [M+H]⁺, 363.0 [M+H+2]⁺.

### Step 2: Preparation of [3-(dimethylamino)azetidin-1-yl]-[6-[4-[(3R)-3-[(2,5,7-trimethyl-[1,2,4]triazolo[1,5-a]pyrimidin-6-yl)oxy]pyrrolidin-1-yl]phenyl]pyridazin-3-yl]methanone (Compound 15)

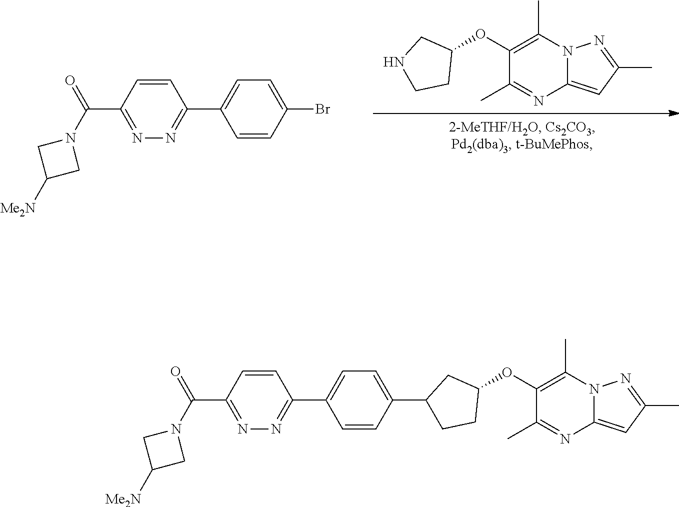 [SMILES: CN(C)C1CN(C(=O)c2ccc(-c3ccc(Br)cc3)nn2)C1 ; Cc1cc2nc(C)c(O[C@@H]3CCNC3)c(C)n2n1 ; Cc1cc2nc(C)c(O[C@@H]3CCC(c4ccc(-c5ccc(C(=O)N6CC(N(C)C)C6)nn5)cc4)C3)c(C)n2n1]

A mixture of [6-(4-bromophenyl)pyridazin-3-yl]-[3-(dimethylamino)azetidin-1-yl]methanone (94 mg, 260.22 μmol, 1 eq), 2,5,7-trimethyl-6-[(3R)-pyrrolidin-3-yl]oxy-[1,2,4]triazolo[1,5-a]pyrimidine (99.99 mg, 312.26 μmol, 1.2 eq, 2HCl), Cs₂CO₃ (423.92 mg, 1.30 mmol, 5 eq), Pd₂(dba)₃ (47.66 mg, 52.04 μmol, 0.2 eq) and ditert-butyl-[2-(o-tolyl)phenyl]phosphane (32.52 mg, 104.09 μmol, 0.4 eq) in 2-MeTHF (1 mL) and H₂O (0.5 mL) was degassed and purged with N₂ for 3 times, and then the mixture was stirred at 80° C. for 1 hr under N₂ atmosphere. LC-MS showed all the bromide was consumed and desired product was detected. The reaction mixture was filtered. The filter cake was purified by prep-HPLC (neutral condition). Compound [3-(dimethylamino)azetidin-1-yl]-[6-[4-[(3R)-3-[(2,5,7-trimethyl-[1,2,4]triazolo[1,5-a]pyrimidin-6-yl)oxy]pyrrolidin-1-yl]phenyl]pyridazin-3-yl]methanone (1.4 mg, 2.48 μmol, 9.52e-1% yield, 93.402% purity) was obtained as a yellow solid. LCMS for product (ESI): m/z 528.3 [M+H]⁺.

¹H NMR (400 MHz, DMSO-d₆) δ=8.24 (d, J=8.8 Hz, 1H), 8.13 (d, J=8.8 Hz, 2H), 8.02 (d, J=8.8 Hz, 1H), 6.77 (d, J=8.8 Hz, 2H), 4.97 (br s, 1H), 4.67 (dd, J=7.6, 10.0 Hz, 1H), 4.42 (dd, J=5.2, 10.8 Hz, 1H), 4.15 (dd, J=7.6, 10.0 Hz, 1H), 3.90 (dd, J=4.8, 10.4 Hz, 1H), 3.72-3.52 (m, 3H), 3.47 (dd, J=4.0, 12.4 Hz, 1H), 3.15-3.09 (m, 1H), 2.58 (s, 3H), 2.52 (s, 3H), 2.46 (s, 3H), 2.43-2.34 (m, 2H), 2.12 (s, 6H).

### Example 16—Synthesis of (R)-(3-(methylsulfonyl)azetidin-1-yl)(6-(4-(3-((2,5,7-trimethyl-[1,2,4]triazolo[1,5-a]pyrimidin-6-yl)oxy)pyrrolidin-1-yl)phenyl)pyridazin-3-yl)methanone (Compound 16)

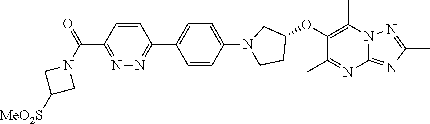 [SMILES: Cc1nc2nc(C)c(O[C@@H]3CCN(c4ccc(-c5ccc(C(=O)N6CC(S(C)(=O)=O)C6)nn5)cc4)C3)c(C)n2n1]

### Step 1: Preparation of Give [6-(4-bromophenyl)pyridazin-3-yl]-(3-methylsulfonylazetidin-1-yl)methanone

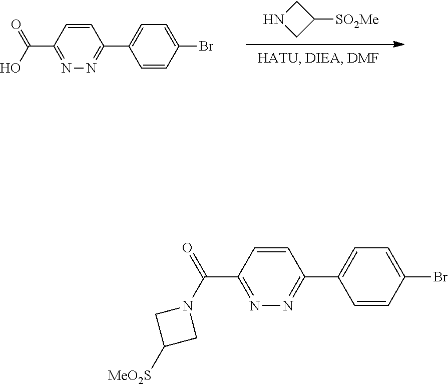 [SMILES: O=C(O)c1ccc(-c2ccc(Br)cc2)nn1 ; CS(=O)(=O)C1CNC1 ; CS(=O)(=O)C1CN(C(=O)c2ccc(-c3ccc(Br)cc3)nn2)C1]

A mixture of 6-(4-bromophenyl)pyridazine-3-carboxylic acid (100 mg, 358.31 μmol, 1 eq), 3-methylsulfonylazetidine (67.65 mg, 394.14 μmol, 1.1 eq, HCl), DIEA (185.24 mg, 1.43 mmol, 249.64 uL, 4 eq), HATU (204.36 mg, 537.46 μmol, 1.5 eq) in DMF (2 mL) was stirred at 20° C. for 4 hr. LC-MS showed all the carboxylic acid was consumed and desired compound was detected. The reaction mixture was quenched by addition water 10 mL at 20° C., and then filtered. The filter cake was washed with MTBE (10 mL) and dried to give [6-(4-bromophenyl)pyridazin-3-yl]-(3-methylsulfonylazetidin-1-yl)methanone (40 mg, crude) was obtained as a brown solid, which was used into the next step without further purification. LCMS for product (ESI): m/z 396.0 [M+H]⁺, 398.0 [M+H+2]⁺.

### Step 2: Preparation of (3-methylsulfonylazetidin-1-yl)-[6-[4-[(3R)-3-[(2,5,7-trimethyl-[1,2,4]triazolo[1,5-a]pyrimidin-6-yl)oxy]pyrrolidin-1-yl]phenyl]pyridazin-3-yl]methanone (Compound 16)

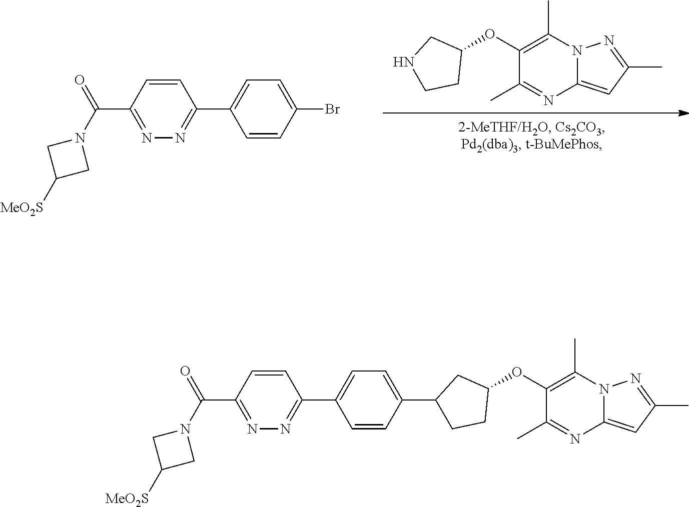 [SMILES: CS(=O)(=O)C1CN(C(=O)c2ccc(-c3ccc(Br)cc3)nn2)C1 ; Cc1cc2nc(C)c(O[C@@H]3CCNC3)c(C)n2n1 ; Cc1cc2nc(C)c(O[C@@H]3CCC(c4ccc(-c5ccc(C(=O)N6CC(S(C)(=O)=O)C6)nn5)cc4)C3)c(C)n2n1]

A mixture of [6-(4-bromophenyl)pyridazin-3-yl]-(3-methylsulfonylazetidin-1-yl)methanone (20 mg, 50.47 μmol, 1 eq), 2,5,7-trimethyl-6-[(3R)-pyrrolidin-3-yl]oxy-[1,2,4]triazolo[1,5-a]pyrimidine (19.39 mg, 60.57 μmol, 1.2 eq, 2HCl), Cs₂CO₃ (82.22 mg, 252.36 μmol, 5 eq), Pd₂(dba)₃ (9.24 mg, 10.09 μmol, 0.2 eq) and ditert-butyl-[2-(o-tolyl)phenyl]phosphane (6.31 mg, 20.19 μmol, 0.4 eq) in 2-MeTHF (1 mL) and H₂O (0.5 mL) was degassed and purged with N₂ for 3 times, and then the mixture was stirred at 80° C. for 1 hr under N₂ atmosphere. LC-MS showed all the bromide was consumed and desired product was detected. The reaction mixture was filtered. The filter cake was purified by prep-HPLC (neutral condition). Compound (3-methylsulfonylazetidin-1-yl)-[6-[4-[(3R)-3-[(2,5,7-trimethyl-[1,2,4]triazolo[1,5-a]pyrimidin-6-yl)oxy]pyrrolidin-1-yl]phenyl]pyridazin-3-yl]methanone (2 mg, 3.55 μmol, 7.04% yield, 100% purity) was obtained as a yellow solid. LCMS for product (ESI): m/z 563.2 [M+H]⁺.

¹H NMR (400 MHz, DMSO-d₆) δ=8.27 (d, J=9.2 Hz, 1H), 8.15 (d, J=8.8 Hz, 2H), 8.06 (d, J=8.8 Hz, 1H), 6.77 (d, J=8.8 Hz, 2H), 5.04-4.94 (m, 2H), 4.87-4.84 (m, 1H), 4.49-4.39 (m, 2H), 4.33-4.30 (m, 1H), 3.72-3.53 (m, 3H), 3.50-3.43 (m, 1H), 3.09 (s, 3H), 2.58 (s, 3H), 2.52 (s, 3H), 2.47 (s, 3H), 2.40-2.37 (m, 2H).

### Example 17—Synthesis of (R)-1-(6-(4-(3-((2,5,7-trimethyl-[1,2,4]triazolo[1,5-a]pyrimidin-6-yl)oxy)pyrrolidin-1-yl)phenyl)pyridazine-3-carbonyl)azetidine-3-carboxamide (Compound 17)

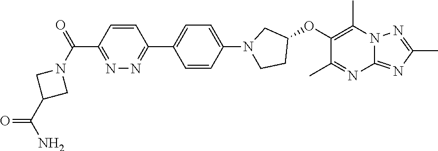 [SMILES: Cc1nc2nc(C)c(O[C@@H]3CCN(c4ccc(-c5ccc(C(=O)N6CC(C(N)=O)C6)nn5)cc4)C3)c(C)n2n1]

### Step 1: Preparation of 1-[6-(4-bromophenyl)pyridazine-3-carbonyl]azetidine-3-carboxamide

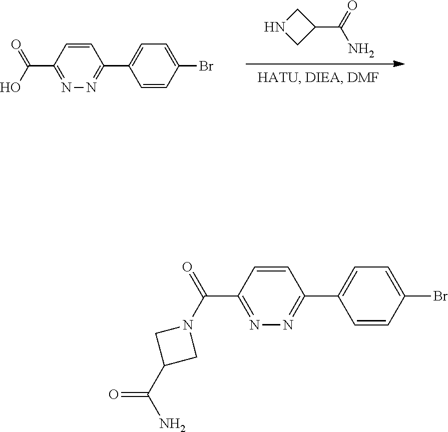 [SMILES: O=C(O)c1ccc(-c2ccc(Br)cc2)nn1 ; NC(=O)C1CNC1 ; NC(=O)C1CN(C(=O)c2ccc(-c3ccc(Br)cc3)nn2)C1]

A mixture of 6-(4-bromophenyl)pyridazine-3-carboxylic acid (100 mg, 358.31 μmol, 1 eq), azetidine-3-carboxamide (39.46 mg, 394.14 μmol, 1.1 eq, HCl), DIEA (185.23 mg, 1.43 mmol, 249.64 uL, 4 eq), HATU (204.36 mg, 537.46 μmol, 1.5 eq) in DMF (2 mL) was stirred at 25° C. for 12 hr. LC-MS showed all the carboxylic acid was consumed and desired compound was detected. The reaction mixture was quenched by addition water 10 mL at 25° C., and then filtered. The filter cake was washed with water (10 mL) and concentrated under reduced pressure to give a residue. Compound 1-[6-(4-bromophenyl)pyridazine-3-carbonyl]azetidine-3-carboxamide (86 mg, 238.10 μmol, 66.45% yield) was obtained as a brown solid, which was used into the next step without further purification. LCMS for product (ESI): m/z 361.0[M+H]⁺, 363.0 [M+H+2]⁺.

### Step 2: Preparation of 1-[6-[4-[(3R)-3-[(2,5,7-trimethyl-[1,2,4]triazolo[1,5-a]pyrimidin-6-yl)oxy]pyrrolidin-1-yl]phenyl]pyridazine-3-carbonyl]azetidine-3-carboxamide (Compound 17)

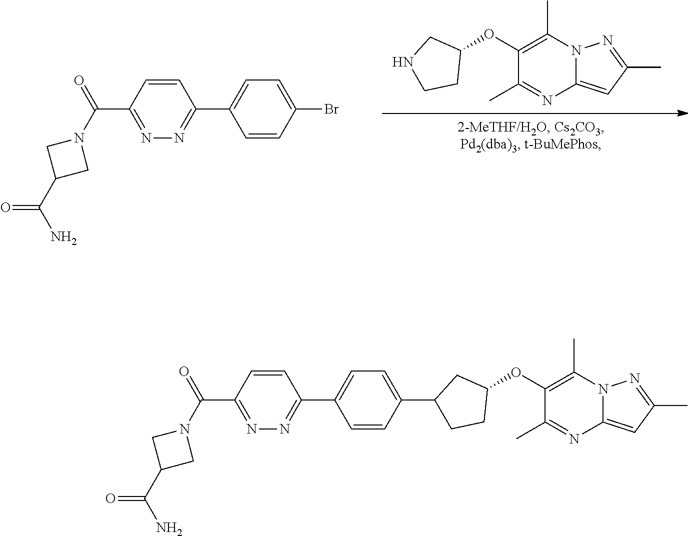 [SMILES: NC(=O)C1CN(C(=O)c2ccc(-c3ccc(Br)cc3)nn2)C1 ; Cc1cc2nc(C)c(O[C@@H]3CCNC3)c(C)n2n1 ; Cc1cc2nc(C)c(O[C@@H]3CCC(c4ccc(-c5ccc(C(=O)N6CC(C(N)=O)C6)nn5)cc4)C3)c(C)n2n1]

A mixture of 1-[6-(4-bromophenyl)pyridazine-3-carbonyl]azetidine-3-carboxamide (86 mg, 238.10 μmol, 1 eq), 2,5,7-trimethyl-6-[(3R)-pyrrolidin-3-yl]oxy-[1,2,4]triazolo[1,5-a]pyrimidine (83.87 mg, 261.91 μmol, 1.1 eq, 2HCl), Cs₂CO₃ (310.31 mg, 952.40 μmol, 4 eq), Pd₂(dba)₃ (21.80 mg, 23.81 μmol, 0.1 eq) and ditert-butyl-[2-(o-tolyl)phenyl]phosphane (14.88 mg, 47.62 μmol, 0.2 eq) in 2-MeTHF (1.5 mL) and H₂O (0.75 mL) was degassed and purged with N₂ for 3 times, and then the mixture was stirred at 80° C. for 2 hr under N₂ atmosphere. LC-MS showed all the bromide was consumed and desired compound was detected. The reaction mixture was filtered and the filter cake was concentrated under reduced pressure to give a residue. The residue was purified by prep-HPLC (neutral condition). Compound 1-[6-[4-[(3R)-3-[(2,5,7-trimethyl-[1,2,4]triazolo[1,5-a]pyrimidin-6-yl)oxy]pyrrolidin-1-yl]phenyl]pyridazine-3-carbonyl]azetidine-3-carboxamide (6 mg, 10.69 μmol, 4.49% yield, 93.998% purity) was obtained as a yellow solid. LCMS for product (ESI): m/z 528.2 [M+H]⁺.

¹H NMR (400 MHz, DMSO-d₆) δ=8.21 (d, J=9.2 Hz, 1H), 8.10 (d, J=8.8 Hz, 2H), 7.99 (d, J=9.2 Hz, 1H), 7.49 (s, 1H), 7.04 (s, 1H), 6.73 (d, J=8.8 Hz, 2H), 4.97-4.91 (m, 1H), 4.75 (t, J=9.6 Hz, 1H), 4.60 (dd, J=6.0, 9.6 Hz, 1H), 4.24-4.15 (m, 1H), 4.09 (dd, J=5.6, 10.0 Hz, 1H), 3.68-3.60 (m, 1H), 3.59-3.51 (m, 2H), 3.46-3.36 (m, 2H), 2.55 (s, 3H), 2.49 (s, 3H), 2.43 (s, 3H), 2.40-2.31 (m, 2H).

### Example 18—Synthesis of (R)—N,N-dimethyl-1-(6-(4-(3-((2,5,7-trimethyl-[1,2,4]triazolo[1,5-a]pyrimidin-6-yl)oxy)pyrrolidin-1-yl)phenyl)pyridazine-3-carbonyl)azetidine-3-carboxamide (Compound 18)

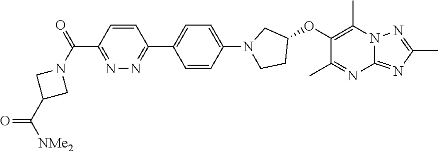 [SMILES: Cc1nc2nc(C)c(O[C@@H]3CCN(c4ccc(-c5ccc(C(=O)N6CC(C(=O)N(C)C)C6)nn5)cc4)C3)c(C)n2n1]

### Step 1: Preparation of 1-[6-(4-bromophenyl)pyridazine-3-carbonyl]-N,N-dimethyl-azetidine-3-carboxamide

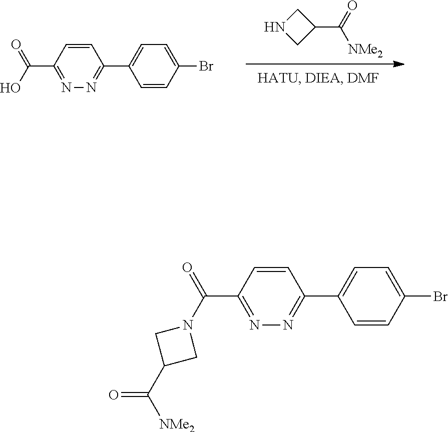 [SMILES: O=C(O)c1ccc(-c2ccc(Br)cc2)nn1 ; CN(C)C(=O)C1CNC1 ; CN(C)C(=O)C1CN(C(=O)c2ccc(-c3ccc(Br)cc3)nn2)C1]

A mixture of 6-(4-bromophenyl)pyridazine-3-carboxylic acid (100 mg, 358.31 μmol, 1 eq), N,N-dimethylazetidine-3-carboxamide; hydrochloride (64.89 mg, 394.14 μmol, 1.1 eq), DIEA (185.24 mg, 1.43 mmol, 249.64 uL, 4 eq), HATU (204.36 mg, 537.46 μmol, 1.5 eq) in DMF (2 mL) was stirred at 20° C. for 4 hr. LC-MS showed all the carboxylic acid was consumed and desired compound was detected. The reaction mixture was quenched by addition water 10 mL at 20° C. and then filtered. The filter cake was washed with MTBE (10 mL) and filter cake was dried to give 1-[6-(4-bromophenyl)pyridazine-3-carbonyl]-N,N-dimethyl-azetidine-3-carboxamide (66 mg, crude) as a brown solid, which was used into the next step without further purification. LCMS for product (ESI): m/z 389.0 [M+H]⁺, 391.0 [M+H+2]⁺.

### Step 2: Preparation of N,N-dimethyl-1-[6-[4-[(3R)-3-[(2,5,7-trimethyl-[1,2,4]triazolo[1,5-a]pyrimidin-6-yl)oxy]pyrrolidin-1-yl]phenyl]pyridazine-3-carbonyl]azetidine-3-carboxamide (Compound 18)

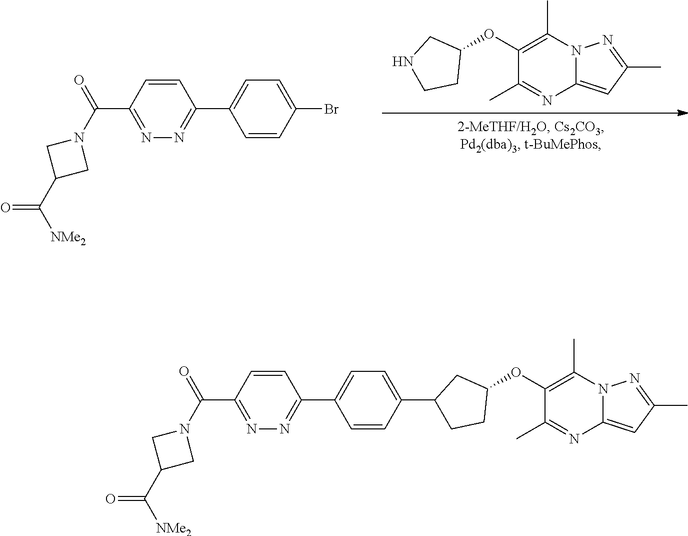 [SMILES: CN(C)C(=O)C1CN(C(=O)c2ccc(-c3ccc(Br)cc3)nn2)C1 ; Cc1cc2nc(C)c(O[C@@H]3CCNC3)c(C)n2n1 ; Cc1cc2nc(C)c(O[C@@H]3CCC(c4ccc(-c5ccc(C(=O)N6CC(C(=O)N(C)C)C6)nn5)cc4)C3)c(C)n2n1]

A mixture of 1-[6-(4-bromophenyl)pyridazine-3-carbonyl]-N,N-dimethyl-azetidine-3-carboxamide (66 mg, 169.56 μmol, 1 eq), 2,5,7-trimethyl-6-[(3R)-pyrrolidin-3-yl]oxy-[1,2,4]triazolo[1,5-a]pyrimidine (65.15 mg, 203.47 μmol, 1.2 eq, 2HCl), Cs₂CO₃ (276.23 mg, 847.79 μmol, 5 eq), Pd₂(dba)₃ (31.05 mg, 33.91 μmol, 0.2 eq) and ditert-butyl-[2-(o-tolyl)phenyl]phosphane (21.19 mg, 67.82 μmol, 0.4 eq) in 2-MeTHF (1 mL) and H₂O (0.5 mL) was degassed and purged with N₂ for 3 times, then the mixture was stirred at 80° C. for 1 hr under N₂ atmosphere. LC-MS showed all the bromide was consumed and desired compound was detected. The reaction mixture was filtered. The filter cake was purified by prep-HPLC (neutral condition). Compound N,N-dimethyl-1-[6-[4-[(3R)-3-[(2,5,7-trimethyl-[1,2,4]triazolo[1,5-a]pyrimidin-6-yl)oxy]pyrrolidin-1-yl]phenyl]pyridazine-3-carbonyl]azetidine-3-carboxamide (10 mg, 17.53 μmol, 10.34% yield, 97.386% purity) was obtained as a yellow solid. LCMS for product (ESI): m/z 556.3 [M+H]⁺.

¹H NMR (400 MHz, DMSO-d₆) δ=8.25 (d, J=8.8 Hz, 1H), 8.14 (d, J=8.8 Hz, 2H), 8.03 (d, J=8.8 Hz, 1H), 6.77 (d, J=8.8 Hz, 2H), 4.97 (br s, 1H), 4.91-4.82 (m, 1H), 4.72-4.67 (m, 1H), 4.33-4.27 (m, 1H), 4.25-4.20 (m, 1H), 3.88-3.78 (m, 1H), 3.73-3.53 (m, 3H), 3.51-3.44 (m, 1H), 2.90 (s, 3H), 2.87 (s, 3H), 2.58 (s, 3H), 2.52 (s, 3H), 2.46 (s, 3H), 2.42-2.36 (m, 2H).

### Example 19—Synthesis of (R)-morpholino(6-(4-(3-((2,5,7-trimethyl-[1,2,4]triazolo[1,5-a]pyrimidin-6-yl)oxy)pyrrolidin-1-yl)phenyl)pyridazin-3-yl)methanone (Compound 19)

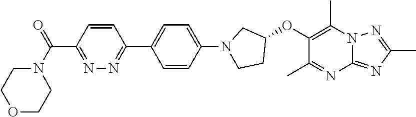 [SMILES: Cc1nc2nc(C)c(O[C@@H]3CCN(c4ccc(-c5ccc(C(=O)N6CCOCC6)nn5)cc4)C3)c(C)n2n1]

### Step 1: Preparation of (6-(4-bromophenyl)pyridazin-3-yl)(morpholino)methanone

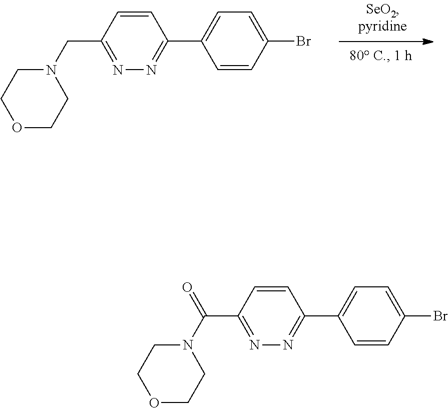 [SMILES: Brc1ccc(-c2ccc(CN3CCOCC3)nn2)cc1 ; O=C(c1ccc(-c2ccc(Br)cc2)nn1)N1CCOCC1]

SeO₂ (1.7 g, 15 mmol) was added into a mixture solution of 4-((6-(4-bromophenyl)pyridazin-3-yl)methyl)morpholine (500 mg, 1.5 mmol) in pyridine (5 ml) at 25° C. and stirred at 80° C. for 1 h. LCMS showed the starting material was consumed and desired product was detected. The reaction mixture was concentrated to remove the solvent and diluted with ethyl acetate (20 ml) and filtered. The filtrate was concentrated to give a residue and purified by column to give (6-(4-bromophenyl)pyridazin-3-yl)(morpholino)methanone (250 mg, 48% yield) as a white solid, which was used into the next step without further purification.

Step 2: Preparation of (R)-morpholino(6-(4-(3-((2,5,7-trimethyl-[1,2,4]triazolo[1,5-a]pyrimidin-6-yl)oxy)pyrrolidin-1-yl)phenyl)pyridazin-3-yl)methanone (Compound 19)

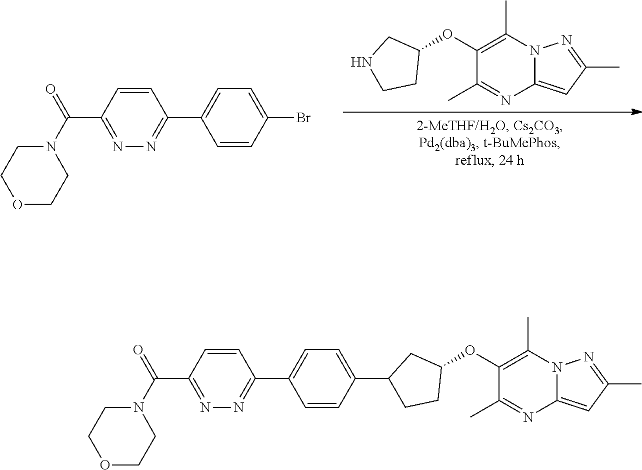 [SMILES: O=C(c1ccc(-c2ccc(Br)cc2)nn1)N1CCOCC1 ; Cc1cc2nc(C)c(O[C@@H]3CCNC3)c(C)n2n1 ; Cc1cc2nc(C)c(O[C@@H]3CCC(c4ccc(-c5ccc(C(=O)N6CCOCC6)nn5)cc4)C3)c(C)n2n1]

A mixture of (6-(4-bromophenyl)pyridazin-3-yl)(morpholino)methanone (0.1 g, 0.29 mmol, 1 eq), 2,5,7-trimethyl-6-[(3R)-pyrrolidin-3-yl]oxy-[1,2,4]triazolo[1,5-a]pyrimidine (0.11 g, 0.35 mmol, 1.2 eq, 2HCl), ditert-butyl-[2-(o-tolyl)phenyl]phosphane (36 mg, 0.12 mmol, 0.4 eq), Cs₂CO₃ (0.47 g, 1.45 mmol, 5 eq) and Pd₂(dba)₃ (50 mg, 0.058 mmol, 0.2 eq) in 2-MeTHF (2 mL) and water (0.4 mL) was de-gassed and then heated to 80° C. for 24 h under N₂. LC-MS showed the desired compound was detected. The reaction mixture was poured into water (5 mL). The mixture was extracted with ethyl acetate (10 mL). The organic phase was washed with brine (5 mL), dried over anhydrous Na₂SO₄, concentrated in vacuum to give the crude product. The crude purified by prep-HPLC (neutral condition) to give (R)-morpholino(6-(4-(3-((2,5,7-trimethyl-[1,2,4]triazolo[1,5-a]pyrimidin-6-yl)oxy)pyrrolidin-1-yl)phenyl)pyridazin-3-yl)methanone (40 mg). LCMS for product (ESI): m/z 515.3 (M+H)⁺.

¹H NMR (400 MHz, CHLOROFORM-d) δ=8.23 (d, J=8.8 Hz, 1H), 8.11 (d, J=8.8 Hz, 2H), 7.83 (d, J=8.8 Hz, 1H), 6.76 (d, J=8.8 Hz, 2H), 4.99-4.96 (m, 1H), 3.71 (s, 4H), 3.65-3.60 (m, 7H), 3.50-3.44 (m, 1H), 2.58 (s, 3H), 2.52 (s, 3H), 2.50 (s, 3H), 2.49-2.45 (m, 2H).

### Example 20—Synthesis of (R)-morpholino(6-(4-(3-((2,5,7-trimethylpyrazolo[1,5-a]pyrimidin-6-yl)oxy)pyrrolidin-1-yl)phenyl)pyridazin-3-yl)methanone (Compound 20)

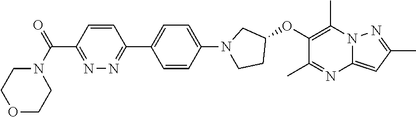 [SMILES: Cc1cc2nc(C)c(O[C@@H]3CCN(c4ccc(-c5ccc(C(=O)N6CCOCC6)nn5)cc4)C3)c(C)n2n1]

### Preparation of (R)-morpholino(6-(4-(3-((2,5,7-trimethylpyrazolo[1,5-a]pyrimidin-6-yl)oxy)pyrrolidin-1-yl)phenyl)pyridazin-3-yl)methanone

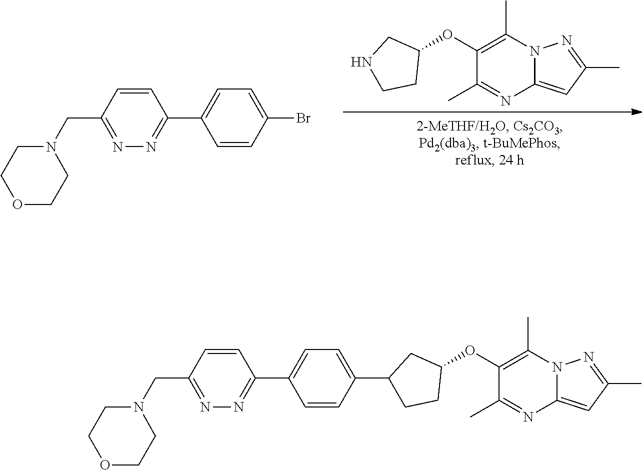 [SMILES: Brc1ccc(-c2ccc(CN3CCOCC3)nn2)cc1 ; Cc1cc2nc(C)c(O[C@@H]3CCNC3)c(C)n2n1 ; Cc1cc2nc(C)c(O[C@@H]3CCC(c4ccc(-c5ccc(CN6CCOCC6)nn5)cc4)C3)c(C)n2n1]

A mixture of (6-(4-bromophenyl)pyridazin-3-yl)(morpholino)methanone (0.1 g, 0.29 mmol, 1 eq), (R)-2,5,7-trimethyl-6-(pyrrolidin-3-yloxy)pyrazolo[1,5-a]pyrimidine (0.11 g, 0.35 mmol, 1.2 eq, 2HCl), ditert-butyl-[2-(o-tolyl)phenyl]phosphane (36 mg, 0.12 mmol, 0.4 eq), Cs₂CO₃ (0.47 g, 1.45 mmol, 5 eq) and Pd₂(dba)₃ (50 mg, 0.058 mmol, 0.2 eq) in 2-MeTHF (2 mL) and water (0.4 mL) was de-gassed and then heated to 80° C. for 24 h under N₂. LC-MS showed the desired compound was detected. The reaction mixture was poured into water (5 mL). The mixture was extracted with ethyl acetate (10 mL). The organic phase was washed with brine (5 mL), dried over anhydrous Na₂SO₄, concentrated in vacuum to give the crude product. The crude purified by prep-HPLC (neutral condition) to give (R)-morpholino(6-(4-(3-((2,5,7-trimethylpyrazolo[1,5-a]pyrimidin-6-yl)oxy)pyrrolidin-1-yl)phenyl)pyridazin-3-yl)methanone (45 mg). LCMS for product (ESI): m/z 514.3 (M+H)⁺.

¹H NMR (400 MHz, CHLOROFORM-d) δ=8.24 (d, J=8.8 Hz, 1H), 8.12 (d, J=8.8 Hz, 2H), 7.83 (d, J=8.8 Hz, 1H), 6.77 (d, J=8.8 Hz, 2H), 6.37 (s, 1H), 4.9s (br s, 1H), 3.72 (s, 4H), 3.66-3.56 (m, 7H), 3.50-3.48 (m, 1H), 2.54 (s, 3H), 2.45 (s, 3H), 2.33 (s, 3H), 2.31-2.28 (m, 2H).

### Example 21—Synthesis of (R)-6-((3-(5-(3-((2,5,7-trimethyl-[1,2,4]triazolo[1,5-a]pyrimidin-6-yl)oxy)pyrrolidin-1-yl)pyrimidin-2-yl)bicyclo[1.1.1]pentan-1-yl)methyl)-2-oxa-6-azaspiro[3.3]heptane (Compound 21)

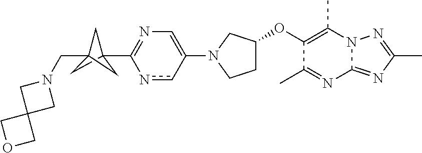

### Step 1: Preparation of (6-(4-bromophenyl)pyridazin-3-yl)(morpholino)methanone

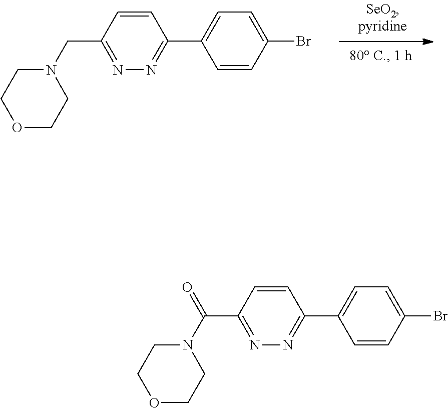 [SMILES: Brc1ccc(-c2ccc(CN3CCOCC3)nn2)cc1 ; O=C(c1ccc(-c2ccc(Br)cc2)nn1)N1CCOCC1]

SeO₂ (1.7 g, 15 mmol) was added into a mixture solution of 4-((6-(4-bromophenyl)pyridazin-3-yl)methyl)morpholine

### Step 2: Preparation of tert-butyl-dimethyl-[[3-[5-[(3R)-3-[(2,5,7-trimethyl-[1,2,4]triazolo[1,5-a]pyrimidin-6-yl)oxy]pyrrolidin-1-yl]pyrimidin-2-yl]-1-bicyclo[1.1.1]pentanyl]methoxy]silane

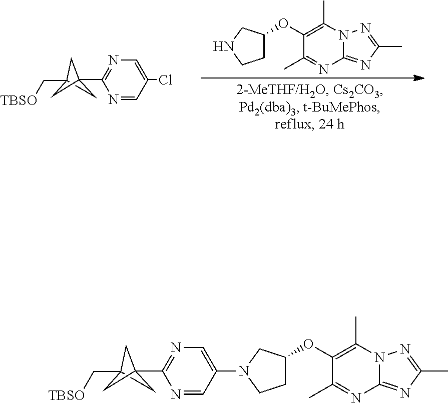 [SMILES: CC(C)(C)[Si](C)(C)OCC12CC(c3ncc(Cl)cn3)(C1)C2 ; Cc1nc2nc(C)c(O[C@@H]3CCNC3)c(C)n2n1 ; Cc1nc2nc(C)c(O[C@@H]3CCN(c4cnc(C56CC(CO[Si](C)(C)C(C)(C)C)(C5)C6)nc4)C3)c(C)n2n1]

A mixture of tert-butyl-[[3-(5-chloropyrimidin-2-yl)-1-bicyclo[1.1.1]pentanyl]methoxy]-dimethyl-silane (2.4 g, 7.39 mmol, 1 eq), 2,5,7-trimethyl-6-[(3R)-pyrrolidin-3-yl]oxy-[1,2,4]triazolo[1,5-a]pyrimidine (2.84 g, 8.86 mmol, 1.2 eq, 2HCl), ditert-butyl-[2-(o-tolyl)phenyl]phosphane (923.09 mg, 2.95 mmol, 0.4 eq), Cs₂CO₃ (12.03 g, 36.93 mmol, 5 eq) and Pd₂(dba)₃ (1.35 g, 1.48 mmol, 0.2 eq) in 2-MeTHF (20 mL) and water (10 mL) was de-gassed and then heated to 80° C. for 24 h under N₂. LC-MS showed the desired compound was detected. The reaction mixture was poured into water (20 mL). The mixture was extracted with ethyl acetate (50 mL). The organic phase was washed with brine (50 mL), dried over anhydrous Na₂SO₄, concentrated in vacuum to give the crude product tert-butyl-dimethyl-[[3-[5-[(3R)-3-[(2,5,7-trimethyl-[1,2,4]triazolo[1,5-a]pyrimidin-6-yl)oxy]pyrrolidin-1-yl]pyrimidin-2-yl]-1-bicyclo[1.1.1]pentanyl]methoxy]silane (1.3 g, 2.43 mmol, yield 32.85%, 48% purity) as a brown solid, which was used into the next step without purification.

### Step 3: Preparation of [3-[5-[(3R)-3-[(2,5,7-trimethyl-[1,2,4]triazolo[1,5-a]pyrimidin-6-yl)oxy]pyrrolidin-1-yl]pyrimidin-2-yl]-1-bicyclo[1.1.1]pentanyl]methanol

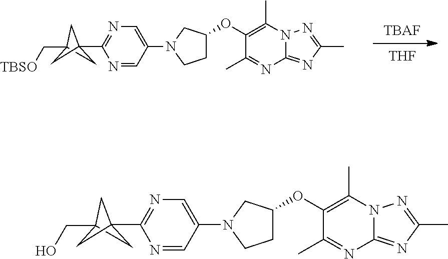 [SMILES: Cc1nc2nc(C)c(O[C@@H]3CCN(c4cnc(C56CC(CO[Si](C)(C)C(C)(C)C)(C5)C6)nc4)C3)c(C)n2n1 ; "TBAF" ; Cc1nc2nc(C)c(O[C@@H]3CCN(c4cnc(C56CC(CO)(C5)C6)nc4)C3)c(C)n2n1 ; "THF"]

To a solution of tert-butyl-dimethyl-[[3-[5-[(3R)-3-[(2,5,7-trimethyl-[1,2,4]triazolo[1,5-a]pyrimidin-6-yl)oxy]pyrrolidin-1-yl]pyrimidin-2-yl]-1-bicyclo[1.1.1]pentanyl]methoxy]silane (1.2 g, 2.24 mmol, 1 eq) in THE (10 mL) was added TBAF (1 M, 5.60 mL, 2.5 eq) at 20° C. The mixture was stirred at 20° C. for 2 h. LC-MS showed the desired compound was detected. The reaction mixture was partitioned between water (20 mL) and DCM (20 mL). The organic phase was separated, washed with brine (20 mL), dried over anhydrous Na₂SO₄, filtered and concentrated under reduced pressure to give a residue. The residue was purified by column chromatography (SiO2, DCM:MeOH=20:1) to give [3-[5-[(3R)-3-[(2,5,7-trimethyl-[1,2,4]triazolo[1,5-a]pyrimidin-6-yl)oxy]pyrrolidin-1-yl]pyrimidin-2-yl]-1-bicyclo[1.1.1]pentanyl]methanol (0.6 g, 1.42 mmol, 63.55% yield) as a yellow solid.

¹H NMR (400 MHz, CHLOROFORM-d) 6=8.08 (s, 2H), 4.88-4.83 (m, 1H), 3.74 (s, 2H), 3.73-3.70 (m, 1H), 3.60-3.44 (m, 3H), 2.66 (s, 3H), 2.61 (s, 3H), 2.61 (s, 3H), 2.54-2.45 (m, 1H), 2.56-2.35 (m, 1H), 2.13 (s, 6H)

### Step 4: Preparation of 6-[(3R)-1-[2-[3-(iodomethyl)-1-bicyclo[1.1.1]pentanyl]pyrimidin-5-yl]pyrrolidin-3-yl]oxy-2,5,7-trimethyl-[1,2,4]triazolo[1,5-a]pyrimidine

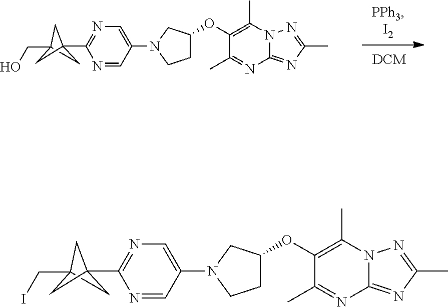 [SMILES: Cc1nc2nc(C)c(O[C@@H]3CCN(c4cnc(C56CC(CO)(C5)C6)nc4)C3)c(C)n2n1 ; Cc1nc2nc(C)c(O[C@@H]3CCN(c4cnc(C56CC(CI)(C5)C6)nc4)C3)c(C)n2n1]

To a solution of [3-[5-[(3R)-3-[(2,5,7-trimethyl-[1,2,4]triazolo[1,5-a]pyrimidin-6-yl)oxy]pyrrolidin-1-yl]pyrimidin-2-yl]-1-bicyclo[1.1.1]pentanyl]methanol (100 mg, 237.25 mol, 1 eq) and PPh3 (186.68 mg, 711.75 μmol, 3 eq) in DCM (5 mL) was added 12 (120.43 mg, 474.50 μmol, 95.58 uL, 2 eq) and imidazole (48.45 mg, 711.75 μmol, 3 eq) at 0° C. The mixture was stirred at 20° C. for 2 h. LCMS showed the desired compound was detected. The reaction was quenched by 1N HCl and then extracted with DCM (10 mL). The organic phase was washed with brine (5 mL), dried over anhydrous Na₂SO₄, filtered and concentrated in vacuum to give the crude product 6-[(3R)-1-[2-[3-(iodomethyl)-1-bicyclo[1.1.1]pentanyl]pyrimidin-5-yl]pyrrolidin-3-yl]oxy-2,5,7-trimethyl-[1,2,4]triazolo[1,5-a]pyrimidine (126 mg, 237.11 μmol, 99.94% yield) as a yellow solid, which was used directly without purification.

### Step 5: Preparation of 6-[[3-[5-[(3R)-3-[(2,5,7-trimethyl-[1,2,4]triazolo[1,5-a]pyrimidin-6-yl)oxy]pyrrolidin-1-yl]pyrimidin-2-yl]-1-bicyclo[1.1.1]pentanyl]methyl]-2-oxa-6-azaspiro[3.3]heptane

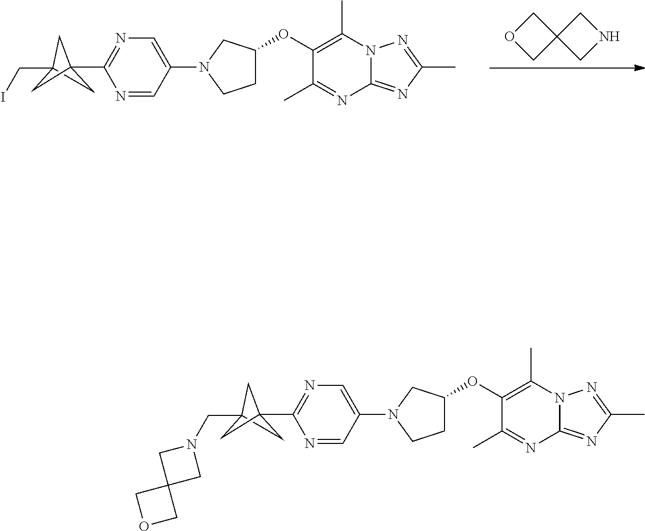 [SMILES: Cc1nc2nc(C)c(O[C@@H]3CCN(c4cnc(C56CC(CI)(C5)C6)nc4)C3)c(C)n2n1 ; C1NCC12COC2 ; Cc1nc2nc(C)c(O[C@@H]3CCN(c4cnc(C56CC(CN7CC8(COC8)C7)(C5)C6)nc4)C3)c(C)n2n1]

A mixture of 2-oxa-6-azaspiro[3.3]heptane; oxalic acid (124.59 mg, 658.65 μmol, 5 eq) and K₂CO₃ (91.03 mg, 658.65 μmol, 5 eq) in DMF (2 mL) was stirred at 20° C. for 0.5 h. Then 6-[(3R)-1-[2-[3-(iodomethyl)-1-bicyclo[1.1.1]pentanyl]pyrimidin-5-yl]pyrrolidin-3-yl]oxy-2,5,7-trimethyl-[1,2,4]triazolo[1,5-a]pyrimidine (70 mg, 131.73 μmol, 1 eq) was added into the above solution at 20° C. The mixture was stirred at 20° C. for 12 h. LCMS showed the desired compound was detected. The reaction mixture was purified by prep-HPLC (neutral condition) to give 6-[[3-[5-[(3R)-3-[(2,5,7-trimethyl-[1,2,4]triazolo[1,5-a]pyrimidin-6-yl)oxy]pyrrolidin-1-yl]pyrimidin-2-yl]-1-bicyclo[1.1.1]pentanyl]methyl]-2-oxa-6-azaspiro[3.3]heptane (13 mg, 25.35 μmol, 19.24% yield, 98% purity). LCMS for product (ESI): m/z 503.2 (M+H)⁺. SFC for product: ee=100%.

¹H NMR (400 MHz, METHANOL-d₄) δ=8.10 (s, 2H), 5.01 (s, 1H), 4.75 (s, 4H), 3.78-3.69 (m, 1H), 3.63-3.54 (m, 2H), 3.51-3.44 (m, 5H), 2.67 (s, 3H), 2.62 (s, 2H), 2.60 (s, 3H), 2.54 (s, 3H), 2.52-2.47 (m, 1H), 2.42-2.32 (m, 1H), 2.08 (s, 6H).

### Example 22—Synthesis of (R)-6-((1-(2-(3-((3-methoxyazetidin-1-yl)methyl)bicyclo[1.1.1]pentan-1-yl)pyrimidin-5-yl)pyrrolidin-3-yl)oxy)-2,5,7-trimethyl-[1,2,4]triazolo[1,5-a]pyrimidine (Compound 22)

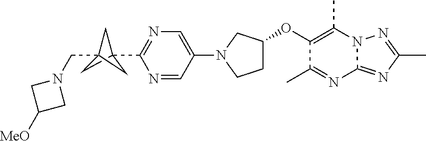

### Preparation of (R)-6-((1-(2-(3-((3-methoxyazetidin-1-yl)methyl)bicyclo[1.1.1]pentan-1-yl)pyrimidin-5-yl)pyrrolidin-3-yl)oxy)-2,5,7-trimethyl-[1,2,4]triazolo[1,5-a]pyrimidine

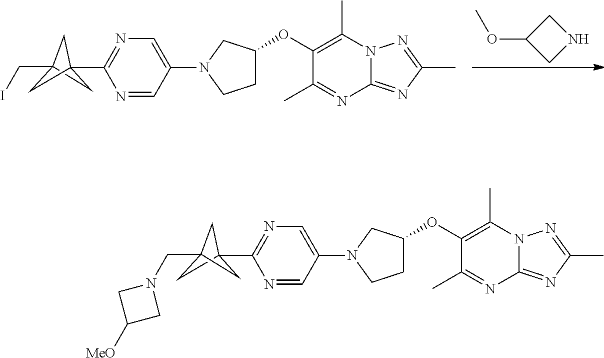

A mixture of 3-methoxyazetidine (58.14 mg, 470.46 μmol, 5 eq) and TEA (57.13 mg, 564.56 μmol, 78.58 uL, 6 eq) in DMF (2 mL) was stirred at 20° C. for 0.5 h. Then 6-[(3R)-1-[2-[3-(iodomethyl)-1-bicyclo[1.1.1]pentanyl]pyrimidin-5-yl]pyrrolidin-3-yl]oxy-2,5,7-trimethyl-[1,2,4]triazolo[1,5-a]pyrimidine (50 mg, 94.09 μmol, 1 eq) was added into the above solution at 20° C. The mixture was stirred at 20° C. for 2 h. LC-MS showed the desired compound was detected. The reaction was quenched by ice slowly and then extracted with DCM (10 mL). The organic phase was washed with brine (5 mL), dried over anhydrous Na₂SO₄, filtered and concentrated in vacuo to give the residue. The residue was purified by prep-HPLC (neutral condition) to give 6-[(3R)-1-[2-[3-[(3-methoxyazetidin-1-yl)methyl]-1-bicyclo[1.1.1]pentanyl]pyrimidin-5-yl]pyrrolidin-3-yl]oxy-2,5,7-trimethyl-[1,2,4]triazolo[1,5-a]pyrimidine (10 mg, 20.18 μmol, 21.45% yield, 99% purity). LCMS for product (ESI): m/z 491.3 (M+H)⁺. SFC for product: ee=100%

¹H NMR (400 MHz, METHANOL-d₄) δ=8.10 (s, 2H), 5.01 (s, 1H), 4.08 (q, J=6.0 Hz, 1H), 3.78-3.65 (m, 3H), 3.64-3.53 (m, 2H), 3.51-3.45 (m, 1H), 3.26 (s, 3H), 3.08-3.02 (m, 2H), 2.70 (s, 2H), 2.67 (s, 3H), 2.59 (s, 3H), 2.53 (s, 3H), 2.52-2.46 (m, 1H), 2.36 (m, 1H), 2.08 (s, 6H).

### Example 23—Synthesis of (R)-4-((3-(5-(3-((2,5,7-trimethyl-[1,2,4]triazolo[1,5-a]pyrimidin-6-yl)oxy)pyrrolidin-1-yl)pyrimidin-2-yl)bicyclo[1.1.1]pentan-1-yl)methyl)morpholine (Compound 23)

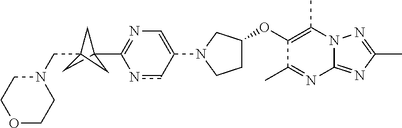

### Preparation of (R)-4-((3-(5-(3-((2,5,7-trimethyl-[1,2,4]triazolo[1,5-a]pyrimidin-6-yl)oxy)pyrrolidin-1-yl)pyrimidin-2-yl)bicyclo[1.1.1]pentan-1-yl)methyl)morpholine

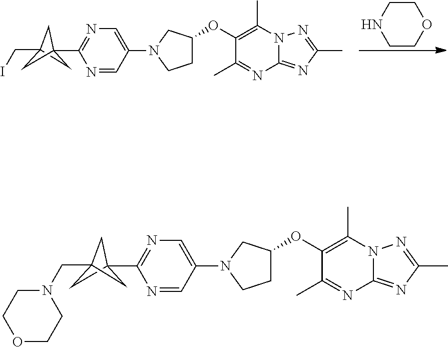 [SMILES: Cc1nc2nc(C)c(O[C@@H]3CCN(c4cnc(C56CC(CI)(C5)C6)nc4)C3)c(C)n2n1 ; C1COCCN1 ; Cc1nc2nc(C)c(O[C@@H]3CCN(c4cnc(C56CC(CN7CCOCC7)(C5)C6)nc4)C3)c(C)n2n1]

A mixture of 6-[(3R)-1-[2-[3-(iodomethyl)-1-bicyclo[1.1.1]pentanyl]pyrimidin-5-yl]pyrrolidin-3-yl]oxy-2,5,7-trimethyl-[1,2,4]triazolo[1,5-a]pyrimidine (60 mg, 120.10 μmol, 1 eq) in DMF (2 mL) was added morpholine (104.63 mg, 1.20 mmol, 105.69 uL, 10 eq) at 20° C. The mixture was stirred at 20° C. for 12 h. LCMS showed the desired compound was detected. The residue was purified by prep-HPLC (TFA condition) to give 4-[[3-[5-[(3R)-3-[(2,5,7-trimethyl-[1,2,4]triazolo[1,5-a]pyrimidin-6-yl)oxy]pyrrolidin-1-yl]pyrimidin-2-yl]-1-bicyclo[1.1.1]pentanyl]methyl]morpholine (11 mg, 15.00 μmol, 12.49% yield, 98% purity, 2TFA). LCMS for product (ESI): m/z 491.2 (M+H)⁺. SFC for product: ee=100%.

¹H NMR (400 MHz, METHANOL-d₄) 6=8.14 (s, 2H), 5.02 (s, 1H), 4.16-3.98 (m, 2H), 3.92-3.79 (m, 2H), 3.78-3.70 (m, 1H), 3.66-3.53 (m, 4H), 3.49 (dd, J=4.0, 11.7 Hz, 1H), 3.44 (s, 2H), 3.28-3.14 (m, 2H), 2.68 (s, 3H), 2.59 (s, 3H), 2.54 (s, 3H), 2.52-2.48 (m, 1H), 2.44-2.34 (m, 1H), 2.32 (s, 6H).

### Example 24—Synthesis of (R)-(2-oxa-6-azaspiro[3.3]heptan-6-yl)(3-(5-(3-((2,5,7-trimethyl-[1,2,4]triazolo[1,5-a]pyrimidin-6-yl)oxy)pyrrolidin-1-yl)pyrimidin-2-yl)bicyclo[1.1.1]pentan-1-yl)methanone (Compound 24)

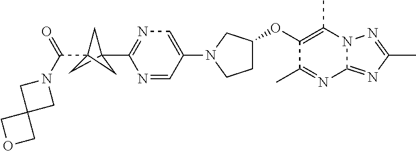

### Step 1. Preparation of 3-[5-[(3R)-3-[(2,5,7-trimethyl-[1,2,4]triazolo[1,5-a]pyrimidin-6-yl)oxy]pyrrolidin-1-yl]pyrimidin-2-yl]bicyclo[1.1.1]pentane-1-carboxylic acid

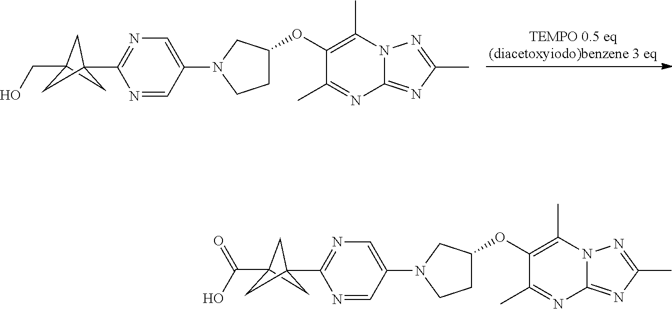 [SMILES: Cc1nc2nc(C)c(O[C@@H]3CCN(c4cnc(C56CC(CO)(C5)C6)nc4)C3)c(C)n2n1 ; Cc1nc2nc(C)c(O[C@@H]3CCN(c4cnc(C56CC(C(=O)O)(C5)C6)nc4)C3)c(C)n2n1]

A mixture of [3-[5-[(3R)-3-[(2,5,7-trimethyl-[1,2,4]triazolo[1,5-a]pyrimidin-6-yl)oxy]pyrrolidin-1-yl]pyrimidin-2-yl]-1-bicyclo[1.1.1]pentanyl]methanol (0.2 g, 474.50 μmol, 1 eq) in DCM (1 mL) was added TEMPO (37.31 mg, 237.25 μmol, 0.5 eq) and [acetoxy(phenyl)-X³-iodanyl] acetate (458.51 mg, 1.42 mmol, 3 eq) at 20° C. The mixture was stirred at 20° C. for 2 h. LC-MS showed the desired compound was detected. The reaction mixture was partitioned between (5 mL) and water (5 mL). The organic phase was separated, washed with brine (5 mL), dried over Na₂SO₄, filtered and concentrated under reduced pressure to give a residue. The residue was purified by reversed-phase column (TFA condition) to give 3-[5-[(3R)-3-[(2,5,7-trimethyl-[1,2,4]triazolo[1,5-a]pyrimidin-6-yl)oxy]pyrrolidin-1-yl]pyrimidin-2-yl]bicyclo[1.1.1]pentane-1-carboxylic acid (0.15 g, 344.45 μmol, 72.59% yield).

¹H NMR (400 MHz, DMSO-d₆) δ=8.09 (s, 2H), 4.96 (s, 1H), 3.62-3.42 (m, 4H), 2.57 (s, 3H), 2.52 (s, 3H), 2.46 (s, 3H), 2.34-2.20 (m, 2H), 2.00 (s, 6H)

### Step 2. Preparation of 2-oxa-6-azaspiro[3.3]heptan-6-yl-[3-[5-[(3R)-3-[(2,5,7-trimethyl-[1,2,4]triazolo[1,5-a]pyrimidin-6-yl)oxy]pyrrolidin-1-yl]pyrimidin-2-yl]-1-bicyclo[1.1.1]pentanyl]methanone

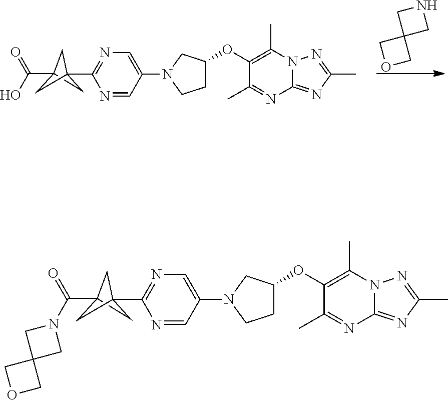

A solution of 3-[5-[(3R)-3-[(2,5,7-trimethyl-[1,2,4]triazolo[1,5-a]pyrimidin-6-yl)oxy]pyrrolidin-1-yl]pyrimidin-2-yl]bicyclo[1.1.1]pentane-1-carboxylic acid (40 mg, 91.85 mol, 1 eq), DIEA (35.61 mg, 275.56 μmol, 48.00 uL, 3 eq) and HATU (41.91 mg, 110.22 mol, 1.2 eq) in DMF (1 mL) was stirred at 0° C. for 0.5 h. Then 2-oxa-6-azaspiro[3.3]heptane (9.11 mg, 91.85 μmol, 1 eq) was added into the above solution at 0° C. The mixture was stirred at 20° C. for 2 h. LC-MS showed the desired compound was detected. The reaction mixture was purified by prep-HPLC (neutral condition) to give 2-oxa-6-azaspiro[3.3]heptan-6-yl-[3-[5-[(3R)-3-[(2,5,7-trimethyl-[1,2,4]triazolo[1,5-a]pyrimidin-6-yl)oxy]pyrrolidin-1-yl]pyrimidin-2-yl]-1-bicyclo[1.1.1]pentanyl]methanone (6 mg, 11.50 μmol, 12.52% yield, 99% purity). LCMS for product (ESI): m/z 517.3 (M+H)⁺. SFC for product: ee=100%.

¹H NMR (400 MHz, METHANOL-d₄) δ=8.12 (s, 2H), 5.01 (s, 1H), 4.83-4.78 (m, 4H), 4.57 (s, 2H), 4.19 (s, 2H), 3.79-3.70 (m, 1H), 3.64-3.54 (m, 2H), 3.52-3.45 (m, 1H), 2.67 (s, 3H), 2.60 (s, 3H), 2.54 (s, 3H), 2.53-2.50 (m, 1H), 2.42 (s, 6H), 2.40-2.31 (m, 1H).

### Example 25—Synthesis of (R)-(3-methoxyazetidin-1-yl)(3-(5-(3-((2,5,7-trimethyl-[1,2,4]triazolo[1,5-a]pyrimidin-6-yl)oxy)pyrrolidin-1-yl)pyrimidin-2-yl)bicyclo[1.1.1]pentan-1-yl)methanone (Compound 25)

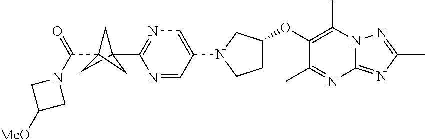

### Preparation of (3-methoxyazetidin-1-yl)-[3-[5-[(3R)-3-[(2,5,7-trimethyl-[1,2,4]triazolo[1,5-a]pyrimidin-6-yl)oxy]pyrrolidin-1-yl]pyrimidin-2-yl]-1-bicyclo[11.1.1]pentanyl]methanone

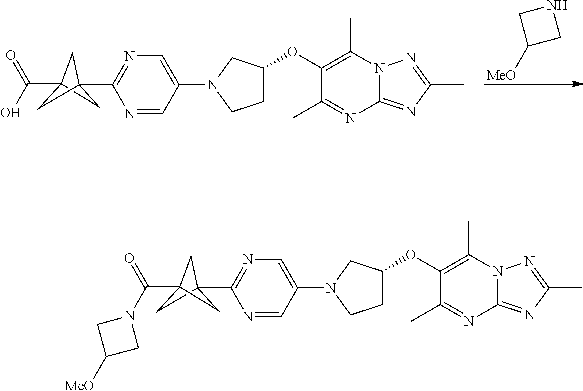 [SMILES: Cc1nc2nc(C)c(O[C@@H]3CCN(c4cnc(C56CC(C(=O)O)(C5)C6)nc4)C3)c(C)n2n1 ; COC1CNC1 ; COC1CN(C(=O)C23CC(c4ncc(N5CC[C@@H](Oc6c(C)nc7nc(C)nn7c6C)C5)cn4)(C2)C3)C1]

A solution of 3-[5-[(3R)-3-[(2,5,7-trimethyl-[1,2,4]triazolo[1,5-a]pyrimidin-6-yl)oxy]pyrrolidin-1-yl]pyrimidin-2-yl]bicyclo[1.1.1]pentane-1-carboxylic acid (40 mg, 91.85 mol, 1 eq), DIEA (35.61 mg, 275.56 μmol, 48.00 uL, 3 eq) and HATU (41.91 mg, 110.22 mol, 1.2 eq) in DMF (1 mL) was stirred at 0° C. for 0.5 h. Then 3-methoxyazetidine (11.35 mg, 91.85 μmol, 1 eq) was added into the above solution at 0° C. The mixture was stirred at 20° C. for 2 h. LC-MS showed the desired compound was detected. The reaction mixture was purified by prep-HPLC (neutral condition) to give (3-methoxyazetidin-1-yl)-[3-[5-[(3R)-3-[(2,5,7-trimethyl-[1,2,4]triazolo[1,5-a]pyrimidin-6-yl)oxy]pyrrolidin-1-yl]pyrimidin-2-yl]-1-bicyclo[1.1.1]pentanyl]methanone (5 mg, 9.81 μmol, 10.68% yield, 99% purity) as a yellow solid. LCMS for product (ESI): m/z 505.3 (M+H)⁺. SFC for product: ee=100%.

¹H NMR (400 MHz, METHANOL-d₄) δ=8.12 (s, 2H), 5.01 (s, 1H), 4.60-4.54 (m, 1H), 4.32-4.23 (m, 1H), 4.23-4.17 (m, 2H), 3.88-3.81 (m, 1H), 3.78-3.70 (m, 1H), 3.65-3.54 (m, 2H), 3.52-3.44 (m, 1H), 3.32 (s, 3H), 2.67 (s, 3H), 2.60 (s, 3H), 2.56-2.52 (m, 3H), 2.52-2.47 (m, 1H), 2.43 (s, 6H), 2.40-2.31 (m, 1H)

### Example 26—Synthesis of (R)-morpholino(3-(5-(3-((2,5,7-trimethyl-[1,2,4]triazolo[1,5-a]pyrimidin-6-yl)oxy)pyrrolidin-1-yl)pyrimidin-2-yl)bicyclo[1.1.1]pentan-1-yl)methanone (Compound 26)

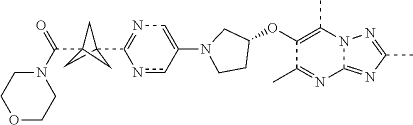

### Preparation of morpholino-[3-[5-[(3R)-3-[(2,5,7-trimethyl-[1,2,4]triazolo[1,5-a]pyrimidin-6-yl)oxy]pyrrolidin-1-yl]pyrimidin-2-yl]-1-bicyclo[1.1.1]pentanyl]methanone

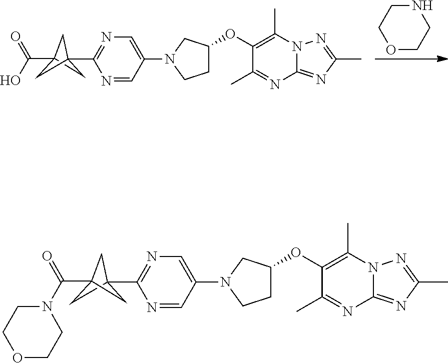

A mixture of 3-[5-[(3R)-3-[(2,5,7-trimethyl-[1,2,4]triazolo[1,5-a]pyrimidin-6-yl)oxy]pyrrolidin-1-yl]pyrimidin-2-yl]bicyclo[1.1.1]pentane-1-carboxylic acid (30 mg, 68.89 mol, 1 eq), DIEA (26.71 mg, 206.67 μmol, 36.00 uL, 3 eq) and HATU (31.43 mg, 82.67 μmol, 1.2 eq) in DMF (1 mL) was stirred at 0° C. for 0.5 h. Then morpholine (6.00 mg, 68.89 μmol, 6.06 uL, 1 eq) was added into the above mixture at 0° C. The mixture was stirred at 20° C. for 2 h. LC-MS showed the desired compound was detected. The reaction mixture was purified by prep-HPLC (neutral condition) to give morpholino-[3-[5-[(3R)-3-[(2,5,7-trimethyl-[1,2,4]triazolo[1,5-a]pyrimidin-6-yl)oxy]pyrrolidin-1-yl]pyrimidin-2-yl]-1-bicyclo[1.1.1]pentanyl]methanone (5 mg, 9.81 μmol, 14.24% yield, 99% purity). LCMS for product (ESI): m/z 505.3 (M+H)⁺. SFC for product: ee=100%.

¹H NMR (400 MHz, METHANOL-d₄) δ=8.13 (s, 2H), 5.01 (s, 1H), 3.79-3.72 (m, 3H), 3.72-3.64 (m, 4H), 3.64-3.52 (m, 4H), 3.52-3.45 (m, 1H), 2.67 (s, 3H), 2.60 (s, 3H), 2.54 (s, 3H), 2.53-2.51 (m, 1H), 2.49 (s, 6H), 2.41-2.32 (m, 1H).

### Example 27—Synthesis of (R)-6-((3-(4-(3-((2,5,7-trimethyl-[1,2,4]triazolo[1,5-a]pyrimidin-6-yl)oxy)pyrrolidin-1-yl)phenyl)bicyclo[1.1.1]pentan-1-yl)methyl)-2-oxa-6-azaspiro[3.3]heptane (Compound 27)

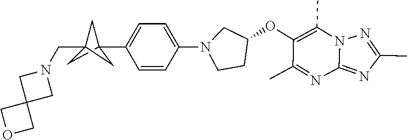

Compound 27 is prepared in a similar way as Compound 26.

### Example 28—Synthesis of (R)-6-((1-(4-(3-((3-methoxyazetidin-1-yl)methyl)bicyclo[1.1.1]pentan-1-yl)phenyl)pyrrolidin-3-yl)oxy)-2,5,7-trimethyl-[1,2,4]triazolo[1,5-a]pyrimidine (Compound 28)

Compound 28 is prepared in a similar way as Compound 26.

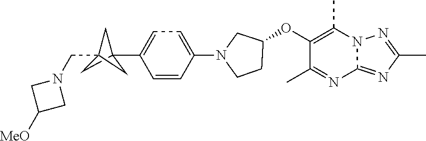

### Example 29—Synthesis of (R)-4-((3-(4-(3-((2,5,7-trimethyl-[1,2,4]triazolo[1,5-a]pyrimidin-6-yl)oxy)pyrrolidin-1-yl)phenyl)bicyclo[1.1.1]pentan-1-yl)methyl)morpholine (Compound 29)

Compound 29 is prepared in a similar way as Compound 26.

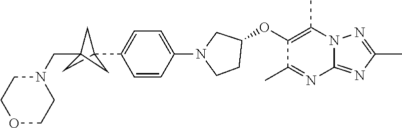

### Example 30—Synthesis of (R)-(2-oxa-6-azaspiro[3.3]heptan-6-yl)(3-(4-(3-((2,5,7-trimethyl-[1,2,4]triazolo[1,5-a]pyrimidin-6-yl)oxy)pyrrolidin-1-yl)phenyl)bicyclo[1.1.1]pentan-1-yl)methanone (Compound 30)

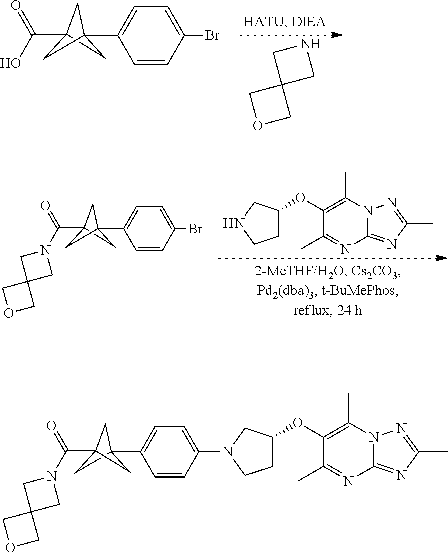

Compound 30 is prepared in a similar way as Compound 26.

### Example 31—Synthesis of (R)-(3-methoxyazetidin-1-yl)(3-(4-(3-((2,5,7-trimethyl-[1,2,4]triazolo[1,5-a]pyrimidin-6-yl)oxy)pyrrolidin-1-yl)phenyl)bicyclo[1.1.1]pentan-1-yl)methanone (Compound 31)

Compound 31 is prepared in a similar way as Compound 26.

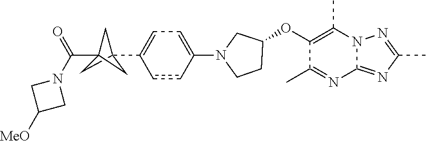

### Example 32—Synthesis of (R)-morpholino(3-(4-(3-((2,5,7-trimethyl-[1,2,4]triazolo[1,5-a]pyrimidin-6-yl)oxy)pyrrolidin-1-yl)phenyl)bicyclo[1.1.1]pentan-1-yl)methanone (Compound 32)

Compound 32 is prepared in a similar way as Compound 26.

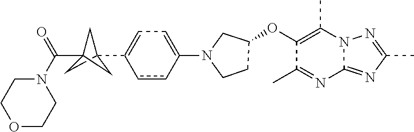

### Example 33—Synthesis of (2-oxa-6-azaspiro[3.3]heptan-6-yl)((1R,4r)-4-(4-((R)-3-((2,5,7-trimethyl-[1,2,4]triazolo[1,5-a]pyrimidin-6-yl)oxy)pyrrolidin-1-yl)phenyl)cyclohexyl)methanone (Compound 33)

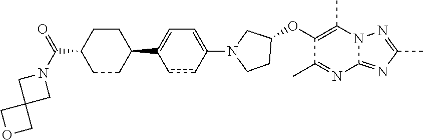

### Step 1. Preparation of [4-(4-chlorophenyl)cyclohexyl]-(2-oxa-6-azaspiro[3.3]heptan-6-yl)methanone

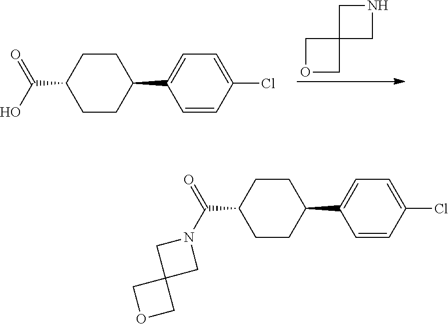

To a solution of 4-(4-chlorophenyl)cyclohexanecarboxylic acid (1 g, 4.19 mmol, 1 eq), 2-oxa-6-azaspiro[3.3]heptane; oxalic acid (792.60 mg, 4.19 mmol, 1 eq) and DIEA (1.62 g, 12.57 mmol, 2.19 mL, 3 eq) in THE (10 mL) was added dropwise T3P (4.00 g, 6.29 mmol, 3.74 mL, 50% purity, 1.5 eq) at 20° C. The reaction mixture was stirred at 20° C. for 2 h. LC-MS showed the desired compound was detected. The reaction mixture was partitioned between EA (20 mL) and water (20 mL). The organic phase was separated, washed with brine (20 mL), dried over Na₂SO₄, filtered and concentrated under reduced pressure to give a residue. The residue was purified by column chromatography (SiO₂, Petroleum ether/Ethyl acetate=10/1 to 1/1) to give [4-(4-chlorophenyl)cyclohexyl]-(2-oxa-6-azaspiro[3.3]heptan-6-yl)methanone (0.6 g, 1.88 mmol, 44.77% yield).

¹H NMR (400 MHz, CHLOROFORM-d) δ=7.27-7.23 (m, 2H), 7.14-7.10 (m, 2H), 4.84-4.76 (m, 4H), 4.33 (s, 2H), 4.15 (s, 2H), 2.52 (tt, J=3.2, 12.0 Hz, 1H), 2.18 (tt, J=3.6, 12.0 Hz, 1H), 2.00-1.90 (m, 2H), 1.85-1.81 (m, 2H), 1.73-1.60 (m, 2H), 1.49-1.36 (m, 2H)

### Step 2. Preparation of 2-oxa-6-azaspiro[3.3]heptan-6-yl-[4-[4-[(3R)-3-[(2,5,7-trimethyl-[1,2,4]triazolo[1,5-a]pyrimidin-6-yl)oxy]pyrrolidin-1-yl]phenyl]cyclohexyl]methanone

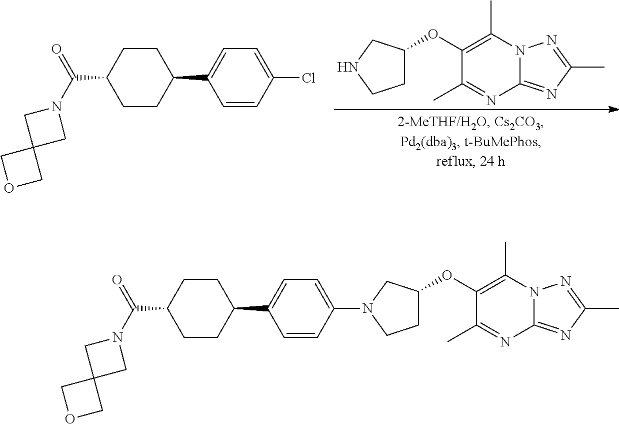 [SMILES: O=C([C@H]1CC[C@H](c2ccc(Cl)cc2)CC1)N1CC2(COC2)C1 ; Cc1nc2nc(C)c(O[C@@H]3CCNC3)c(C)n2n1 ; Cc1nc2nc(C)c(O[C@@H]3CCN(c4ccc([C@H]5CC[C@H](C(=O)N6CC7(COC7)C6)CC5)cc4)C3)c(C)n2n1]

A mixture of [4-(4-chlorophenyl)cyclohexyl]-(2-oxa-6-azaspiro[3.3]heptan-6-yl)methanone (0.3 g, 938.01 μmol, 1 eq), 2,5,7-trimethyl-6-[(3R)-pyrrolidin-3-yl]oxy-[1,2,4]triazolo[1,5-a]pyrimidine (360.44 mg, 1.13 mmol, 1.2 eq, 2HCl), ditert-butyl-[2-(o-tolyl)phenyl]phosphane (117.23 mg, 375.21 μmol, 0.4 eq), Cs₂CO₃ (1.53 g, 4.69 mmol, 5 eq) and Pd₂(dba)₃ (171.79 mg, 187.60 μmol, 0.2 eq) in 2-MeTHF (10 mL) and H2O (5 mL) was de-gassed and then heated to 80° C. for 24 h under N₂. LC-MS showed the desired compound was detected. The reaction mixture was poured into H₂O (20 mL). The mixture was extracted with ethyl acetate (10 mL). The organic phase was washed with brine (10 mL), dried over anhydrous Na₂SO₄, concentrated in vacuum to give a residue. The residue was purified by prep-HPLC (neutral condition) to give 2-oxa-6-azaspiro[3.3]heptan-6-yl-[4-[4-[(3R)-3-[(2,5,7-trimethyl-[1,2,4]triazolo[1,5-a]pyrimidin-6-yl)oxy]pyrrolidin-1-yl]phenyl]cyclohexyl]methanone (19 mg, 34.01 μmol, 3.63% yield, 95% purity). LCMS for product (ESI): m/z 531.3 (M+H)⁺. SFC for product: ee=100%.

¹H NMR (400 MHz, CHLOROFORM-d) δ=7.10 (d, J=8.4 Hz, 2H), 6.53 (d, J=8.4 Hz, 2H), 4.86-4.77 (m, 5H), 4.35 (s, 2H), 4.16 (s, 2H), 3.74-3.64 (m, 1H), 3.52-3.45 (m, 2H), 3.43-3.38 (m, 1H), 2.66 (s, 3H), 2.62 (s, 3H), 2.61 (s, 3H), 2.54-2.38 (m, 2H), 2.33-2.24 (m, 1H), 2.23-2.13 (m, 1H), 2.01-1.92 (m, 2H), 1.87-1.78 (m, 2H), 1.74-1.65 (m, 2H), 1.47-1.37 (m, 2H)

### Example 34—Synthesis of (3-methoxyazetidin-1-yl)((1R,4r)-4-(4-((R)-3-((2,5,7-trimethyl-[1,2,4]triazolo[1,5-a]pyrimidin-6-yl)oxy)pyrrolidin-1-yl)phenyl)cyclohexyl)methanone (Compound 34)

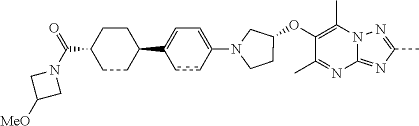

### Step 1. Preparation of [4-(4-chlorophenyl)cyclohexyl]-(3-methoxyazetidin-1-yl)methanone

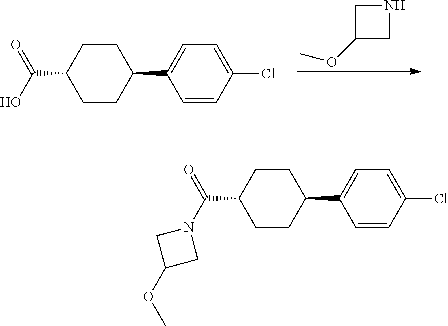

To a solution of 4-(4-chlorophenyl)cyclohexanecarboxylic acid (1 g, 4.19 mmol, 1 eq), 3-methoxyazetidine; hydrochloride (517.80 mg, 4.19 mmol, 1 eq) and DIEA (1.62 g, 12.57 mmol, 2.19 mL, 3 eq) in THE (10 mL) was added dropwise T3P (4.00 g, 6.29 mmol, 3.74 mL, 50% purity, 1.5 eq) at 20° C. The reaction mixture was stirred at 20° C. for 2 h. LC-MS showed the desired compound was detected. The reaction mixture was partitioned between EA (20 mL) and water (20 mL). The organic phase was separated, washed with brine (20 ml), dried over Na₂SO₄, filtered and concentrated under reduced pressure to give a residue. The residue was purified by column chromatography (SiO₂, Petroleum ether/Ethyl acetate=10/1 to 1/1) to give [4-(4-chlorophenyl)cyclohexyl]-(3-methoxyazetidin-1-yl)methanone (0.7 g, 2.27 mmol, 54.27% yield).

¹H NMR (400 MHz, CHLOROFORM-d) δ=7.31-7.27 (m, 2H), 7.18-7.14 (m, 2H), 4.40-4.31 (m, 1H), 4.29-4.15 (m, 2H), 4.11-4.04 (m, 1H), 3.96-3.87 (m, 1H), 3.35 (s, 3H), 2.57 (tt, J=3.2, 12.4 Hz, 1H), 2.25 (tt, J=3.6, 12.0 Hz, 1H), 2.05-1.94 (m, 2H), 1.94-1.83 (m, 2H), 1.74 (s, 1H), 1.72-1.66 (m, 1H), 1.53-1.40 (m, 2H).

### Step 2. Preparation of (3-methoxyazetidin-1-yl)-[4-[4-[(3R)-3-[(2,5,7-trimethyl-[1,2,4]triazolo[1,5-a]pyrimidin-6-yl)oxy]pyrrolidin-1-yl]phenyl]cyclohexyl]methanone

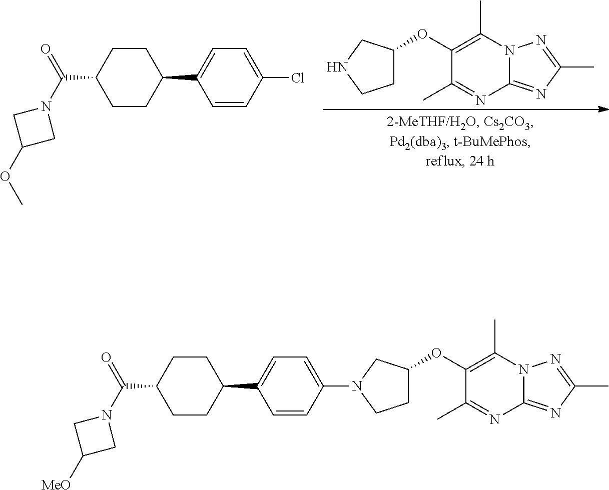 [SMILES: COC1CN(C(=O)[C@H]2CC[C@H](c3ccc(Cl)cc3)CC2)C1 ; Cc1nc2nc(C)c(O[C@@H]3CCNC3)c(C)n2n1 ; COC1CN(C(=O)[C@H]2CC[C@H](c3ccc(N4CC[C@@H](Oc5c(C)nc6nc(C)nn6c5C)C4)cc3)CC2)C1]

A mixture of [4-(4-chlorophenyl)cyclohexyl]-(3-methoxyazetidin-1-yl)methanone (300 mg, 974.61 μmol, 1 eq), 2,5,7-trimethyl-6-[(3R)-pyrrolidin-3-yl]oxy-[1,2,4]triazolo[1,5-a]pyrimidine (374.51 mg, 1.17 mmol, 1.2 eq, 2HCl), ditert-butyl-[2-(o-tolyl)phenyl]phosphane (121.80 mg, 389.85 μmol, 0.4 eq), Cs₂CO₃ (1.59 g, 4.87 mmol, 5 eq) and Pd₂(dba)₃ (178.49 mg, 194.92 μmol, 0.2 eq) in 2-MeTHF (10 mL) and H₂O (5 mL) was de-gassed and then heated to 80° C. for 24 h under N₂. LC-MS showed the desired compound was detected. The reaction mixture was poured into H₂O (20 mL). The mixture was extracted with ethyl acetate (10 mL). The organic phase was washed with brine (10 mL), dried over anhydrous Na₂SO₄, concentrated in vacuum to give a residue. The residue was purified by prep-HPLC (neutral condition) to give (3-methoxyazetidin-1-yl)-[4-[4-[(3R)-3-[(2,5,7-trimethyl-[1,2,4]triazolo[1,5-a]pyrimidin-6-yl)oxy]pyrrolidin-1-yl]phenyl]cyclohexyl]methanone (19 mg, 33.34 μmol, 3.42% yield, 91% purity). LCMS for product (ESI): m/z 519.3 (M+H)⁺. SFC for product: ee=100%

¹H NMR (400 MHz, CHLOROFORM-d) δ=7.10 (d, J=8.4 Hz, 2H), 6.53 (d, J=8.4 Hz, 2H), 4.86-4.76 (m, 1H), 4.33 (m, 1H), 4.26-4.19 (m, 1H), 4.19-4.13 (m, 1H), 4.05 (m, 1H), 3.89 (m, 1H), 3.74-3.63 (m, 1H), 3.51-3.44 (m, 2H), 3.44-3.37 (m, 1H), 3.32 (s, 3H), 2.66 (s, 3H), 2.62 (s, 3H), 2.61 (s, 3H), 2.53-2.46 (m, 1H), 2.46-2.39 (m, 1H), 2.34-2.24 (m, 1H), 2.24-2.16 (m, 1H), 1.97 (m, 2H), 1.91-1.80 (m, 2H), 1.76-1.66 (m, 2H), 1.50-1.40 (m, 2H).

### Example 35—Synthesis of morpholino((1R,4r)-4-(4-((R)-3-((2,5,7-trimethyl-[1,2,4]triazolo[1,5-a]pyrimidin-6-yl)oxy)pyrrolidin-1-yl)phenyl)cyclohexyl)methanone (Compound 35)

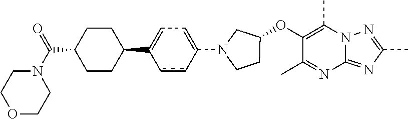

Step 1. Preparation of [4-(4-chlorophenyl)cyclohexyl]-morpholino-methanone

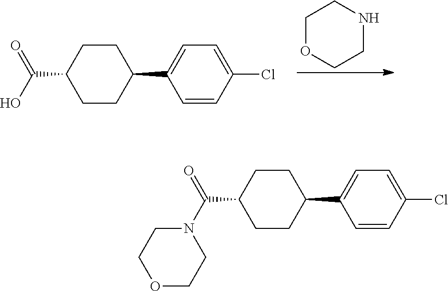

A mixture of morpholine (438.04 mg, 5.03 mmol, 442.46 uL, 1.2 eq), DIEA (1.62 g, 12.57 mmol, 2.19 mL, 3 eq) and 4-(4-chlorophenyl)cyclohexanecarboxylic acid (1 g, 4.19 mmol, 1 eq) in THF (10 mL) was added dropwise T3P (4.00 g, 6.29 mmol, 3.74 mL, 50% purity, 1.5 eq) at 20° C. and stirred at 20° C. for 2 h. LC-MS showed the desired compound was detected. The reaction mixture was partitioned between EA (20 mL) and water (20 mL). The organic phase was separated, washed with brine (20 mL), dried over Na₂SO₄, filtered and concentrated under reduced pressure to give a residue. The residue was purified by column chromatography (SiO2, Petroleum ether/Ethyl acetate=10/1 to 1/1) to give [4-(4-chlorophenyl)cyclohexyl]-morpholino-methanone (0.8 g, 2.60 mmol, 62.03% yield) as a white solid.

¹H NMR (400 MHz, CHLOROFORM-d) δ=7.27-7.23 (m, 2H), 7.15-7.10 (m, 2H), 3.72-3.60 (m, 6H), 3.53 (d, J=3.9 Hz, 2H), 2.61-2.46 (m, 2H), 2.01-1.93 (m, 2H), 1.91-1.82 (m, 2H), 1.80-1.69 (m, 2H), 1.45 (dq, J=3.5, 12.8 Hz, 2H)

### Step 2. Preparation of morpholino-[4-[4-[(3R)-3-[(2,5,7-trimethyl-[1,2,4]triazolo[1,5-a]pyrimidin-6-yl)oxy]pyrrolidin-1-yl]phenyl]cyclohexyl]methanone

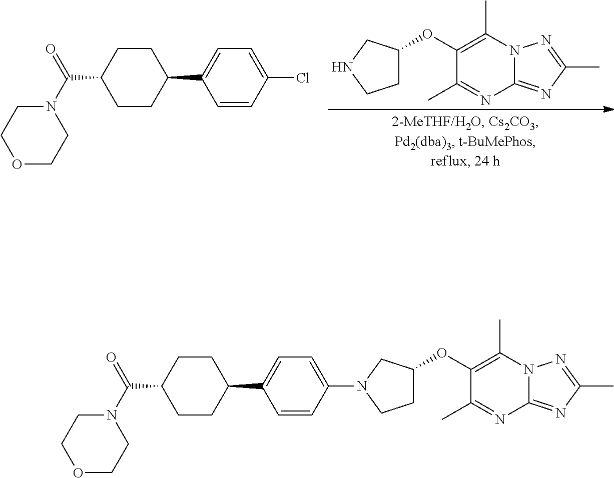 [SMILES: O=C([C@H]1CC[C@H](c2ccc(Cl)cc2)CC1)N1CCOCC1 ; Cc1nc2nc(C)c(O[C@@H]3CCNC3)c(C)n2n1 ; Cc1nc2nc(C)c(O[C@@H]3CCN(c4ccc([C@H]5CC[C@H](C(=O)N6CCOCC6)CC5)cc4)C3)c(C)n2n1]

A mixture of [4-(4-chlorophenyl)cyclohexyl]-morpholino-methanone (300 mg, 974.61 mol, 1 eq), 2,5,7-trimethyl-6-[(3R)-pyrrolidin-3-yl]oxy-[1,2,4]triazolo[1,5-a]pyrimidine (374.51 mg, 1.17 mmol, 1.2 eq, 2HCl), ditert-butyl-[2-(o-tolyl)phenyl]phosphane (121.80 mg, 389.85 μmol, 0.4 eq), Cs₂CO₃ (1.59 g, 4.87 mmol, 5 eq) and Pd₂(dba)₃ (178.49 mg, 194.92 mol, 0.2 eq) in 2-MeTHF (10 mL) and H₂O (5 mL) was de-gassed and then heated to 80° C. for 24 h under N₂. LC-MS showed the desired compound was detected. The reaction mixture was poured into H₂O (20 mL). The mixture was extracted with ethyl acetate (10 mL). The organic phase was washed with brine (10 mL), dried over anhydrous Na₂SO₄, concentrated in vacuum to give a residue. The residue was purified by prep-HPLC (neutral condition) to give morpholino-[4-[4-[(3R)-3-[(2,5,7-trimethyl-[1,2,4]triazolo[1,5-a]pyrimidin-6-yl)oxy]pyrrolidin-1-yl]phenyl]cyclohexyl]methanone (13 mg, 23.06 μmol, 2.37% yield, 92% purity) as an off-white solid. LCMS for product (ESI): m/z 519.3 (M+H)⁺. SFC for product: ee=100%.

¹H NMR (400 MHz, CHLOROFORM-d) δ=7.11 (d, J=8.4 Hz, 2H), 6.54 (d, J=8.4 Hz, 2H), 4.81 (m, 1H), 3.69 (m, 5H), 3.66 (m, 2H), 3.55 (m, 2H), 3.52-3.45 (m, 2H), 3.44-3.38 (m, 1H), 2.66 (s, 3H), 2.62 (s, 3H), 2.61 (s, 3H), 2.55-2.48 (m, 2H), 2.47-2.39 (m, 1H), 2.33-2.22 (m, 1H), 1.99 (m, 2H), 1.91-1.83 (m, 2H), 1.82-1.69 (m, 2H), 1.51-1.41 (m, 2H).

### PHARMACEUTICAL COMPOSITIONS

### Example A-1: Parenteral Pharmaceutical Composition

To prepare a parenteral pharmaceutical composition suitable for administration by injection (subcutaneous, intravenous), 1-1000 mg of a water-soluble salt of a compound described herein, or a stereoisomer, or pharmaceutically acceptable salt or solvate thereof, is dissolved in sterile water and then mixed with 10 mL of 0.9% sterile saline. A suitable buffer is optionally added as well as optional acid or base to adjust the pH. The mixture is incorporated into a dosage unit form suitable for administration by injection.

### Example A-2: Oral Solution

To prepare a pharmaceutical composition for oral delivery, a sufficient amount of a compound described herein, or a pharmaceutically acceptable salt thereof, is added to water (with optional solubilizer(s), optional buffer(s) and taste masking excipients) to provide a 20 mg/mL solution.

### Example A-3: Oral Tablet

A tablet is prepared by mixing 20-50% by weight of a compound described herein, or a pharmaceutically acceptable salt thereof, 20-50% by weight of microcrystalline cellulose, and 1-10% by weight of magnesium stearate or other appropriate excipients. Tablets are prepared by direct compression. The total weight of the compressed tablets is maintained at 100-500 mg.

### Example A-4: Oral Capsule

To prepare a pharmaceutical composition for oral delivery, 1-1000 mg of a compound described herein, or a pharmaceutically acceptable salt thereof, is mixed with starch or other suitable powder blend. The mixture is incorporated into an oral dosage unit such as a hard gelatin capsule, which is suitable for oral administration.

In another embodiment, 1-1000 mg of a compound described herein, or a pharmaceutically acceptable salt thereof, is placed into Size 4 capsule, or size 1 capsule (hypromellose or hard gelatin) and the capsule is closed.

### BIOLOGICAL EXAMPLES

### Example B-1: MCT4 and MCT1 Transport Assays

The following in vitro MCT4 and MCT1 transport assays were used to measure the effect of the compounds on L-lactate influx into two different cell lines:
a) MDA-MB-231 cells—a triple negative human breast cancer cell line expressing exclusively MCT4 (confirmed by Western blotting) was used for the MCT4 transport assay,
b) BT-20 cells—a triple negative human breast cancer cell line expressing exclusively MCT1 (confirmed by Western blotting) was used for the

MCT1 transport assay.

The compounds' efficacy in both assays was determined by calculating their IC₅₀ value (the concentration of test compound that inhibited 50% of biological activity).

MCT4 is predominantly involved in cellular efflux of lactate from the cell, preventing intracellular acidification. In the MCT4 in vitro transport system described here it was possible to drive influx of lactate in the cells and test the compounds efficacy in preventing this process. MCT1 is involved in influx-efflux of lactate, and because it is closely related to MCT4, it is a major selectivity target for MCT4 inhibitor. The MCT1 in vitro transport system described here measured the influx of lactate in the cells and ability of test compounds in preventing this process.

### Transport Assay

### Cell Line and Media

MDA-MB-231 (ATCC); Media: RPMI1640+10% FCS+1% PS.

BT-20 (ATCC); Media: EMEM+10% FBS+1% PS

### Reagents

| | Name | Cat# | Vendor |
|---|---|---|---|
| Medium | RPMI1640 | 22400105 | Invitrogen |
| | EMEM | 30-2003 | ATCC |
| | FBS | SV30087.03 | Hyclone |
| | PS | SV30010 | Hyclone |
| Reagent | Syrosingopine | B1323-5 | BioVision |
| | AZD3965 | S7339 | Selleck |
| | BCECF AM | B1170 | Life technology |
| | Sodium L-lactate | L7022-10G | Sigma |
| | D-(+)-Glucose | G7021-1KG | Sigma |
| | KCl | 9333 | Sigma |
| | NaCl | S7653 | Sigma |
| | CaCl2 | 6991 | Sigma |
| | MgCl2 | M1028 | Sigma |
| | HEPES | 15630080 | Gibco |
| Consumables | 96-well plate Tips | 9000-0911 | Molecular Devices |
| | 96-well plate | 655090 | Greiner |
| | V 96-well plate | P-96-450V-C-S | Axygen |

### Assay Buffer

Tyrode's Solution pH7.4 (NaCl 119 mM; KC1 5 mM; HEPES pH7.4 25 mM; CaCl2 2 mM; MgCl2 2 mM; Glucose 6 g/L)

### Equipment

### FlexStation III (Molecular Devices)

### ECHO 555 (Labcyte)

### Bravo (Agilent)

### Incubator (Thermo-HEPA CLASS 100)

### Vi-cell XR Cell Viability Analyzer (Beckman Coulter)

### Assay Protocol

The following assay was performed to assess MCT4 (MDA-MB-231 cell line) or MCT1 (BT-20 cell line) activity by monitoring the intracellular pH change that accompanies lactate/proton transport using the pH-sensitive fluorescent dye BCECF-AM.

Compound preparation: The compounds were serially diluted in 100% DMSO 1:3 for 8 pts and transferred (500 nl) to a 96-well plate by Echo.

Cell preparation: Cells were briefly rinsed with 10 ml of PBS solution to remove all traces of serum, trypsinized (0.05% Trypsin-EDTA) at 25° C. for 2-3 minutes and washed twice with Tyrode's solution. The cell viability and concentration were determined using Vi-Cell. Cells were resuspended in Tyrode's solution.

Cells were loaded with the pH sensitive dye BCECF-AM (5 uM final concentration from the 30 mM DMSO stock solution) at 37° C. for 30 minutes before being washed twice to remove excess dye and resuspended to 2 million/ml cells in Tyrode's solution. Cells were seeded into a 96-well plate (200,000 cells/well) directly onto the test compound or a reference compound for 10 minutes at 37° C. Following incubation with test compounds, 25 ul of 30 mM Sodium-L-lactate was added. This addition was performed on the FlexStation III as a part of a real-time kinetic protocol. The ratio of cellular fluorescence at 535 nm after excitation at 490 nm/440 nm was measured over time for baseline (pre-lactate addition) and post-lactate addition for 60 sec with or without compounds present. Inhibitory concentration response curves were generated for compounds and their IC₅₀ values calculated.

IC₅₀ of the L-lactate transport assays for representative compounds are described in Table 2. The potencies are divided into four criteria: + means that IC₅₀ is greater than 1,000 nM; ++ means that IC₅₀ is between 500 nM and 1,000 nM; +++ means IC₅₀ is between 100 nM and 499 nM; ++++ means IC₅₀ is below 100 nM. Inactive compounds are designated NA. In some embodiments, compounds designated with IC₅₀ “+” may have IC₅₀s between 1 μM and 30 μM.

TABLE 2

| Compound No. | MCT4 IC₅₀ (nM) | MCT1 IC₅₀ (nM) |
|---|---|---|
| Compound 1 | ++++ | + |
| Compound 2 | +++ | + |
| Compound 3 | + | + |
| Compound 4 | ++++ | + |
| Compound 5 | ++++ | + |
| Compound 6 | ++++ | + |
| Compound 7 | ++++ | + |
| Compound 8 | ++++ | + |
| Compound 9 | ++++ | + |
| Compound 10 | ++++ | + |
| Compound 11 | +++ | + |
| Compound 12 | ++++ | + |
| Compound 13 | ++++ | + |
| Compound 14 | ++++ | + |
| Compound 15 | ++++ | + |
| Compound 16 | ++++ | + |
| Compound 17 | ++ | + |
| Compound 18 | ++++ | + |
| Compound 19 | ++++ | + |
| Compound 20 | ++++ | + |
| Compound 21 | ++ | + |
| Compound 22 | ++++ | + |
| Compound 23 | ++++ | + |
| Compound 24 | + | + |
| Compound 25 | ++ | + |
| Compound 26 | +++ | + |
| Compound 33 | ++++ | + |
| Compound 34 | ++++ | + |
| Compound 35 | ++++ | + |

The examples and embodiments described herein are for illustrative purposes only and various modifications or changes suggested to persons skilled in the art are to be included within the spirit and purview of this application and scope of the appended claims.

What is claimed is:

1. A compound that has the structure of Formula (I-A), or a stereoisomer, or pharmaceutically acceptable salt or solvate thereof: 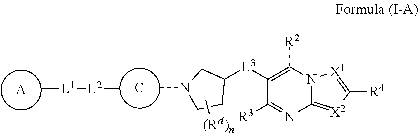
wherein:
X¹ is N or CR¹;
X² is N or CR¹; provided that both X¹ and X² are not N at the same time;
each of R¹, R², R³, and R⁴ is independently H, halogen, —CN, —OH, C₁-C₅alkyl, C₁-C₅alkoxy, C₁-C₅fluoroalkyl, or C₁-C₅fluoroalkoxy;
Ring A is a monocyclic C₃-C₈heterocycloalkyl with at least one N atom, or a bicyclic C₅-C₁₀heterocycloalkyl with at least one N atom, wherein ring A is unsubstituted or substituted with 1, 2, 3, or 4 R^{a};
each R^{a} is independently selected from the group consisting of hydrogen, —CN, halogen, —OR¹¹, —N(R¹¹)₂, —S(═O)₂(R¹²), —N(R¹¹)—S(═O)₂(R¹²), —C(═O)N(R¹¹)₂, —NR¹¹—C(═O)R¹¹, —C(═O)R¹¹, —C(═O)OR¹¹, —NR¹¹—C(═O)OR¹¹, —OC(═O)N(R¹¹)₂, C₁-C₄alkyl, C₂-C₄alkenyl, C₂-C₄alkynyl, C₁-C₄deuteroalkyl, C₁-C₄deuteroalkoxy, C₁-C₄fluoroalkyl, C₁-C₄fluoroalkoxy, C₁-C₄heteroalkyl, or substituted or unsubstituted monocyclic C₂-C₅heterocycloalkyl;
or two R^{a} that are attached to the same carbon atom are taken together with the carbon atom to form a substituted or unsubstituted monocyclic C₃-C₆cycloalkyl or a substituted or unsubstituted monocyclic C₃-C₅heterocycloalkyl;
L¹ is —CR⁵R⁶— or —NR⁷—;
R⁵ and R⁶ are each independently H or C₁-C₅alkyl;
or R⁵ and R⁶ are taken together with the carbon atom to which they are attached to form —C(═O)—;
R⁷ is H or C₁-C₅alkyl;
L² is C₁-C₄alkyl, C₂-C₄alkenyl, or C₂-C₄alkynyl;
or L² is a ring B;
ring B is a monocyclic C₃-C₈cycloalkyl, phenyl, bicyclic C₅-C₁₂cycloalkyl, monocyclic C₃-C₈heterocycloalkyl, bicyclic C₅-C₁₀heterocycloalkyl, or monocyclic heteroaryl, wherein B is unsubstituted or is substituted with 1, 2, 3, or 4 R^{b};
each R^{b} is independently selected from the group consisting of hydrogen, —CN, halogen, —OR¹¹, —N(R¹¹)₂, —S(═O)₂(R¹²), —N(R¹¹)—S(═O)₂(R¹²), —C(═O)N(R¹¹)₂, —NR¹¹—C(═O)R¹¹, —C(═O)R¹¹, —C(═O)OR¹¹, —NR¹¹—C(═O)OR¹¹, —OC(═O)N(R¹¹)₂, C₁-C₄alkyl, C₂-C₄alkenyl, C₂-C₄alkynyl, C₁-C₄deuteroalkyl, C₁-C₄deuteroalkoxy, C₁-C₄fluoroalkyl, C₁-C₄fluoroalkoxy, C₁-C₄heteroalkyl, or substituted or unsubstituted monocyclic C₂-C₅heterocycloalkyl;
Ring C is selected from the group consisting of phenyl or a 6-membered heteroaryl with 1-3N atoms, wherein Ring C is unsubstituted or is substituted with 1, 2, 3, or 4 R^{c};
each R^{c} is independently selected from the group consisting of hydrogen, —CN, halogen, —OR¹¹, —N(R¹¹)₂, —S(═O)₂(R¹²), —N(R¹¹)—S(═O)₂(R¹²), —C(═O)N(R¹¹)₂, —NR¹¹—C(═O)R¹¹, —C(═O)R¹¹, —C(═O)OR¹¹, —NR¹¹—C(═O)OR¹¹, —OC(═O)N(R¹¹)₂, C₁-C₄alkyl, C₂-C₄alkenyl, C₂-C₄alkynyl, C₁-C₄deuteroalkyl, C₁-C₄deuteroalkoxy, C₁-C₄fluoroalkyl, C₁-C₄fluoroalkoxy, C₁-C₄heteroalkyl, or substituted or unsubstituted monocyclic C₃-C₅heterocycloalkyl;
each R^{d} is independently selected from the group consisting of hydrogen, —CN, halogen, —OR¹¹, C₁-C₄alkyl, C₂-C₄alkenyl, C₂-C₄alkynyl, C₁-C₄deuteroalkyl, C₁-C₄deuteroalkoxy, C₁-C₄fluoroalkyl, C₁-C₄fluoroalkoxy, or C₁-C₄heteroalkyl;
L³ is —O—, —NR⁸—, or —CR⁹R¹⁰—;
R⁸ is H or C₁-C₅alkyl;
R⁹ and R¹⁰ are each independently H or C₁-C₅alkyl;
each R¹¹ is independently selected from the group consisting of hydrogen, C₁-C₄alkyl, C₁-C₄deuteroalkyl, C₁-C₄fluoroalkyl, C₁-C₄heteroalkyl, substituted or unsubstituted C₃-C₆cycloalkyl, substituted or unsubstituted C₃-C₅heterocycloalkyl, substituted or unsubstituted phenyl, or substituted or unsubstituted monocyclic heteroaryl;
each R¹² is independently selected from the group consisting of C₁-C₄alkyl, C₁-C₄deuteroalkyl, C₁-C₄fluoroalkyl, C₁-C₄heteroalkyl, substituted or unsubstituted C₃-C₆cycloalkyl, substituted or unsubstituted C₃-C₅heterocycloalkyl, substituted or unsubstituted phenyl, or substituted or unsubstituted monocyclic heteroaryl; and
n is 0, 1, or 2.

2. The compound of claim 1, or a stereoisomer, or pharmaceutically acceptable salt or solvate thereof, wherein:
X¹ is N; and
X² is CR¹.

3. The compound of claim 1, or a stereoisomer, or pharmaceutically acceptable salt or solvate thereof, wherein:
X¹ is CR¹; and
X² is N.

4. The compound of claim 1, or a stereoisomer, or pharmaceutically acceptable salt or solvate thereof, wherein:
X¹ is CR¹; and
X² is CR¹.

5. A compound that has the structure of Formula (I-B), or a stereoisomer, or pharmaceutically acceptable salt or solvate thereof: 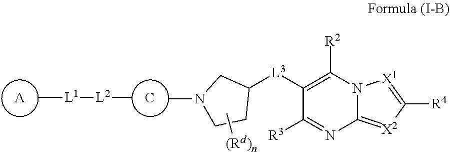
wherein:
X¹ is N or CR¹;
X² is N or CR¹;
each of R¹, R², R³, and R⁴ is independently H, halogen, —CN, —OH, C₁-C₅alkyl, C₁-C₅alkoxy, C₁-C₅fluoroalkyl, or C₁-C₅fluoroalkoxy;
Ring A is a monocyclic C₃-C₈heterocycloalkyl with at least one N atom, or a bicyclic C₅-C₁₀heterocycloalkyl with at least one N atom, wherein ring A is unsubstituted or substituted with 1, 2, 3, or 4 R^{a};
each R^{a} is independently selected from the group consisting of hydrogen, —CN, halogen, —OR¹¹, —N(R¹¹)₂, —S(═O)₂(R¹²), —N(R¹¹)—S(═O)₂(R¹²), —C(═O)N(R¹¹)₂, —NR¹¹—C(═O)R¹¹, —C(═O)R¹¹, —C(═O)OR¹¹, —NR¹¹—C(═O)OR¹¹, —OC(═O)N(R¹¹)₂, C₁-C₄alkyl, C₂-C₄alkenyl, C₂-C₄alkynyl, C₁-C₄deuteroalkyl, C₁-C₄deuteroalkoxy, C₁-C₄fluoroalkyl, C₁-C₄fluoroalkoxy, C₁-C₄heteroalkyl, or substituted or unsubstituted monocyclic C₂-C₅heterocycloalkyl;
or two R^{a} that are attached to the same carbon atom are taken together with the carbon atom to form a substituted or unsubstituted monocyclic C₃-C₆cycloalkyl or a substituted or unsubstituted monocyclic C₃-C₅heterocycloalkyl;
L¹ is —CR⁵R⁶— or —NR⁷—;
R⁵ and R⁶ are each independently H or C₁-C₅alkyl;
or R⁵ and R⁶ are taken together with the carbon atom to which they are attached to form —C(═O)—;
L² is C₁-C₄alkyl, C₂-C₄alkenyl, C₂-C₄alkynyl;
or L² is a ring B;
ring B is a monocyclic C₃-C₈cycloalkyl, bicyclic C₅-C₁₂cycloalkyl, monocyclic C₃-C₈heterocycloalkyl, or bicyclic C₅-C₁₀heterocycloalkyl, wherein ring B is unsubstituted or is substituted with 1, 2, 3, or 4 R^{b};
or ring B is phenyl or monocyclic heteroaryl, wherein ring B is unsubstituted or is substituted with 1, 2, 3, or 4 R^{b}; and L¹ is —CR⁵R⁶— or —NR⁷—, and R⁵ and R⁶ are taken together with the carbon atom to which they are attached to form —C(═O)—;
R⁷ is H or C₁-C₅alkyl;
ring B is a monocyclic C₃-C₈cycloalkyl, phenyl, bicyclic C₅-C₁₂cycloalkyl, monocyclic C₃-C₈heterocycloalkyl, bicyclic C₅-C₁₀heterocycloalkyl, or monocyclic heteroaryl, wherein ring B is unsubstituted or is substituted with 1, 2, 3, or 4 R^{b};
each R^{b} is independently selected from the group consisting of hydrogen, —CN, halogen, —OR¹¹, —N(R¹¹)₂, —S(═O)₂(R¹²), —N(R¹¹)—S(═O)₂(R¹²), —C(═O)N(R¹¹)₂, —NR¹¹—C(═O)R¹¹, —C(═O)R¹¹, —C(═O)OR¹¹, —NR¹¹—C(═O)OR¹¹, —OC(═O)N(R¹¹)₂, C₁-C₄alkyl, C₂-C₄alkenyl, C₂-C₄alkynyl, C₁-C₄deuteroalkyl, C₁-C₄deuteroalkoxy, C₁-C₄fluoroalkyl, C₁-C₄fluoroalkoxy, C₁-C₄heteroalkyl, or substituted or unsubstituted monocyclic C₂-C₅heterocycloalkyl;
Ring C is selected from the group consisting of phenyl or a 6-membered heteroaryl with 1-3N atoms, wherein Ring C is unsubstituted or is substituted with 1, 2, 3, or 4 R^{c};
each R^{c} is independently selected from the group consisting of hydrogen, —CN, halogen, —OR¹¹, —N(R¹¹)₂, —S(═O)₂(R¹²), —N(R¹¹)—S(═O)₂(R¹²), —C(═O)N(R¹¹)₂, —NR¹¹—C(═O)R¹¹, —C(═O)R¹¹, —C(═O)OR¹¹, —NR¹¹—C(═O)OR¹¹, —OC(═O)N(R¹¹)₂, C₁-C₄alkyl, C₂-C₄alkenyl, C₂-C₄alkynyl, C₁-C₄deuteroalkyl, C₁-C₄deuteroalkoxy, C₁-C₄fluoroalkyl, C₁-C₄fluoroalkoxy, C₁-C₄heteroalkyl, or substituted or unsubstituted monocyclic C₃-C₅heterocycloalkyl;
each R^{d} is independently selected from the group consisting of hydrogen, —CN, halogen, —OR¹¹, C₁-C₄alkyl, C₂-C₄alkenyl, C₂-C₄alkynyl, C₁-C₄deuteroalkyl, C₁-C₄deuteroalkoxy, C₁-C₄fluoroalkyl, C₁-C₄fluoroalkoxy, or C₁-C₄heteroalkyl;
L³ is —O—, —NR⁸—, or —CR⁹R¹⁰—;
R⁸ is H or C₁-C₅alkyl;
R⁹ and R¹⁰ are each independently H or C₁-C₅alkyl;
each R¹¹ is independently selected from the group consisting of hydrogen, C₁-C₄alkyl, C₁-C₄deuteroalkyl, C₁-C₄fluoroalkyl, C₁-C₄heteroalkyl, substituted or unsubstituted C₃-C₆cycloalkyl, substituted or unsubstituted C₃-C₅heterocycloalkyl, substituted or unsubstituted phenyl, or substituted or unsubstituted monocyclic heteroaryl;
each R¹² is independently selected from the group consisting of C₁-C₄alkyl, C₁-C₄deuteroalkyl, C₁-C₄fluoroalkyl, C₁-C₄heteroalkyl, substituted or unsubstituted C₃-C₆cycloalkyl, substituted or unsubstituted C₃-C₅heterocycloalkyl, substituted or unsubstituted phenyl, or substituted or unsubstituted monocyclic heteroaryl; and
n is 0, 1, or 2;
provided that when X¹ and X² are both N, R², R³, and R⁴ are each methyl, L¹ is —CR⁵R⁶—, R⁵ and R⁶ are each independently H or C₁-C₅alkyl, L² is ring B, and L³ is —O—, then
(i) ring A is a 4-membered heterocycloalkyl or a spiro bicyclic C₅-C₈heterocycloalkyl, or (ii) ring B is not phenyl or monocyclic heteroaryl.

6. A compound that has the structure of Formula (I-C), or a stereoisomer, or pharmaceutically acceptable salt or solvate thereof: 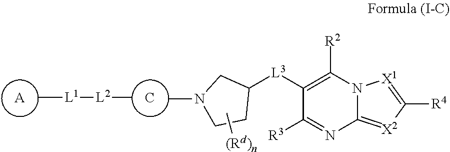
wherein:
X¹ is N or CR¹;
X² is N or CR¹;
each of R¹, R², R³, and R⁴ is independently H, halogen, —CN, —OH, C₁-C₅alkyl, C₁-C₅alkoxy, C₁-C₅fluoroalkyl, or C₁-C₅fluoroalkoxy;
Ring A is a monocyclic C₃-C₈heterocycloalkyl with at least one N atom, or a bicyclic C₅-C₁₀heterocycloalkyl with at least one N atom, wherein ring A is unsubstituted or substituted with 1, 2, 3, or 4 R^{a};
each R^{a} is independently selected from the group consisting of hydrogen, —CN, halogen, —OR¹¹, —N(R¹¹)₂, —S(═O)₂(R¹²), —N(R¹¹)—S(═O)₂(R¹²), —C(═O)N(R¹¹)₂, —NR¹¹—C(═O)R¹¹, —C(═O)R¹¹, —C(═O)OR¹¹, —NR¹¹—C(═O)OR¹¹, —OC(═O)N(R¹¹)₂, C₁-C₄alkyl, C₂-C₄alkenyl, C₂-C₄alkynyl, C₁-C₄deuteroalkyl, C₁-C₄deuteroalkoxy, C₁-C₄fluoroalkyl, C₁-C₄fluoroalkoxy, C₁-C₄heteroalkyl, or substituted or unsubstituted monocyclic C₂-C₅heterocycloalkyl;
or two R^{a} that are attached to the same carbon atom are taken together with the carbon atom to form a substituted or unsubstituted monocyclic C₃-C₆cycloalkyl or a substituted or unsubstituted monocyclic C₃-C₅heterocycloalkyl;
L¹ is —CR⁵R⁶— or —NR⁷—;
R⁵ and R⁶ are taken together with the carbon atom to which they are attached to form —C(═O)—;
R⁷ is H or C₁-C₅alkyl;
L² is C₁-C₄alkyl, C₂-C₄alkenyl, or C₂-C₄alkynyl;
or L² is a ring B;
ring B is a monocyclic C₃-C₈cycloalkyl, phenyl, bicyclic C₅-C₁₂cycloalkyl, monocyclic C₃-C₈heterocycloalkyl, bicyclic C₅-C₁₀heterocycloalkyl, or monocyclic heteroaryl, wherein B is unsubstituted or is substituted with 1, 2, 3, or 4 R^{b};
each R^{b} is independently selected from the group consisting of hydrogen, —CN, halogen, —OR¹¹, —N(R¹¹)₂, —S(═O)₂(R¹²), —N(R¹¹)—S(═O)₂(R¹²), —C(═O)N(R¹¹)₂, —NR¹¹—C(═O)R¹¹, —C(═O)R¹¹, —C(═O)OR¹¹, —NR¹¹—C(═O)OR¹¹, —OC(═O)N(R¹¹)₂, C₁-C₄alkyl, C₂-C₄alkenyl, C₂-C₄alkynyl, C₁-C₄deuteroalkyl, C₁-C₄deuteroalkoxy, C₁-C₄fluoroalkyl, C₁-C₄fluoroalkoxy, C₁-C₄heteroalkyl, or substituted or unsubstituted monocyclic C₂-C₅heterocycloalkyl;
Ring C is selected from the group consisting of phenyl or a 6-membered heteroaryl with 1-3N atoms, wherein Ring C is unsubstituted or is substituted with 1, 2, 3, or 4 R^{c};
each R^{c} is independently selected from the group consisting of hydrogen, —CN, halogen, —OR¹¹, —N(R¹¹)₂, —S(═O)₂(R¹²), —N(R¹¹)—S(═O)₂(R¹²), —C(═O)N(R¹¹)₂, —NR¹¹—C(═O)R¹¹, —C(═O)R¹¹, —C(═O)OR¹¹, —NR¹¹—C(═O)OR¹¹, —OC(═O)N(R¹¹)₂, C₁-C₄alkyl, C₂-C₄alkenyl, C₂-C₄alkynyl, C₁-C₄deuteroalkyl, C₁-C₄deuteroalkoxy, C₁-C₄fluoroalkyl, C₁-C₄fluoroalkoxy, C₁-C₄heteroalkyl, or substituted or unsubstituted monocyclic C₃-C₅heterocycloalkyl;
each R^{d} is independently selected from the group consisting of hydrogen, —CN, halogen, —OR¹¹, C₁-C₄alkyl, C₂-C₄alkenyl, C₂-C₄alkynyl, C₁-C₄deuteroalkyl, C₁-C₄deuteroalkoxy, C₁-C₄fluoroalkyl, C₁-C₄fluoroalkoxy, or C₁-C₄heteroalkyl;
L³ is —O—, —NR⁸—, or —CR⁹R¹⁰—;
R⁸ is H or C₁-C₅alkyl;
R⁹ and R¹⁰ are each independently H or C₁-C₅alkyl;
each R¹¹ is independently selected from the group consisting of hydrogen, C₁-C₄alkyl, C₁-C₄deuteroalkyl, C₁-C₄fluoroalkyl, C₁-C₄heteroalkyl, substituted or unsubstituted C₃-C₆cycloalkyl, substituted or unsubstituted C₃-C₅heterocycloalkyl, substituted or unsubstituted phenyl, or substituted or unsubstituted monocyclic heteroaryl;
each R¹² is independently selected from the group consisting of C₁-C₄alkyl, C₁-C₄deuteroalkyl, C₁-C₄fluoroalkyl, C₁-C₄heteroalkyl, substituted or unsubstituted C₃-C₆cycloalkyl, substituted or unsubstituted C₃-C₅heterocycloalkyl, substituted or unsubstituted phenyl, or substituted or unsubstituted monocyclic heteroaryl; and
n is 0, 1, or 2.

7. The compound of any one of claims 1-6, or a stereoisomer, or pharmaceutically acceptable salt or solvate thereof, wherein L¹ is —C(═O)—.

8. The compound of any one of claims 1-6, or a stereoisomer, or pharmaceutically acceptable salt or solvate thereof, wherein L¹ is —NR⁷—.

9. The compound of any one of claims 1-8, or a stereoisomer, or pharmaceutically acceptable salt or solvate thereof, wherein:
ring A is azetidinyl, pyrrolidinyl, or a monocyclic 6-membered C₄-C₅heterocycloalkyl, or a bicyclic C₅-C₁₀heterocycloalkyl that is a fused bicyclic C₅-C₈heterocycloalkyl, bridged bicyclic C₅-C₈heterocycloalkyl, or spiro bicyclic C₅-C₈heterocycloalkyl, wherein ring A is unsubstituted or substituted with 1, 2, 3, or 4 R^{a}.

10. The compound of any one of claims 1-9, or a stereoisomer, or pharmaceutically acceptable salt or solvate thereof, wherein:
ring A is azetidinyl, pyrrolidinyl, morpholinyl, thiomorpholinyl, piperidinyl, piperazinyl, or a bicyclic C₅-C₁₀heterocycloalkyl that is a fused bicyclic C₅-C₈heterocycloalkyl, bridged bicyclic C₅-C₈heterocycloalkyl, or spiro bicyclic C₅-C₈heterocycloalkyl, wherein ring A is unsubstituted or substituted with 1, 2, 3, or 4 R^{a}.

11. The compound of any one of claims 1-10, or a stereoisomer, or pharmaceutically acceptable salt or solvate thereof, wherein:
ring A is azetidinyl, morpholinyl, piperidinyl, piperazinyl, or a spiro bicyclic C₅-C₈heterocycloalkyl, wherein ring A is unsubstituted or substituted with 1, 2, 3, or 4 R^{a}.

12. The compound of any one of claims 1-11, or a stereoisomer, or pharmaceutically acceptable salt or solvate thereof, wherein:
ring A is morpholinyl, piperidinyl, or piperazinyl, wherein ring A is unsubstituted or substituted with 1, 2, 3, or 4 R^{a}.

13. The compound of any one of claims 1-12, or a stereoisomer, or pharmaceutically acceptable salt or solvate thereof, wherein:
ring A is

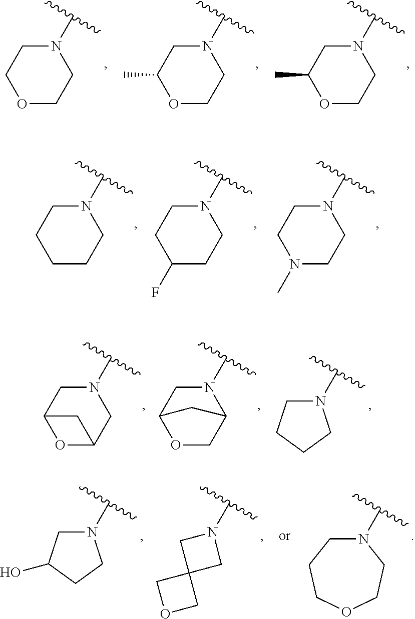

14. The compound of any one of claims 1-10, or a stereoisomer, or pharmaceutically acceptable salt or solvate thereof, wherein:
ring A is 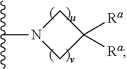
u is 1 or 2; and
v is 1 or 2.

15. A compound that has the structure of Formula (I-D), or a stereoisomer, or pharmaceutically acceptable salt or solvate thereof: 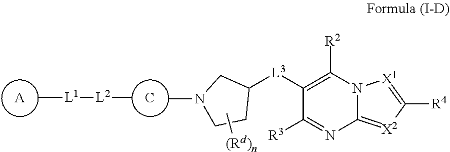
wherein:
each of X¹ and X² is independently N or CR¹;
each of R¹, R², R³, and R⁴ is independently H, halogen, —CN, —OH, C₁-C₅alkyl, C₁-C₅alkoxy, C₁-C₅fluoroalkyl, or C₁-C₅fluoroalkoxy;
Ring A is an azetidinyl or spiro bicyclic C₅-C₈heterocycloalkyl, wherein ring A is unsubstituted or substituted with 1, 2, 3, or 4 R^{a};
each R^{a} is independently selected from the group consisting of hydrogen, —CN, halogen, —OR¹¹, —N(R¹¹)₂, —S(═O)₂(R¹²), —N(R¹¹)—S(═O)₂(R¹²), —C(═O)N(R¹¹)₂, C₁-C₄alkyl, C₂-C₄alkenyl, C₂-C₄alkynyl, C₁-C₄deuteroalkyl, C₁-C₄deuteroalkoxy, C₁-C₄fluoroalkyl, C₁-C₄fluoroalkoxy, C₁-C₄heteroalkyl, or substituted or unsubstituted monocyclic C₂-C₅heterocycloalkyl;
or two R^{a} that are attached to the same carbon atom are taken together with the carbon atom to form a substituted or unsubstituted monocyclic C₃-C₆cycloalkyl or a substituted or unsubstituted monocyclic C₃-C₅heterocycloalkyl;
L¹ is —CR⁵R⁶— or —NR⁷—;
R⁵ and R⁶ are each independently H or C₁-C₅alkyl;
or R⁵ and R⁶ are taken together with the carbon atom to which they are attached to form —C(═O)—;
R⁷ is H or C₁-C₅alkyl;
L² is C₁-C₄alkyl, C₂-C₄alkenyl, or C₂-C₄alkynyl;
or L² is a ring B;
ring B is a monocyclic C₃-C₈cycloalkyl, phenyl, bicyclic C₅-C₁₂cycloalkyl, monocyclic C₃-C₈heterocycloalkyl, bicyclic C₅-C₁₀heterocycloalkyl, or monocyclic heteroaryl, wherein B is unsubstituted or is substituted with 1, 2, 3, or 4 R^{b};
each R^{b} is independently selected from the group consisting of hydrogen, —CN, halogen, —OR¹¹, —N(R¹¹)₂, —S(═O)₂(R¹²), —N(R¹¹)—S(═O)₂(R¹²), —C(═O)N(R¹¹)₂, —NR¹¹—C(═O)R¹¹, —C(═O)R¹¹, —C(═O)OR¹¹, —NR¹¹—C(═O)OR¹¹, —OC(═O)N(R¹¹)₂, C₁-C₄alkyl, C₂-C₄alkenyl, C₂-C₄alkynyl, C₁-C₄deuteroalkyl, C₁-C₄deuteroalkoxy, C₁-C₄fluoroalkyl, C₁-C₄fluoroalkoxy, C₁-C₄heteroalkyl, or substituted or unsubstituted monocyclic C₂-C₅heterocycloalkyl;
Ring C is selected from the group consisting of phenyl or a 6-membered heteroaryl with 1-3N atoms, wherein Ring C is unsubstituted or is substituted with 1, 2, 3, or 4 R^{c};
each R^{c} is independently selected from the group consisting of hydrogen, —CN, halogen, —OR¹¹, —N(R¹¹)₂, —S(═O)₂(R¹²), —N(R¹¹)—S(═O)₂(R¹²), —C(═O)N(R¹¹)₂, —NR¹¹—C(═O)R¹¹, —C(═O)R¹¹, —C(═O)OR¹¹, —NR¹¹—C(═O)OR¹¹, —OC(═O)N(R¹¹)₂, C₁-C₄alkyl, C₂-C₄alkenyl, C₂-C₄alkynyl, C₁-C₄deuteroalkyl, C₁-C₄deuteroalkoxy, C₁-C₄fluoroalkyl, C₁-C₄fluoroalkoxy, C₁-C₄heteroalkyl, or substituted or unsubstituted monocyclic C₃-C₅heterocycloalkyl;
each R^{d} is independently selected from the group consisting of hydrogen, —CN, halogen, —OR¹¹, C₁-C₄alkyl, C₂-C₄alkenyl, C₂-C₄alkynyl, C₁-C₄deuteroalkyl, C₁-C₄deuteroalkoxy, C₁-C₄fluoroalkyl, C₁-C₄fluoroalkoxy, or C₁-C₄heteroalkyl;
L³ is —O—, —NR⁸—, or —CR⁹R¹⁰—;
R⁸ is H or C₁-C₅alkyl;
R⁹ and R¹⁰ are each independently H or C₁-C₅alkyl;
each R¹¹ is independently selected from the group consisting of hydrogen, C₁-C₄alkyl, C₁-C₄deuteroalkyl, C₁-C₄fluoroalkyl, C₁-C₄heteroalkyl, substituted or unsubstituted C₃-C₆cycloalkyl, substituted or unsubstituted C₃-C₅heterocycloalkyl, substituted or unsubstituted phenyl, or substituted or unsubstituted monocyclic heteroaryl;
each R¹² is independently selected from the group consisting of C₁-C₄alkyl, C₁-C₄deuteroalkyl, C₁-C₄fluoroalkyl, C₁-C₄heteroalkyl, substituted or unsubstituted C₃-C₆cycloalkyl, substituted or unsubstituted C₃-C₅heterocycloalkyl, substituted or unsubstituted phenyl, or substituted or unsubstituted monocyclic heteroaryl; and
n is 0, 1, or 2.

16. The compound of any one of claims 5-15, or a stereoisomer, or pharmaceutically acceptable salt or solvate thereof, wherein:
X¹ is N; and
X² is N.

17. The compound of any one of claims 5-15, or a stereoisomer, or pharmaceutically acceptable salt or solvate thereof, wherein:
X¹ is N; and X² is CR¹;
or X¹ is CR¹; and X² is N;
or X¹ is CR¹; and X² is CR¹.

18. The compound of any one of claims 1-17, or a stereoisomer, or pharmaceutically acceptable salt or solvate thereof, wherein:
each of R², R³, and R⁴ is methyl.

19. The compound of any one of claims 1-18, or a stereoisomer, or pharmaceutically acceptable salt or solvate thereof, wherein:
L³ is —O—.

20. The compound of any one of claims 1-19, or a stereoisomer, or pharmaceutically acceptable salt or solvate thereof, wherein:
n is 0.

21. The compound of any one of claims 1-7 and 9-20, or a stereoisomer, or pharmaceutically acceptable salt or solvate thereof, having the structure of Formula (I-E):

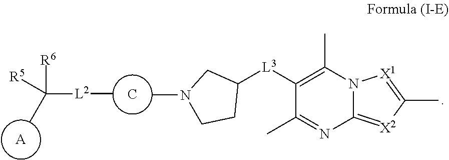

22. The compound of any one of claims 1-7 and 9-21, or a stereoisomer, or pharmaceutically acceptable salt or solvate thereof, wherein:
R⁵ and R⁶ are each independently H or —CH₃;
or R⁵ and R⁶ are taken together with the carbon atom to which they are attached to form —C(═O)—.

23. The compound of any one of claims 1-7 and 9-22, or a stereoisomer, or pharmaceutically acceptable salt or solvate thereof, having the structure of Formula (I-F):

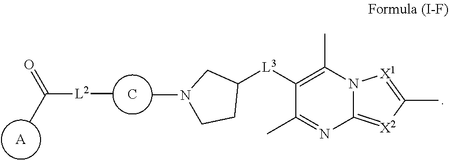

24. The compound of any one of claims 1-11 or 14-23, or a stereoisomer, or pharmaceutically acceptable salt or solvate thereof, wherein:
ring A is 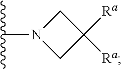
one R^{a} is H, and the other R^{a} is H, halogen, —CN, —OH, —N(R¹³)₂, —OC(═O)(R¹²), —CO₂R¹³, —C(═O)N(R¹³)₂, —NR¹³C(═O)(R¹²), —NR¹⁵C(═O)O(R¹²), —OC(═O)N(R¹³)₂, —NR¹³C(═O)N(R¹³)₂, C₁-C₄alkyl, C₂-C₄alkenyl, C₂-C₄alkynyl, C₁-C₄alkoxy, C₁-C₄deuteroalkyl, C₁-C₄deuteroalkoxy, C₁-C₄fluoroalkyl, C₁-C₄fluoroalkoxy, C₁-C₄heteroalkyl, or substituted or unsubstituted monocyclic C₂-C₅heterocycloalkyl;
or both R^{a} are taken together with the carbon atom to form a C₃-C₆cycloalkyl or a C₂-C₅heterocycloalkyl.

25. The compound of claim 24, or a stereoisomer, or pharmaceutically acceptable salt or solvate thereof, wherein:
one R^{a} is H, and the other R^{a} is H, F, Cl, Br, —CN, —OH, —OCH₃, —OCD₃, —OCFH₂, —OCHF₂, —OCF₃, —S(═O)₂CH₃, —NH₂, —NH(CH₃), —N(CH₃)₂, —C(═O)NH₂, —C(═O)N(CH₃)₂, —CH₃, —CH₂CH₃, —CH(CH₃)₂, —C(CH₃)₃, —CD₃, —CFH₂, —CHF₂, or —CF₃;
or both R^{a} are taken together with the carbon atom to form a cyclobutyl, cyclopentyl, cyclohexyl, azetidinyl, oxetanyl, thietanyl, pyrrolidinyl, or piperidinyl.

26. The compound according to claim 24 or 25, or a stereoisomer, or pharmaceutically acceptable salt or solvate thereof, wherein:
one R^{a} is H, and the other R^{a} is hydrogen, —OCH₃, —OCD₃, —OCF₃, —S(═O)₂CH₃, —NH₂, —NH(CH₃), —N(CH₃)₂, —C(═O)NH₂, —C(═O)N(CH₃)₂, —CH₃, —CD₃, or —CF₃.

27. The compound of any one of claims 24-26, or a stereoisomer, or pharmaceutically acceptable salt or solvate thereof, wherein:

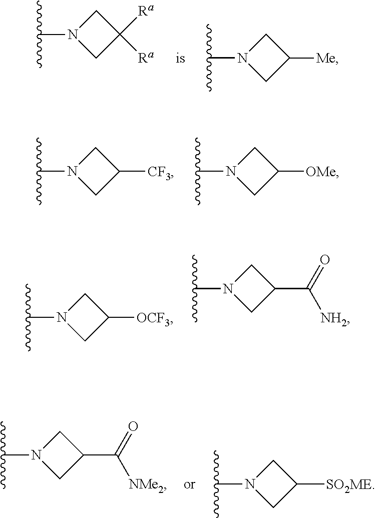

28. The compound of any one of claims 24-27, or a stereoisomer, or pharmaceutically acceptable salt or solvate thereof, wherein:

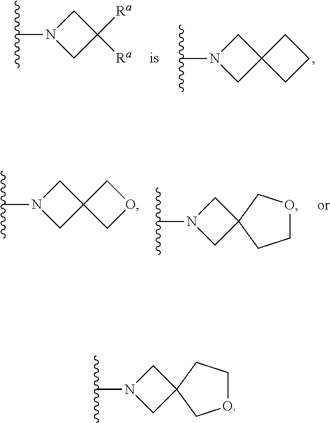

29. The compound of any one of claims 1-28, or a stereoisomer, or pharmaceutically acceptable salt or solvate thereof, wherein:
L² is

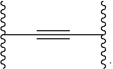 [SMILES: *C#C*]

30. The compound of any one of claims 1-28, or a stereoisomer, or pharmaceutically acceptable salt or solvate thereof, wherein:
L² is a ring B;
ring B is a monocyclic C₃-C₈cycloalkyl, bicyclic C₅-C₁₂cycloalkyl, monocyclic C₃-C₈heterocycloalkyl, or bicyclic C₅-C₁₀heterocycloalkyl, wherein ring B is unsubstituted or is substituted with 1, 2, 3, or 4 R^{b}.

31. The compound of claim 30, or a stereoisomer, or pharmaceutically acceptable salt or solvate thereof, wherein
ring B is 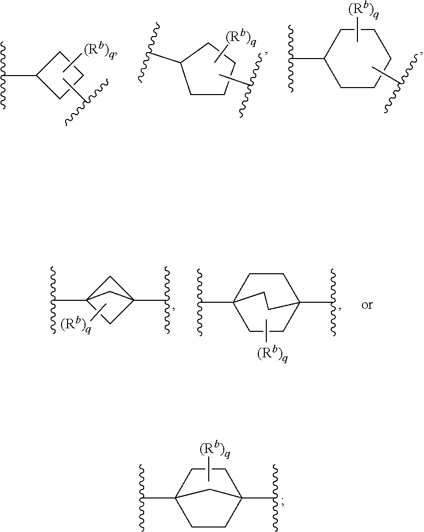
and
q is 0, 1, 2, 3, or 4.

32. The compound of claim 30, or a stereoisomer, or pharmaceutically acceptable salt or solvate thereof, wherein:
ring B is or

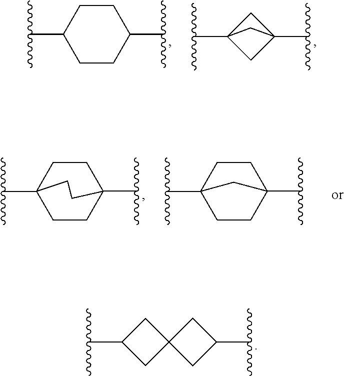

33. The compound of any one of claims 1-28, or a stereoisomer, or pharmaceutically acceptable salt or solvate thereof, wherein:
L² is a ring B;
ring B is phenyl or 6-membered monocyclic heteroaryl, wherein ring B is unsubstituted or is substituted with 1, 2, 3, or 4 R^{b}.

34. The compound of claim 33, or a stereoisomer, or pharmaceutically acceptable salt or solvate thereof, wherein:
ring B is 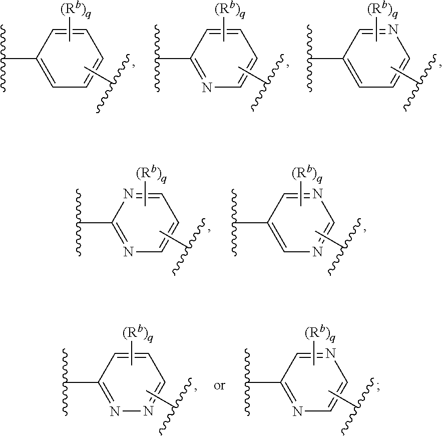
and
q is 0, 1, or 2.

35. The compound according to claim 33 or 34, or a stereoisomer, or pharmaceutically acceptable salt or solvate thereof, wherein:
ring B is 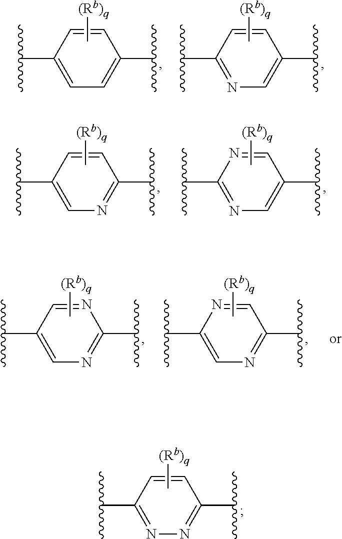
and
q is 0, 1, or 2.

36. The compound of any one of claims 1-35, or a stereoisomer, or pharmaceutically acceptable salt or solvate thereof, wherein:
Ring C is phenyl, pyridinyl, pyrazinyl, pyrimidinyl, or pyridazinyl, wherein Ring C is unsubstituted or is substituted with 1, 2, 3, or 4 R^{c}.

37. The compound of any one of claims 1-36, or a stereoisomer, or pharmaceutically acceptable salt or solvate thereof, wherein:
Ring C is

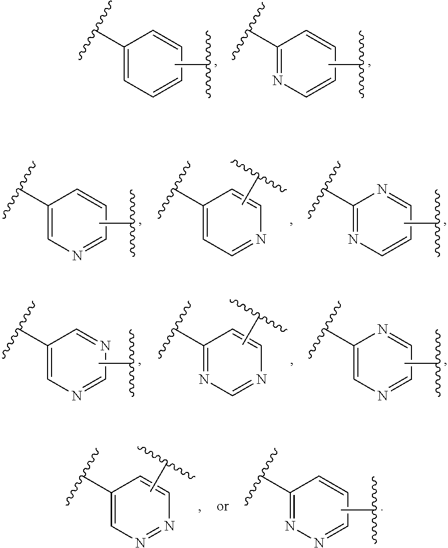

38. The compound of any one of claims 1-37, or a stereoisomer, or pharmaceutically acceptable salt or solvate thereof, wherein:
ring C is

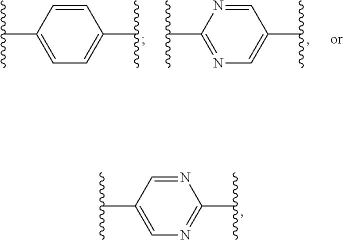

39. A compound that has a structure selected from Table 1, or a pharmaceutically acceptable salt, or solvate thereof.

40. A pharmaceutical composition comprising a compound of any one of claims 1-39, or a stereoisomer, or pharmaceutically acceptable salt or solvate thereof, and at least one pharmaceutically acceptable excipient.

41. The pharmaceutical composition of claim 40, wherein the pharmaceutical composition is formulated for administration to a mammal by oral administration, intravenous administration, or subcutaneous administration.

42. The pharmaceutical composition of claim 40, wherein the pharmaceutical composition is in the form of a tablet, a pill, a capsule, a liquid, a suspension, a dispersion, a solution, or an emulsion.

43. A method of modulating the activity of the monocarboxylic acid transporter 4 (MCT4) in a mammal comprising administering to the mammal a compound of any one of claims 1-39, or any pharmaceutically acceptable salt or solvate thereof.

44. A method of treating or preventing a disease or disorder in a mammal that is mediated by the action of the monocarboxylic acid transporter 4 (MCT4) comprising administering to the mammal a compound of any one of claims 1-39, or any pharmaceutically acceptable salt or solvate thereof.

45. A method for treating or preventing cancer in a mammal, the method comprising administering to the mammal a compound of any one of claims 1-39, or any pharmaceutically acceptable salt or solvate thereof.

46. The method of claim 45, wherein the cancer is a solid tumor.

47. The method of claim 46, wherein the solid tumor is a glycolytic tumor.

48. The method of claim 45, wherein the cancer is bladder cancer, colon cancer, brain cancer, breast cancer, endometrial cancer, heart cancer, kidney cancer, lung cancer, liver cancer, uterine cancer, blood and lymphatic cancer, ovarian cancer, pancreatic cancer, prostate cancer, thyroid cancer, or skin cancer.

49. The method of claim 45, wherein the cancer is bladder cancer, breast cancer, colon cancer, or lung cancer.

50. The method of claim 45, wherein the cancer is a sarcoma, carcinoma, or lymphoma.

51. The method of any one of claims 43-50, furthering comprising administering at least one additional therapy to the mammal.

52. The method of any one of claims 43-51, wherein the mammal is a human.
